(19) United States
(12) Patent Application Publication
(10) Pub. No.: US 2026/0027162 A1
(43) Pub. Date: Jan. 29, 2026

(54) MUCISPIRILLUM COMPOSITIONS AND CANCER TREATMENT METHODS THEREOF

(71) Applicant: PRESIDENT AND FELLOWS OF HARVARD COLLEGE, Cambridge, MA (US)

(72) Inventors: Wendy Sarah GARRETT, Cambridge, MA (US); Lior LOBEL, Cambridge, MA (US)

(73) Assignee: PRESIDENT AND FELLOWS OF HARVARD COLLEGE, Cambridge, MA (US)

(21) Appl. No.: 18/994,319

(22) PCT Filed: Jul. 14, 2023
(86) PCT No.: PCT/US2023/070197

§ 371 Date: Jan. 14, 2025

Related U.S. Application Data
(60) Provisional application No. 63/389,382, filed on Jul. 15, 2022.

(51) Int. Cl. A61K 35/74; A23L 33/135; A61K 9/48; A61K 45/06; A61K 47/18; A61P 35/00; G01N 33/569

(52) U.S. Cl. CPC A61K 35/74; A23L 33/135; A61K 9/48; A61K 45/06; A61K 47/183; A61P 35/00; G01N 33/56911; G01N 2333/195; G01N 2800/52

(57) ABSTRACT

The technology described herein is directed to compositions comprising Mucispirillum (e.g., M. schaedleri). Also described herein are methods of treating cancer using Mucispirillum compositions; a diet high in sulfur amino acids (SAA) or a supplement comprising SAAs; and/or XCL1 polypeptides (or XCR1 agonists). Also described herein are cancer treatment stratification methods related to detection of the level of M. schaedleri, XCL1 polypeptide, NKTs, and/or CD103+ cDC1s, and alteration or stratification of treatment accordingly.

### CROSS-REFERENCE TO RELATED APPLICATIONS

This application claims benefit under 35 U.S.C. § 119(e) of U.S. Provisional Application No. 63/389,382 filed Jul. 15, 2022, the contents of which are incorporated herein by reference in their entirety.

### GOVERNMENT SUPPORT

This invention was made with Government support under CA202704 and CA154426 awarded by National Institutes of Health. The Government has certain rights in the invention.

### SEQUENCE LISTING

The instant application contains a Sequence Listing which has been submitted in XML format via Patent Center and is hereby incorporated by reference in its entirety. Said XML copy, created on Jul. 6, 2023, is named 002806-099880WOPT_SL.xml and is 61,077 bytes in size.

### TECHNICAL FIELD

The technology described herein relates to compositions comprising Mucispirillum and associated methods of treating cancer therewith.

### BACKGROUND

Colorectal cancer (CRC) is the second leading cause of cancer deaths worldwide, and its global incidence is rising. While immunotherapies, specifically immune checkpoint inhibitors (ICI), have been a therapeutic breakthrough for many cancers, the vast majority of CRC is not ICI-responsive, due to proficient DNA mismatch repair and the colon's tolerogenic immune tone. Beyond tumoral-cell intrinsic factors like DNA mismatch repair (MMR) status, the gut microbiota also influences responsiveness to immunotherapy treatments and is an environmental factor for CRC development. The gut microbiota has been associated with ICI treatment efficacy, and different bacterial species have been identified as mediators of responsiveness. See e.g., Andrews et al., Nat Med 27, 1432-1441 (2021); Frankel et al., Neoplasia 19, 848-855 (2017); Gopalakrishnan et al., Science 359, 97-103 (2018); Lee et al., Nat Microbiol 6, 277-288 (2021); Matson et al., Science 359, 104-108 (2018); Routy et al., Science 359, 91-97 (2018); the contents of each of which are incorporated herein by reference in their entireties. There is need for additional therapeutics for ICI-non-responsive, immunologically cold cancers, such as CRC.

### SUMMARY

The technology described herein is directed to compositions comprising Mucispirillum (e.g., M. schaedleri). The disclosure describes how a diet high in sulfur amino acids can increase the gut level of M. schaedleri, which in turn increases XCL1 secretion by NKT cells. Increased XCL1 secretion by NKT cells increases CD103+ conventional dendritic cells (cDC1) number and/or activation, e.g., in tumor-draining lymph nodes, thus recruiting and activating CD8+ T cells, which have an anti-tumor immune activity (see e.g., FIG. 13). Accordingly, described herein are methods of treating cancer using Mucispirillum compositions as described herein; a diet high in sulfur amino acids (SAA) or a supplement comprising SAAs; and/or XCL1 polypeptides (or XCR1 agonists). Also described herein are cancer treatment stratification methods related to detection of the level of M. schaedleri, XCL1 polypeptide, NKTs, and/or CD103+ cDC1s, and alteration or stratification of treatment accordingly.

Accordingly, in one aspect described herein is a composition comprising Mucispirillum schaedleri (M. schaedleri) bacteria formulated for delivery to the intestine.

In some embodiments of any of the aspects, the M. schaedleri bacteria are living or inactivated.

In some embodiments of any of the aspects, the M. schaedleri bacteria are in dried viable form.

In some embodiments of any of the aspects, the M. schaedleri bacteria are encapsulated.

In some embodiments of any of the aspects, the M. schaedleri bacteria are comprised in an enteric capsule.

In some embodiments of any of the aspects, the M. schaedleri bacteria are maintained in an anaerobic state in the formulation.

In some embodiments of any of the aspects, the M. schaedleri bacteria are in admixture with a prebiotic.

In some embodiments of any of the aspects, the M. schaedleri bacteria are in admixture with a sulfur amino acid.

In some embodiments of any of the aspects, the sulfur amino acid is methionine, cysteine or a derivative thereof.

In some embodiments of any of the aspects, the M. schaedleri bacteria are formulated in a food composition.

In some embodiments of any of the aspects, the food composition is supplemented with a sulfur amino acid and/or a prebiotic.

In some embodiments of any of the aspects, the composition further comprises 1 to 20 additional species of bacteria.

In some embodiments of any of the aspects, the composition comprises no more than 20 species of bacteria.

In one aspect described herein is a composition comprising live M. schaedleri bacteria, dead M. schaedleri bacteria, conditioned M. schaedleri culture medium, or an organic solvent extract of conditioned M. schaedleri culture medium or a fraction thereof that promotes XCL1 secretion by NKT cells, wherein the composition is formulated for delivery to the intestine.

In some embodiments of any of the aspects, the M. schaedleri bacteria, medium or solvent extract are in dried form.

In some embodiments of any of the aspects, the M. schaedleri bacteria, medium or extract is/are encapsulated.

In some embodiments of any of the aspects, the M. schaedleri bacteria are comprised in an enteric capsule.

In some embodiments of any of the aspects, the M. schaedleri bacteria are maintained an anaerobic state in the formulation.

In some embodiments of any of the aspects, the M. schaedleri bacteria, medium or extract is/are in admixture with a prebiotic and/or a sulfur amino acid or derivative thereof.

In some embodiments of any of the aspects, the conditioned M. schaedleri culture medium, or the organic solvent extract of conditioned M. schaedleri culture medium or a fraction thereof comprises at least one metabolite selected from Table 3 or Table 9 of U.S. Provisional Application No. 63/389,382, or FIG. 4H, FIG. 4I, or FIG. 16 herein.

In some embodiments of any of the aspects, the conditioned M. schaedleri culture medium, or the organic solvent extract of conditioned M. schaedleri culture medium or a fraction thereof comprises at least one metabolite selected from the group consisting of: succinic acid; propionic acid; nicotinic acid; aconitic acid (cis and/or trans); pentadecanoic acid; itaconic acid; 16-hydroxyhexadecanoic acid; C₈H₁₅NO₃S; crotonic acid; myristic acid; 17-hydroxyheptadecanoic acid (C₁₇H₃₄O₃); and 15-hydroxpentadecanoic acid (C₁₅H₃₀O₃).

In some embodiments of any of the aspects, the conditioned M. schaedleri culture medium, or the organic solvent extract of conditioned M. schaedleri culture medium or a fraction thereof comprises at least one metabolite selected from the group consisting of: succinic acid; nicotinic acid; aconitic acid (cis and/or trans); pentadecanoic acid; itaconic acid; 16-hydroxyhexadecanoic acid; and crotonic acid.

In one aspect described herein is a food composition comprising a composition as described herein.

In some embodiments of any of the aspects, the food composition further comprises 1 to 20 additional species of bacteria.

In one aspect described herein is a method of treating cancer or promoting anti-tumor immune activity, the method comprising administering to a subject in need thereof a composition as described herein.

In some embodiments of any of the aspects, the cancer is colon cancer.

In some embodiments of any of the aspects, the method further comprises administering an immune checkpoint inhibitor.

In one aspect described herein is a method of promoting responsiveness to immune checkpoint inhibitor tumor therapy, the method comprising administering to a subject in need thereof a composition as described herein.

In some embodiments of any of the aspects, the method further comprises administering an immune checkpoint inhibitor.

In some embodiments of any of the aspects, the subject has colon cancer.

In some embodiments of any of the aspects, the subject's cancer has been determined to be resistant to immune checkpoint inhibitor therapy.

In some embodiments of any of the aspects, the composition promotes XCL1 secretion by NKT cells.

In one aspect described herein is a method of increasing CD103+ conventional dendritic cells (cDC1), the method comprising administering to a subject in need thereof a composition as described herein.

In some embodiments of any of the aspects, the cDC1s are associated with a tumor.

In some embodiments of any of the aspects, the tumor is a colon cancer.

In some embodiments of any of the aspects, the method further comprises administering a sulfur amino acid.

In some embodiments of any of the aspects, the method further comprises administering an immune checkpoint inhibitor.

In one aspect described herein is a method of increasing XCL1 secretion by NKT cells, the method comprising administering to a subject in need thereof a composition as described herein.

In some embodiments of any of the aspects, the subject has cancer.

In some embodiments of any of the aspects, the subject has colon cancer.

In one aspect described herein is a method of increasing CD8+ T cell infiltration in a colorectal tumor, the method comprising administering to a subject in need thereof a composition as described herein.

In some embodiments of any of the aspects, the cDC1s are associated with a tumor.

In some embodiments of any of the aspects, the tumor is a colon cancer.

In some embodiments of any of the aspects, the method further comprises administering a sulfur amino acid.

In some embodiments of any of the aspects, the method further comprises administering an immune checkpoint inhibitor.

In one aspect described herein is a method of increasing CD8+ T cell infiltration in a colorectal tumor, the method comprising administering a diet high in sulfur amino acids (SAA) or a supplement comprising SAAs to a subject in need thereof.

In some embodiments of any of the aspects, the diet high in sulfur amino acids comprises greater than 0.04 grams of SAA per kilogram body weight per day.

In some embodiments of any of the aspects, the method further comprises administering a composition as described herein to the subject.

In one aspect described herein is a method of establishing or maintaining a tumor-suppressive gut environment in a subject in need thereof, the method comprising administering a diet high in sulfur amino acids (SAA) or a supplement comprising SAAs.

In some embodiments of any of the aspects, the diet high in sulfur amino acids or a supplement comprising SAAs comprises greater than 0.04 grams of SAA per kilogram body weight per day.

In some embodiments of any of the aspects, the method further comprises administering a composition as described herein to the subject.

In one aspect described herein is a method of treating cancer, the method comprising administering to a subject in need thereof an XCL1 polypeptide.

In some embodiments of any of the aspects, the cancer is colon cancer.

In some embodiments of any of the aspects, the XCL1 polypeptide is administered to the gut.

In one aspect described herein is a method of treating cancer, the method comprising administering to a subject in need thereof a microorganism engineered to express XCL1 polypeptide.

In one aspect described herein is a method of treating cancer, the method comprising administering to a subject in need thereof an agonist of the XCL1 receptor, XCR1.

In some embodiments of any of the aspects, the XCR1 agonist comprises SEQ ID NOs: 9-11 or an amino acid sequence that is at least 95% identical and maintains its function.

In one aspect described herein is a method of treating cancer in a subject in need thereof, the method comprising: detecting the level of M. schaedleri in a sample from the subject; administering a cancer immunotherapeutic agent if the level of M. schaedleri is at or above a pre-determined threshold; and administering a cancer immunotherapeutic agent and a composition as described herein if the level of M. schaedleri is below a pre-determined threshold.

In one aspect described herein is a method of treating cancer in a subject in need thereof, the method comprising: obtaining results from an assay detecting the level of M. schaedleri in a sample from the subject; administering a cancer immunotherapeutic agent if the level of M. schaedleri is at or above a pre-determined threshold; and administering a cancer immunotherapeutic agent and a composition as described herein if the level of M. schaedleri is below a pre-determined threshold.

In one aspect described herein is a method of treating cancer in a subject in need thereof, the method comprising: detecting the level of XCL1 polypeptide, NKTs, and/or CD103+ cDC1s in a sample from the subject; administering a cancer immunotherapeutic agent if the level of XCL1 polypeptide, NKTs, and/or CD103+ cDC1s is at or above a pre-determined threshold; and administering a cancer immunotherapeutic agent and a composition as described herein if the level of XCL1 polypeptide, NKTs, and/or CD103+ cDC1s is below a pre-determined threshold.

In one aspect described herein is a method of treating cancer in a subject in need thereof, the method comprising: obtaining results from an assay detecting the level of XCL1 polypeptide, NKTs, and/or CD103+ cDC1s in a sample from the subject; administering a cancer immunotherapeutic agent if the level of XCL1 polypeptide, NKTs, and/or CD103+ cDC1s is at or above a pre-determined threshold; and administering a cancer immunotherapeutic agent and a composition as described herein if the level of XCL1 polypeptide, NKTs, and/or CD103+ cDC1s is below a pre-determined threshold.

In one aspect described herein is a method of stratifying a subject for cancer treatment, the method comprising: detecting the level of M. schaedleri in a sample from the subject; and classifying the subject as high risk if the level of M. schaedleri is below a pre-determined threshold; or classifying the subject as low risk if the level of M. schaedleri is at or above a pre-determined threshold.

In one aspect described herein is a method of stratifying a subject for cancer treatment, the method comprising: obtaining results from an assay detecting the level of M. schaedleri in a sample from the subject; and classifying the subject as high risk if the level of M. schaedleri is below a pre-determined threshold; or classifying the subject as low risk if the level of M. schaedleri is at or above a pre-determined threshold.

In one aspect described herein is a method of stratifying a subject for cancer treatment, the method comprising: detecting the level of XCL1 polypeptide, NKTs, and/or CD103+ cDC1s in a sample from the subject; and classifying the subject as high risk if the level of XCL1 polypeptide, NKTs, and/or CD103+ cDC1s is below a pre-determined threshold; or classifying the subject as low risk if the level of XCL1 polypeptide, NKTs, and/or CD103+ cDC1s is at or above a pre-determined threshold.

In one aspect described herein is a method of stratifying a subject for cancer treatment, the method comprising: obtaining results from an assay detecting the level of XCL1 polypeptide, NKTs, and/or CD103+ cDC1s in a sample from the subject; and classifying the subject as high risk if the level of XCL1 polypeptide, NKTs, and/or CD103+ cDC1s is below a pre-determined threshold; or classifying the subject as low risk if the level of XCL1 polypeptide, NKTs, and/or CD103+ cDC1s is at or above a pre-determined threshold.

In some embodiments of any of the aspects, the subject has colon cancer.

In some embodiments of any of the aspects, the method further comprises administering the composition as described herein.

In some embodiments of any of the aspects, the method further comprises administering a sulfur amino acid.

In some embodiments of any of the aspects, the cancer immunotherapeutic agent is selected from the group consisting of: an immune checkpoint inhibitor; chemotherapy; a dendritic cell vaccine; chimeric antigen receptor T cells (CAR-T); and NKT cell-based therapies.

In some embodiments of any of the aspects, the cancer immunotherapeutic agent comprises an immune checkpoint inhibitor.

In some embodiments of any of the aspects, the method further comprises administering an immune checkpoint inhibitor.

In some embodiments of any of the aspects, the method further comprises administering a diet high in sulfur amino acids or a supplement comprising SAAs.

In some embodiments of any of the aspects, the method results in higher treatment efficacy compared to a method of treating without first: detecting the level of M. schaedleri; obtaining results from an assay detecting the level of M. schaedleri; detecting the level of XCL1 polypeptide, NKTs, and/or CD103+ cDC1s; or obtaining results from an assay detecting the level of XCL1 polypeptide, NKTs, and/or CD103+ cDC1s.

In some embodiments of any of the aspects, the method results in higher treatment efficacy compared to a method of treating without first stratifying the subject.

In some embodiments of any of the aspects, the method results in lower treatment complications compared to a method of treating without first detecting the level of M. schaedleri, obtaining results from an assay detecting the level of M. schaedleri, detecting the level of XCL1 polypeptide, NKTs, and/or CD103+ cDC1s or obtaining results from an assay detecting the level of XCL1 polypeptide, NKTs, and/or CD103+ cDC1s.

In some embodiments of any of the aspects, the method results in lower treatment complications compared to a method of treating without first stratifying the subject.

In one aspect described herein is an enteric delivery formulation comprising at least one metabolite selected from Table 3 or Table 9 of U.S. Provisional Application No. 63/389,382, or FIG. 4H, FIG. 4I, or FIG. 16 herein.

In one aspect described herein is an enteric delivery formulation comprising at least one metabolite selected from the group consisting of succinic acid; propionic acid; nicotinic acid; aconitic acid (cis and/or trans); pentadecanoic acid; itaconic acid; 16-hydroxyhexadecanoic acid; C₈H₁₅NO₃S; crotonic acid; myristic acid; 17-hydroxyheptadecanoic acid (C₁₇H₃₄O₃); and 15-hydroxpentadecanoic acid (C₁₅H₃₀O₃).

In one aspect described herein is an enteric delivery formulation comprising at least one metabolite selected from the group consisting of succinic acid; nicotinic acid; aconitic acid (cis and/or trans); pentadecanoic acid; itaconic acid; 16-hydroxyhexadecanoic acid; and crotonic acid.

In some embodiments of any of the aspects, the enteric delivery formulation is formulated for delivery to the intestine.

### BRIEF DESCRIPTION OF THE DRAWINGS

FIG. 1A-1J is a series of schematics, images and graphs showing that dietary sulfur amino acids (Saa) modulate tumor progression in mouse CRC models. FIG. 1A shows MetaCyc pathways (with top four enlarged on the right) in immune checkpoint inhibitors (ICI) responder and non-responder microbiomes, ordered by q-values from least-square linear regression analysis, for the renal cell cancer (RCC) cohort of Routy; see e.g., Routy et al., Science 359, 91-97 (2018); Caspi et al., Nucleic Acids Res 42, D459-71 (2014); the contents of each of which are incorporated herein by reference in their entireties. FIG. 1B shows MetaCyc pathways ranked by meta-analysis effect size from linear mixed-effects model for ICI responder (n=203) and non-responder microbiomes (n=134). FIG. 1C shows enrichment of the PWY-821 superpathway of Saa biosynthesis in stool metagenomes of anti-PD-1 treatment responders vs. non-responders (left column) and enrichment of 15 bacterial Saa biosynthesis genes from PWY-821 (right) across eight patient cohorts. FIG. 1D shows PWY-821 and genes as in FIG. 1C for stool metagenomes from colorectal cancer (CRC) and colonic adenoma patients vs. healthy controls across nine patient cohorts. FIG. 1E shows a schematic of the experimental set-up (top panel) and tumor volume over time in wild type (WT) born in-house mice fed Saa diets and flank-injected with MC38 cells (bottom left) and representative pictures of tumors (bottom right). FIG. 1F shows MC38 tumor weights from FIG. 1E at day 12. FIG. 1G and FIG. 1H show a schematic of the experimental set-up (top panel of FIG. 1G) and tumor volume over time in WT born in-house mice fed Saa diets, flank-injected with MC38 cells and treated intraperitoneally (i.p.) with α-PD-1 or isotype Abs (bottom panel of FIG. 1G) and MC38 tumor weight at day 13 (FIG. 1H). FIG. 1I shows a schematic of the experimental set-up (top panel), representative colon photographs from CDX2-Cre Ape^{flox/-} (cAPC) mice fed low or high Saa diets with tumors highlighted by black circles (middle panel), and data on dysplastic and neoplastic lesion grade and numbers (bottom panel). Each column represents data from a single mouse, and number of each lesion type are shown in the boxes. FIG. 1J shows colon tumor weight from cAPC mice fed Saa diets from FIG. 11 at week 12. Each symbol represents data from an individual mouse in FIG. 1F, FIG. 1H, and FIG. 1J. * P value<0.05, ** P value<0.01, *** P value<0.001. Error bars represent standard error of the mean (SEM). One-way ANOVA with FDR correction was performed for FIG. 1E, FIG. 1G, and FIG. 1H. Mann-Whitney test was performed for FIG. 1F, and FIG. 1J, and Boschloo's test for FIG. 1I. Data represent two independent experiments for FIG. 1E, FIG. 1F, FIG. 1G and FIG. 1H, and four independent experiments for FIG. 1I and FIG. 1J.

FIG. 2A-2H is a series of schematics, images, and graphs showing that high Saa diet-fed mice have higher Mucispirillum schaedleri abundance and thicker mucus. FIG. 2A shows a volcano plot showing enrichment of microbial taxa from 16S rRNA amplicon profiling in the cecal contents of cAPC mice fed low versus high Saa diet using Microbiome Multivariable Associations with Linear Models (MaAsLin 2) regression models; see e.g., Mallick et al., PLoS Comput Biol 17, e1009442 (2021), the contents of which are incorporated herein by reference in their entirety. FIG. 2B shows reverse transcription quantitative polymerase chain reaction (RT-qPCR) analysis of M. schaedleri 16S rRNA in cecal contents DNA from cAPC BIH mice, WT BIH MC38-injected mice and WT ASF mice. FIG. 2C shows the relative abundance of M. schaedleri-specific reads in 16S ribosomal RNA (rRNA) amplicon sequence data from tissue biopsies of healthy controls (“normal”) or patients with colonic adenoma. FIG. 2D shows representative images of Muc2 immunofluorescence staining coupled with bacterial fluorescence in situ hybridization (FISH) staining in colon tissue sections of altered Schaedler flora (ASF) mice fed low Saa or high Saa diets (pink fluorescence: M. schaedleri probe, blue fluorescence: universal bacterial probe, green fluorescence: Muc2; gray: DAPI). FIG. 2E shows mucus layer measurements from images (see e.g., FIG. 2F-2G). Each symbol represents data from an individual mouse (average of 5 field of views per mouse). FIG. 2F-2G show representative images of Alcian Blue staining of mucus thickness measurements in distal colon tissue sections from WT GF mice (FIG. 2F) and WT ASF mice (FIG. 2G). FIG. 2H shows mucus layer thickness measurements. Each symbol represents data from an individual mouse (average of 23 field of views per mouse). * P value<0.05, ** P value<0.01, *** P value<0.001. Error bars represent standard error of the mean (SEM). Mann-Whitney test was performed for FIG. 2B, FIG. 2C, FIG. 2E, and FIG. 2H. Data represent three independent experiments for FIG. 2A and FIG. 2B, and two independent experiments for FIG. 2D-2H.

FIG. 3A-3I is a series of schematics, images, and graphs showing that dietary Saa and M. schaedleri expanded CD8 T cells in tumors, and M. schaedleri monocolonization was sufficient for increased cDC1 in the tumor-draining lymph nodes (TDLN). FIG. 3A shows flow cytometry data representing relative frequencies of CD8⁺ T-cells in cAPC tumors. FIG. 3B shows representative immunofluorescence images of colon tumors sections of cAPC mice fed low or high Saa diets. The right-side images are higher magnifications of regions indicated by white rectangles on the left. FIG. 3C shows quantification of CD3⁻CD8⁻/CD3⁻ ratio in images. Each symbol represents data from an individual mouse (average of 4 fields per mouse). FIG. 3D shows flow cytometry data representing co-inhibitory receptor expression in CD8⁺ T cells from tumors from cAPC mice fed the indicated dicts, relative frequencies (left) and numbers (right). FIG. 3E shows the frequencies of IFNγ⁺ (left) and GZMB(right) CD8⁺ T cells from tumors from cAPC mice fed the diets. FIG. 3F-3G show representative data of cDC1 (CD103⁺CD11b⁻), CD103⁻CD11b⁺, and CD103⁺CD11b⁺ cells from the TDLN of cAPC mice fed low or high Saa diets; FIG. 3F shows representative flow cytometry plots, and FIG. 3G shows frequencies and numbers of the cDC1 (CD103⁺CD11b⁻), CD103⁻CD11b⁺, and CD103⁺CD11b⁺ cells. FIG. 3H show frequencies (left) and numbers (right) of cDC1 (CD103⁺CD11b⁻), CD103⁻CD11b⁺ and CD103⁺CD11b⁺ cells from the MLN of GF, M. schaedleri-monocolonized, or A. muciniphila-monocolonized mice fed the high Saa diet. Each symbol represents data from an individual mouse. Left-right order of bars in each group correspondences to left-right order of legend. FIG. 31 shows frequencies of cDC1 and CD103⁻ CD11b⁺ and CD103⁺CD11b⁻ cells from the mesentery lymph nodes (MLN) of WT mice fed the low Saa diet and gavaged with brain-heart infusion media (mBHI) or M. schaedleri conditioned media (CM) three times per week. Each symbol represents data from an individual mouse. * P value<0.05, ** P value<0.01, *** P value<0.001. Error bars represent standard error of the mean (SEM). Mann-Whitney test was performed for FIG. 3A, FIG. 3C, FIG. 3D, FIG. 3E, FIG. 3G and FIG. 31, and One-Way ANOVA with FDR correction was performed for FIG. 3H. Data represent three independent experiments for FIG. 3A-3G and two independent experiments for FIG. 3H and FIG. 31.

FIG. 4A-4K is a series of schematics and graphs showing that high Saa diet and M. schaedleri induced XCL1 secretion from NKT cells and promoted an activated state in cDC1s, which correlated with increased survival in CRC patients. FIG. 4A-4C shows experimental schemes and dysplastic and neoplastic lesion grade and numbers from Zbtb46-DTR cAPC mice fed the high Saa diet and injected i.p. with PBS or diphtheria toxin (FIG. 4A), for cAPC Batf3^{-/-} mice fed low or high Saa diet (FIG. 4B) and from cAPC mice fed high Saa diet and treated with either α-XCL1 Ab or isotype control (FIG. 4C). Each column represents data from an individual mouse. FIG. 4D shows ex vivo secretion of XCL1 from natural killer (NK) and natural killer T (NKT) cells sorted from MLN of born in-house (BIH) mice fed the Saa diets. FIG. 4E shows frequencies and numbers of NKT from TDLN of cAPC mice fed the Saa diets. Each symbol represents data from an individual mouse. FIG. 4F shows secretion of XCL1 from GW1 NKT cells following overnight stimulation with bacterial conditioned media (CM). FIG. 4G shows secretion of XCL1 from human peripheral blood-derived NKT cells following overnight stimulation with bacterial CM. FIG. 4H shows volcano plot of identified metabolic features. 36 metabolic features enriched in M. schaedleri CM are shown. Black text: definitively identified metabolites; grey text: manually curated metabolites with convincing library match, labeling: chemical formulae shown for putative identification. FIG. 4I shows a heat map showing abundance of 36 M. schaedleri CM-enriched metabolic features. FIG. 4J shows relative abundances of genes differentially expressed (P<0.05, AvgLogFC>1.2 or <0.8) by Model-based Analysis of Single Cell Transcriptomics (MAST) in cDC1 from TDLN of cAPC mice fed Saa diets. FIG. 4F shows survival curves of CRC patients from the Tissue Cancer Gene Atlas dataset of colon and rectal adenocarcinoma tumor (TCGA-COAD/READ) data stratified by tumoral gene expression similarity to cDC1 from cAPC mice fed Saa diets. In FIG. 4F, “low activation” corresponds to low sulfur markers dendritic cells (LSMD-DC), and “high activation” corresponds to high sulfur markers dendritic cells (HSME-DC). * P value<0.05, ** P value<0.01, *** P value<0.001. Error bars represent standard error of the mean (SEM). Boschloo's test was performed for FIG. 4A, FIG. 4B and FIG. 4C. One-Way ANOVA with FDR correction was performed for FIG. 4D, FIG. 4F and FIG. 4G; Mann-Whitney test was performed for FIG. 4E; MAST analysis was performed for FIG. 4J, and Cox proportional hazards model was performed for FIG. 4K. Data represent four independent experiments for FIG. 4A-4C, three independent experiments for FIG. 4D, FIG. 4E, and FIG. 4F; and two independent experiments for FIG. 4G.

FIG. 5A-5G is a series of graphs showing dietary Saa effects on tumor growth in GF cAPC mice, the cecal microbiome, mucus layer thickness and M. schaedleri detection in human stool samples. FIG. 5A shows the dysplastic and neoplastic grade of lesions in GF cAPC mice fed Saa diets. Each column represents data from an individual mouse. FIG. 5B shows alpha diversity analyses (Chao1 and Shannon) on cecal 16S rRNA gene amplicon samples from cAPC mice fed Saa diets. Each symbol represents data from an individual mouse. FIG. 5C and FIG. 5D show principal coordinate analyses (PCoA) of 16S rDNA amplicons from cAPC mice fed low or high Saa diets using Weighted Unifrac (FIG. 5C) or Bray-Curtis (FIG. 5D) methods. Each symbol represents data from an individual mouse. FIG. 5E shows distribution of averaged bacterial phyla abundances in cAPC mice cecal samples; n=6 for each diet, 3 cages per condition. Top-down order of phyla bars correspondences to top-down order of legend. FIG. 5F shows 16S rRNA amplicon abundance of M. schaedleri in cecal samples from cAPC mice fed the Saa diets. Each symbol represents data from an individual mouse. FIG. 5G shows levels of FITC-dextran in serum samples of WT BIH mice fed low or high Saa diets for 2 weeks and measured 3 h after FITC-dextran gavage. Error bars represent standard error of the mean (SEM). * P value<0.05, *** P value<0.001. Boschloo's test was performed for FIG. 5A; Mann-Whitney test was performed for FIG. 5F, and FIG. 5G. Data represent four independent experiments for FIG. 5A, three independent experiments for FIG. 5B-5F, and two independent experiments for FIG. 5G.

FIG. 6A-6E is a series of graphs showing the effects of dietary Saa on CD8⁺ T-cells and their expression of immune co-inhibitory receptors in colon LP and TDLN from cAPC mice. FIG. 6A-6B show flow cytometry analysis of frequencies and numbers of CD8⁺ T-cells from tumors (FIG. 6A) and PD-1⁺, LAG-3⁺, CTLA-4⁺, and TIM3⁺ (FIG. 6B) CD8⁺ T-cells in TDLN in low or high Saa diet-fed cAPC mice. FIG. 6C-6D show flow cytometry analysis of colon LP frequencies and numbers of CD8⁺ T-cells (FIG. 6C) and PD-1⁺, LAG-3⁺, CTLA-4⁺ and TIM3⁻ (FIG. 6D) from low or high Saa diet-fed cAPC mice. FIG. 6E shows quantification of CD3⁺CD8⁺/CD3⁺ ratio in images of healthy colonic tissue from cAPC mice. Each symbol represents data from an individual mouse. Error bars represent standard error of the mean (SEM). * P value<0.05, ** P value<0.01. Mann-Whitney test was performed for FIG. 6A-6E. Data represent three independent experiments for FIG. 6A-6E.

FIG. 7A-7D is a series of graphs showing T cell receptor sequencing (TCR-Seq) analysis of CD8⁺ T cells from tumors of cAPC mice fed Saa diets. FIG. 7A shows the profile of TCRα and TCRβ clonotype repertoires of intratumoral CD8⁺ T-cells. Each symbol represents data from an individual mouse. Left-right order of bars in each group corresponds to top-down order of legend. FIG. 7B shows an tSNE plot of tumor CD8⁺ T-cell TCRα and TCRβ diversities. Ellipse overlays represent 95% confidence interval. FIG. 7C and FIG. 7D show alpha diversity of TCRα and TCRβ clonotypes based on observed clonotypes (FIG. 7C) or Chao1 index (FIG. 7D). Each symbol represents data from an individual mouse. Error bars represent standard error of the mean (SEM). Visualization and statistical tests were performed using the immnuarch R/CRAN package pipeline.

FIG. 8A-8F is a series of plots showing that flow cytometry gating schemes for FIG. 2, FIG. 3, FIG. 6, FIG. 9, FIG. 10, and FIG. 11. FIG. 8A shows the gating scheme of single live CD45⁺ cells, serving as the starting cell population for subsequent gating. FIG. 8B shows gating of CD8⁺ T-cells and their expression of immune-checkpoint receptors. FIG. 8C shows gating of ex vivo CD8⁺ T cells expression of IFNγ and GZMB. FIG. 8D shows the gating scheme of CD4⁺ T-cell populations. FIG. 8E shows the gating scheme of CD103/CD11b expressing dendritic cells. FIG. 8F shows the gating scheme of NK and NKT cells. Polygons indicate the gates.

FIG. 9A-9D is a series of graphs showing CD4⁺ T cell profiling in the MLN and colonic LP of WT B1H (M. schaedleri-harboring) mice fed Saa diets. FIG. 9A-9B show frequencies (FIG. 9A) and numbers (FIG. 9B) of total CD4⁺ T-cells and Th1 cells, Th2 cells, Th17 cells, and Foxp3⁺ Tregs (in the MLN of WT BIH mice fed Saa diets. FIG. 9C-9D show frequencies (FIG. 9C) and numbers (FIG. 9D) of total CD4⁺ T-cells and Th1 cells, Th2 cells, Th17 cells, and Foxp3⁺ Tregs in the colonic LP of WT BIH mice fed Saa diets. Each symbol represents data from an individual mouse. Error bars represent standard error of the mean (SEM). * P value<0.05. Mann-Whitney test was performed for FIG. 9A-9D. Data represent three independent experiments for FIG. 9A-9D.

FIG. 10A-10J is a series of graphs showing myeloid cell profiling in cAPC, WT BIH, gnotobiotic, and cDC1-depleted mice fed Saa diets. FIG. 10A-10B show-frequencies and numbers of cDC1 (CD103⁺CD11b⁻), CD103⁻CD1 b⁺, and CD103⁺CD11b⁺ cells from the colonic LP (FIG. 10A) and tumors (FIG. 10B) of cAPC mice fed low or high Saa diet. FIG. 10C-10D show the frequencies and numbers of cDC1 (CD103⁺CD11b⁻), CD103⁻CD11b⁺, and CD103⁺CD11b⁺ cells from the MLN (FIG. 10C) and colonic LP (FIG. 10D) of WT BIH mice fed low or high Saa diet. FIG. 10E-10F show the frequencies and numbers of cDC1, CD103⁻CD11b⁺, and CD103⁺CD11b⁺ cells from the MLN (FIG. 10E) or LP (FIG. 10F) of WT GF mice fed low or high Saa diet. FIG. 10G shows a correlation plot of cDC1 frequencies and M. schaedleri abundance in cecal contents of mice. The black line represents a linear trend line, and the gray region represents the 95% confidence interval. FIG. 10H-10I show the frequencies and numbers of cDC1, CD103⁻CD11b⁺, and CD103⁺CD11b⁺ cells from the MLN (FIG. 10H) or LP (FIG. 10I) of WT ASF mice fed low or high Saa diet. FIG. 10J shows the frequencies and numbers of cDC1, CD103⁻CD11b⁺, and CD103⁺CD11b⁺ cells from the LP of GF, M. schaedleri-monocolonized, or A. muciniphila-monocolonized mice fed high Saa diet. Left-right order of bars in each group correspondences to left-right order of legend. In all plots, each dot represents an individual mouse. Error bars represent standard error of the mean (SEM). * P value<0.05, ** P value<0.01, *** P value<0.001. One-Way ANOVA with FDR correction was performed for FIG. 10J; Spearman correlation test was performed for FIG. 10G; and Mann-Whitney test was performed for FIG. 10A-10F and FIG. 10H-10I. Data represent three independent experiments for FIG. 10A-10I; FIG. 10J which represents two independent experiments.

FIG. 11A-11J is a series of graphs showing myeloid cell profiling in cAPC, WT bred in-house (BIH), gnotobiotic, and cDC1-depleted mice fed Saa diets. FIG. 11A shows the frequencies and numbers of cDC1 and CD103⁻CD11b⁺ cells from the TDLN of Zbtb46-DTR cAPC mice fed the high Saa diet and injected with PBS or diphtheria toxin (DT). FIG. 11B shows the relative abundance of M. schaedleri in cecal contents of Zbtb46-DTR cAPC mice fed high Saa diet and injected with PBS or DT. FIG. 11C shows the frequencies and numbers of cDC1 and CD103⁻CD11b⁺ cells from the TDLN of cAPC Batf3^{-/-} mice fed the low or high Saa diet. FIG. 11D shows the relative abundance of M. schaedleri in cecal contents of cAPC Batf3^{-/-} mice fed low or high Saa diet. FIG. 11E shows the expression of dendritic cell activation markers CD80 and CD86 on splenic cDC1 after in vitro overnight incubation with sterile mBHI or abiotic bacterial CM. FIG. 11F shows the proliferation of OT-I CD8⁺ T cells following a 3 day co-culture with cDC1 loaded with OVA protein and treated with sterile medium or abiotic bacterial CM. FIG. 11G shows the expression of IFNg by OT-1 CD8⁺ T cells following overnight incubation with DCs loaded with OVA protein or OVA peptide and treated with sterile medium or abiotic bacterial CM. FIG. 11H shows the serum concentration of XCL1 in cAPC mice fed high Saa diet and injected with α-XCL1 or isotype. FIG. 11I shows the frequencies and numbers of cDC1 and CD103⁻CD11b⁺ cells from the TDLN of cAPC mice fed high Saa diet and injected with α-XCL1 or isotype Abs. FIG. 11J shows the frequency and numbers of NK cells in the TDLN of cAPC mice fed Saa diets. In all plots, each symbol represents an individual mouse. Error bars represent standard error of the mean (SEM). * P value<0.05, ** P value<0.01, *** P value<0.001. Mann-Whitney test was performed for FIG. 11A-11D and FIG. 11H-11J. One-way ANOVA test was used for FIG. 11E, FIG. 11F and FIG. 11G. Data represent four independent experiments for FIG. 11A-11D, three independent experiments for FIG. 11E and FIG. 11H-11J, and two independent experiments for FIG. 11F and FIG. 11G.

FIG. 12A-12D is a series of graphs showing dendritic cell cluster identification in TDLN of cAPC mice fed low or high Saa diet. FIG. 12A shows a Uniform Manifold Approximation and Projection (UMAP) plot of TDLN CD11c⁺MHCII^{high}CD64⁻ cell clusters from cAPC mice. FIG. 12B shows violin plots of cDC1 marker gene expression. FIG. 12C shows a heatmap of dendritic cell (DC) cluster markers from TDLN of cAPC mice fed Saa diets. FIG. 12D shows fast gene sets enrichments analysis (fgsea) of the cDC1 cluster from TDLN of cAPC mice fed Saa diets. Only pathways with p_{adj}<0.05 are shown.

FIG. 13 shows a graphical model illustration of the diet-microbe-host interaction in CRC. Dietary Saa modulate the levels of the colonic mucus layer, leading to an expansion of M. schaedleri in high Saa diet-fed mice. M. schaedleri exerts its immunomodulatory effects on NKT cells in the tumor draining lymph nodes (TDLN), resulting in increased XCL1 expression and enhanced cDC1 recruitment. cDC1 present and cross-present to T cells in the TDLN, leading to their activation and migration to the tumor site to restrict tumor growth. Death of tumor cells is represented by a dark brown color.

FIG. 14A-14B is a series of schematics and graphs showing that a switch to high Saa diet attenuates tumor growth. FIG. 14A shows a schematic of the experimental design (upper panel); the line graph (lower panel) shows tumor volume over time in WT born in-house mice fed standard mouse chow and switched to Saa diets 5 days post flank injection with MC38. FIG. 14B is a box-and-whisker plot showing MC38 tumor weights at 12 days post injection.

FIG. 15 is a schematic showing a comparison of 16S sequences of the “Lior” M. schaedleri strain used in the Examples described herein and M. schaedleri ASF457 16S sequences deposited in GENBANK (see e.g., SEQ ID NOs: 4, 41-49).

FIG. 16 is plot showing untargeted LC-MS/MS metabolomics on the abiotic M. schaedleri CM samples (n=4 independent samples) along with the medium control without M. schaedleri (n=2 independent samples). Of the 4,263 features detected, relatively few (12 are shown in FIG. 16) were enriched in the CM and had a 2 or higher fold change, a p-value below 0.05, one-way ANOVA with post-hoc Tukey's HSD test. Identified features are depicted and labeled in the volcano plot (see also FIG. 411).

FIG. 17 is a bar graph showing an in vitro system using the mouse NKT cell line GW1 to screen the identified metabolites (see e.g., FIG. 16) for their ability to stimulate NKT XCL1 production detected by ELISA. The singly screened metabolites demonstrated some activity above the medium control (denoted by the solid horizontal line).

FIG. 18 is a bar graph showing an in vitro system using the human NKT cell to screen the identified metabolites (see e.g., FIG. 16) for their ability to stimulate NKT XCL1 production detected by ELISA. Four pools of metabolites were generated based on their chemical similarity and solubility. The level of XCL1 elicited by the medium control is denoted by the solid horizontal line. IL-12 and plate-bound anti-CD3/CD28 are positive controls. Pool 1, 3, and 4 had activity above that of the medium control. Pool 1=propionic and cis and trans aconitic acids. Pool 2=crotonic, succinic, and itaconic acids. Pool 3=nicotinic acid. Pool 4=myristic, pentadecanoic, 15-hydroxpentadecanoic, 16-hydroxyhexadecanoic, and 17-hydroxyheptadecanoic acids.

### DETAILED DESCRIPTION

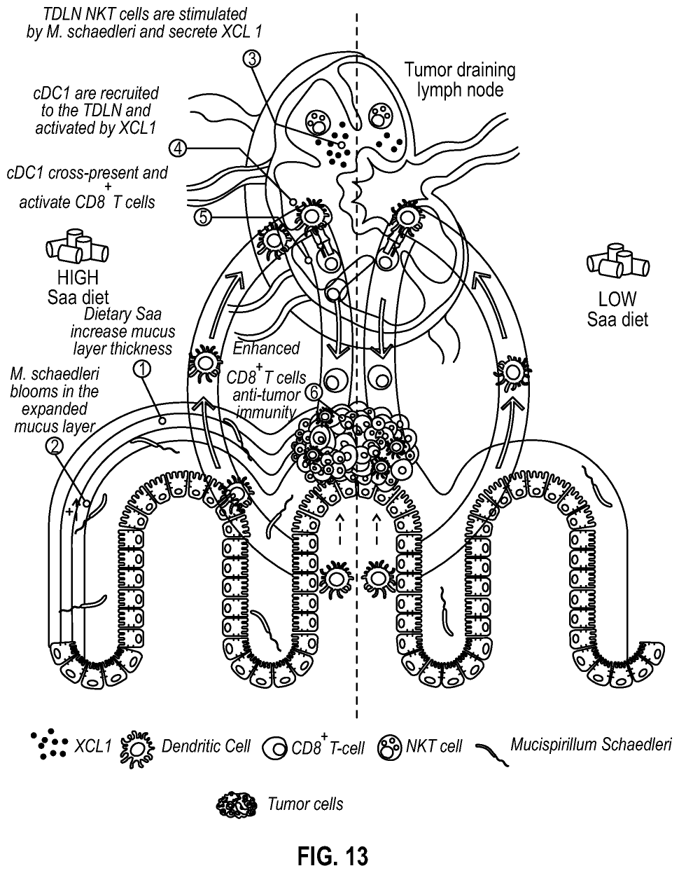

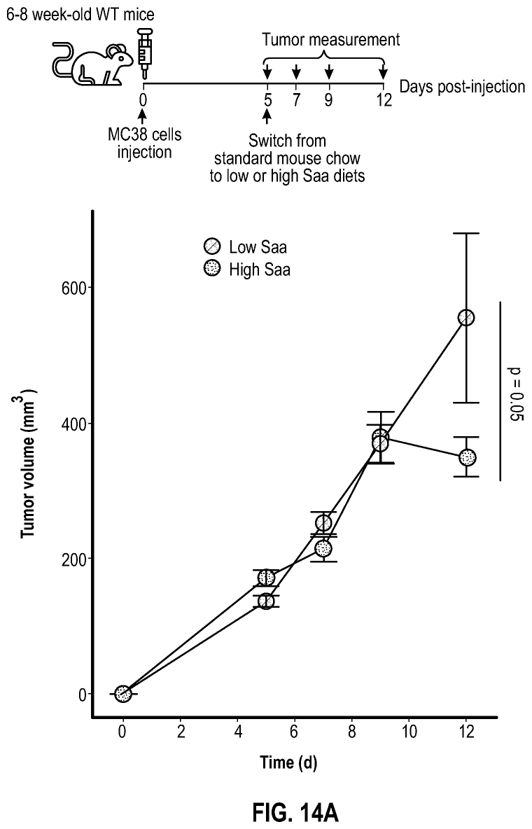

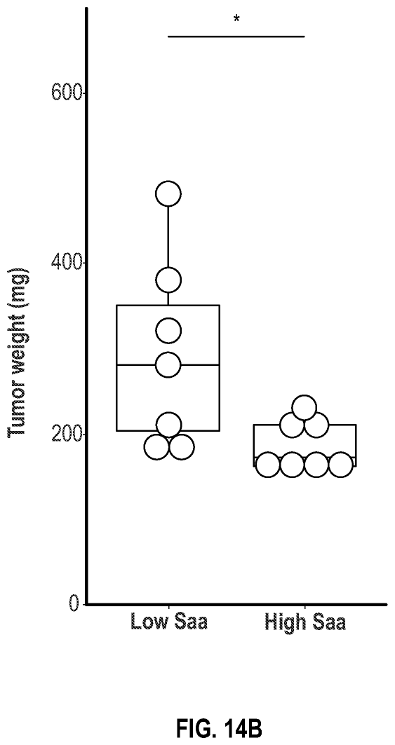

Embodiments of the technology described herein are directed to compositions comprising Mucispirillum (e.g., M. schaedleri). The disclosure describes how a diet high in sulfur amino acids can increase the gut level of M. schaedleri, which in turn increases XCL1 secretion by NKT cells. Increased XCL1 secretion by NKT cells increases CD103+ conventional dendritic cells (cDC1) number and/or activation, e.g., in tumor-draining lymph nodes, thus recruiting and activating CD8+ T cells, which have an anti-tumor immune activity (see e.g., FIG. 13). Accordingly, described herein are methods of treating cancer using Mucispirillum compositions as described herein; a diet high in sulfur amino acids (SAA) or a supplement comprising SAAs; and/or XCL1 polypeptides (or XCR1 agonists). Also described herein are cancer treatment stratification methods related to detection of the level of M. schaedleri, XCL1 polypeptide, NKTs, and/or CD103+ cDC1s, and alteration or stratification of treatment accordingly.

### Compositions

In multiple aspects described herein are compositions comprising Mucispirillum bacteria. In one aspect, described herein is a composition comprising Mucispirillum bacteria formulated for delivery to the intestine. In some embodiments of any of the aspects, the composition is formulated for delivery to the intestine via oral administration. In some embodiments of any of the aspects, the composition comprises an enteric coating or similar to survive the acidity of the stomach and permit delivery into the small or large intestine. In one aspect, described herein is a composition comprising Mucispirillum schaedleri (M. schaedleri) bacteria formulated for delivery to the intestine. In some embodiments, the M. schaedleri are formulated for delivery to the small intestine, duodenum, jejunum, ileum, cecum, ileocecum, appendix, ascending colon, transverse colon, descending colon, sigmoid colon, rectum, or anus.

Mucispirillum is a genus in the phylum Deferribacteres. It is represented by the single species Mucispirillum schaedleri. Mucispirillum is a spiral-shaped bacterium found in the mucus layer of the gastrointestinal tract of some rodents and considered a commensal. This species has been found in cockroaches, mice, turkeys, dogs, pigs, goats, termites, and humans. Mucispirillum is anaerobic and does not form spores. Mucispirillum is motile, flagellated and can have the ability to move through mucus.

In some embodiments of any of the aspects, the M. schaedleri bacteria are M. schaedleri strain ASF457 bacteria. In some embodiments of any of the aspects, the M. schaedleri bacteria comprise a 16S sequence comprising SEQ ID NO: 4 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to SEQ ID NO: 4. In some embodiments of any of the aspects, the M. schaedleri bacteria comprise a 16S sequence comprising SEQ ID NO: 4 or a nucleic acid sequence that is at least 95%, or more, identical to SEQ ID NO: 4. In some embodiments of any of the aspects, the M. schaedleri bacteria comprise a 16S sequence comprising SEQ ID NO: 4 or a nucleic acid sequence that is at least 97%, or more, identical to SEQ ID NO: 4.

“Lior” Mucispirillum schaedleri 16S ribosomal
RNA (see e.g., Example 1)
SEQ ID NO: 4
AAATTGTATGGAGAGTTTGATCCTGGCTCAGAACGAACGCTGGCGGCGTG
CTTAACACATGCAAGTCAGGGAGAAAGTCTCTTCGGGGATGATTAAACCG
GCGCACGGGTGAGTAACACGTGAGTGACCTGCCTTTTAGACTGGAACAAC
TTACCGAAAGGTGAGCTAATGCCGGATGAGTTATATAAGTGCATGTTTAT
ATAGGAAAAGTTGGGGAGACCTGACGCTGAAAGATGGACTCGCGTCCCAT
TAGCTAGTTGGTAGGGTAATGGCCTACCAAGGCGACGATGGGTAGCCGGC
CTGAGAGGGTGGCCGGCCACACTGGGACTGAGACACGGCCCAGACTCCTA
CGGGAGGCAGCAGTGGGGAATTTTGCGCAATGCTCGTAAGAGTGACGCAG
CGACGCCGCGTGAATGACGAAGGCCTTCGGGTCGTAAAGTTCTTTCGACA
GGGAAGAAAATGCCTATAAGTAACTGTGTATGTATTGACGGTACCTGTAT
AAGCAGCCCCGGCTAACTCCGTGCCAGCAGCCGCGGTAATACGGAGGGGG
CGAGCGTTGTTCGGAGTGACTGGGCGTAAAGAGCACGTAGGCGGTGTTGT
AAGTCATTAGTCAAAGACTAGAGCTCAACTTTAGTAAGGCTAGTGATACT
ATAATACTAGAGTATCAGAGAGGATTGCAGAATTCCTGGTGTAGCGGTGA
AATGCGTAGATATCAGGAGGAATACCGTTAGCGAAGGCGGCAATCTGGCT
GGAAACTGACGCTGAGGTGCGAAAGCGTGGGTAGCAAACAGGATTAGATA
CCCTGGTAGTCCACGCTGTAAACGATGGATGCTAGGTGTTGGGCTTTTAA
GTTCAGTGCCGCAGCAAACGCGATAAGCATCCCGCCTGGGGAGTACGTTT
GCAAGAATGAAACTCAAAGGAATTGACGGGGGCCCGCACAAGCGGTGGAG
CACGTGGTTTAATTCGATGCTAACCGAAGAACCTTACCTGGGTTTGACAT
CCACAGAAGGCGTTAGAGATAATGCTGTGCCTGATTTATCAGGAGCTGTG
AGACAGGTGCTGCATGGCTGTCGTCAGCTCGTGCCGTGAGGTGTTGGGTT
AAGTCCCGCAACGAGCGCAACCCCTATTTCCAGTTGCTAACGGTTGAAGC
TGAGCACTCTGGAGAGACTGCCAGCGATAAGCTGGAGGAAGGTGGGGACG
ACGTCAAGTCATCATGGCCCTTATGTCCAGGGCTACACACGTGCTACAAT
GGCATAATCAGAGGGAAGCATCTCCGCAAGGATAAGCGAATCTCATAAAT
TATGTCTCAGTTCAGATTGCAGTCTGCAACTCGACTGCATGAAGTCGGAA
TCGCTAGTAATCGCAGATCAGCAAAGCTGCGGTGAATACGTTCCCGGGCC
TTGTACACACCGCCCGTCACACCACGGGAGTCGGTCGCGCCTGAAGCCGG
TGGCCTATCAGTAATGGGGGAGCCGTCTATGGCGAGATTGGTAACTGGGG
TGAAGTCGTAACAAGGTAGCCGTACCGGAAGGTGTGGCTGGATCACCTCC
TTTCTA,

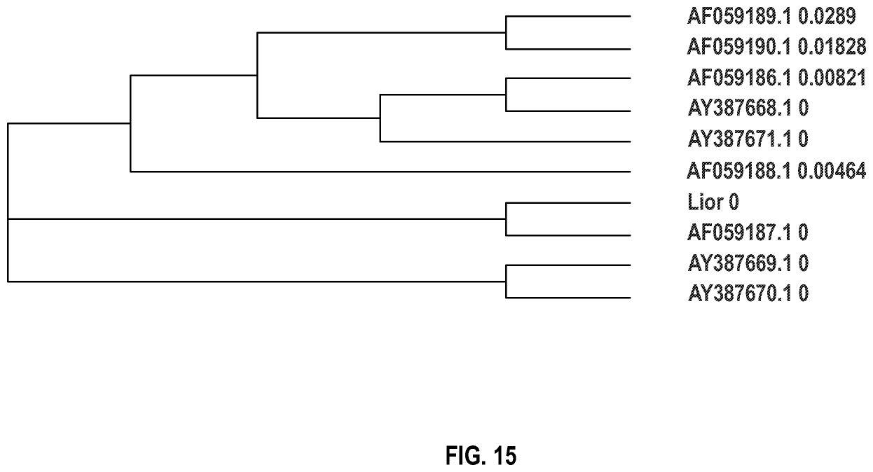

In some embodiments of any of the aspects, the M. schaedleri bacteria comprise a 16S sequence comprising one of SEQ ID NOs: 4, 40-49 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to one of SEQ ID NOs: 4, 40-49 (see e.g., FIG. 15). In some embodiments of any of the aspects, the M. schaedleri bacteria comprise a 16S sequence comprising one of SEQ ID NOs: 4, 40-49 or a nucleic acid sequence that is at least 95%, or more, identical to one of SEQ ID NOs: 4, 40-49. In some embodiments of any of the aspects, the M. schaedleri bacteria comprise a 16S sequence comprising one of SEQ ID NOs: 4, 40-49 or a nucleic acid sequence that is at least 97%, or more, identical to one of SEQ ID NOs: 4, 40-49.

Mucispirillum schaedleri strain HRI I17 16S ribosomal
RNA, partial sequence, NCBI Reference Sequence:
NR_042896.1, 1471 nucleotides (nt)
SEQ ID NO: 40
aacgaacgctggcggcgtgcttaacacatgcaagtcagggagaaagtctcttcggggatgatt
aaaccggcgcacgggtgagtaacacgtgagtgacctgccttttagactggaacaacttaccga
aaggtgagctaatgccggatgagttatataagtgcatgtttatataggaaaagttggggagac
ctgacgctgaaagatggactcgcgtcccattagctagttggtagggtaatggcctaccaaggc
gacgatgggtagccggcctgagagggtggccggccacactgggactgagacacggcccagact
cctacgggaggcagcagtggggaattttgcgcaatgctcgtaagagtgacgcagcgacgccgc
gtgaatgacgaaggccttcgggtcgtaaagttctttcgacagggaagaaaatgcctataagta
actgtgtatgtattgacggtacctgtataagcagccccggctaactccgtgccagcagccgcg
gtaatacggagggggcgagcgttgttcggagtgactgggcgtaaagagcacgtaggcggtgtt
gtaagtcattagtcaaagactagagctcaactttagtaaggctagtgatactataatactaga
gtatcagagaggattgcagaattcctggtgtagcggtgaaatgcgtagatatcaggaggaata
ccgttagcgaaggcggcaatctggctggaaactgacgctgaggtgcgaaagcgtgggtagcaa
acaggattagataccctggtagtccacgctgtaaacgatggatgetaggtgttgggcttttaa
gttcagtgccgcagcaaacgcgataagcatcccgcctggggagtacgtttgcaagaatgaaac
tcaaaggaattgacgggggcccgcacaagcggtggagcacgtggtttaattcgatgctaaccg
aagaaccttacctgggtttgacatccacagaaggcgttagagataatgctgtgcctgatttat
caggagctgtgagacaggtgctgcatggctgtcgtcagctcgtgccgtgaggtgttgggttaa
gtcccgcaacgagcgcaacccctatttccagttgctaacggttgaagctgagcactctggaga
gactgccagcgataagctggaggaaggtggggacgacgtcaagtcatcatggcccttatgtcc
agggctacacacgtgctacaatggcataatcagagggaagcatctccgcaaggataagcgaat
ctcataaattatgtctcagttcagattgcagtctgcaactcgactgcatgaagtcggaatcgc
tagtaatcgcagatcagcaaagctgcggtgaatacgttcccgggccttgtacacaccgcccgt
cacaccacgggagtcggtcgcgcctgaagccggtggcctatcagtaatgggggagccgtctat
ggcgagattggtaactggggtg,
GENBANK accession no. AF059186.1 Flexistipes group
bacterium UNSW2.6liv 16S ribosomal RNA gene, partial
sequence
SEQ ID NO: 41
AACGAACGCTGGCGGCGTGCTTAACACATGCAAGTCAGGGAGAAAGTCTNTTTGGGGATGATT
AAACCGGCGCACGGGTGAGTAACACGTGAGTGACCTGCCTTTTAGACTGGAACAACTTACCGA
AAGGTGAGCTAATGCCGGATAAGTTATATAAGTGCATGTTTATATAGGAAAAGTTGGGGAGAC
CTGACGCTGAAAGATGGACTCGCGTCCCATTAGCTAGTTGGGAGGGTAATGGCCTACCAAGGC
GACGATGGGTAGCCGGCCTGAGAGGGTGGCCGGCCACACTGGGACTGAGACACGGCCCAGACT
CCTACGGGAGGCAGCAGTGGGGAATTTTGCGCAATGCTCGTAAGAGTGACGCAGCGACGCCGC
GTGAATGACGAAGGCCTTCGGGTCGTAAAGTTCTTTCGACAGGGAAGAAAATGCCTATAAGTA
ACTGTGTATGTATTGACGGTACCTGTATAAGCAGCCCCGGCTAACTCCGTGCCAGCAGCCGCG
GTAATACGGAGGGGGCGAGCGTTGTTCGGAGTGACTGGGCGTAAAGAGCACGTAGGCGGTGTT
GTAAGTCATTAGTCAAAGACTAGAGCTCAACTTTAGTAAGGCTAGTGATACTATAATACTAGA
GTATCAGAGAGGATTGCAGAATTCCTGGTGTAGCGGTGAAATGCGTAGATATCAGGAGGAATA
CCGTTAGCGAAGGCGGCAATCTGGCTGGAAACTGACGCTGAGTGCGAAAGCGTGGTAGCAACA
GGATTAGATACCCTGGTAGTCCACGCTGTAAACGATGGATGCTAGGTGTTGGGCTTTTAAGTT
CAGTGCCGCAGCAAACGCGATAAGCATCCCGCCTGGGGAGTACGTTTGCAAGAATGAAACTCA
AAGGAATTGACGGGGGCCCGCACAAGCGGTGGAGCACGTGGTTTAATTCGATGCTAACCGAAG
AACCTTACCTGGGTTTGACATCCACAGAAGGCGTTAGAGATAATGCTGTGCCTGATTTATCAG
GAGCTGTGAGACAGGTGCTGCATGGCTGTCGTCAGCTCGTGCCGTGAGGTGTTGGGTTAAGTC
CCGCAACGAGCGCAACCCCTATTTCCAGTTGCTAACGGTTGAAGCTGAGCACTCTGGAGAGAC
TGCCAGCGATAAGCTGGAGGAAGGTGGGGACGATGTCAAGTCATCATGGCCCTTATGTCCAGG
GCTACACACGTGCTACAATGGCATAATCAGAGGGAAGCATCTCCGCAAGGATAAGCGAATCTC
ATAAATTATGTCTCAGTTCAGATTGCAGTCTGCAACTCGACTGCATGAAGTCGGAATCGCTAG
TAATCGCAGATCAGCAAAGCTGCGGTGAATACGTTCCCGGGCCTTGTACACACCGCCCGTCAC
ACCACGGGAGTCGGTCGCGCCTGAAGCCGGTGGCCTATCAGTAATGGGGGAGCCGTCTATGGC
GAGATTGGTAACTGGGGTG,
GENBANK accession no. AF059187.1 Flexistipes group
bacterium HRIlcae 16S ribosomal RNA gene, partial
sequence
SEQ ID NO: 42
AACGAACGCTGGCGGCGTGCTTAACACATGCAAGTCAGGGAGAAAGTCTCTTCGGGGATGATT
AAACCGGCGCACGGGTGAGTAACACGTGAGTGACCTGCCTTTTAGACTGGAACAACTTACCGA
AAGGTGAGCTAATGCCGGATGAGTTATATAAGTGCATGTTTATATAGGAAAAGTTGGGGAGAC
CTGACGCTGAAAGATGGACTCGCGTCCCATTAGCTAGTTGGTAGGGTAATGGCCTACCAAGGC
GACGATGGGTAGCCGGCCTGAGAGGGTGGCCGGCCACACTGGGACTGAGACACGGCCCAGACT
CCTACGGGAGGCAGCAGTGGGGAATTTTGCGCAATGCTCGTAAGAGTGACGCAGCGACGCCGC
GTGAATGACGAAGGCCTTCGGGTCGTAAAGTTCTTTCGACAGGGAAGAAAATGCCTATAAGTA
ACTGTGTATGTATTGACGGTACCTGTATAAGCAGCCCCGGCTAACTCCGTGCCAGCAGCCGCG
GTAATACGGAGGGGGCGAGCGTTGTTCGGAGTGACTGGGCGTAAAGAGCACGTAGGCGGTGTT
GTAAGTCATTAGTCAAAGACTAGAGCTCAACTTTAGTAAGGCTAGTGATACTATAATACTAGA
GTATCAGAGAGGATTGCAGAATTCCTGGTGTAGCGGTGAAATGCGTAGATATCAGGAGGAATA
CCGTTAGCGAAGGCGGCAATCTGGCTGGAAACTGACGCTGAGGTGCGAAAGCGTGGGTAGCAA
ACAGGATTAGATACCCTGGTAGTCCACGCTGTAAACGATGGATGCTAGGTGTTGGGCTTTTAA
GTTCAGTGCCGCAGCAAACGCGATAAGCATCCCGCCTGGGGAGTACGTTTGCAAGAATGAAAC
TCAAAGGAATTGACGGGGGCCCGCACAAGCGGTGGAGCACGTGGTTTAATTCGATGCTAACCG
AAGAACCTTACCTGGGTTTGACATCCACAGAAGGCGTTAGAGATAATGCTGTGCCTGATTTAT
CAGGAGCTGTGAGACAGGTGCTGCATGGCTGTCGTCAGCTCGTGCCGTGAGGTGTTGGGTTAA
GTCCCGCAACGAGCGCAACCCCTATTTCCAGTTGCTAACGGTTGAAGCTGAGCACTCTGGAGA
GACTGCCAGCGATAAGCTGGAGGAAGGTGGGGACGACGTCAAGTCATCATGGCCCTTATGTCC
AGGGCTACACACGTGCTACAATGGCATAATCAGAGGGAAGCATCTCCGCAAGGATAAGCGAAT
CTCATAAATTATGTCTCAGTTCAGATTGCAGTCTGCAACTCGACTGCATGAAGTCGGAATCGC
TAGTAATCGCAGATCAGCAAAGCTGCGGTGAATACGTTCCCGGGCCTTGTACACACCGCCCGT
CACACCACGGGAGTCGGTCGCGCCTGAAGCCGGTGGCCTATCAGTAATGGGGGAGCCGTCTAT
GGCGAGATTGGTAACTGGGGTG,
GENBANK accession no. AF059188.1 Flexistipes group
bacterium HRI3liv 16S ribosomal RNA gene, partial
sequence
SEQ ID NO: 43
AACGAACGCTGGCGGCGTGCTTAACACATGCAAGTCAGGGAGCAAAGTCTCTTCGGGGATGAT
TAAACCGGCGCACGGGTGAGTAACACGTGAGTGACCTGCCTTTTAGACTGGAACAACTTACCG
AAAGGTGAGCTAATGCCGGATGAGTTATATAAGTGCATGTTTATATAGGAAAAGTTGGGGAGA
CCTGACGCTGAAAGATGGACTCGCGTCCCATTAGCTAGTTGGTAGGGTAATGGCCTACCAAGG
CGACGATGGGTAGCCGGCCTGAGAGGGTGGCCGGCCACACTGGGACTGAGACACGCCCAGACT
CTACGGGAGGCAGCAGTGGGGAATTTTGCGCAATGCTCGTAAGAGTGACGCCAGCGACGCCGC
GTGAATGACGAAGGCCTTCGGGTCGTAAAGTTCTTTCGACAGGGAAGAAAATGCCTATAAGTA
ACTGTGTATGTATTGACGGTACCTGTATAAGCAGCCCCGGCTAACTCCGTGCCAGCAGCCGCG
GTAATACGGAGGGGGCGAGCGTTGTTCGGAGTGACTGGGCGTAAAGAGCACGTAGGCGGTGTT
GTAAGTCATTAGTCAAAGACTAGAGCTCAACTTTAGTAAGGCTAGTGATACTATAATACTAGA
GTATCAGAGAGGATTGCAGAATTCCTGGTGTAGCGGTGAAATGCGTAGATATCAGGAGGAATA
CCGTTAGCGAAGGCGGCAATCTGGCTGGAAACTGACGCTGAGGTGCGAAAGCGTGGGTAGCAA
ACAGGATTAGATACCCTGGTAGTCCACGCTGTAAACGATGGATGCTAGGTGTTGGGCTTTTAA
GTTCAGTGCCGCAGCAAACGCGATAAGCATCCCGCCTGGGGAGTACGTTTGCAAGAATGAAAC
TCAAAGGAATTGACGGGGGCCCGCACAAGCGGTGGAGCACGTGGTTTAATTCGATGCTAACCG
AAGAACCTTACCTGGGTTTGACATCCACAGAAGGCGTTAGAGATAATGCTGTGCCTGATTTAT
CAGGAGCTGTGAGACAGGTGCTGCATGGCTGTCGTCAGCTCGTGCCGTGAGGTGTTGGGTTAA
GTCCCGCAACGAGCGCAACCCCTATTTCCAGTTGCTAACGGTTGAAGCTGAGCACTCTGGAGA
GACTGCCAGCGATAAGCTGGAGGAAGGTGGGGACGACGTCAAGTCATCATGGCCCTTATGTCC
AGGGCTACACACGTGCTACAATGGCATAATCAGAGGGAAGCATCTCCGCAAGGATAAGCGAAT
CTCATAAATTATGTCTCAGTTCAGATTGCAGTCTGCAACTCGACTGCATGAAGTCGGAATCGC
TAGTAATCGCAGATCAGCAAAGCTGCGGTGAATACGTTCCCGGGCCTTGTACACACCGCCCGT
CACACCACGGGAGTCGGTCGCGCCTGAAGCCGGTGGCCTATCAGTAATGGGGGAGCCGTCTAT
GGCGAGATTGGTAACTGGGGTG,
GENBANK accession no. AF059189.1 Flexistipes group
bacterium UNSWMCS1 16S ribosomal RNA gene, partial
sequence
SEQ ID NO: 44
AGACGNYGANCTGCGSGTGCTTAACACATGCAAGTCAGGNAGNAAAGNTCCATTTGGAGCGAG
TAAACNCGHGCACGGGTGAGTAACATGTGAGTAACCTNCCTTTTAGACTGGAAYAACTTACCG
AAAGGTGAGCTAATGCCGGATGMATTATRTATCTGCATGGGTATATAGAAAAAGCTGGGGNRA
CCTGACGCTGAGAGATGGACTCGCGTCCCATTAGCTAGTTGGTGGGGTAGTAGGCCTACCAAG
GCGACGATGGGTAGCCGGCCTGAGAGGGTGGCCGGCCACACTGGGACTGAGACACGGCCCAGA
CTCCTACGGGAGGCAGCAGTGGGGAATTTTGCGCAATGCTCGAAAGAGTGACGCAGCGACGCC
GCGTGAATGANGAAGGCCTHCGGGTCGTAAAGTTCTTTCGACAGGGAAGAATTGTGTATANNA
GTAACTGGCTATATATTGACGGTACCTGTATANGCAGCCCCGGCTAACTCCGTGCCAGCAGCC
GCGGTAATACGGAGGGGGCGAGCGTTGBTCGGAGTGACTGGGCGTAAAGAGCACGTAGGCGGT
GTTGTAAGTCATTAGTCAAAGACTAGAGCTCAACTTTAGTAAGGCTAGTGATACTATAGTACT
AGAGTATCAGAGAGGATTGCAGAATTCCTGGTGTAGCGGTGAAATGCGTAGATATCAGGAGGA
ATACCATTAGCGAAGGCGGCGATCTGGCTGGAAACTGACGCTGAGGTGCGAAAGCGTGGGTAG
CAAACAGGATTAGATACCCTGGTAGTCCACGCTGTAAACGATGGATGCTAGGTGTTGGGCTAT
TAAGTTCAGTGCCGCAGCAAACGCGATAAGCATCCCNCCTGGGGAGNTACGTTTGCAAGAATK
NANACTCAAAGGAATTGACGGNGGNCCSCACAAGCGGTGGAGCACGTGGTTTAATTCGATGCT
AACCGAAGAACCTTACCTGGGTTTGACATCCACAGAATACTATAGAGATATGGTAGTGCCTGG
TTTACCAGGAACTGTGAGACAGGTGCTGCATGGCTGTCGTCAGCTCGTGCCGTGAGGTGTTGG
GNTAAGTCCCGCAACGAGCGCAACCCCTATTTCCAGTTGCTAACGGGTTAAGCTGAGCACTCT
GGAGGGACTGCCAGCGATAAGCTGGAGGAAGGTGGGGACGACGTCAAGTCATCATGGCCCTTA
TGTCCAGGGCTACACACGTGCTACAATGGCATAATCAGAGGGAAGCAGCTCCGAGAGGATAAG
CGAATCTCAGAAAGTATGTCTCAGTTCAGATTGCAGTCTGCAACTCGACTGCATGAAGTCGGA
ATCGCTAGTAATCGCAGATCAGCAAAGCTGCGGTGAATACGTTCCCCGGGCCTTGTACACACC
GCCCGTCACACCACGGGAGTCGGTNCGCGCCTGANGCCGGTGGNCTATCAGTMATGGGNRACC
TCNTATGGCGAGATTGGTAACTGGGNTG,
GENBANK accession no. AF059190.1 Flexistipes group
bacterium UNSWRSp12 16S ribosomal RNA gene, partial
sequence
SEQ ID NO: 45
AACACATGCAAGTCAGGGARWAAAGTTTCTTCGGGACGAGNTAAACCGGCGCACGGGTGAGTA
ACACGTGAGTAACCTGCCTTTTAGACTGGAACAACTTACCGAAAGGTGAGCTAATGCCGGATG
AATTATGTAACTGCATGGTTATATAGAAAAAGCTGGGGCGACCTGGTGCTAAAAGATGACTCG
CGTCCCATTAGCTAGTTGGTGGGGTAGAAGCCTACCAAGGCGACGATGGGTAGCCGGCCTGAG
AGGGTGGCCGGCCACACTGGGACTGAGACACGGCCCAGACTCCTACGGGAGGCAGCAGTGGGG
AATTTTGCGCAATGCTCGAGAGAGTGACGCAGCGACGCCGCGTGAATGACGAAGGCCTTCGGG
TCGTAAAGTTCTTTCGACAGGGAAGAATGTGTATGGTAGTAACTGACTATACAGTGACGGTAC
CTGTATAAGCAGCCCCGGCTAACTCCGTGCCAGCAGCCGCGGTAATACGGAGGGGGCGAGCGT
TGTTCGGAGTGACTGGGCGTAAAGAGCACGTAGGCGGTGTTGTAAGTCATTAGTCAAAGGCTA
GAGCTCAACTTTAGTAAGGCTAGTGATACTATAATACTAGAGTATCAGAGAGGATTGCAGAAT
TCCTGGTGTAGCGGTGAAATGCGTAGATATCAGGAGGAATACCGGTAGCGAAGGCGGCAATCT
GGCTGGAAACTGACGCTGAGGTGCGAAAGCGTGGGTAGCAAACAGGATTAGATACCCTGGTAG
TCCACGCTGTAAACGATGGATGCTAGGTGTTGGGCTAATAAGTTCAGTGCCGCAGCAAACGCG
ATAAGCATCCCGCCTGGGGAGTACGTTTGCAAGAATGAAACTCAAAGGAATTGACGGGGGCCG
CACAAGCGGTGGAGCACGTGGTTTAATTCGATGCTAACCGAAGAACCTTACCTGGGTTTGACA
TCCACAGNAATACTATAGAGATATGGTAGAGCCTGATTTATCAGGAACTGTGAGACAGGTGCT
GCATGGCTGTCGTCAGCTCGTGCCGTGAGGTGTTGGGTTAAGTCCCGCAACGAGCGCAACCCC
TATTTCCAGTTGCTAACGGGTAGAGCTGAGCACTCTGGAGAGACTGCCAGCGATAAGCTGGAG
GAAGGTGGGGACGACGTCAAGTCATCATGGCCCTTATGTCCAGGGCTACACACGTGCTACAAT
GGCATAATCAGAGGGAAGCAACTCCGAGAGGATAAGCGAATCTCATAAAGTATGTCTCAGTTC
AGATTGCAGTCTGCAACTCGACTGCATGAAGTCGGAATCGCTAGTAATCGCAGATCAGCAAAG
CTGCGGTGAATACGTTCCCGGGCCTTGTACACACCGCCCGTCACACCACGGGAGTCGGTCGCG
CCTGAAGCCGGTNGSCCTACCCNTTASNRGAGGGANMCGTCTATGGCNNGAYYKGTAACTGGG
GT,
GENBANK accession no. AY387668.1 Mucispirillum
schaedleri strain ABHU I23 16S ribosomal RNA
gene, partial sequence
SEQ ID NO: 46
AACGAACGCTGGCGGCGTGCTTAACACATGCAAGTCAGGGAGAAAGTCTCTTTGGGGATGATT
AAACCGGCGCACGGGTGAGTAACACGTGAGTGACCTGCCTTTTAGACTGGAACAACTTACCGA
AAGGTGAGCTAATGCCGGATAAGTTATATAAGTGCATGTTTATATAGGAAAAGTTGGGGAGAC
CTGACGCTGAAAGATGGACTCGCGTCCCATTAGCTAGTTGGGAGGGTAATGGCCTACCAAGGC
GACGATGGGTAGCCGGCCTGAGAGGGTGGCCGGCCACACTGGGACTGAGACACGGCCCAGACT
CCTACGGGAGGCAGCAGTGGGGAATTTTGCGCAATGCTCGTAAGAGTGACGCAGCGACGCCGC
GTGAATGACGAAGGCCTTCGGGTCGTAAAGTTCTTTCGACAGGGAAGAAAATGCCTATAAGTA
ACTGTGTATGTATTGACGGTACCTGTATAAGCAGCCCCGGCTAACTCCGTGCCAGCAGCCGCG
GTAATACGGAGGGGGCGAGCGTTGTTCGGAGTGACTGGGCGTAAAGAGCACGTAGGCGGTGTT
GTAAGTCATTAGTCAAAGACTAGAGCTCAACTTTAGTAAGGCTAGTGATACTATAATACTAGA
GTATCAGAGAGGATTGCAGAATTCCTGGTGTAGCGGTGAAATGCGTAGATATCAGGAGGAATA
CCGTTAGCGAAGGCGGCAATCTGGCTGGAAACTGACGCTGAGGTGCGAAAGCGTGGGTAGCAA
ACAGGATTAGATACCCTGGTAGTCCACGCTGTAAACGATGGATGCTAGGTGTTGGGCTTTTAA
GTTCAGTGCCGCAGCAAACGCGATAAGCATCCCGCCTGGGGAGTACGTTTGCAAGAATGAAAC
TCAAAGGAATTGACGGGGGCCCGCACAAGCGGTGGAGCACGTGGTTTAATTCGATGCTAACCG
AAGAACCTTACCTGGGTTTGACATCCACAGAAGGCGTTAGAGATAATGCTGTGCCTGATTTAT
CAGGAGCTGTGAGACAGGTGCTGCATGGCTGTCGTCAGCTCGTGCCGTGAGGTGTTGGGTTAA
GTCCCGCAACGAGCGCAACCCCTATTTCCAGTTGCTAACGGTTGAAGCTGAGCACTCTGGAGA
GACTGCCAGCGATAAGCTGGAGGAAGGTGGGGACGATGTCAAGTCATCATGGCCCTTATGTCC
AGGGCTACACACGTGCTACAATGGCATAATCAGAGGGAAGCATCTCCGCAAGGATAAGCGAAT
CTCATAAATTATGTCTCAGTTCAGATTGCAGTCTGCAACTCGACTGCATGAAGTCGGAATCGC
TAGTAATCGCAGATCAGCAAAGCTGCGGTGAATACGTTCCCGGGCCTTGTACACACCGCCCGT
CACACCACGGGAGTCGGTCGCGCCTGAAGCCGGTGGCCTATCAGTAATGGGGGAGCCGTCTAT
GGCGAGATTGGTAACTGGGGTG,
GENBANK accession no. AY387669.1 Mucispirillum
schaedleri strain HRI I12 16S ribosomal RNA gene,
partial sequence
SEQ ID NO: 47
AACGAACGCTGGCGGCGTGCTTAACACATGCAAGTCAGGGAGAAAGTCTCTTCGGGGATGATT
AAACCGGCGCACGGGTGAGTAACACGTGAGTGACCTGCCTTTTAGACTGGAACAACTTACCGA
AAGGTGAGCTAATGCCGGATGAGTTATATAAGTGCATGTTTATATAGGAAAAGTTGGGGAGAC
CTGACGCTGAAAGATGGACTCGCGTCCCATTAGCTAGTTGGTAGGGTAATGGCCTACCAAGGC
GACGATGGGTAGCCGGCCTGAGAGGGTGGCCGGCCACACTGGGACTGAGACACGGCCCAGACT
CCTACGGGAGGCAGCAGTGGGGAATTTTGCGCAATGCTCGTAAGAGTGACGCAGCGACGCCGC
GTGAATGACGAAGGCCTTCGGGTCGTAAAGTTCTTTCGACAGGGAAGAAAATGCCTATAAGTA
ACTGTGTATGTATTGACGGTACCTGTATAAGCAGCCCCGGCTAACTCCGTGCCAGCAGCCGCG
GTAATACGGAGGGGGCGAGCGTTGTTCGGAGTGACTGGGCGTAAAGAGCACGTAGGCGGTGTT
GTAAGTCATTAGTCAAAGACTAGAGCTCAACTTTAGTAAGGCTAGTGATACTATAATACTAGA
GTATCAGAGAGGATTGCAGAATTCCTGGTGTAGCGGTGAAATGCGTAGATATCAGGAGGAATA
CCGTTAGCGAAGGCGGCAATCTGGCTGGAAACTGACGCTGAGGTGCGAAAGCGTGGGTAGCAA
ACAGGATTAGATACCCTGGTAGTCCACGCTGTAAACGATGGATGCTAGGTGTTGGGCTTTTAA
GTTCAGTGCCGCAGCAAACGCGATAAGCATCCCGCCTGGGGAGTACGTTTGCAAGAATGAAAC
TCAAAGGAATTGACGGGGGCCCGCACAAGCGGTGGAGCACGTGGTTTAATTCGATGCTAACCG
AAGAACCTTACCTGGGTTTGACATCCACAGAAGGCGTTAGAGATAATGCTGTGCCTGATTTAT
CAGGAGCTGTGAGACAGGTGCTGCATGGCTGTCGTCAGCTCGTGCCGTGAGGTGTTGGGTTAA
GTCCCGCAACGAGCGCAACCCCTATTTCCAGTTGCTAACGGTTGAAGCTGAGCACTCTGGAGA
GACTGCCAGCGATAAGCTGGAGGAAGGTGGGGACGACGTCAAGTCATCATGGCCCTTATGTCC
AGGGCTACACACGTGCTACAATGGCATAATCAGAGGGAAGCATCTCCGCAAGGATAAGCGAAT
CTCATAAATTATGTCTCAGTTCAGATTGCAGTCTGCAACTCGACTGCATGAAGTCGGAATCGC
TAGTAATCGCAGATCAGCAAAGCTGCGGTGAATACGTTCCCGGGCCTTGTACACACCGCCCGT
CACACCACGGGAGTCGGTCGCGCCTGAAGCCGGTGGCCTATCAGTAATGGGGGAGCCGTCTAT
GGCGAGATTGGTAACTGGGGTG,
GENBANK accession no. AY387670.1 Mucispirillum
schaedleri strain HRI I17 16S ribosomal RNA gene,
partial sequence
SEQ ID NO: 48
AACGAACGCTGGCGGCGTGCTTAACACATGCAAGTCAGGGAGAAAGTCTCTTCGGGGATGATT
AAACCGGCGCACGGGTGAGTAACACGTGAGTGACCTGCCTTTTAGACTGGAACAACTTACCGA
AAGGTGAGCTAATGCCGGATGAGTTATATAAGTGCATGTTTATATAGGAAAAGTTGGGGAGAC
CTGACGCTGAAAGATGGACTCGCGTCCCATTAGCTAGTTGGTAGGGTAATGGCCTACCAAGGC
GACGATGGGTAGCCGGCCTGAGAGGGTGGCCGGCCACACTGGGACTGAGACACGGCCCAGACT
CCTACGGGAGGCAGCAGTGGGGAATTTTGCGCAATGCTCGTAAGAGTGACGCAGCGACGCCGC
GTGAATGACGAAGGCCTTCGGGTCGTAAAGTTCTTTCGACAGGGAAGAAAATGCCTATAAGTA
ACTGTGTATGTATTGACGGTACCTGTATAAGCAGCCCCGGCTAACTCCGTGCCAGCAGCCGCG
GTAATACGGAGGGGGCGAGCGTTGTTCGGAGTGACTGGGCGTAAAGAGCACGTAGGCGGTGTT
GTAAGTCATTAGTCAAAGACTAGAGCTCAACTTTAGTAAGGCTAGTGATACTATAATACTAGA
GTATCAGAGAGGATTGCAGAATTCCTGGTGTAGCGGTGAAATGCGTAGATATCAGGAGGAATA
CCGTTAGCGAAGGCGGCAATCTGGCTGGAAACTGACGCTGAGGTGCGAAAGCGTGGGTAGCAA
ACAGGATTAGATACCCTGGTAGTCCACGCTGTAAACGATGGATGCTAGGTGTTGGGCTTTTAA
GTTCAGTGCCGCAGCAAACGCGATAAGCATCCCGCCTGGGGAGTACGTTTGCAAGAATGAAAC
TCAAAGGAATTGACGGGGGCCCGCACAAGCGGTGGAGCACGTGGTTTAATTCGATGCTAACCG
AAGAACCTTACCTGGGTTTGACATCCACAGAAGGCGTTAGAGATAATGCTGTGCCTGATTTAT
CAGGAGCTGTGAGACAGGTGCTGCATGGCTGTCGTCAGCTCGTGCCGTGAGGTGTTGGGTTAA
GTCCCGCAACGAGCGCAACCCCTATTTCCAGTTGCTAACGGTTGAAGCTGAGCACTCTGGAGA
GACTGCCAGCGATAAGCTGGAGGAAGGTGGGGACGACGTCAAGTCATCATGGCCCTTATGTCC
AGGGCTACACACGTGCTACAATGGCATAATCAGAGGGAAGCATCTCCGCAAGGATAAGCGAAT
CTCATAAATTATGTCTCAGTTCAGATTGCAGTCTGCAACTCGACTGCATGAAGTCGGAATCGC
TAGTAATCGCAGATCAGCAAAGCTGCGGTGAATACGTTCCCGGGCCTTGTACACACCGCCCGT
CACACCACGGGAGTCGGTCGCGCCTGAAGCCGGTGGCCTATCAGTAATGGGGGAGCCGTCTAT
GGCGAGATTGGTAACTGGGGTG,
GENBANK accession no. AY387671.1 Mucispirillum
schaedleri strain UNSW I23 16S ribosomal RNA gene,
partial sequence
SEQ ID NO: 49
AACGAACGCTGGCGGCGTGCTTAACACATGCAAGTCAGGGAGAAAGTCTCTTTGGGGATGATT
AAACCGGCGCACGGGTGAGTAACACGTGAGTGACCTGCCTTTTAGACTGGAACAACTTACCGA
AAGGTGAGCTAATGCCGGATAAGTTATATAAGTGCATGTTTATATAGGAAAAGTTGGGGAGAC
CTGACGCTGAAAGATGGACTCGCGTCCCATTAGCTAGTTGGGAGGGTAATGGCCTACCAAGGC
GACGATGGGTAGCCGGCCTGAGAGGGTGGCCGGCCACACTGGGACTGAGACACGGCCCAGACT
CCTACGGGAGGCAGCAGTGGGGAATTTTGCGCAATGCTCGTAAGAGTGACGCAGCGACGCCGC
GTGAATGACGAAGGCCTTCGGGTCGTAAAGTTCTTTCGACAGGGAAGAAAATGCCTATAAGTA
ACTGTGTATGTATTGACGGTACCTGTATAAGCAGCCCCGGCTAACTCCGTGCCAGCAGCCGCG
GTAATACGGAGGGGGCGAGCGTTGTTCGGAGTGACTGGGCGTAAAGAGCACGTAGGCGGTGTT
GTAAGTCATTAGTCAAAGACTAGAGCTCAACTTTAGTAAGGCTAGTGATACTATAATACTAGA
GTATCAGAGAGGATTGCAGAATTCCTGGTGTAGCGGTGAAATGCGTAGATATCAGGAGGAATA
CCGTTAGCGAAGGCGGCAATCTGGCTGGAAACTGACGCTGAGGTGCGAAAGCGTGGGTAGCAA
ACAGGATTAGATACCCTGGTAGTCCACGCTGTAAACGATGGATGCTAGGTGTTGGGCTTTTAA
GTTCAGTGCCGCAGCAAACGCGATAAGCATCCCGCCTGGGGAGTACGTTTGCAAGAATGAAAC
TCAAAGGAATTGACGGGGGCCCGCACAAGCGGTGGAGCACGTGGTTTAATTCGATGCTAACCG
AAGAACCTTACCTGGGTTTGACATCCACAGAAGGCGTTAGAGATAATGCTGTGCCTGATTTAT
CAGGAGCTGTGAGACAGGTGCTGCATGGCTGTCGTCAGCTCGTGCCGTGAGGTGTTGGGTTAA
GTCCCGCAACGAGCGCAACCCCTATTTCCAGTTGCTAACGGTTGAAGCTGAGCACTCTGGAGA
GACTGCCAGCGATAAGCTGGAGGAAGGTGGGGACGATGTCAAGTCATCATGGCCCTTATGTCC
AGGGCTACACACGTGCTACAATGGCATAATCAGAGGGAAGCATCTCCGCAAGGATAAGCGAAT
CTCATAAATTATGTCTCAGTTCAGATTGCAGTCTGCAACTCGACTGCATGAAGTCGGAATCGC
TAGTAATCGCAGATCAGCAAAGCTGCGGTGAATACGTTCCCGGGCCTTGTACACACCGCCCGT
CACACCACGGGAGTCGGTCGCGCCTGAAGCCGGTGGCCTATCAGTAATGGGGGAGCCGTCTAT
GGCGAGATTGGTAACTGGGGTG,

In some embodiments of any of the aspects, the composition comprises about 10¹-10¹² M. schaedleri cells/mL, e.g., about 10¹ cells/mL, about 10² cells/mL, about 10³ cells/mL, about 10⁴ cells/mL, about 10⁵ cells/mL, about 10⁶ cells/mL, about 10⁷ cells/mL, about 10⁸ cells/mL, about 10⁹ cells/mL, about 10¹⁰ cells/mL, about 10¹¹ cells/mL, about 10¹² cells/mL, or more. In some embodiments of any of the aspects, the composition comprises about 10¹-10¹² colony forming units (CFUs; e.g., as a measurement of viable bacterial cells) of M. schaedleri, e.g., about 10¹ CFU/mL, about 10² CFU/mL, about 10³ CFU/mL, about 10⁴ CFU/mL, about 10⁵ CFU/mL, about 10⁶ CFU/mL, about 10⁷ CFU/mL, about 10⁸ CFU/mL, about 10⁹ CFU/mL, about 10¹⁰ CFU/mL, about 10¹¹ CFU/mL, about 10¹² CFU/mL, or more.

In some embodiments of any of the aspects, the composition comprises about 10¹-10² M. schaedleri cells/g, e.g., about 10¹ cells/g, about 10² cells/g, about 10³ cells/g, about 10⁴ cells/g, about 10⁵ cells/g, about 10⁶ cells/g, about 10⁷ cells/g, about 10⁸ cells/g, about 10⁹ cells/g, about 10¹⁰ cells/g, about 10¹¹ cells/g, about 10¹² cells/g, or more. In some embodiments of any of the aspects, the composition comprises about 10¹-10² colony forming units (CFUs; e.g., as a measurement of viable bacterial cells) of M. schaedleri, e.g., about 10¹ CFU/g, about 10² CFU/g, about 10³ CFU/g, about 10⁴ CFU/g, about 10⁵ CFU/g, about 10⁶ CFU/g, about 10⁷ CFU/g, about 10⁸ CFU/g, about 10⁹ CFU/g, about 10¹⁰ CFU/g, about 10¹¹ CFU/g, about 10¹² CFU/g, or more.

In some embodiments of any of the aspects, the composition comprises a Mucispirillum schaedleri culture (e.g., O.D.₆₀₀ₙₘ˜0.8) that is centrifuged, and the bacterial pellet is resuspended in a pharmaceutically acceptable carrier. In some embodiments of any of the aspects, the composition comprises a 5 mL Mucispirillum schaedleri culture (e.g., O.D.₆₀₀ₙₘ˜0.8) that is centrifuged, and the bacterial pellet is resuspended in a 1 mL pharmaceutically acceptable carrier, with each unit dose of the composition comprising 100 μL of the solution.

In some embodiments of any of the aspects, the M. schaedleri bacteria are living. In some embodiments of any of the aspects, the M. schaedleri bacteria are inactivated. Non-limiting examples of bacterial inactivation methods include ethanol (e.g., 40% ethanol), ultraviolet light irradiation, heating, or autoclaving. As a non-limiting example, the bacterial inactivation method comprises heating the bacteria at a temperature of at least 100° C. (e.g., at least 105° C., at least 110° C., at least 115° C., at least 120° C., or more) for at least 10 minutes (e.g., at least 15 minutes, at least 20 minutes, at least 30 minutes, at least 60 minutes or more). As another non-limiting example, the bacterial inactivation method comprises heating the bacteria at a temperature of at least 70° C. (e.g., at least 75° C., at least 80° C., at least 90° C., at least 95° C., or more) for at least 40 minutes (e.g., at least 45 minutes, at least 50 minutes, at least 60 minutes or more). As a non-limiting example, the bacterial inactivation method comprises autoclaving the bacteria at a temperature of at least 120° C. (e.g., at least 121° C., at least 125° C., at least 130° C., or more) for at least 30 minutes (e.g., at least 40 minutes, at least 45 minutes, at least 50 minutes, at least 60 minutes, or more) by using saturated steam under at least 15 psi of pressure (e.g., at least 20 psi, at least 25 psi, at least 30 psi).

In some embodiments of any of the aspects, the M. schaedleri bacteria are in dried viable form. In some embodiments of any of the aspects, the M. schaedleri bacteria are spray-dried or freeze-dried viable bacteria. In some embodiments of any of the aspects, the M. schaedleri bacteria are non-viable. In some embodiments of any of the aspects, the composition comprises fresh M. schaedleri bacteria, viable M. schaedleri bacteria, freeze-dried M. schaedleri bacteria, spray-dried M. schaedleri bacteria, non-viable M. schaedleri bacteria, or any combination thereof.

In one aspect, described herein is a composition comprising conditioned M. schaedleri culture medium, wherein the composition is formulated for delivery to the intestine. In some embodiments of any of the aspects, the conditioned culture media can be prepared by incubating the M. schaedleri bacteria in culture media for a predetermined amount of anaerobic incubation time, e.g., about 6 hours, about 12 hours, about 18 hours, about 24 hours, about 36 hours, about 2 days, about 3 days, about 4 days, about 5 days or more. In some embodiments of any of the aspects, the culture medium is modified brain-heart infusion (mBHI) medium. In some embodiments of any of the aspects, the mBHI medium comprises 37 g BHI, 5 g yeast extract, 2 mg vitamin K, 5 mg hemin, 0.5 g L-cysteine and 150 ml fetal bovine serum per 1 L water. The medium can be pH adjusted to 7.2 and filtered through 0.2 μm.

In some embodiments of any of the aspects, the conditioned culture medium is prepared by removing the M. schaedleri bacteria from the conditioned culture medium after the predetermined amount of anaerobic incubation time has elapsed; for example, the M. schaedleri bacteria can be removed by centrifugation and removal of the supernatant from the bacterial pellet. In some embodiments of any of the aspects, the conditioned culture medium is prepared by inactivating the M. schaedleri bacteria from the conditioned culture media after the predetermined amount of anaerobic incubation time has elapsed; for example, the M. schaedleri bacteria can be inactivated by ethanol (e.g., 40% ethanol), ultraviolet light irradiation, heating, or autoclaving.

As a non-limiting example, the bacterial inactivation method comprises heating the bacteria at a temperature of at least 100° C. (e.g., at least 105° C., at least 110° C., at least 115° C., at least 120° C., or more) for at least 10 minutes (e.g., at least 15 minutes, at least 20 minutes, at least 30 minutes, at least 60 minutes or more). As another non-limiting example, the bacterial inactivation method comprises heating the bacteria at a temperature of at least 70° C. (e.g., at least 75° C., at least 80° C., at least 90° C., at least 95° C., or more) for at least 40 minutes (e.g., at least 45 minutes, at least 50 minutes, at least 60 minutes or more). As a non-limiting example, the bacterial inactivation method comprises autoclaving the bacteria at a temperature of at least 120° C. (e.g., at least 121° C., at least 125° C., at least 130° C., or more) for at least 30 minutes (e.g., at least 40 minutes, at least 45 minutes, at least 50 minutes, at least 60 minutes, or more) by using saturated steam under at least 15 psi of pressure (e.g., at least 20 psi, at least 25 psi, at least 30 psi).

In one aspect, described herein is a composition comprising an organic solvent extract of conditioned M. schaedleri culture medium, wherein the composition is formulated for delivery to the intestine. Non-limiting examples of organic solvents to use for extraction include methanol, chloroform, ethyl acetate, ethanol, acetone, or any combination thereof. In some embodiments of any of the aspects, the organic solvent extract of conditioned M. schaedleri culture medium is prepared by methanol:chloroform extraction (see e.g., Materials and Methods in Example 1). In some embodiments of any of the aspects, the organic solvent extract of conditioned M. schaedleri culture medium comprises organic compounds (e.g., organic solvent soluble, non-polar) secreted or otherwise produced by M. schaedleri. In some embodiments of any of the aspects, the conditioned M. schaedleri culture medium or the organic solvent extract of conditioned M. schaedleri culture medium is heat-treated, e.g., incubation for at least 10 min at a temperature of at least 100° C.

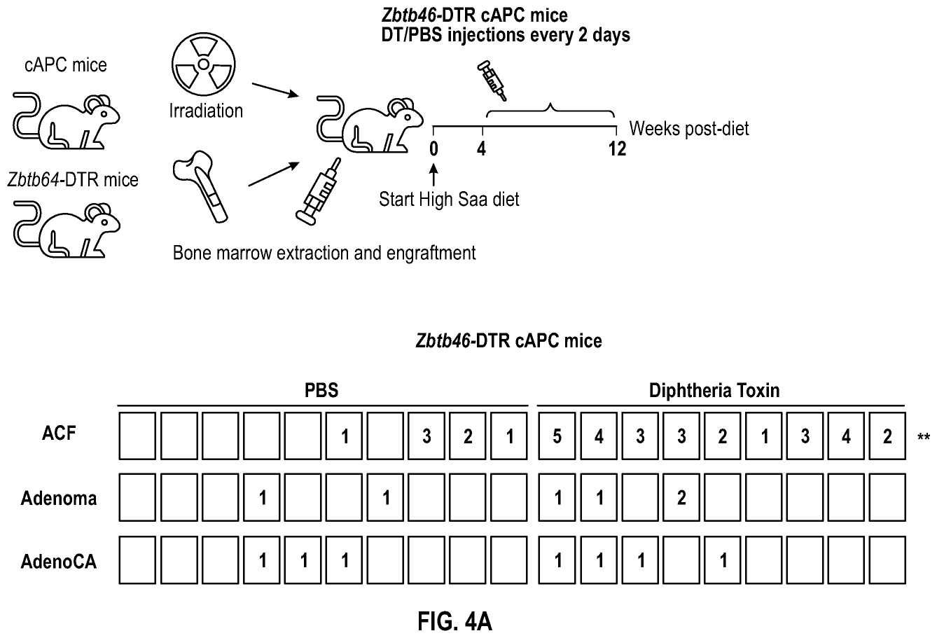

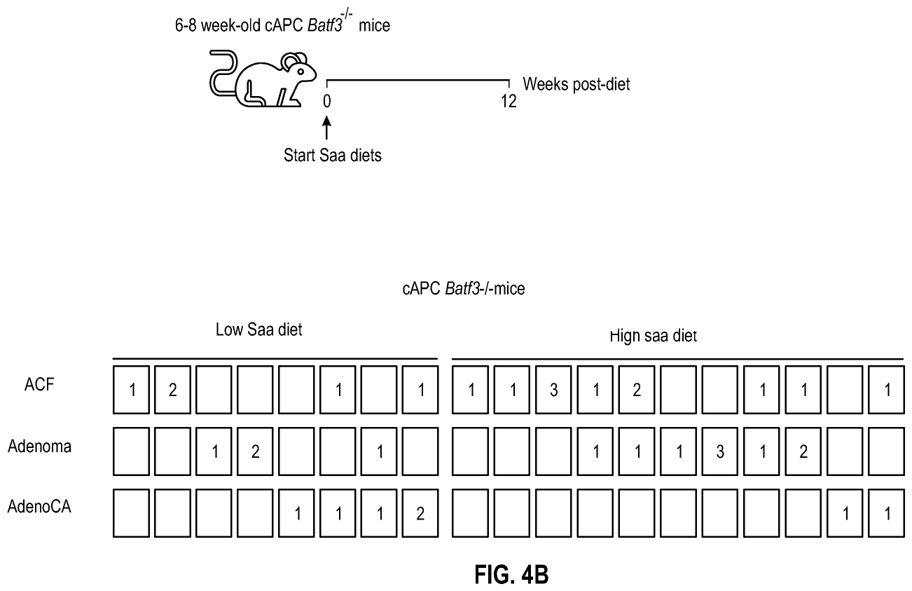

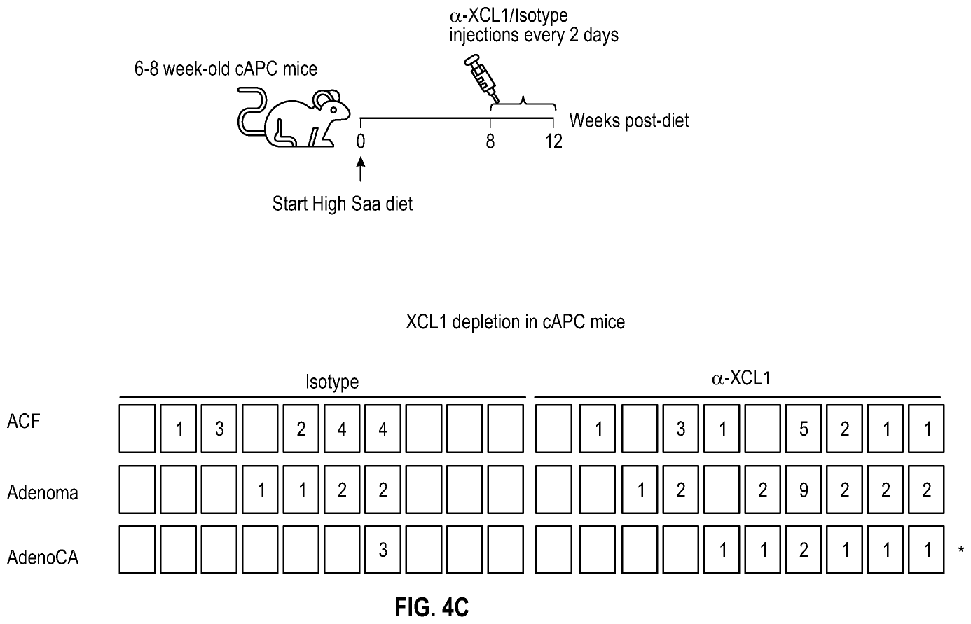

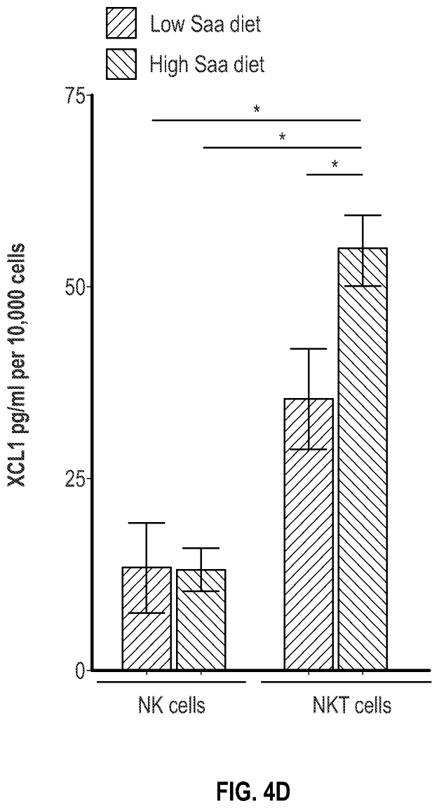

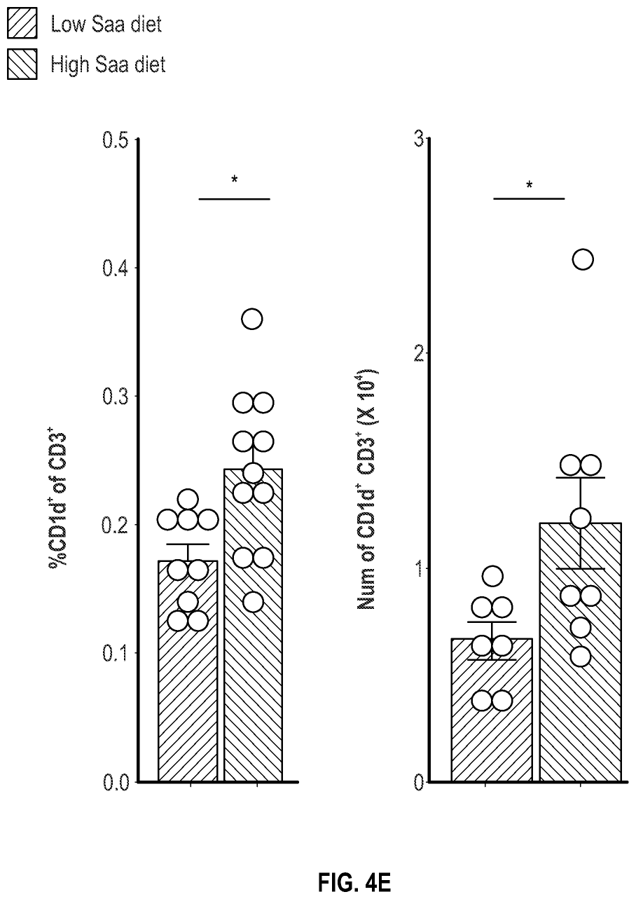

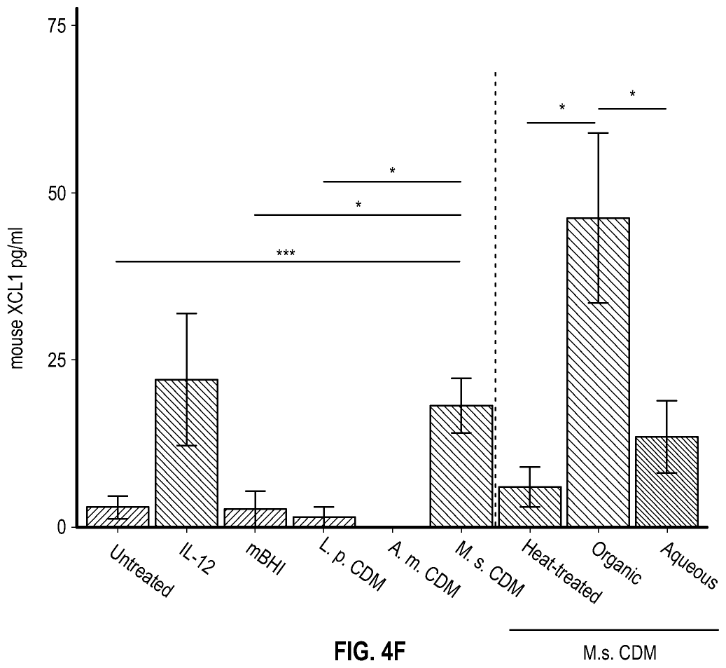

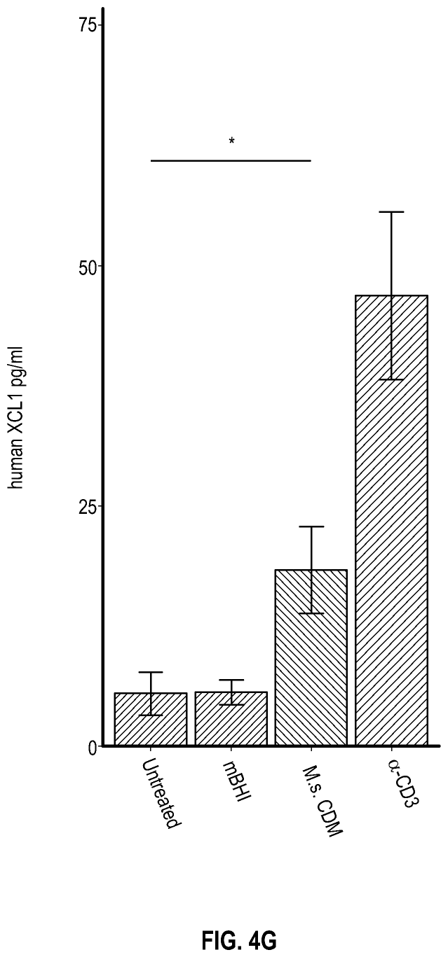

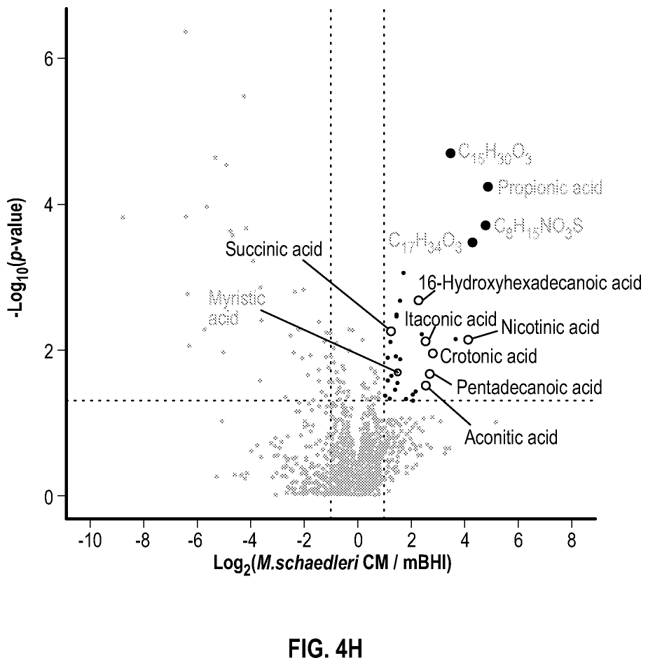

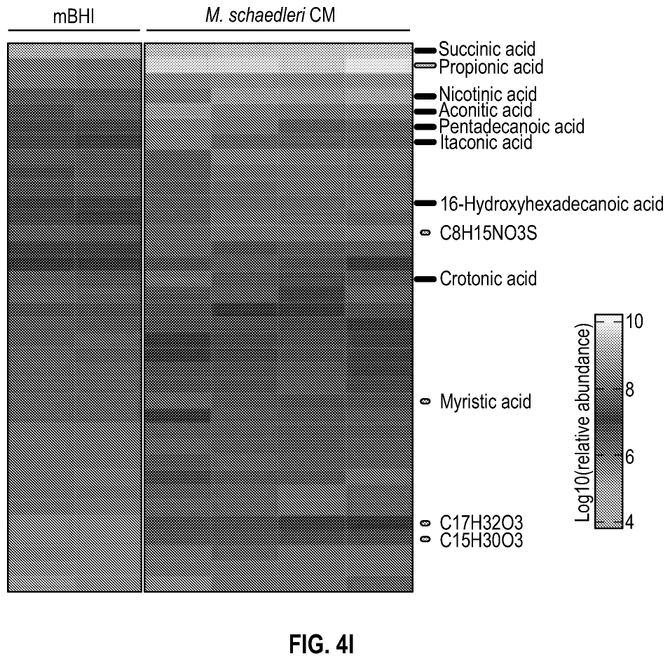

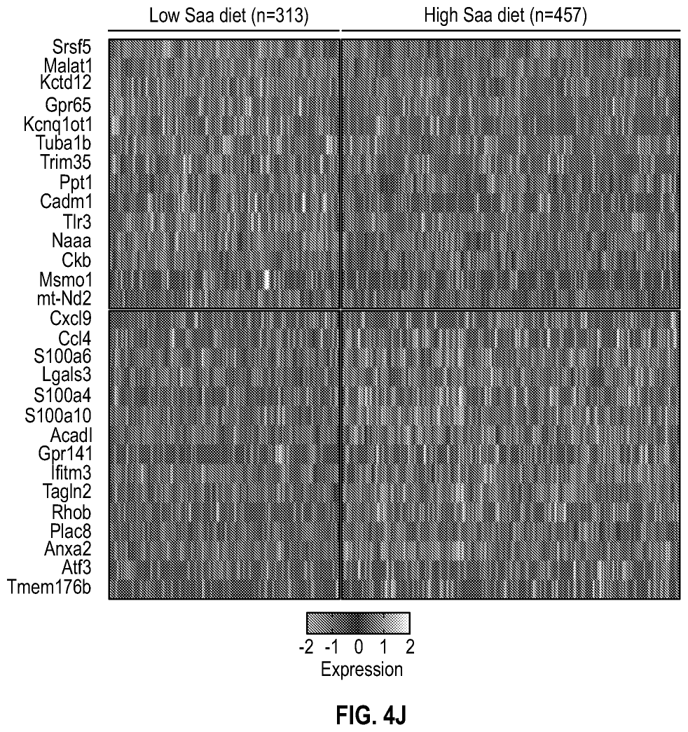

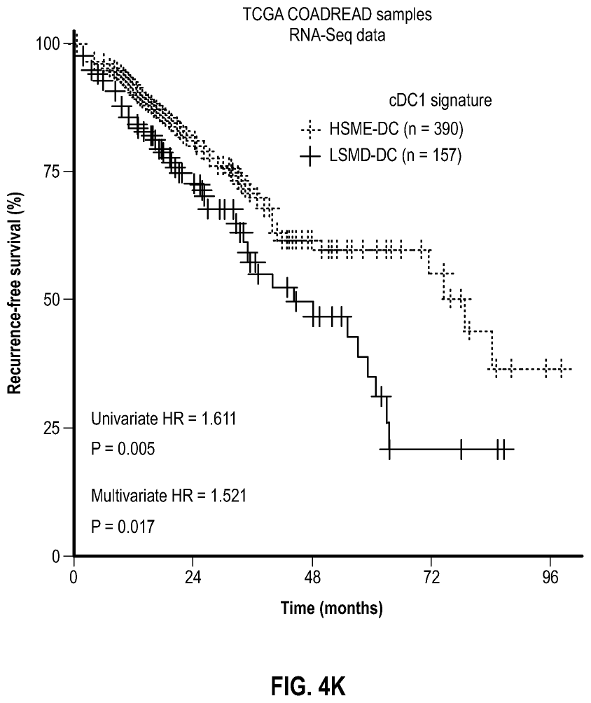

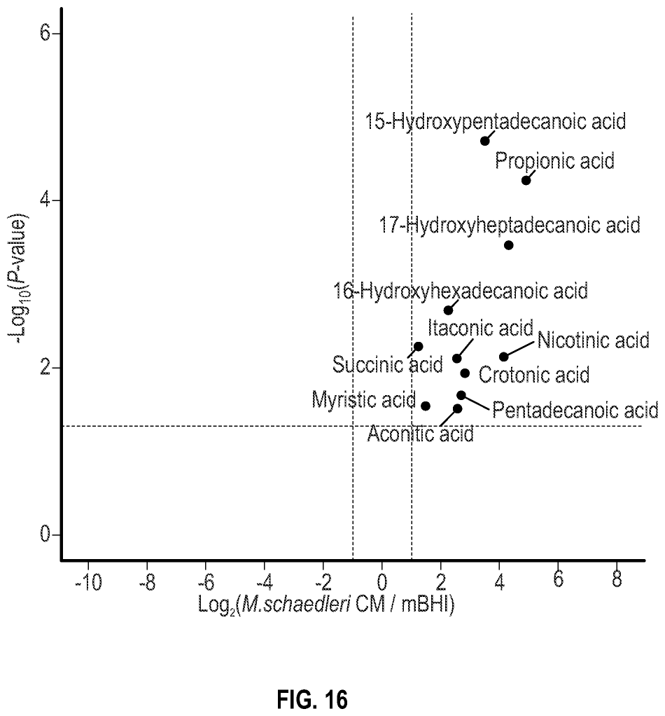

In some embodiments of any of the aspects, the conditioned M. schaedleri culture medium, or the organic solvent extract of conditioned M. schaedleri culture medium or a fraction thereof comprises at least one metabolite selected from Table 3 or Table 9 of U.S. Provisional Application No. 63/389,382, or FIG. 4H, FIG. 4I or FIG. 16 herein.

In some embodiments of any of the aspects, the conditioned M. schaedleri culture medium, or the organic solvent extract of conditioned M. schaedleri culture medium or a fraction thereof comprises at least one metabolite (e.g., at least 1, at least 2, at least 3, at least 4, at least 5, at least 6, at least 7, at least 8, at least 9, at least 10, at least 11, or 12 metabolites) selected from the group consisting of: succinic acid; propionic acid; nicotinic acid; aconitic acid (cis and/or trans); pentadecanoic acid; itaconic acid; 16-hydroxyhexadecanoic acid; C₈H₁₅NO₃S; crotonic acid; myristic acid; 17-hydroxyheptadecanoic acid (C₁₇H₃₄O₃); and 15-hydroxpentadecanoic acid (C₁₅H₃₀O₃).

In some embodiments of any of the aspects, the conditioned M. schaedleri culture medium, or the organic solvent extract of conditioned M. schaedleri culture medium or a fraction thereof comprises at least most 12 metabolites (e.g., at most 1, at most 2, at most 3, at most 4, at most 5, at most 6, at most 7, at most 8, at most 9, at most 10, at most 11, or at most 12 metabolites) selected from the group consisting of: succinic acid; propionic acid; nicotinic acid; aconitic acid (cis and/or trans); pentadecanoic acid; itaconic acid; 16-hydroxyhexadecanoic acid; C₈H₁₅NO₃S; crotonic acid; myristic acid; 17-hydroxyheptadecanoic acid (C₁₇H₃₄O₃); and 15-hydroxpentadecanoic acid (C₁₅H₃₀O₃).

In some embodiments of any of the aspects, the conditioned M. schaedleri culture medium, or the organic solvent extract of conditioned M. schaedleri culture medium or a fraction thereof comprises at most 7 metabolites (e.g., at most 1, at most 2, at most 3, at most 4, at most 5, at most 6, or at most 7 metabolites) selected from the group consisting of: succinic acid; nicotinic acid; aconitic acid (cis and/or trans); pentadecanoic acid; itaconic acid; 16-hydroxyhexadecanoic acid; and crotonic acid.

In some embodiments of any of the aspects, the conditioned M. schaedleri culture medium, or the organic solvent extract of conditioned M. schaedleri culture medium or a fraction thereof comprises succinic acid; propionic acid; nicotinic acid; aconitic acid (cis and/or trans); pentadecanoic acid; itaconic acid; 16-hydroxyhexadecanoic acid; crotonic acid; myristic acid; 17-hydroxyheptadecanoic acid (C₁₇H₃₄O₃); 15-hydroxpentadecanoic acid (C₁₅H₃₀O₃), or any combination thereof (see e.g., Formulas 4-15 in Table 3, respectively).

TABLE 3
Exemplary Metabolites

| Metabolite | Formula # | Structure |
|---|---|---|
| succinic acid | 4 | 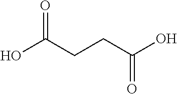 |
| propionic acid | 5 | 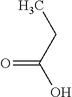 |
| nicotinic acid | 6 | 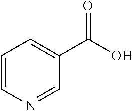 |
| aconitic acid (cis and/or trans) | 7, 8 | 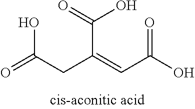 [SMILES: O=C(O)/C=C(/CC(=O)O)C(=O)O] |
| | | 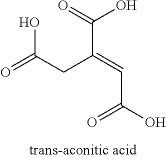 [SMILES: O=C(O)/C=C(\CC(=O)O)C(=O)O] |
| pentadecanoic acid | 9 | 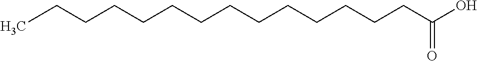 [SMILES: CCCCCCCCCCCCCCC(=O)O] |
| itaconic acid | 10 | 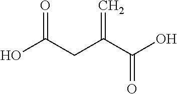 |
| 16-hydroxyhexadecanoic acid | 11 | 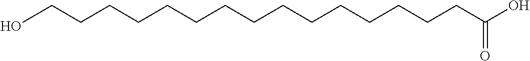 [SMILES: O=C(O)CCCCCCCCCCCCCCCO] |
| crotonic acid | 12 | 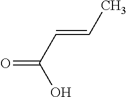 |
| myristic acid | 13 | 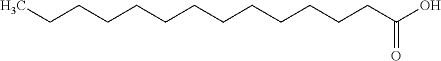 [SMILES: CCCCCCCCCCCCCC(=O)O] |
| 17-hydroxyheptadecanoic acid (C₁₇H₃₄O₃) | 14 | 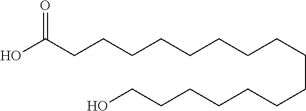 [SMILES: O=C(O)CCCCCCCCCCCCCCCCO] |
| 15-hydroxpentadecanoic acid (C₁₅H₃₀O₃) | 15 | 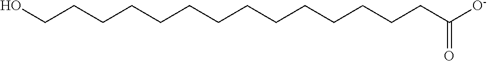 [SMILES: O=C([O-])CCCCCCCCCCCCCCO] |

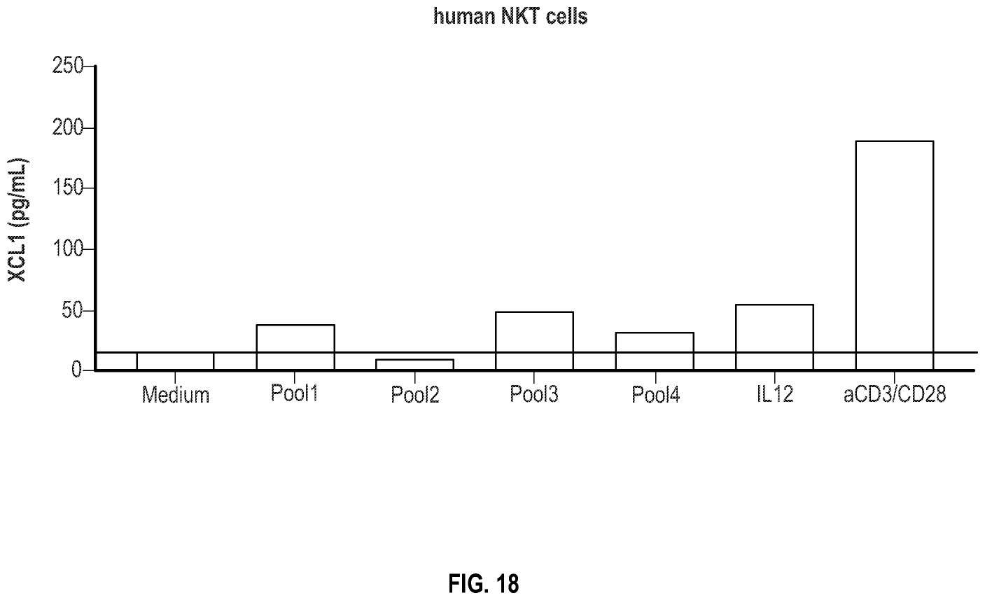

In some embodiments of any of the aspects, the conditioned M. schaedleri culture medium, or the organic solvent extract of conditioned M. schaedleri culture medium or a fraction thereof comprises propionic acid, cis-aconitic acid, and/or trans-aconitic acid, or any combination thereof (see e.g., Pool 1 of FIG. 18). In some embodiments of any of the aspects, the conditioned M. schaedleri culture medium, or the organic solvent extract of conditioned M. schaedleri culture medium or a fraction thereof comprises crotonic acid, succinic acid, and/or itaconic acid, or any combination thereof (see e.g., Pool 2 of FIG. 18). In some embodiments of any of the aspects, the conditioned M. schaedleri culture medium, or the organic solvent extract of conditioned M. schaedleri culture medium or a fraction thereof comprises nicotinic acid (see e.g., Pool 3 of FIG. 18). In some embodiments of any of the aspects, the conditioned M. schaedleri culture medium, or the organic solvent extract of conditioned M. schaedleri culture medium or a fraction thereof comprises myristic acid, pentadecanoic acid, 15-hydroxpentadecanoic acid, 16-hydroxyhexadecanoic acid, and/or 17-hydroxyheptadecanoic acid, or any combination thereof (see e.g., Pool 4 of FIG. 18).

In some embodiments of any of the aspects, the composition comprising the M. schaedleri bacteria, medium and/or solvent extract promotes XCL1 secretion by NKT cells. X-C Motif Chemokine Ligand 1 (XCL1) is also known as lymphotactin, lymphotoxin, or small inducible cytokine subfamily C, member 1. XCL1 is a chemokine, functioning in inflammatory and immunological responses, inducing leukocyte migration and activation. XCL1 contributes to chemotaxis in CD8+ T cells. NK cells release XCL1 along with IFN-γ and some other chemokines upon encountering certain bacteria, and CD8+ cells work together to cross-present antigen and communicate CD8+ T cell activation. In some embodiments of any of the aspects, the XCL1 secretion further activates cDC1s, e.g., in tumor-draining lymph nodes, which can ultimately lead to enhanced CD8+ T cell anti-tumor response. In some embodiments of any of the aspects, the composition comprising the M. schaedleri bacteria, medium and/or solvent extract further comprises an XCL1 polypeptide (e.g., SEQ ID NOs: 5-6)

In some embodiments of any of the aspects, XCL1 comprises SEQ ID NO: 5 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to SEQ ID NO: 5 that maintains its function (e.g., binding to and/or activation of XCR1). In some embodiments of any of the aspects, the mature human XCL1 peptide comprises residues 22-114 or residues 22-93 of SEQ ID NO: 5.

lymphotactin (XCL1) precursor, Homo sapiens,
NCBI Reference Sequence: NP_002986.1,
114 amino acids (aa)
SEQ ID NO: 5
MRLLILALLGICSLTAYIVEGVGSEVSDKRTCVSLTTQRLPVSRIKTYTI
TEGSLRAVIFITKRGLKVCADPQATWVRDVVRSMDRKSNTRNNMIQTKPT
GTQQSTNTAVTLTG,

In some embodiments of any of the aspects, XCL1 comprises SEQ ID NO: 6 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to SEQ ID NO: 6 that maintains its function (e.g., binding to and/or activation of XCR1). In some embodiments of any of the aspects, the mature mouse XCL1 peptide comprises residues 22-114 or residues 22-93 of SEQ ID NO: 6.

lymphotactin (XCL1) precursor, Mus musculus,
GenBank: AAA56752.1, 114 aa
SEQ ID NO: 6
MRLLLLTFLGVCCLTPWVVEGVGTEVLEESSCVNLQTQRLPVQKIKTYII
WEGAMRAVIFVTKRGLKICADPEAKWVKAAIKTVDGRASTRKNMAETVPT
GAQRSTSTAVTLTG,

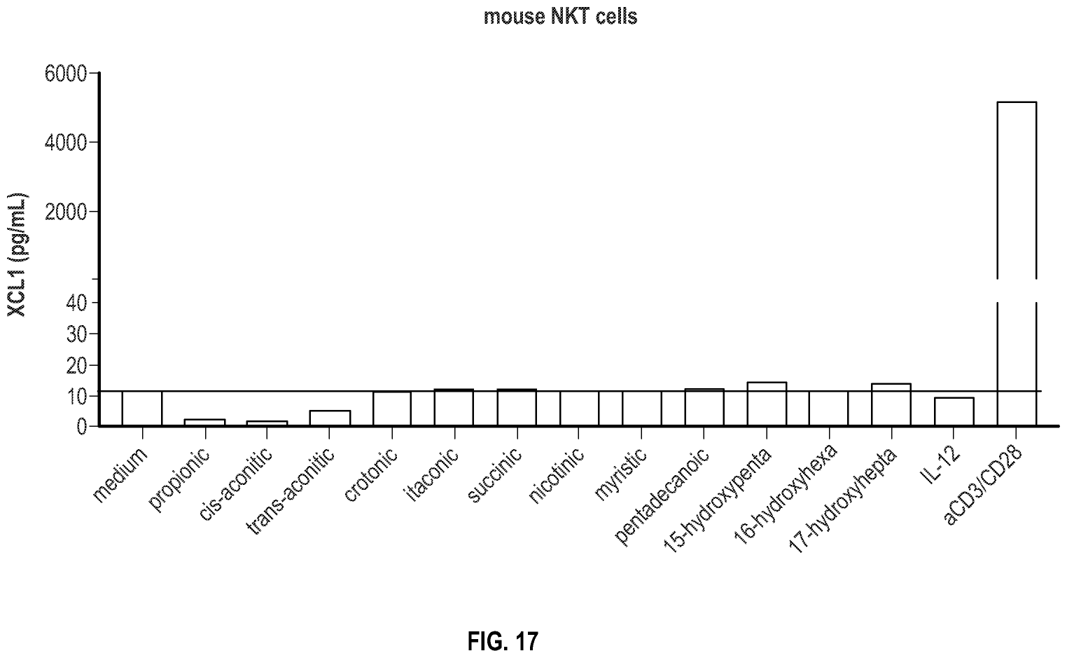

In some embodiments of any of the aspects, the composition comprising the M. schaedleri bacteria, medium and/or solvent extract increases NKT cell secretion of XCL1 by at least 100% (see e.g., FIG. 4F-4G, FIG. 17-18). In some embodiments of any of the aspects, the composition comprising the M. schaedleri bacteria, medium and/or solvent extract increases NKT cell secretion of XCL1 by at least 10%, at least 20%, at least 30%, at least 40%, at least 50%, at least 60%, at least 70%, at least 80%, at least 90%, at least 100%, at least 150%, at least 200%, at least 300%, or at least 400%, or more, as compared to NKT cells not exposed to the composition.

In some embodiments of any of the aspects, the M. schaedleri bacteria, medium and/or solvent extract are in dried form, e.g., spray-dried or freeze-dried. In some embodiments of any of the aspects, the M. schaedleri bacteria, medium and/or solvent extract are encapsulated. In some embodiments of any of the aspects, the M. schaedleri bacteria, medium and/or solvent extract are comprised in an enteric capsule. In some embodiments of any of the aspects, the composition comprises an enteric coating or similar to survive the acidity of the stomach and permit delivery into the small or large intestine. In some embodiments of any of the aspects, the composition is formulated for delivery in a capsule, an enteric capsule, a tablet, a caplet, a pill, a pressed pill, a troche, a lozenge, a powder, a granule, a nutraceutical, a medical food, a sachet, a liquid, a suspension, an oil suspension, a gel, a geltab, a semisolid, or any combination thereof.

In some embodiments of any of the aspects, the M. schaedleri bacteria, medium and/or solvent extract are maintained an anaerobic state in the formulation. As used herein, the term “anaerobic state” refers to levels of oxygen (e.g., dissolved or gaseous) at or below those normally found in the human intestinal lumen. As a non-limiting example, an anaerobic formulation of the M. schaedleri bacteria, medium and/or solvent extract can be prepared by purging oxygen from the formulation using an inert gas, such as nitrogen. In some embodiments of any of the aspects, the anaerobic formulation comprising the M. schaedleri bacteria, medium and/or solvent extract comprises no detectable dissolved or gaseous oxygen or substantially no dissolved or gaseous oxygen. In some embodiments of any of the aspects, the anacrobic formulation comprising the M. schaedleri bacteria, medium and/or solvent extract comprises at most 0.01%, at most 0.1%, or at most 1% dissolved or gaseous oxygen.

In some embodiments of any of the aspects, the M. schaedleri bacteria, medium and/or solvent extract are in admixture with a prebiotic. Non-limiting examples of prebiotics include amino acids (e.g., arginine, glutarate, and ornithine), short-chain fatty acids (SCFAs; e.g., acetate, propionate, butyrate), biotin, fructooligosaccharide, galactooligosaccharides, hemi celluloses (e.g., arabinoxylan, xylan, xyloglucan, and glucomannan), inulin, chitin, lactulose, mannan oligosaccharides, oligofructose-enriched inulin, gums (e.g., guar gum, gum arabic and carrageenan), oligofructose, oligodextrose, tagatose, resistant maltodextrins (e.g., resistant starch), trans-galactooligosaccharide, pectins (e.g., xylogalactouronan, citrus pectin, apple pectin, and rhamnogalacturonan-I), dietary fibers (e.g., soy fiber, sugarbeet fiber, pea fiber, corn bran, and oat fiber) xylooligosaccharides, polyamines (such as but not limited to spermidine and putrescine).

In some embodiments of any of the aspects, the M. schaedleri bacteria, medium and/or solvent extract are in admixture with a sulfur amino acid (SAA). In some embodiments of any of the aspects, the sulfur amino acid is methionine, cysteine or a derivative thereof. In some embodiments of any of the aspects, the sulfur amino acid is methionine, cysteine, homocysteine, taurine or a derivative thereof. In some embodiments of any of the aspects, the composition comprises a combination of any one of methionine, cysteine, homocysteine, and/or taurine (see e.g., Table 9). In some embodiments of any of the aspects, the composition comprises methionine. In some embodiments of any of the aspects, the composition comprises cysteine. In some embodiments of any of the aspects, the composition comprises homocysteine. In some embodiments of any of the aspects, the composition comprises taurine.

Methionine derivatives or cysteine derivatives necessarily include sulfur, but can differ from the structure of methionine and cysteine, and can include homocysteine or taurine. In some embodiments, the methionine derivative or the cysteine derivative is: ribose-cysteine, ribose-methionine, N-acetylcysteine, or acetylcysteine.

As used herein the term “methionine derivative” refers to an amino acid derivative resulting from reaction of methionine at the amino group or the carboxy group, or from the replacement of any hydrogen of methionine by a heteroatom; the definition normally excludes peptides containing methionine residues. Non-limiting examples of methionine derivatives include: ribose-methionine; (2S)-2-[[[4-[[(2R)-2-amino-3-mercaptopropyl]amino]-2-phenylphenyl]-oxomethyl]amino]-4-(methylthio)butanoic acid; 2-(1,3-benzothiazol-2-ylamino)-4-(methylthio)butanoic acid; 2-[(6-bromo-4-quinazolinyl)amino]-4-(methylthio)butanoic acid; 2-[[(4-ethylphenyl)-oxomethyl]amino]-4-(methylthio)butanoic acid methyl ester; 2-amino-4-(methylsulfanyl)-N-(2-naphthyl)butanamide; N-acetylmethionine; D-methionine; L-methionine; L-methionine methylsulfonium iodide; 1-Methionine, trimethylsilyl ester; methionine S-oxide; methionine sulfone; methionine sulfoximine; methioninehydroxamic acid; N-(1-Deoxy-1-fructosyl)methionine; N-[(2S)-2-Hydroxypropanoyl]methionine; N-Formyl-DL-methionine; N-Oleoyl methionine; or peptidyl-methionine. In some embodiments, the methionine derivative is ribose-methionine.

As used herein the term “cysteine derivative” refers to an amino acid derivative resulting from reaction of cysteine at the amino group, carboxy group, or thiol group, or from the replacement of any hydrogen of cysteine by a heteroatom; the definition normally excludes peptides containing cysteine residues. Non-limiting examples of cysteine derivatives include: ribose-cysteine; N-acetylcysteine; acetylcysteine; (2R; 2'S)-Isobuteine; 2-Amino-3-(hydroxysulfonylthio)propionic acid; 2-Amino-3-{[(1E)-3-(prop-2-ene-1-sulfinyl)prop-1-en-1-yl]disulfanyl}propanoic acid; 2-Ammonio-3-disulfanylpropanoate; N-acetyl-S-(1Z)-propenyl-cysteine-sulfoxide; S-(5-acetamido-2-hydroxyphenyl)cysteine; S-2-chloroethylcysteine; S-propylcysteine; D-cysteine derivative; L-cysteine derivative; allocystathionine; allylcysteine; cysteic acid; cysteinyl-amino acid; cystine (cysteine dimer); gamma-glutamylcysteinylglutamate; grixazone B; hawkinsin; L-cysteine-glycine; peptidyl-cysteine; prenylcysteine; S-(3-Oxo-3-carboxy-n-propyl)cysteine; S-(Allylthio)-L-cysteine; S-acetamidomethylcysteine; S-Cysteinosuccinic acid; or trans-S-(1-Propenyl)-L-cysteine. In some embodiments, the cysteine derivative is ribose-cysteine; N-acetylcysteine; or acetylcysteine.

TABLE 9
Exemplary SAAs in the composition (“x”
indicates inclusion in the composition)

| methionine | cysteine | homocysteine | taurine |
|---|---|---|---|
| X | | | |
| | X | | |
| X | X | | |
| | | X | |
| X | | X | |
| | X | X | |
| X | X | X | |
| | | | X |
| X | | | X |
| | X | | X |
| X | X | | X |
| | | X | X |
| X | | X | X |
| | X | X | X |
| X | X | X | X |

In some embodiments of any of the aspects, the composition comprises at least 2.0 g SAA(s) (e.g., methionine, cysteine, homocysteine, and/or taurine or a derivative thereof). In some embodiments of any of the aspects, the composition comprises at least 2.4 g SAA(s). In some embodiments of any of the aspects, the composition comprises at least 5 g, at least 10 g, at least 20 g, at least 30 g, at least 40 g, at least 50 g, at least 60 g, at least 70 g, at least 80 g, at least 90 g, at least 100 g, at least 150 g, at least 200 g, at least 300 g, or at least 400 g SAA(s). In some embodiments of any of the aspects, the composition comprises at most 5 g, at most 10 g, at most 20 g, at most 30 g, at least 40 g, at most 50 g, at most 60 g, at most 70 g, at most 80 g, at most 90 g, at most 100 g, at most 150 g, at most 200 g, at most 300 g, or at most 400 g SAA(s). In some embodiments of any of the aspects, the composition comprises 2.0 g-400 g SAA(s). In some embodiments of any of the aspects, the composition comprises 2.0 g-400 g SAA(s), 2.0 g-300 g SAA(s), 2.0 g-200 g SAA(s), 2.0 g-100 g SAA(s), 10.0 g-400 g SAA(s), 10 g-100 g SAA(s), 50 g-400 g SAA(s), 100 g-400 g SAA(s), 200 g-400 g SAA(s), or 300 g-400 g SAA(s).

In some embodiments of any of the aspects, the composition further comprises 1 to 20 additional species of bacteria. In some embodiments of any of the aspects, the composition further comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 additional species of bacteria. In some embodiments of any of the aspects, the composition comprises no more than 20 species of bacteria. In some embodiments of any of the aspects, the composition comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, or at most 20 species of bacteria, including the M. schaedleri bacteria.

In some embodiments of any of the aspects, the additional bacteria in the composition, other than the M. schaedleri bacteria, are derived from a source such as an environmental isolate, a commercially available isolate, at least a portion of a human microbiota sample, at least a portion of a non-human mammal (e.g., mouse) microbiota sample, an isolate from a human microbiota sample, an isolate from a non-human mammal microbiota sample, and the like. Although the benefit of specific microbiota species depends on the specific indication, non-limiting examples of bacterial genera that can be beneficial (e.g., to the human gastrointestinal system) and included in the composition include: Akkermansia; Alistipes; Bacillus; Bacteroides; Bifidobacterium; Blautia; Clostridium; Collinsella; Eggerthella; Enterococcus; Eubacterium; Faecalibacterium; Fusobacterium; Gemmiger; Lactobacillus; Parabacteroides; Paraprevotella; Phascolarctobacterium: Peptococcus; Peptostreptococcus; Prevotella; Roseburia; Ruminococcus; Ruthenibacterium; Streptococcus; and Subdoligranulum.

In some embodiments of any of the aspects, the composition is substantially free of pathogens. As used herein, the term “substantially” refers to the complete or nearly complete extent or degree. For example, a composition that is “substantially” free from pathogens would mean that the composition either completely or nearly completely does not comprise any pathogens, e.g., does not comprise any viable pathogens. The exact allowable degree of deviation from absolute completeness may in some cases depend on the specific context. However, generally speaking the nearness of completion will be so as to have the same overall result as if absolute and total completion were obtained. In some embodiments of any of the aspects, the pathogens in the therapeutic composition are not present or are reduced to an extent acceptable for human administration, e.g., as determined by the FDA.

As used herein, the term “pathogen” refers to any infectious microbes causing disease in an organism. In one embodiment, the pathogens comprise bacteria, fungi, archaea (e.g., methanogens, halophiles, thermophiles, and psychrophiles), protists (e.g., Plasmodium, Entamoeba histolytica, Trypanosoma brucei, Giardia lamblia), viruses, prions (e.g., PrPres and PrPSc), microscopic plants (e.g., Shewanella algae, Shewanella putrefaciens, and Shewanella xiamenensis), and/or microscopic animals/parasites (e.g., plankton, planarian, helminths, schistosomes, and trypanosomes). In some embodiments, the pathogenic viruses include but are not limited to RNA viruses such as flaviviruses, picornaviruses, rhabdoviruses, filoviruses, retroviruses (including lentiviruses), or DNA viruses such as adenoviruses, poxviruses, herpes viruses, cytomegaloviruses, hepadnaviruses, or others.

Non-limiting examples of pathogenic bacteria include spirochetes (e.g. Borrelia), actinomycetes (e.g. Actinomyces), mycoplasmas, Rickettsias, Gram negative aerobic rods, Gram negative aerobic cocci, Gram negative facultatively anaerobic rods (e.g. Erwinia and Yersinia), Gram-negative cocci, Gram negative coccobacilli, Gram positive cocci (e.g. Staphylococcus and Streptococcus), endospore-forming rods, and endospore-forming cocci. Further non-limiting examples of bacterial pathogens include certain species of Bacillus, Brucella, Burkholderia, Francisella, Yersinia, Streptococcus, Haemophilus, Nisseria, Listeria, Clostridium, Klebsiella, Legionella, Escherichia (e.g., E. coli), Mycobacterium, Staphylococcus, Campylobacter, Vibrio, and Salmonella, as well as drug and multidrug resistant strains and highly virulent strains of these pathogenic bacteria. Non-limiting examples of known food-borne bacterial pathogens include certain species of Salmonella, Clostridium, Campylobacter spp., Staphylococcus, Salmonella, Escherichia (e.g., E. coli), and Listeria. In some embodiments, non-limiting examples of bacterial pathogens include Bacillus anthracis, Brucella abortus, Brucella melitensis, Brucella suis, Burkholderia mallei, Burkholderia pseudomallei, Francisella tularensis, Yersinia pestis, Streptococcus Group A and B, MRSA, Streptococcus pneumonia, Haemophilus influenza, Nisseria meningitides, Listeria monocytegenes, Clostridium difficile, Klebsiella, highly virulent pathogenic strains of E. coli, Mycobacterium tuberculosis, Staphylococcus aureus, Campylobacter spp, Salmonella spp, and Clostridium perfringens, as well as drug and multidrug resistant strains and highly virulent strains of these pathogenic bacteria. In some embodiments, non-limiting examples of known food-borne bacterial pathogens include Salmonella, non typhoidal Clostridium perfringens. Campylobacter spp., Staphylococcus aureus, Salmonella, nontyphoidal, Campylobacter spp., E. coli (STEC) 0157, and Listeria monocytogenes.

In some embodiments of any of the aspects, the composition is substantially free of human pathogens (e.g., as described above or known in the art). In some embodiments of any of the aspects, the composition is substantially free of non-human mammal pathogens. In some embodiments of any of the aspects, the composition is substantially free of non-human mammal pathogens that can infect and/or cause disease in humans.

In some embodiments of any of the aspects, the M. schaedleri bacteria, medium and/or solvent extract are formulated in a food composition. In some embodiments of any of the aspects, the food composition comprises a yogurt or a yogurt beverage. In some embodiments of any of the aspects, the M. schaedleri bacteria, medium and/or solvent extract are formulated in a medical food. In some embodiments of any of the aspects, the M. schaedleri bacteria, medium and/or solvent extract are formulated in a supplement. In one aspect, described herein is a food composition comprising an M. schaedleri bacterium, medium and/or solvent extract as described herein. In one aspect, described herein is a medical food comprising an M. schaedleri bacterium, medium and/or solvent extract as described herein. In one aspect, described herein is a supplement comprising an M. schaedleri bacterium, medium and/or solvent extract as described herein. In some embodiments of any of the aspects, the food composition, medical food, or supplement is supplemented with a sulfur amino acid and/or a prebiotic. In some embodiments of any of the aspects, the food composition, medical food, or supplement further comprises 1 to 20 additional species of bacteria.

In some embodiments, the technology described herein relates to a pharmaceutical composition comprising the M. schaedleri bacterium, medium and/or solvent extract as described herein, and optionally a pharmaceutically acceptable carrier. In some embodiments, the active ingredients of the pharmaceutical composition comprise the M. schaedleri bacterium, medium and/or solvent extract as described herein. In some embodiments, the active ingredients of the pharmaceutical composition consist essentially of the M. schaedleri bacterium, medium and/or solvent extract as described herein. In some embodiments, the active ingredients of the pharmaceutical composition consist of the M. schaedleri bacterium. medium and/or solvent extract as described herein.

In some embodiments, the technology described herein relates to a M. schaedleri bacterium, medium and/or solvent extract pharmaceutical composition as described herein further comprising sulfur amino acids (SAAs). In some embodiments, the active ingredients of the pharmaceutical composition comprise SAAs as described herein. In some embodiments, the active ingredients of the pharmaceutical composition consist essentially of SAAs as described herein. In some embodiments, the active ingredients of the pharmaceutical composition consist of SAAs as described herein.

Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids; (23) serum component, such as serum albumin, HDL and LDL; (24) C₂-C₁₂ alcohols; and (25) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. the M. schaedleri bacteria, medium and/or solvent extract and/or SAAs, as described herein.

Conventional dosage forms generally provide rapid or immediate release of the active ingredients from the formulation. Depending on the pharmacology and pharmacokinetics of the active ingredients, use of conventional dosage forms can lead to wide fluctuations in the concentrations of the active ingredients in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control an active ingredient's onset of action, duration of action, levels (e.g., gastrointestinal levels) within the therapeutic window, and peak levels (e.g., gastrointestinal levels). In particular, controlled- or extended-release dosage forms or formulations can be used to ensure that the maximum effectiveness of an active ingredients is achieved while minimizing potential adverse effects and safety concerns, which can occur both from under-dosing the active ingredient (i.e., going below the minimum therapeutic levels) as well as exceeding the toxicity level for the active ingredient. In some embodiments, the M. schaedleri composition can be administered in a sustained release formulation.

Controlled-release pharmaceutical products have a common goal of improving therapy over that achieved by their non-controlled release counterparts. Ideally, the use of an optimally designed controlled-release preparation in medical treatment is characterized by a minimum of active ingredient being employed to cure or control the condition in a minimum amount of time. Advantages of controlled-release formulations include: 1) extended activity of the active ingredient; 2) reduced dosage frequency; 3) increased patient compliance; 4) usage of less total pharmaceutical composition; 5) reduction in local or systemic side effects; 6) minimization of active ingredient accumulation; 7) reduction in level (e.g., gastrointestinal level) fluctuations; 8) improvement in efficacy of treatment; 9) reduction of potentiation or loss of active ingredient activity; and 10) improvement in speed of control of diseases or conditions. See e.g., Kim, Chemg-ju, Controlled Release Dosage Form Design, 2 (Technomic Publishing, Lancaster, Pa.: 2000).

Most controlled-release formulations are designed to initially release an amount of active ingredient that promptly produces the desired therapeutic effect, and gradually and continually release other amounts of active ingredient to maintain this level of therapeutic or prophylactic effect over an extended period of time. In order to maintain this constant level of active ingredient in the body, the active ingredient must be released from the dosage form at a rate that will replace the amount of active ingredient being metabolized, excreted from the body, and/or otherwise inactivated. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the compositions described herein. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropyl methylcellulose, other polymer matrices, gels, permeable membranes, osmotic systems (such as OROS® (Alza Corporation, Mountain View, Calif. USA)), or a combination thereof to provide the desired release profile in varying proportions.

### Treatment Methods

The compositions described herein can be administered to a subject in need thereof, for instance for: treating cancer, promoting anti-tumor immune activity, promoting responsiveness to immune checkpoint inhibitor tumor therapy, increasing CD103+ conventional dendritic cells (cDC1), increasing XCL1 secretion by NKT cells, increasing CD8+ T cell infiltration in a colorectal tumor, establishing or maintaining a tumor-suppressive gut environment, and/or cancer treatment stratification. Such methods can comprise administration of one or more of the following: a Mucispirillum composition as described herein, a diet high in sulfur amino acids (SAA) (or a supplement comprising SAAs), and/or an XCL1 polypeptide (or XCR1 agonist). In some embodiments of any of the aspects, the method comprises administration of a Mucispirillum composition as described herein; a diet high in sulfur amino acids (SAA) (or a supplement comprising SAAs); an XCL1 polypeptide (or XCR1 agonist); a Mucispirillum composition as described herein and a diet high in sulfur amino acids (SAA) (or a supplement comprising SAAs); a Mucispirillum composition as described herein and an XCL1 polypeptide (or XCR1 agonist); a diet high in sulfur amino acids (SAA) (or a supplement comprising SAAs) and an XCL1 polypeptide (or XCR1 agonist); or a Mucispirillum composition as described herein, a diet high in sulfur amino acids (SAA) (or a supplement comprising SAAs), and an XCL1 polypeptide (or XCR1 agonist).

In one aspect of any of the embodiments, described herein is a method of treating cancer, promoting anti-tumor immune activity, promoting responsiveness to immune checkpoint inhibitor tumor therapy, increasing CD103+ conventional dendritic cells (cDC1), increasing XCL1 secretion by NKT cells, increasing CD8+ T cell infiltration in a colorectal tumor, and/or establishing or maintaining a tumor-suppressive gut environment in a subject in need thereof, the method comprising administering a Mucispirillum composition as described herein, a diet high in sulfur amino acids (SAA) (or a supplement comprising SAAs), and/or an XCL1 polypeptide (or XCR1 agonist).

In some embodiments, the method of treatment can comprise prescribing the subject a treatment as disclosed herein, in place of administering said treatment. As used herein, the term “prescribing” refers to advising and/or authorizing the use of a treatment for the subject. e.g., in writing. In one aspect of any of the embodiments, described herein is a method of treating cancer, promoting anti-tumor immune activity, promoting responsiveness to immune checkpoint inhibitor tumor therapy, increasing CD103+ conventional dendritic cells (cDC1), increasing XCL1 secretion by NKT cells, and/or establishing or maintaining a tumor-suppressive gut environment in a subject in need thereof, the method comprising prescribing a Mucispirillum composition as described herein, a diet high in sulfur amino acids (SAA) (or a supplement comprising SAAs), and/or an XCL1 polypeptide (or XCR1 agonist).

In some embodiments, the method of treatment can comprise first diagnosing a subject or patient who can benefit from treatment by a composition described herein. In some embodiments, such diagnosis comprises detecting or measuring, e.g., a low level of Mucispirillum (e.g., Mucispirillum schaedleri), a low level of sulfur amino acids (SAA), a low level of XCL1 (RNA or protein), a low level of CD103+ conventional dendritic cells (cDC1), a low level of XCL1-expressing NKT cells, a low level of anti-tumor immune activity, or a low level of responsiveness to immune checkpoint inhibitor tumor therapy, and the like, in a sample from the subject or patient, each of which are examples of an abnormal level of each analyte. In some embodiments, the method further comprises administering to the patient a Mucispirillum composition as described herein, a diet high in sulfur amino acids (SAA) (or a supplement comprising SAAs), and/or an XCL1 polypeptide (or an XCR1 agonist).

In some embodiments of any of the aspects, a low level of Mucispirillum (e.g., Mucispirillum schaedleri) is less than that measured in a normal control, e.g., as measured in the gut, feces, or a mucosal sample. In some embodiments of any of the aspects, the Mucispirillum (e.g., Mucispirillum schaedleri) level is measured using 16S rRNA abundance determined using 16S sequencing, or the level is determined using bacterial culture (e.g., CFU/mL). As a non-limiting example, the relative 16S rRNA abundance of Mucispirillum in the feces of a normal control (e.g., a healthy mouse control) is from 1E-5 to 0.05 (e.g., 1.33E-05 to 0.0441517) or from 0.01 to 0.05 (e.g., 0.01117764 to 0.0441517). In some embodiments of any of the aspects, a low level of Mucispirillum in the feces is less than 0.01, less than 0.009, less than 0.008, less than 0.007, less than 0.006, less than 0.005, less than 0.004, less than 0.003, less than 0.002, less than 1E-3, less than 1E-4, less than 1E-5, or less than 1E-6 relative 16S rRNA abundance of Mucispirillum in the feces. In some embodiments of any of the aspects, a low level of Mucispirillum is 0 to 0.01 relative 16S rRNA abundance of Mucispirillum in the feces. In some embodiments of any of the aspects, a low level of Mucispirillum is 0 to 1E-5 relative 16S rRNA abundance of Mucispirillum in the feces.

In some embodiments of any of the aspects, the level of Mucispirillum (e.g., Mucispirillum schaedleri) is measured in an intestinal sample, such as a mucosal sample or a lumen sample, as there can be a high prevalence of Mucispirillum in human mucosal biopsy samples (e.g., average 42% prevalence) but a low prevalence in fecal specimens (e.g., 3% prevalence). In some embodiments, the intestinal sample is selected from the group consisting of jejunum lumen, jejunum mucosa, terminal ileum lumen, terminal ileum mucosa, cecum lumen, cecum mucosa, ascending colon mucosa, transverse colon mucosa, descending colon lumen, and descending colon mucosa. In some embodiments, the intestinal sample is from the cecum lumen.

As a non-limiting example, the relative 16S rRNA abundance of Mucispirillum schaedleri in an intestinal sample of a normal control (e.g., a healthy human control) can range from 0.02 to 1.0, depending on the sample site: e.g., 0.2 (jejunum lumen), 0.4 (jejunum mucosa), 0.1 (terminal ileum lumen), 0.1 (terminal ileum mucosa), 1 (cecum lumen), 0.02 (cccum mucosa), 0.05 (ascending colon mucosa), 0.08 (transverse colon mucosa), 0.08 (descending colon lumen), 0.2 (descending colon mucosa), or 0.02 (stool). In some embodiments of any of the aspects, a low level of Mucispirillum is less than 1.0, less than 0.9, less than 0.8, less than 0.7, less than 0.6, less than 0.5, less than 0.4, less than 0.3, less than 0.2, less than 0.1, less than 0.09, less than 0.08, less than 0.07, less than 0.06, less than 0.05, less than 0.04, less than 0.03, less than 0.02, less than 0.01 relative 16S rRNA abundance of Mucispirillum in an intestinal sample. In some embodiments of any of the aspects, a low level of Mucispirillum is 0-0.01 relative 16S rRNA abundance of Mucispirillum in an intestinal sample.

As another non-limiting example, the relative 16S rRNA abundance of Mucispirillum schaedleri in an intestinal sample of a normal control (e.g., a healthy human control) is 0.06% (e.g., 0.057%), or 0.001%-1.4% (e.g., 0.001%-1.319%), or 0.001%-9.6% (e.g., 0.001%-9.524%). In some embodiments of any of the aspects, a low level of Mucispirillum schaedleri is less than 0.06%, less than 0.05%, less than 0.04%, less than 0.03%, less than 0.02%, less than 0.01%, less than 0.009%, less than 0.008%, less than 0.007%, less than 0.006%, less than 0.005%, less than 0.004%, less than 0.003%, less than 0.002%, or less than 0.001% relative 16S rRNA abundance of Mucispirillum schaedleri in an intestinal sample. In some embodiments of any of the aspects, a low level of Mucispirillum schaedleri is 0%-0.001% relative 16S rRNA abundance of Mucispirillum schaedleri in an intestinal sample. In some embodiments of any of the aspects, a low level of Mucispirillum schaedleri is 0%-0.06% relative 16S rRNA abundance of Mucispirillum schaedleri in an intestinal sample. See e.g., Herp et al., Cell Host & Microbe 25(5): 681-694 (2019); Zmora et al., “Personalized gut mucosal colonization resistance to empiric probiotics is associated with unique host and microbiome features,” Cell 174 (2018): 1388-1405; the contents of each of which are incorporated herein by reference in their entireties.

In some embodiments, a low level of sulfur amino acids (SAA) is less than that measured in a normal control, e.g., as measured in the plasma, gut or serum. As a non-limiting example, the level of methionine in the plasma of a normal control (e.g., a normal human control) is 14-48 μmol/L (1.40-4.80 μmol/dL) methionine. In some embodiments of any of the aspects, a low level of methionine is less than 14 mol/L, less than 13 μmol/L, less than 12 μmol/L, less than 11 gmol/L, less than 10 μmol/L, less than 5 mol/L in a plasma sample. In some embodiments of any of the aspects, a low level of methionine is 0-14 μmol/L methionine in a plasma sample.

As a non-limiting example, the level of cysteine can be measured using a cysteine derivative such as cystine. Cystine is the oxidized disulfide form of cysteine (Cys) and is the predominant form of cysteine in the blood due to its greater relative stability. Cystine is derived from dietary protein and formed endogenously from cysteine. In some embodiments of any of the aspects, the level of cystine in the plasma of a normal control (e.g., a normal human control) is 0.8-27.5 μmol/L cystine. In some embodiments of any of the aspects, a low level of cysteine (e.g., cystine) is less than 0.8 mol/L, less than 0.7 μmol/L, less than 0.6 μmol/L, less than 0.5 μmol/L, less than 0.4 μmol/L, less than 0.3 μmol/L, less than 0.2 μmol/L, less than 0.1 μmol/L in a plasma sample. In some embodiments of any of the aspects, a low level of cysteine (e.g., cystine) is 0-0.8 μmol/L cystine in a plasma sample.

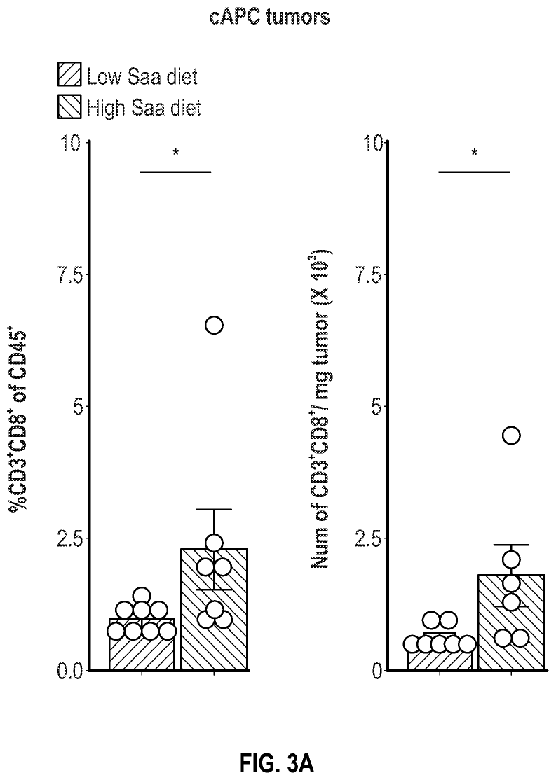

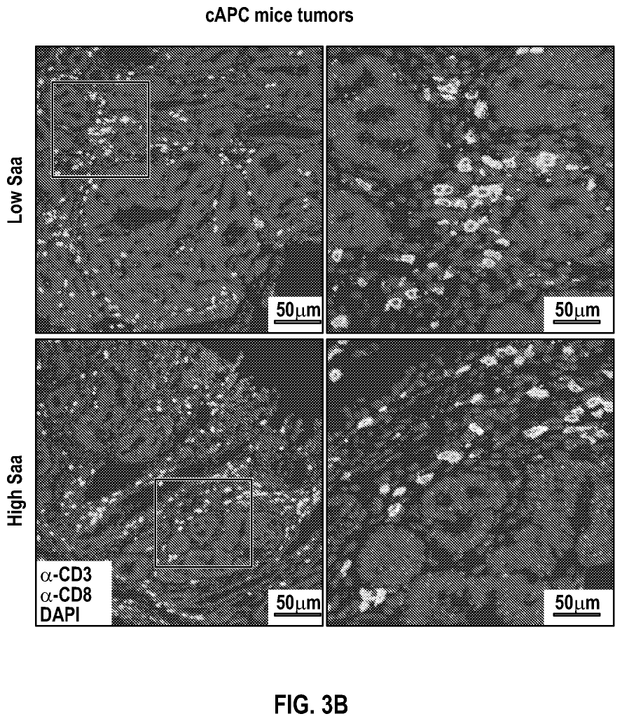

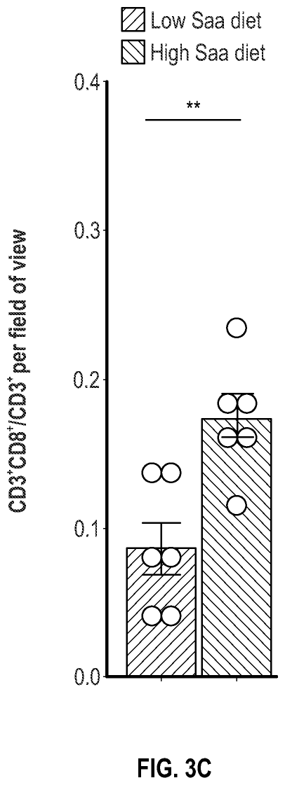

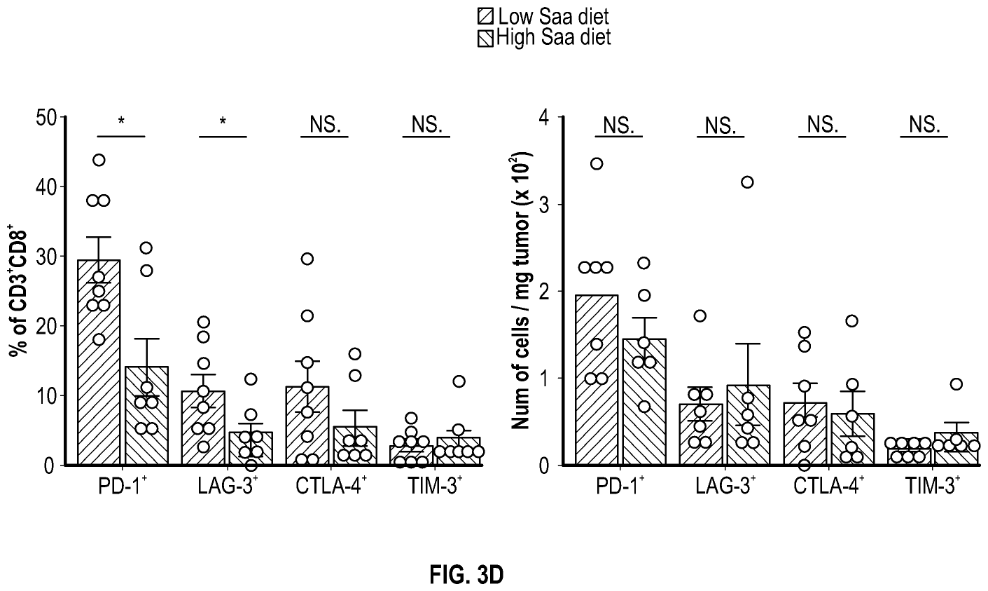

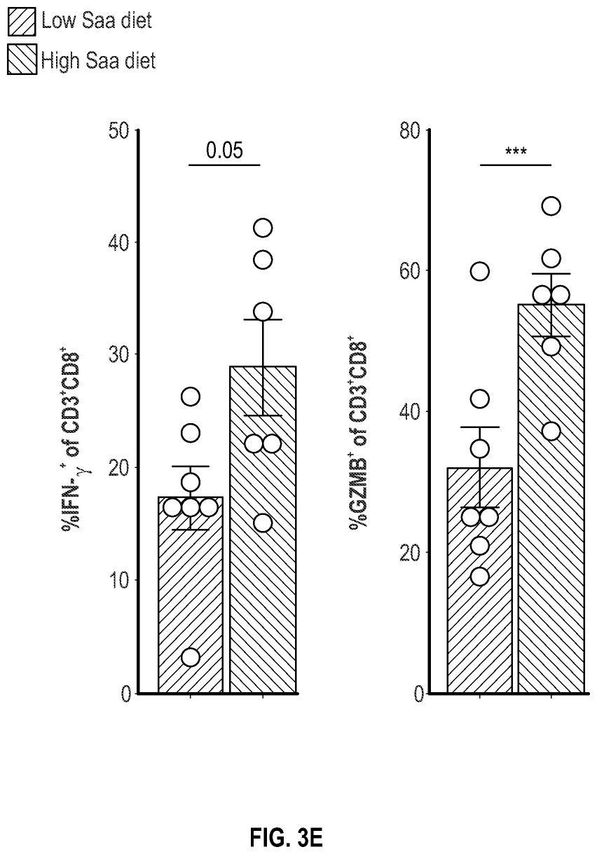

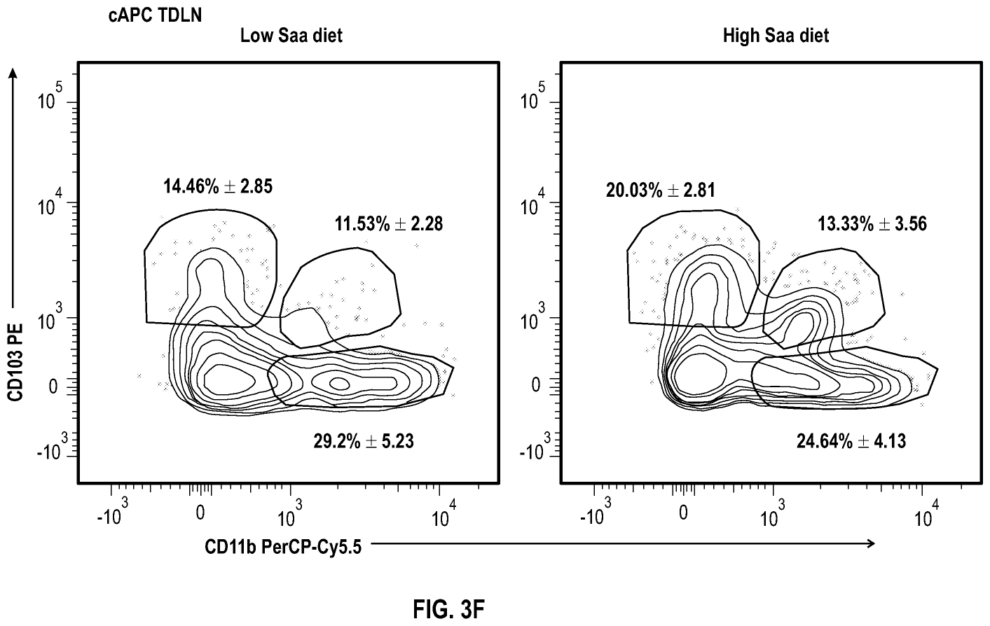

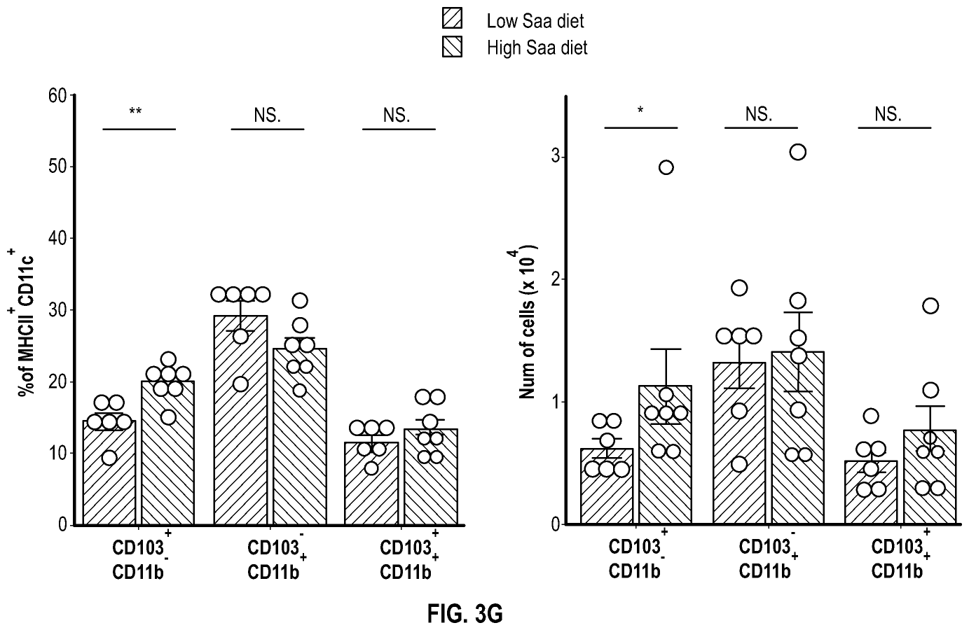

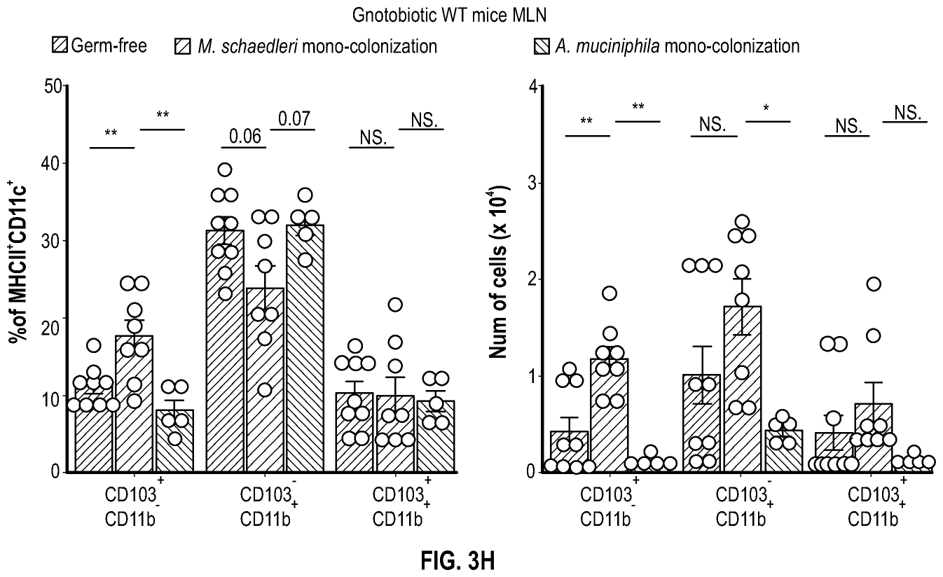

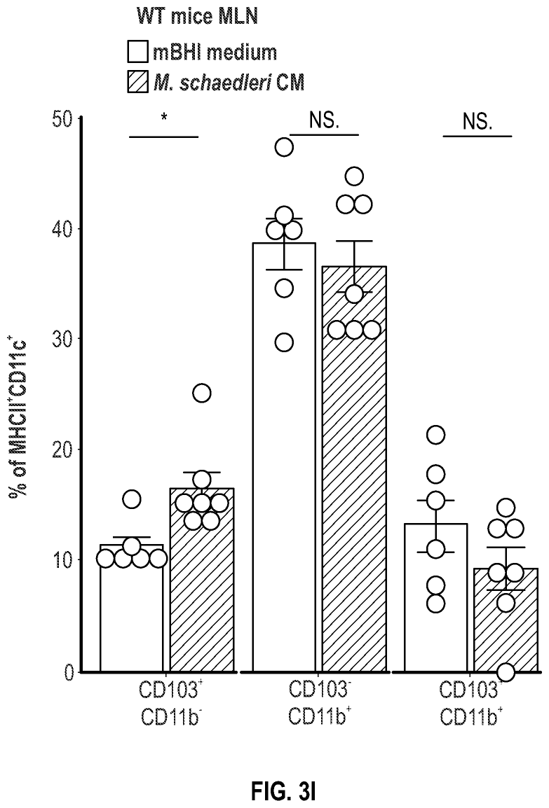

In some embodiments of any of the aspects, a low level of XCL1 (RNA or protein) is less than 10 pg/mL-30 pg/mL secreted XCL1 polypeptide (see e.g., FIG. 4D, FIG. 4F-4G, FIG. 17-18), e.g., as measured in the gut or serum. In some embodiments of any of the aspects, a low level of CD103+ conventional dendritic cells (cDC1) is less than 10-15% of MHCII+CD11c+ cells that are CD103+CD11 b− or less than 0.5×10⁴-1×10⁴ CD103+CD11 b− cells (see e.g., FIG. 3G-3I, FIG. 27C-27G). In some embodiments of any of the aspects, a low level of NKT cells (e.g., XCL1-expressing NKT cells) is less than 0.25% of CD3+ cells that are CD1d-tetramer-binding NKT cells or less than 1×10⁴ NKT cells (see e.g., FIG. 33D), e.g., as measured in the gut, tumor-draining lymph nodes, or tumor.

As used herein, the term “anti-tumor immune activity” refers to an immune-mediated attack on tumor cells or tissue. Anti-tumor immune activity, e.g., as mediated by the compositions and methods described herein, can comprise a shift in the tumor microenvironment from an immunosuppressive immune profile to an immune profile that permits and/or promotes immune-mediated attack on tumor cells or tissue. An immunosuppressive immune profile can be characterized by the presence and/or activation of regulatory T cells (Tregs), regulatory B cells (Bregs), exhausted T cells, increased expression of immune checkpoint proteins, and/or decreased activation of immune cells (e.g., cDC1s, NKTs, CD8+ T cells, etc.). A shift in the tumor microenvironment that permits and/or promotes immune-mediated attack on tumor cells or tissue includes increased infiltration or activity of activated immune cells (e.g., antigen-presenting cells such as cDC1s; NKTs; CD8+ T cells, etc.), a decrease in the local concentration or activation of Tregs or Bregs, and decreased expression of immune checkpoint proteins. In some embodiments of any of the aspects, a low level of anti-tumor immune activity is a low level of CD8+ T cell anti-tumor immunity, such as less than 0.1 CD3+CD8+ cells per CD3+ cells per field of view, less than 20% CD3+CD8+ cells that are IFN-gamma+ (see e.g., FIG. 3D), or less than 30% CD3+CD8+ cells that are granzyme-B+ (see e.g., FIG. 3C-3E).

In some embodiments of any of the aspects, a low level of responsiveness to immune checkpoint inhibitor (ICI) tumor therapy is when, despite administration of an ICI (e.g., known to target a checkpoint molecule expressed on the tumor), tumor growth (e.g., tumor volume, tumor mass) is not slowed by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more, relative to non-treatment with the ICI. Alternatively, or in addition, “low responsiveness” is when tumor-infiltrating lymphocytes (TILs) do not increase following CPI administration and/or when activated CD8+ TILs (e.g., IFNγ⁺ and/or GZMB⁺) do not increase following CPI administration.

In some embodiments of any of the aspects, the frequencies of CD8+ T cells can be measured in a tumor or a tumor draining lymph node. In some embodiments of any of the aspects, a low level of anti-tumor immune activity or a low level of responsiveness to immune checkpoint inhibitor tumor therapy can be associated with no change or an increase of a measure of a tumor (e.g., tumor weight or tumor volume) or of symptom(s) or complication(s) associated with a tumor or cancer.

In some embodiments, the subject has previously been determined to have an abnormal level of an analyte described herein relative to a reference. In some embodiments, the reference level can be the level in a sample of similar cell type, sample type, sample processing, and/or obtained from a subject of similar age, sex and other demographic parameters as the sample/subject. In some embodiments, the test sample and control reference sample are of the same type, that is, obtained from the same biological source, and comprising the same composition, e.g. the same number and type of cells.

The term “sample” or “test sample” as used herein denotes a sample taken or isolated from a biological organism, e.g., a blood or plasma sample from a subject. In some embodiments of any of the aspects, the technology described herein encompasses several examples of a biological sample. In some embodiments of any of the aspects, the biological sample is cells, or tissue, or peripheral blood, or bodily fluid. Exemplary biological samples include, but are not limited to, a biopsy, a tumor sample, biofluid sample; blood; serum; plasma; urine; semen; mucus; tissue biopsy; organ biopsy; synovial fluid; bile fluid; cerebrospinal fluid; mucosal secretion; effusion; sweat; saliva; and/or tissue sample etc. The term also includes a mixture of the above-mentioned samples. The term “test sample” also includes untreated or pretreated (or pre-processed) biological samples. In some embodiments of any of the aspects, a test sample can comprise cells from a subject.

In some embodiments of any of the aspects, the step of determining if the subject has an abnormal level of an analyte described herein can comprise i) obtaining or having obtained a sample from the subject and ii) performing or having performed an assay on the sample obtained from the subject to determine/measure the level of the analyte in the subject. In some embodiments of any of the aspects, the step of determining if the subject has an abnormal level of an analyte described herein can comprise performing or having performed an assay on a sample obtained from the subject to determine/measure the level of analyte in the subject. In some embodiments of any of the aspects, the step of determining if the subject has an abnormal level of an analyte described herein can comprise ordering or requesting an assay on a sample obtained from the subject to determine/measure the level of the analyte in the subject. In some embodiments of any of the aspects, the step of determining if the subject has an abnormal level of an analyte described herein can comprise receiving the results of an assay on a sample obtained from the subject to determine/measure the level of the analyte in the subject. In some embodiments of any of the aspects, the step of determining if the subject has an abnormal level of an analyte described herein can comprise receiving a report, results, or other means of identifying the subject as a subject with a decreased level of the analyte.

In one aspect of any of the embodiments, described herein is a method of treating cancer, promoting anti-tumor immune activity, promoting responsiveness to immune checkpoint inhibitor tumor therapy, increasing CD103+ conventional dendritic cells (cDC1), increasing XCL1 secretion by NKT cells, and/or establishing or maintaining a tumor-suppressive gut environment in a subject in need thereof, the method comprising: a) determining if the subject has an abnormal level of an analyte described herein; and b) instructing or directing that the subject be administered a Mucispirillum composition as described herein, a diet high in sulfur amino acids (SAA) (or a supplement comprising SAAs), and/or an XCL1 polypeptide herein if the level of the analyte is abnormal (e.g., decreased) relative to a reference. In some embodiments of any of the aspects, the step of instructing or directing that the subject be administered a particular treatment can comprise providing a report of the assay results. In some embodiments of any of the aspects, the step of instructing or directing that the subject be administered a particular treatment can comprise providing a report of the assay results and/or treatment recommendations in view of the assay results.

### Mucispirillum Methods

In multiple aspects described herein are methods of using the Mucispirillum compositions described herein for methods including but not limited to methods of treating cancer, promoting anti-tumor immune activity, promoting responsiveness to immune checkpoint inhibitor tumor therapy, increasing CD103+ conventional dendritic cells (cDC1), increasing XCL1 secretion by NKT cells, and/or increasing CD8+ T cell infiltration in a colorectal tumor.

In one aspect, described herein is a method of treating cancer or promoting anti-tumor immune activity, the method comprising administering to a subject in need thereof a Mucispirillum composition as described herein. In one aspect, described herein is a method of treating colon cancer, the method comprising administering to a subject in need thereof a Mucispirillum composition as described herein. In one aspect, described herein is a method of promoting anti-tumor immune activity, the method comprising administering to a subject in need thereof a Mucispirillum composition as described herein. In one aspect, described herein is a method of increasing CD8+ T cell infiltration in a colorectal tumor, the method comprising administering to a subject in need thereof a Mucispirillum composition as described herein.

In one aspect, described herein is a method of treating cancer, promoting anti-tumor immune activity, promoting responsiveness to immune checkpoint inhibitor tumor therapy, increasing CD103+ conventional dendritic cells (cDC1), increasing XCL1 secretion by NKT cells, and/or increasing CD8+ T cell infiltration in a colorectal tumor, the method comprising prescribing a Mucispirillum composition as described herein to a subject in need thereof.

In one aspect, described herein is a method of promoting responsiveness to immune checkpoint inhibitor tumor therapy, the method comprising administering to a subject in need thereof a Mucispirillum composition as described herein. In some embodiments of any of the aspects, the method further comprises administering an immune checkpoint inhibitor. In one aspect, described herein is a method of promoting responsiveness to immune checkpoint inhibitor tumor therapy, the method comprising administering to a subject in need thereof an immune checkpoint inhibitor (ICI) and a Mucispirillum composition as described herein. In some embodiments of any of the aspects, the ICI is administered before administration of the Mucispirillum composition. In some embodiments of any of the aspects, the ICI is administered after administration of the Mucispirillum composition. In some embodiments of any of the aspects, the ICI is administered at the same time as administration of the Mucispirillum composition. In some embodiments of any of the aspects. the subject has cancer. In some embodiments of any of the aspects, the subject has colon cancer. In some embodiments of any of the aspects, the subject has a cancer of mucosal epithelial tissue. In some embodiments of any of the aspects, the subject's cancer has been determined to be resistant to immune checkpoint inhibitor therapy.

In one aspect, described herein is a method of increasing XCL1 secretion by NKT cells, the method comprising administering to a subject in need thereof a Mucispirillum composition as described herein. In some embodiments of any of the aspects, the subject has cancer. In some embodiments of any of the aspects, the XCL1 secretion recruits cDCs and/or activates cDC1s. In some embodiments of any of the aspects, the subject has colon cancer. In some embodiments of any of the aspects, the subject has a cancer of mucosal epithelial tissue.

In one aspect, described herein is a method of increasing CD103+ conventional dendritic cells (cDC1), the method comprising administering to a subject in need thereof a Mucispirillum composition as described herein. In some embodiments of any of the aspects, the administration increases CD103+CD11b⁻ conventional dendritic cells (cDC1). In some embodiments of any of the aspects, the administration increases the number of cDCs. In some embodiments of any of the aspects, the administration increases the number of cDCs in tumor draining lymph nodes (TDLNs). In some embodiments of any of the aspects, the administration increases the activation of cDCs (e.g., increased expression of the XCL1 receptor XCR1). In some embodiments of any of the aspects, the administration increases the activation of cDCs (e.g., increased expression of the XCL1 receptor XCR1) in tumor draining lymph nodes (TDLNs). In some embodiments of any of the aspects, the cDCs are recruited to tumor draining lymph nodes (TDLNs) and/or activated by XCL1. In some embodiments of any of the aspects, the cDC1s are associated with a tumor. In some embodiments of any of the aspects, the cDC1s are associated with tumor draining lymph nodes (TDLNs). In some embodiments of any of the aspects, the tumor is a colon cancer. In some embodiments of any of the aspects, the tumor is a tumor of mucosal epithelial tissue. In some embodiments of any of the aspects, the tumor is an adenoma, which is a tumor that is not cancer, which starts in gland-like cells of the epithelial tissue. In some embodiments of any of the aspects, the tumor is a carcinoma, which is a cancer that begins in the skin or in tissues that line or cover internal organs.

In some embodiments of any of the aspects, the cancer is colon cancer. In some embodiments of any of the aspects, the cancer is colorectal cancer (CRC). In some embodiments of any of the aspects, the cancer is a cancer of mucosal epithelial tissue or a cancer of the mucosa. As used herein, the term “mucosa” refers to a mucous membrane or a membrane rich in mucous glands that lines body passages and cavities (e.g., the digestive or respiratory tracts) which connect directly or indirectly with the exterior. In some embodiments of any of the aspects, the cancer is a cancer of the gastrointestinal tract, including but not limited to: oral cancer, esophageal cancer, gastric (stomach) cancer, small intestine cancer, colorectal cancer, or anal cancer. In some embodiments of any of the aspects, the cancer is a cancer of the respiratory tract, including but not limited to: lung cancer, throat cancer, or bronchial adenoma.

In some embodiments of any of the aspects, the method further comprises administering an immune checkpoint inhibitor. In some embodiments of any of the aspects, the immune checkpoint inhibitor comprises an immune checkpoint inhibitor antibody. In some embodiments of any of the aspects, the checkpoint inhibitor immunotherapy is an inhibitor of a checkpoint molecule selected from the group consisting of programmed cell death 1 (PD-1), programmed death-ligand 1 (PD-L1), cytotoxic T-lymphocyte-associated protein 4 (CTLA-4), Adenosine A2A receptor (A2AR), CD276, CD39, CD73, B7 family immune checkpoint molecules, V-set domain-containing T-cell activation inhibitor 1 (B7H4), B and T Lymphocyte Attenuator (BTLA), Indoleamine 2,3-dioxygenase (IDO), Killer-cell Immunoglobulin-like Receptor (KIR), Lymphocyte Activation Gene-3 (LAG-3), nicotinamide adenine dinucleotide phosphate NADPH oxidase isoform 2 (NOX2), T-cell Immunoglobulin domain and Mucin domain 3 (TIM-3), T cell immunoreceptor with Ig and ITIM domains (TIGIT), V-domain Ig suppressor of T cell activation (VISTA), and Sialic acid-binding immunoglobulin-type lectin 7 (SIGLEC7).

Non-limiting examples of immune checkpoint inhibitors (ICIs) include: pembrolizumab (Keytrudar), nivolumab (Opdivo®), cemiplimab (Libtayo®), spartalizumab, camrelizumab (AiRuiKa™), sintilimab (TYVYT®), tislelizumab, toripalimab (Tuoyi™), dostarlimab (JEMPERLI), INCMGA00012, AMP-224, AMP-514 (MEDI0608), atezolizumab (Tecentriq®), avelumab (Bavencio®), envafolimab (KN035), cosibelimab (CK-301), AUNP12, CA-170, BMS-986189, BMS-936559 (MDX-1105), durvalumab (IMFINZI®), tremelimumab, and ipilimumab (Yervoy®). See e.g., US Patents U.S. Pat. Nos. 5,811,097, 5,855,887, 6,051,227, 6,682,736, 6,984,720, 7,595,048, 7,605,238, 7,943,743, 8,008,449, 8,217,149, 8,354,509, 8,383,796, 8,728,474, 8,735,553, 8,779,105, 8,779,108, 8,907,053, 8,900,587, 8,952,136, 9,067,999, 9,073,994, 9,683,048, 9,987,500, U.S. Ser. No. 10/160,736, U.S. Ser. No. 10/316,089, U.S. Ser. No. 10/441,655, U.S. Ser. No. 10/590,199, U.S. Ser. No. 11/225,522, US Patent Publication US2014341917; Storz et al., MAbs. 2016 January; 8(1): 10-26; the contents of each of which are incorporated herein by reference in their entireties.

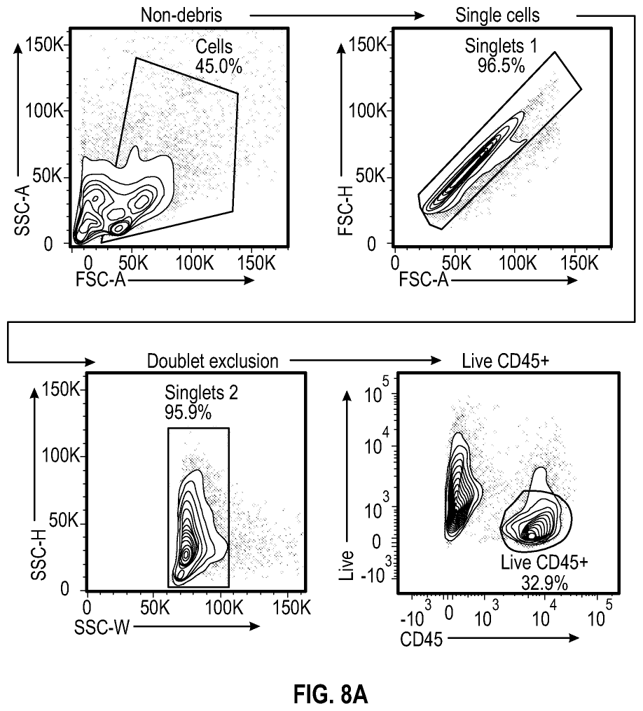

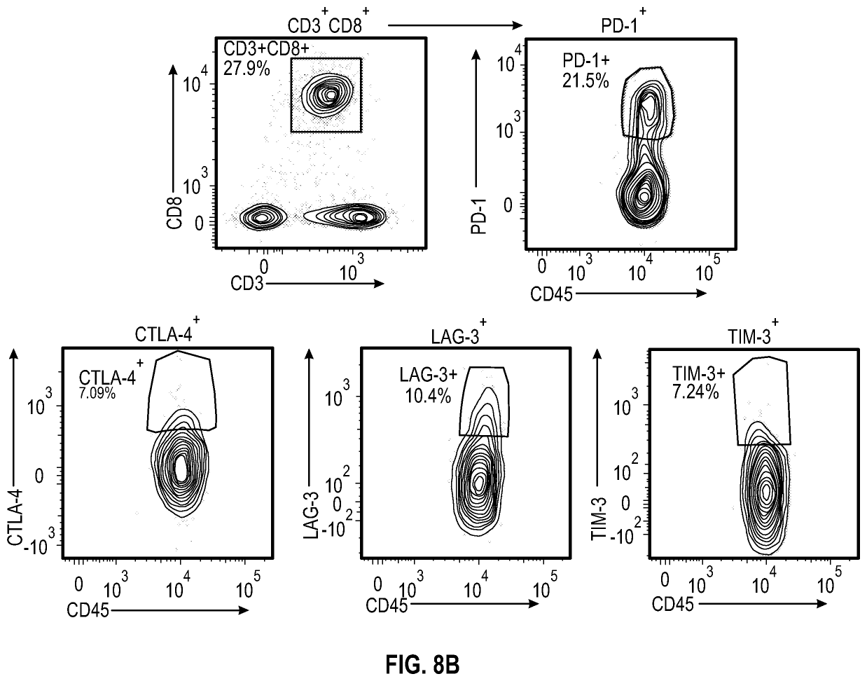

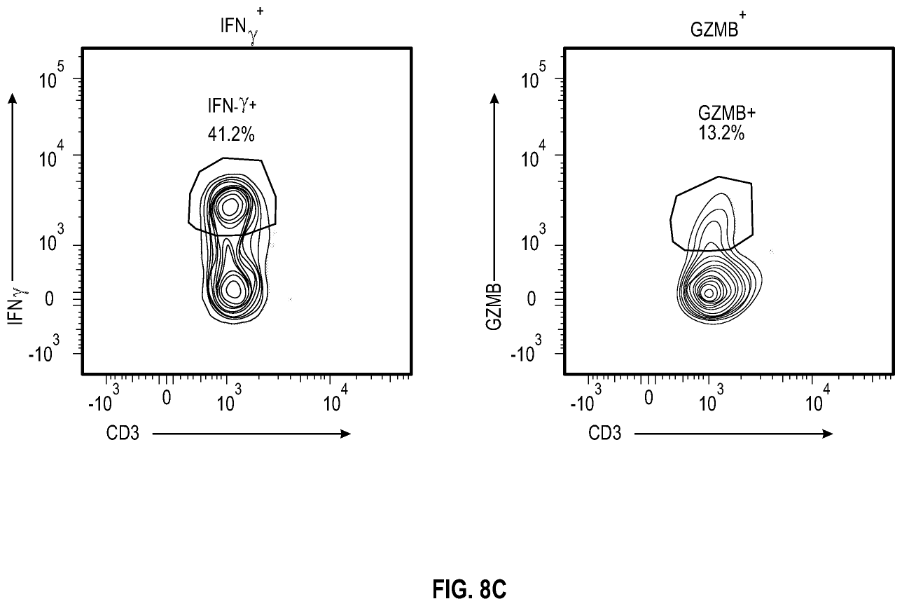

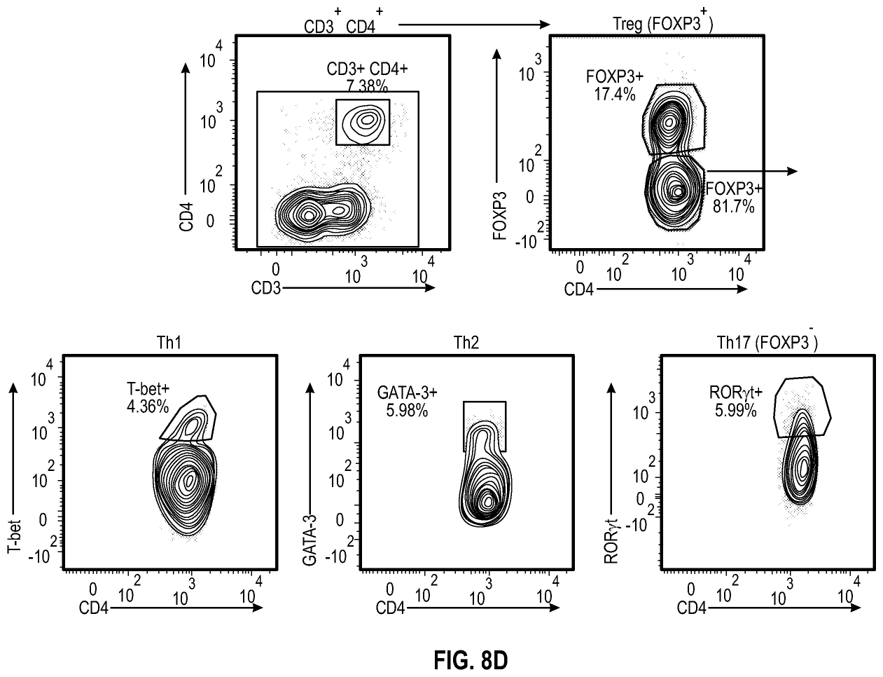

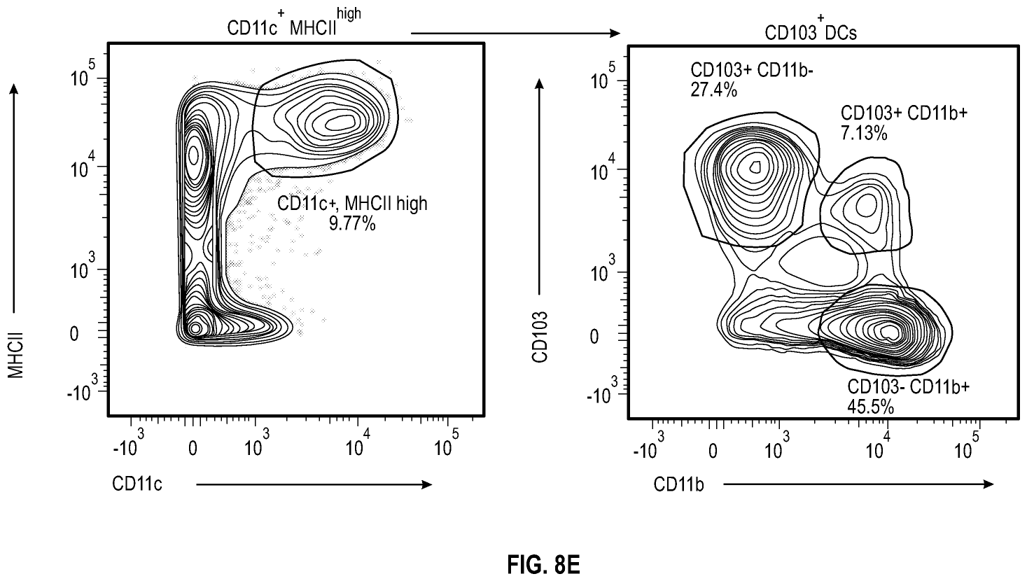

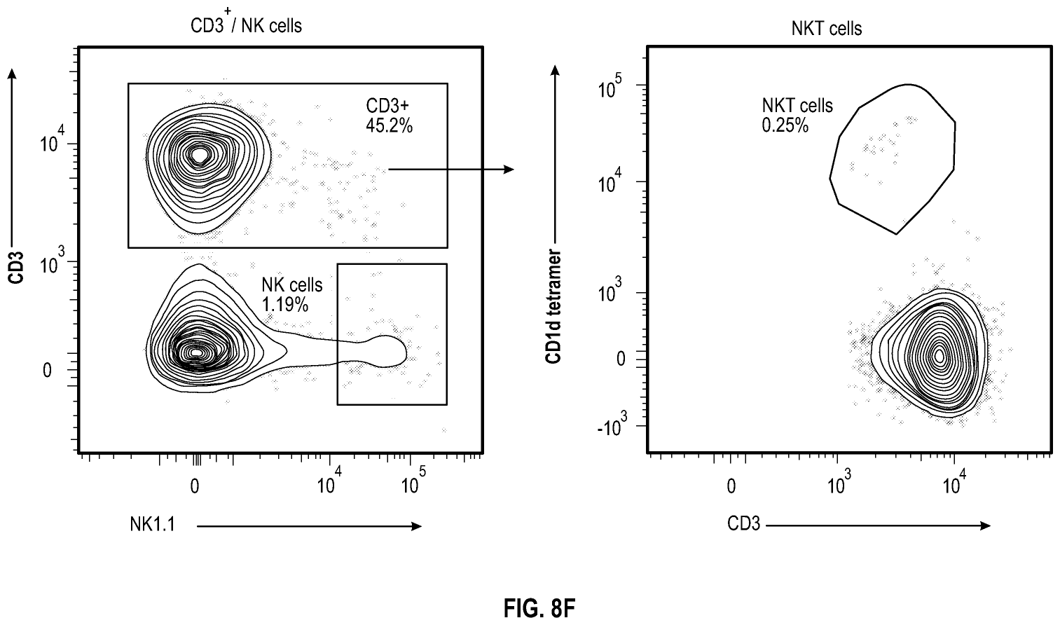

In some embodiments of any of the aspects, administration of the Mucispirillum composition promotes XCL1 secretion by NKT cells. In some embodiments of any of the aspects, the Mucispirillum composition increases NKT cell secretion of XCL1 by at least 100% (see e.g., FIG. 4F-4G, FIG. 17-18). In some embodiments of any of the aspects, the Mucispirillum composition increases NKT cell secretion of XCL1 by at least 10%, at least 20%, at least 30%, at least 40%, at least 50%, at least 60%, at least 70%, at least 80%, at least 90%, at least 100%, at least 150%, at least 200%, at least 300%, or at least 400%, or more, as to NKT cells not exposed to the composition. In some embodiments of any of the aspects, XCL1 secretion is measured using an XCL1-specific ELISA, e.g., R&D SYSTEMS MOUSE XCL1/LYMPHOTACTIN DUOSET ELISA KIT, ONESTEP MOUSE XCL1 ELISA KIT (ABCAM), ONESTEP HUMAN XCL1 ELISA (ABCAM). In some embodiments of any of the aspects, the NKT cells are measured and/or isolated using flow cytometry, such as by using CD1d-tetramer-binding CD3+ cells (see e.g., FIG. 8F, FIG. 30E for gating strategy of NKT cells). In some embodiments of any of the aspects, the NKT cells can be grown from a cell line (e.g., GW1 NKT cells).

In some embodiments of any of the aspects, administration of the Mucispirillum composition increases the number and/or activation of cDC1s by at least 50% (see e.g., FIG. 3G-3I, FIG. 27C-27F), e.g., in tumor draining lymph nodes. In some embodiments of any of the aspects, administration of the Mucispirillum composition increases the number and/or activation of cDC1s by at least 10%, at least 20%. at least 30%, at least 40%, at least 50%, at least 60%, at least 70%, at least 80%, at least 90%, at least 100%, at least 150%, at least 200%, at least 300%, or at least 400%, or more, as compared to a subject not administered the Mucispirillum composition. In some embodiments of any of the aspects, cDC1 number is measured by quantifying the number of CD103+CD1 b− cells from the population of MHCII+ CDC11c+ dendritic cells using flow cytometry (see e.g., FIG. 8E, FIG. 30D for gating strategy of CD103/CD11b expressing dendritic cells). In some embodiments of any of the aspects, cDC1 activation can be measured by quantifying the number of cDC1 cells that have migrated from the intestine (e.g., lamina propria) into a tumor-draining lymph node. In some embodiments of any of the aspects, cDC1 activation by XCL1 can be measured by quantification of XCR1 RNA or protein expression. Additional non-limiting examples of activation markers for cDC1s include Clec9a and Irf8.

In some embodiments of any of the aspects, administration of the Mucispirillum composition increases the number and/or activation of CD8+ T cells by at least 50%. In some embodiments of any of the aspects, administration of the Mucispirillum composition increases the number and/or activation of CD8+ T cells by at least 10%, at least 20%, at least 30%, at least 40%, at least 50%, at least 60%, at least 70%, at least 80%, at least 90%, at least 100%, at least 150%, at least 200%, at least 300%, or at least 400%, or more, as compared to a subject not administered the Mucispirillum composition. In some embodiments of any of the aspects, administration of the Mucispirillum composition increases CD8+ T cell infiltration in a colorectal tumor. In some embodiments of any of the aspects, the number of CD8+ T cells is quantified using flow cytometry in a tumor draining lymph node or a tumor. In some embodiments of any of the aspects, the activation of CD8+ T cells is quantified by measuring increased expression of effectors such as IFNγ or GZMB or by measuring decreased expression of immune-checkpoint receptors such as PD-1, LAG-3, TIM-3, or CTLA-4 (see e.g., FIG. 8B, FIG. 30B for gating strategy of CD8+ T-cells and their expression of immune-checkpoint receptors, and their expression of IFNγ and GZMB).

In some embodiments of any of the aspects, the method further comprises administering a sulfur amino acid (SAA). In some embodiments of any of the aspects, the sulfur amino acid (SAA) is administered at or above the recommended daily intake. The recommended daily intake for methionine is 10.4 mg per kilogram of body weight or 4.5 mg per pound. A person weighing 70 kg (˜154 pounds) should consume at least 728 mg of methionine per day. The recommended daily intake of cysteine is 4.1 mg per kilogram of body weight or 1.9 mg per pound. A person weighing 70 kg (˜154 pounds) should consume at least 287 mg of cysteine per day. In some embodiments of any of the aspects, administration of the SAA comprises administering a food naturally high in SAAs, a food supplemented with SAAs, a supplement comprising SAA(s), or a pharmaceutical composition comprising SAAs.

In some embodiments of any of the aspects, the method further comprises administering a food high in sulfur amino acids, such as a food that is naturally high in sulfur amino acids. Non-limiting examples of food naturally high in sulfur amino acids (e.g., methionine and/or cysteine) include: poultry such as turkey (e.g., ground turkey; e.g., 931 mg methionine per 100 g ground turkey, 128% recommended daily/dietary intake (RDI for methionine)) or chicken (e.g., 40-195% RDI for methionine; e.g., lean chicken breast; e.g., 336 mg cysteine per 100 g lean chicken breast; 117% RDI for cysteine); red meat such as beef (e.g., skirt steak; e.g., 931 mg methionine per 100 g skirt steak; 124% RDI for methionine; 345 mg cysteine per 100 g skirt steak; 120% RDI for cysteine), lamb, veal, or buffalo; fish or seafood such as tuna (e.g., 885 mg methionine per 100 g tuna; 122% RDI for methionine; e.g., 321 mg cysteine per 100 g tuna; 112% RDI for cysteine), grouper, salmon, snapper, tilapia, Mahi-mahi, or clams; pork such as pork chops (e.g., 850 mg methionine per 100 g pork chop; 117% RDI for methionine; 350 mg cysteine per 100 g pork chop; 122% RDI for cysteine), pork ribs, lean ham, pork bratwurst, ground pork, salami, roast ham, sausage links, or bacon; soy products such as firm tofu (e.g., 211 mg methionine per 100 g firm tofu; 29% RDI for methionine), boiled soybeans, cooked soybean sprouts, or soymilk; cow milk products such as cow milk (e.g., 88 mg methionine per 100 g milk; 12% RDI for methionine), yogurt (e.g., 52 mg cysteine per 100 g yogurt; 18% RDI for cysteine), or buttermilk; cheese products such as ricotta (e.g., 284 mg methionine per 100 g ricotta; 39% RDI for methionine), Swiss cheese (e.g., 290 mg cysteine per 100 g Swiss cheese; 133% 101 for cysteine), parmesan, gruyere, gouda, or fontina; nuts or seeds such as Brazil nuts (e.g., 1124 mg methionine per 100 g Brazil nuts; 154% RDI for methionine), sunflower seeds (e.g., 383 mg cysteine per 100 g sunflower seeds; 133% RDI for cysteine), hemp seeds, squash seeds, pumpkin seeds, chia seeds, sesame seeds, flax seeds, cashews, pistachios, or peanuts; beans such as large white beans (e.g., 146 mg methionine per 100 g large white beans; 20% RDI for methionine), lentils (e.g., 118 mg cysteine per 100 g lentils; 41% RDI for cysteine), navy beans, kidney beans, black beans, great northern beans, pinto beans, split peas; grains such as quinoa (e.g., 96 mg methionine per 100 g quinoa; 13% RDI for methionine), oatmeal (e.g., 97 mg cysteine per 100 g oatmeal; 34% RDI for cysteine), teff, wild rice, kamut, rice, or whole wheat pasta; or chicken eggs (e.g., 292 mg cysteine per 100 g eggs; 102% RDI for cysteine).

In some embodiments of any of the aspects, the method further comprises administering a food that has been supplemented with at least one sulfur amino acid. In some embodiments of any of the aspects, the food has been supplemented with at least 2.0 g, at least 5 g, at least 10 g, at least 20 g, at least 30 g, at least 40 g, at least 50 g, at least 60 g, at least 70 g, at least 80 g, at least 90 g, at least 100 g, at least 150 g, at least 200 g, at least 300 g, or at least 400 g SAA(s). In some embodiments of any of the aspects, the food has been supplemented with 2.0 g-400 g SAA(s), 2.0 g-300 g SAA(s), 2.0 g-200 g SAA(s), 2.0 g-100 g SAA(s), 10.0 g-400 g SAA(s), 10 g-100 g SAA(s), 50 g-400 g SAA(s), 100 g-400 g SAA(s), 200 g-400 g SAA(s), or 300 g-400 g SAA(s).

In some embodiments of any of the aspects, the method further comprises administering a supplement comprising at least one sulfur amino acid. In some embodiments of any of the aspects, the supplement comprises at least 2.0 g, at least 5 g, at least 10 g, at least 20 g, at least 30 g, at least 40 g, at least 50 g, at least 60 g, at least 70 g, at least 80 g, at least 90 g, at least 100 g, at least 150 g, at least 200 g, at least 300 g, or at least 400 g SAA(s). In some embodiments of any of the aspects, the supplement comprises 2.0 g-400 g SAA(s), 2.0 g-300 g SAA(s), 2.0 g-200 g SAA(s), 2.0 g-100 g SAA(s), 10.0 g-400 g SAA(s), 10 g-100 g SAA(s), 50 g-400 g SAA(s), 100 g-400 g SAA(s), 200 g-400 g SAA(s), or 300 g-400 g SAA(s). In some embodiments of any of the aspects, the sulfur amino acid is methionine, cysteine or a derivative thereof. In some embodiments of any of the aspects, the sulfur amino acid is methionine, cysteine, homocysteine, taurine or a derivative thereof. In some embodiments of any of the aspects, the composition comprises a combination of any one of methionine, cysteine, homocysteine, and/or taurine (see e.g., Table 9).

In some embodiments of any of the aspects, the method further comprises administering at least 2.0 g SAA(s) (e.g., methionine, cysteine, homocysteine, and/or taurine or a derivative thereof). In some embodiments of any of the aspects, the method further comprises administering at least 2.4 g SAA(s). In some embodiments of any of the aspects, the method further comprises administering at least 5 g, at least 10 g, at least 20 g, at least 30 g, at least 40 g, at least 50 g, at least 60 g, at least 70 g, at least 80 g, at least 90 g, at least 100 g, at least 150 g, at least 200 g, at least 300 g, or at least 400 g SAA(s). In some embodiments of any of the aspects, the method further comprises administering at most 5 g, at most 10 g, at most 20 g, at most 30 g, at least 40 g, at most 50 g, at most 60 g, at most 70 g, at most 80 g, at most 90 g, at most 100 g, at most 150 g, at most 200 g, at most 300 g, or at most 400 g SAA(s). In some embodiments of any of the aspects, the method further comprises administering 2.0 g-400 g SAA(s). In some embodiments of any of the aspects, the method further comprises administering 2.0 g-400 g SAA(s), 2.0 g-300 g SAA(s), 2.0 g-200 g SAA(s), 2.0 g-100 g SAA(s), 10.0 g-400 g SAA(s), 10 g-100 g SAA(s), 50 g-400 g SAA(s), 100 g-400 g SAA(s), 200 g-400 g SAA(s), or 300 g-400 g SAA(s).

In some embodiments of any of the aspects, the method comprises administering SAA(s) (e.g., in a food naturally high in SAAs, a food supplemented with SAAs, a supplement comprising SAA(s), or a pharmaceutical composition comprising SAAs) once a day, twice a day, three times a day (e.g., with meals), four times a day, five times a day or more over a period of 1 week, 2 weeks, 3 weeks, 4 weeks, 1 month, 2 months, 3 months, 4 months, 5 months, or 6 months, or more. In some embodiments of any of the aspects, administration of SAAs corresponds to the duration of cancer treatment. In some embodiments of any of the aspects, administration of SAAs is continued past the cessation of cancer treatment.

In some embodiments of any of the aspects, the method further comprises administering at least a second composition as described herein in addition to the Mucispirillum composition. In some embodiments of any of the aspects, the Mucispirillum composition is co-administered with a diet high in sulfur amino acids (SAA) (or a supplement comprising SAAs) and/or an XCL1 polypeptide (or XCR1 agonist). In some embodiments of any of the aspects, the Mucispirillum composition is co-administered with a diet high in sulfur amino acids (SAA) (or a supplement comprising SAAs). In some embodiments of any of the aspects, the Mucispirillum composition is co-administered with an XCL1 polypeptide (or XCR1 agonist). In some embodiments of any of the aspects, the Mucispirillum composition is co-administered with a diet high in sulfur amino acids (SAA) (or a supplement comprising SAAs) and an XCL1 polypeptide (or XCR1 agonist). See e.g., Table 10 for exemplary treatment combinations. In some embodiments of any of the aspects, the treatment combinations can be administered sequentially or concurrently.

TABLE 10
Exemplary treatment combinations (“x” indicates
inclusion in the treatment method)

| | high SAA | XCL1 | immune |
|---|---|---|---|
| Mucispirillum | diet (or SAA | polypeptide or | checkpoint |
| composition | supplement) | XCR1 agonist | inhibitor |
| X | | | |
| | X | | |
| X | X | | |
| | | X | |
| X | | X | |
| | X | X | |
| X | X | X | |
| | | | X |
| X | | | X |
| | X | | X |
| X | X | | X |
| | | X | X |
| X | | X | X |
| | X | X | X |
| X | X | X | X |

### SAA Diet or Supplementation Methods

In one aspect, described herein is a method of establishing or maintaining a tumor-suppressive gut environment in a subject in need thereof, the method comprising administering a diet high in sulfur amino acids (SAA) (or a supplement comprising SAAs). In one aspect, described herein is a method of establishing a tumor-suppressive gut environment in a subject in need thereof, the method comprising administering a diet high in sulfur amino acids (SAA) (or a supplement comprising SAAs). In one aspect, described herein is a method of maintaining a tumor-suppressive gut environment in a subject in need thereof, the method comprising administering a diet high in sulfur amino acids (SAA) (or a supplement comprising SAAs). In one aspect, described herein is a method of increasing CD8+ T cell infiltration in a colorectal tumor, the method comprising administering a diet high in sulfur amino acids (SAA) (or a supplement comprising SAAs) to a subject in need thereof. As used herein, the term “tumor-suppressive gut environment” refers to a gut environment (e.g., host gut cells, host immune cells, and/or associated gut microbiota) that is associated with tumor suppression; as described herein, increased gut levels of sulfur amino acids (SAA), Mucispirillum, XCL1, XCL1-expressing NKTs, and/or CD103+ conventional dendritic cells (cDC1) can be associated with tumor suppression (see e.g., FIG. 13). In some embodiments of any of the aspects, the method establishes or maintains a colon cancer tumor-suppressive gut environment in a subject in need thereof.

As used herein, the term “diet high in sulfur amino acids” refers to a diet comprising at or higher than the recommended daily intake (e.g., for a person weighing 70 kg, at least 728 mg of methionine per day and at least 287 mg of cysteine per day). In some embodiments of any of the aspects, the diet high in sulfur amino acids comprises a food naturally high in SAAs as described herein or a food supplemented with SAAs. As such, the diet high in sulfur amino acids includes not only a diet comprising increased amounts of SAAs as described herein, but also a diet in which one or more SAAs are provided as supplements. In this embodiment, SAAs can be provided as a supplement to a normal diet, or to augment a diet high or enriched in SAAs.

In some embodiments of any of the aspects, the method comprises administering a food that has been supplemented with at least one sulfur amino acid. In some embodiments of any of the aspects, the food has been supplemented with at least 2.0 g, at least 5 g, at least 10 g, at least 20 g, at least 30 g, at least 40 g, at least 50 g, at least 60 g, at least 70 g, at least 80 g, at least 90 g, at least 100 g, at least 150 g, at least 200 g, at least 300 g, or at least 400 g SAA(s). In some embodiments of any of the aspects, the food has been supplemented with 2.0 g-400 g SAA(s), 2.0 g-300 g SAA(s), 2.0 g-200 g SAA(s), 2.0 g-100 g SAA(s), 10.0 g-400 g SAA(s), 10 g-100 g SAA(s), 10 g-20 g SAA(s), 20 g-30 g SAA(s), 30 g-40 g SAA(s), 40 g-50 g SAA(s), 50 g-400 g SAA(s), 100 g-400 g SAA(s), 200 g-400 g SAA(s), or 300 g-400 g SAA(s). In some embodiments of any of the aspects, the food has been supplemented with about 15 g L-methionine and/or about 8 g L-cysteine. In some embodiments of any of the aspects, the food has been supplemented with about 15 g L-methionine and/or about 8 g L-cystine (see e.g., Table 2).

In some embodiments of any of the aspects, the method comprises administering a supplement comprising at least one sulfur amino acid. In some embodiments of any of the aspects, the supplement comprises at least 2.0 g, at least 5 g, at least 10 g, at least 20 g, at least 30 g, at least 40 g, at least 50 g, at least 60 g, at least 70 g, at least 80 g, at least 90 g, at least 100 g, at least 150 g, at least 200 g, at least 300 g, or at least 400 g SAA(s). In some embodiments of any of the aspects, the supplement comprises 2.0 g-400 g SAA(s), 2.0 g-300 g SAA(s), 2.0 g-200 g SAA(s), 2.0 g-100 g SAA(s), 10.0 g-400 g SAA(s), 10 g-100 g SAA(s), 10 g-20 g SAA(s), 20 g-30 g SAA(s), 30 g-40 g SAA(s), 40 g-50 g SAA(s), 50 g-400 g SAA(s), 100 g-400 g SAA(s), 200 g-400 g SAA(s), or 300 g-400 g SAA(s). In some embodiments of any of the aspects, the supplement comprises about 15 g L-methionine and/or about 8 g L-cysteine. In some embodiments of any of the aspects, the supplement comprises about 15 g L-methionine and/or about 8 g L-cystine (see e.g., Table 2).

In some embodiments of any of the aspects, the diet high in sulfur amino acids (or a supplement comprising SAAs) comprises elevated levels of methionine, cysteine or a derivative thereof relative to a diet that is not high in sulfur amino acids or relative to a normal or typical diet as described herein. In some embodiments of any of the aspects, the diet high in sulfur amino acids (or a supplement comprising SAAs) comprises elevated levels of methionine, cysteine, homocysteine, taurine or a derivative thereof relative to a diet that is not high in sulfur amino acids. In some embodiments of any of the aspects, the diet high in sulfur amino acids (or a supplement comprising SAAs) comprises elevated levels of a combination of any one of methionine, cysteine, homocysteine, and/or taurine (see e.g., Table 9).

In some embodiments of any of the aspects, a diet low in sulfur amino acids comprises 0.01 grams to 0.04 grams of SAA per kilogram body weight (of the subject) per day. In some embodiments of any of the aspects, the diet high in sulfur amino acids comprises greater than 0.04 grams of SAA per kilogram body weight (of the subject) per day. In some embodiments of any of the aspects, the diet high in sulfur amino acids comprises at least 6 grams of SAA per kilogram body weight per day.

In some embodiments of any of the aspects, the diet high in sulfur amino acids comprises at least 0.04 g, at least 0.05 g, at least 0.06 g, at least 0.07 g, at least 0.08 g, at least 0.09 g, at least 0.1 g, at least 0.2 g, at least 0.3 g, at least 0.4 g, at least 0.5 g, at least 0.6 g, at least 0.7 g. at least 0.8 g. at least 0.9 g, at least 1 g, at least 2 g, at least 3 g, at least 4 g, at least 5 g, at least 6 g or more of SAA per kilogram body weight (of the subject) per day. In some embodiments of any of the aspects, the diet high in sulfur amino acids comprises at most 0.05 g, at most 0.06 g, at most 0.07 g, at most 0.08 g, at most 0.09 g, at most 0.1 g, at most 0.2 g, at most 0.3 g, at most 0.4 g, at most 0.5 g, at most 0.6 g, at most 0.7 g, at most 0.8 g, at most 0.9 g, at most 1 g, at most 2 g, at most 3 g, at most 4 g, at most 5 g, at most 6 g of SAA per kilogram body weight (of the subject) per day. In some embodiments of any of the aspects, the diet high in sulfur amino acids comprises 0.04 g-0.1 g, 0.1 g-1.0 g, 1.0 g-6.0 g, 0.04 g-1.0 g, 0.04 g-6.0 g, or 0.1 g-6.0 g of SAA per kilogram body weight (of the subject) per day.

Assuming an average body mass of 60 kg, in some embodiments of any of the aspects, the diet high in SAAs comprises greater than 2.4 grams of SAA per day. In some embodiments of any of the aspects, the diet high in SAAs comprises at least 5 g, at least 10 g, at least 20 g, at least 30 g, at least 40 g, at least 50 g, at least 60 g, at least 70 g, at least 80 g, at least 90 g, at least 100 g, at least 150 g, at least 200 g, at least 300 g, or at least 400 g SAA(s) per day. In some embodiments of any of the aspects, the diet high in SAAs comprises at most 5 g, at most 10 g, at most 20 g, at most 30 g, at least 40 g, at most 50 g, at most 60 g, at most 70 g, at most 80 g, at most 90 g, at most 100 g, at most 150 g, at most 200 g, at most 300 g, or at most 400 g SAA(s) per day. In some embodiments of any of the aspects, the diet high in SAAs comprises 2.0 g-400 g SAA(s) per day. In some embodiments of any of the aspects, the diet high in SAAs comprises 2.0 g-400 g SAA(s), 2.0 g-300 g SAA(s), 2.0 g-200 g SAA(s), 2.0 g-100 g SAA(s), 10.0 g-400 g SAA(s), 10 g-100 g SAA(s), 50 g-400 g SAA(s), 100 g-400 g SAA(s), 200 g-400 g SAA(s), or 300 g-400 g SAA(s) per day. In some embodiments of any of the aspects, the method further comprises administering at least a second treatment as described herein in addition to the diet high in sulfur amino acids (SAA) (see e.g., Table 10).

In some embodiments of any of the aspects, administration of the diet high in sulfur amino acids (or a supplement comprising SAAs) promotes XCL1 secretion by NKT cells. In some embodiments of any of the aspects, the diet high in sulfur amino acids increases NKT cell secretion of XCL1 by at least 100%. In some embodiments of any of the aspects, the diet high in sulfur amino acids increases NKT cell secretion of XCL1 by at least 10%, at least 20%, at least 30%, at least 40%, at least 50%, at least 60%, at least 70%, at least 80%, at least 90%, at least 100%, at least 150%, at least 200%, at least 300%, or at least 400%, or more, as compared to NKT cell secretion of XCL1 in a subject not administered the diet high in sulfur amino acids.

In some embodiments of any of the aspects, administration of the diet high in sulfur amino acids (or a supplement comprising SAAs) increases the number and/or activation of cDC1s by at least 25%, e.g., in tumor draining lymph nodes (see e.g., FIG. 3G-3I, FIG. 27C-27F). In some embodiments of any of the aspects, administration of the diet high in sulfur amino acids increases the number and/or activation of cDC1s by at least 5%, at least 10%, at least 15%, at least 20%, at least 25%, at least 30%, at least 35%, at least 40%, at least 50%, at least 60%, at least 70%, at least 80%, at least 90%, at least 100%, at least 150%, at least 200%, at least 300%, or at least 400%, or more, as compared to a subject not administered the diet high in sulfur amino acids. In some embodiments of any of the aspects, cDC1 number is measured by quantifying the number of CD103+CD11b− cells from the population of MHCII+ CDC11c+ dendritic cells using flow cytometry (see e.g., FIG. 8E, FIG. 30D for gating strategy of CD103/CD11b expressing dendritic cells). In some embodiments of any of the aspects, cDC1 activation can be measured by quantifying the number of cDC1 cells that have migrated from the intestine (e.g., lamina propria) into a tumor-draining lymph node. In some embodiments of any of the aspects, cDC1 activation by XCL1 can be measured by quantification of XCR1 RNA or protein expression. Additional non-limiting examples of activation markers for cDC1s include Clec9a and Irf8.

In some embodiments of any of the aspects, administration of the diet high in sulfur amino acids (or a supplement comprising SAAs) increases the number and/or activation of CD8+ T cells by at least 50% (see e.g., FIG. 3B-3E). In some embodiments of any of the aspects, administration of the diet high in sulfur amino acids increases the number and/or activation of CD8+ T cells by at least 10%, at least 20%, at least 30%, at least 40%, at least 50%, at least 60%, at least 70%, at least 80%, at least 90%, at least 100%, at least 150%, at least 200%, at least 300%, or at least 400%, or more, as compared to a subject not administered the diet high in sulfur amino acids. In some embodiments of any of the aspects, the number of CD8+ T cells is quantified using flow cytometry in a tumor draining lymph node or a tumor. In some embodiments of any of the aspects, the activation of CD8+ T cells is quantified by measuring increased expression of effectors such as IFNγ or GZMB or by measuring decreased expression of immune-checkpoint receptors such as PD-1, LAG-3, TIM-3, or CTLA-4 (see e.g., FIG. 8B, FIG. 30B for gating strategy of CD8+ T-cells and their expression of immune-checkpoint receptors, and their expression of IFNγ and GZMB).

### XCL1 Polypeptide or XCR1 Agonist Methods

In one aspect, described herein is a method of treating cancer, the method comprising administering to a subject in need thereof an XCL1 polypeptide (see e.g., SEQ ID NOs: 5-6). In one aspect, described herein is a method of treating cancer, the method comprising administering to a subject in need thereof an XCR1 agonist. In one aspect, described herein is a method of treating cancer, the method comprising administering to a subject in need thereof an XCL1 polypeptide and an XCR1 agonist. In one aspect, described herein is a method of treating cancer, the method comprising administering to a subject in need thereof a microorganism engineered to express XCL1 polypeptide. In some embodiments of any of the aspects, the cancer is colon cancer.

X-C Motif Chemokine Receptor 1 (XCR1) is the receptor for XCL1 and XCL2 (lymphotactin-1 and lymphotactin-2, respectively). XCR1 can also be referred to as CCXCR1 or G Protein-Coupled Receptor (GPR5). XCR1 is a chemokine receptor belonging to the G protein-coupled receptor superfamily. The family members are characterized by the presence of 7 transmembrane domains and numerous conserved amino acids. XCR1 can be expressed on dendritic cells, such as cDC1 cells. Cross-presenting dendritic cells (DCs) in the spleen develop into XCR1+ DCs in the small intestine, T cell zones of Peyer's patches, and T cell zones and sinuses of mesenteric lymph nodes. XCR1+ DCs specialize in cross-presentations of orally applied antigens.

In some embodiments of any of the aspects, XCR1 comprises one of SEQ ID NO: 7 or SEQ ID NO: 8 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to one of SEQ ID NO: 7 or SEQ ID NO: 8 that maintains its function (e.g., binding to XCL1 and/or XCR1-associated intracellular signaling).

chemokine XC receptor 1 Homo sapiens, NCBI
Reference Sequence: NP_001019815.1, 333 aa
SEQ ID NO: 7
MESSGNPESTTFFYYDLQSQPCENQAWVFATLATTVLYCLVFLLSLVGNS
LVLWVLVKYESLESLTNIFILNLCLSDLVFACLLPVWISPYHWGWVLGDF
LCKLLNMIFSISLYSSIFFLTIMTIHRYLSVVSPLSTLRVPTLRCRVLVT
MAVWVASILSSILDTIFHKVLSSGCDYSELTWYLTSVYQHNLFFLLSLGI
ILFCYVEILRTLFRSRSKRRHRTVKLIFAIVVAYFLSWGPYNFTLFLQTL
FRTQIIRSCEAKQQLEYALLICRNLAFSHCCFNPVLYVFVGVKFRTHLKH
VLRQFWFCRLQAPSPASIPHSPGAFAYEGASFY,
chemokine XC receptor 1 Mus musculus, NCBI
Reference Sequence: NP_035928.2, 338 aa
SEQ ID NO: 8
MDSESDALSIPASRVQMESSTAFYDYHDKLSLLCENNVIFFSTISTIVLY
SLVFLLSLVGNSLVLWVLVKYENLESLTNIFILNLCLSDLMFSCLLPVLI
SAQWSWFLGDFFCKFFNMIFGISLYSSIFFLTIMTIHRYLSVVSPISTLG
IHTLRCRVLVTSCVWAASILFSIPDAVFHKVISLNCKYSEHHGFLASVYQ
HNIFFLLSMGIILFCYVQILRTLFRTRSRQRHRTVRLIFTVVVAYFLSWA
PYNLTLFLKTGIIQQSCESLQQLDIAMIICRHLAFSHCCFNPVLYVFVGI
KFRRHLKHLFQQVWLCRKTSSTVPCSPGTFTYEGPSFY,

In some embodiments of any of the aspects, the XCR1 agonist is a functional variant of XCL1, e.g., human XCL1 or Mus musculus XCL1. In some embodiments of any of the aspects, the XCR1 agonist is a functional variant of XCL1 that further comprises at least one additional disulfide bridge (e.g., at least 2 residues mutated to cysteine). In some embodiments of any of the aspects, the XCR1 agonist is a functional variant of XCL1 (e.g., SEQ ID NO: 5, SEQ ID NO: 6, residues 22-114 of SEQ ID NO: 5, residues 22-93 of SEQ ID NO: 5, residues 22-114 of SEQ ID NO: 6) that comprises at least one of the following mutations: V21C, A59C, V59C, T10C mutation, and/or addition of the “AC” dipeptide at residue 32.

In some embodiments of any of the aspects, the XCR1 agonist comprises an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to one of SEQ ID NO: 5, SEQ ID NO: 6, residues 22-114 of SEQ ID NO: 5, residues 22-93 of SEQ ID NO: 5, residues 22-114 of SEQ ID NO: 6, or residues 22-93 of SEQ ID NO: 6, that maintains its function (e.g., binding to and/or activation of XCR1).

In some embodiments of any of the aspects, the XCR1 agonist is mXCL1-V21C/A59C, which is a highly active form of mXCL1 comprising V21C and A59C mutations (see e.g., SEQ ID NO: 9). In some embodiments of any of the aspects, the XCR1 agonist is CC1 Ltn or CC3 Ltn, which comprise at least one additional disulfide bond in hXCL to restrict XCL1 to a chemokine-like conformation with XCR1 agonist activity (see e.g., SEQ ID NOs: 10-11). In some embodiments of any of the aspects, the XCR1 agonist is selected from the group consisting of mXCL1-V21C/A59C, CC1 Ltn, and CC3 Ltn; see e.g., Matsuo et al., Front Immunol. 2018, 9: 2775; Tuinstra et al., Biochemistry 2007, 46(10): 2564-73; the contents of each of which are incorporated herein by reference in their entireties. In some embodiments of any of the aspects, the XCR1 agonist is selected from the group consisting of SEQ ID NOs: 9-11.

In some embodiments of any of the aspects, the XCR1 agonist comprises one of SEQ ID NOs: 9-11 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to one of SEQ ID NOs: 9-11 that maintains its function (e.g., binding to and/or activation of XCR1).

mXCL1-V21C/A59C, 93 aa, bold underlined
text indicates the V21C and A59C mutations
compared to residues 22-114 of mXCLI (see
e.g., SEQ ID NO: 6)
SEQ ID NO: 9
VGTEVLEESSCVNLQTQRLP [symbol] QKIKTYIIWEGAMRAVIFVTKRGLKICAD
PEAKWVKA [symbol] IKTVDGRASTRKNMAETVPTGAQRSTSTAVTLTG,
CC1 Ltn, 95 aa, bold underlined text
indicates the T10C mutation and addition
of the “AC” dipeptide at residue 32,
compared to residues 22-114 of hXCLI
(see e.g., SEQ ID NO: 5)
SEQ ID NO: 10
VGSEVSDKR [symbol] CVSLTTQRLPVSRIKTYTITEG [symbol] SLRAVIFITKRGLKVC
ADPQATWVRDVVRSMDRKSNTRNNMIQTKPTGTQQSTNTAVTLTG,
CC3 Ltn, 93 aa, bold underlined text
indicates the V21C and V59C mutations,
compared to residues 22-114 of hXCL1
(see e.g., SEQ ID NO: 5)
SEQ ID NO: 11
VGSEVSDKRTCVSLTTQRLP [symbol] SRIKTYTITEGSLRAVIFITKRGLKVCAD
PQATWVRD [symbol] VRSMDRKSNTRNNMIQTKPTGTQQSTNTAVTLTG,

In some embodiments of any of the aspects, the XCL1 polypeptide is administered to the gut. In some embodiments of any of the aspects, the XCR1 agonist is administered to the gut. In some embodiments of any of the aspects, the XCL1 polypeptide is administered using a bacterium (e.g., commensal gut bacteria; e.g., Lactobacillus) engineered to express XCL1 polypeptide and/or an XCR1 agonist. In some embodiments of any of the aspects, the method further comprises administering at least a second composition as described herein in addition to the XCL1 polypeptide or the XCR1 agonist (see e.g., Table 10).

In some embodiments of any of the aspects, administration of the XCL1 polypeptide or the XCR1 agonist increases the number and/or activation of cDC1s by at least 50%, e.g., in tumor draining lymph nodes. In some embodiments of any of the aspects, administration of the XCL1 polypeptide or the XCR1 agonist increases the number and/or activation of cDC1s by at least 10%, at least 20%, at least 30%, at least 40%, at least 50%, at least 60%, at least 70%, at least 80%, at least 90%, at least 100%, at least 150%, at least 200%, at least 300%, or at least 400%, or more, as compared to a subject not administered the XCL1 polypeptide or the XCR1 agonist. In some embodiments of any of the aspects, cDC1 number is measured by quantifying the number of CD103+CD11b− cells from the population of MHCII+ CDC11c+ dendritic cells using flow cytometry (see e.g., FIG. 8E, FIG. 30D for gating strategy of CD103/CD11b expressing dendritic cells). In some embodiments of any of the aspects, cDC1 activation can be measured by quantifying the number of cDC1 cells that have migrated from the intestine (e.g., lamina propria) into a tumor-draining lymph node. In some embodiments of any of the aspects, cDC1 activation by XCL1 can be measured by quantification of XCR1 RNA or protein expression. Additional non-limiting examples of activation markers for cDC1s include Clec9a and Irf8.

In some embodiments of any of the aspects, administration of the XCL1 polypeptide or the XCR1 agonist increases the number and/or activation of CD8+ T cells by at least 50%. In some embodiments of any of the aspects, administration of the XCL1 polypeptide or the XCR1 agonist increases the number and/or activation of CD8+ T cells by at least 10%, at least 20%, at least 30%, at least 40%, at least 50%, at least 60%, at least 70%, at least 80%, at least 90%, at least 100%, at least 150%, at least 200%, at least 300%, or at least 400%, or more, as compared to a subject not administered the XCL1 polypeptide or the XCR1 agonist. In some embodiments of any of the aspects, the number of CD8+ T cells is quantified using flow cytometry in a tumor draining lymph node or a tumor. In some embodiments of any of the aspects, the activation of CD8+ T cells is quantified by measuring increased expression of effectors such as IFNγ or GZMB or by measuring decreased expression of immune-checkpoint receptors such as PD-1, LAG-3, TIM-3, or CTLA-4 (see e.g., FIG. 8B, FIG. 30B for gating strategy of CD8+ T-cells and their expression of immune-checkpoint receptors, and their expression of IFNγ and GZMB).

### Treatment Stratification Methods

In multiple aspects, described herein are methods of treatment stratification related to detection of M. schaedleri. In one aspect, described herein is a method of treating cancer in a subject in need thereof, the method comprising: detecting the level of M. schaedleri in a sample from the subject; administering a cancer immunotherapeutic agent if the level of M. schaedleri is at or above a pre-determined threshold; and administering a cancer immunotherapeutic agent and a Mucispirillum composition as described herein if the level of M. schaedleri is below a pre-determined threshold.

In one aspect, described herein is a method of treating cancer in a subject in need thereof, the method comprising: obtaining results from an assay detecting the level of M. schaedleri in a sample from the subject; administering a cancer immunotherapeutic agent if the level of M. schaedleri is at or above a pre-determined threshold; and administering a cancer immunotherapeutic agent and a Mucispirillum composition as described herein if the level of M. schaedleri is below a pre-determined threshold.

In one aspect, described herein is a method of treating cancer in a subject in need thereof, the method comprising: detecting the level of XCL1 polypeptide, NKTs, and/or CD103+ cDC1s in a sample from the subject; administering a cancer immunotherapeutic agent if the level of XCL1 polypeptide, NKTs, and/or CD103+ cDC1s is at or above a pre-determined threshold; and administering a cancer immunotherapeutic agent and a Mucispirillum composition as described herein if the level of XCL1 polypeptide, NKTs, and/or CD103+ cDC1s is below a pre-determined threshold.

In one aspect, described herein is a method of treating cancer in a subject in need thereof, the method comprising: obtaining results from an assay detecting the level of XCL1 polypeptide, NKTs, and/or CD103+ cDC1s in a sample from the subject; administering a cancer immunotherapeutic agent if the level of XCL1 polypeptide, NKTs, and/or CD103+ cDC1s is at or above a pre-determined threshold; and administering a cancer immunotherapeutic agent and a Mucispirillum composition as described herein if the level of XCL1 polypeptide, NKTs, and/or CD103+ cDC1s is below a pre-determined threshold.

In one aspect, described herein is a method of stratifying a subject for cancer treatment, the method comprising: detecting the level of M. schaedleri in a sample from the subject; and classifying the subject as high risk (e.g., of colorectal cancer symptoms or complications) if the level of M. schaedleri is below a pre-determined threshold; or classifying the subject as low risk (e.g., of colorectal cancer symptoms or complications) if the level of M. schaedleri is at or above a pre-determined threshold.

In one aspect, described herein is a method of stratifying a subject for cancer treatment, the method comprising: obtaining results from an assay detecting the level of M. schaedleri in a sample from the subject; and classifying the subject as high risk (e.g., of colorectal cancer symptoms or complications) if the level of M. schaedleri is below a pre-determined threshold; or classifying the subject as low risk (e.g., of colorectal cancer symptoms or complications) if the level of M. schaedleri is at or above a pre-determined threshold.

In one aspect, described herein is a method of stratifying a subject for cancer treatment, the method comprising: detecting the level of XCL1 polypeptide, NKTs, and/or CD103+ cDC1s in a sample from the subject; and classifying the subject as high risk (e.g., of colorectal cancer symptoms or complications) if the level of XCL1 polypeptide, NKTs, and/or CD103+ cDC1s is below a pre-determined threshold; or classifying the subject as low risk (e.g., of colorectal cancer symptoms or complications) if the level of XCL1 polypeptide, NKTs, and/or CD103+ cDC1s is at or above a pre-determined threshold.

In one aspect, described herein is a method of stratifying a subject for cancer treatment, the method comprising: obtaining results from an assay detecting the level of XCL1 polypeptide, NKTs, and/or CD103+ cDC1s in a sample from the subject; and classifying the subject as high risk (e.g., of colorectal cancer symptoms or complications) if the level of XCL1 polypeptide, NKTs, and/or CD103+ cDC1s is below a pre-determined threshold; or classifying the subject as low risk (e.g., of colorectal cancer symptoms or complications) if the level of XCL1 polypeptide, NKTs, and/or CD103+ cDC1s is at or above a pre-determined threshold.

In some embodiments of any of the aspects, the subject has colon cancer. In some embodiments of any of the aspects, the stratification method further comprises administering a Mucispirillum composition as described herein. In some embodiments of any of the aspects, the stratification method further comprises administering a sulfur amino acid. In some embodiments of any of the aspects, the stratification method further comprises administering a diet high in sulfur amino acids. In some embodiments of any of the aspects, the stratification method further comprises administering a Mucispirillum composition as described herein, a diet high in sulfur amino acids (SAA), and/or an XCL1 polypeptide (or XCR1 agonist), or any combinations thereof (see e.g., Table 10).

In some embodiments of any of the aspects, the cancer immunotherapeutic agent comprises an immune checkpoint inhibitor. In some embodiments of any of the aspects, the stratification method further comprises administering an immune checkpoint inhibitor, non-limiting examples of which are provided herein. In some embodiments of any of the aspects, the cancer immunotherapeutic agent is selected from the group consisting of an immune checkpoint inhibitor; chemotherapy; a dendritic cell vaccine; chimeric antigen receptor T cells (CAR-T); and NKT cell-based therapies. See e.g., Nelson, et al. Cancers vol. 13, 20 5174. 15 Oct. 2021, the contents of which are incorporated herein by reference in their entirety.

In some embodiments of any of the aspects, the treatment(s) described herein (e.g., a Mucispirillum composition as described herein, a diet high in sulfur amino acids (SAA), and/or an XCL1 polypeptide (or XCR1 agonist)) is administered as a monotherapy, e.g., another treatment for the cancer is not administered to the subject. In some embodiments of any of the aspects, the methods described herein can further comprise administering a second agent and/or treatment to the subject, e.g. as part of a combinatorial therapy.

Non-limiting examples of a second agent and/or treatment can include a cancer therapy selected from the group consisting of radiation therapy, surgery, gemcitabine, cisplatin, paclitaxel, carboplatin, bortezomib, AMG479, vorinostat, rituximab, temozolomide, rapamycin, ABT-737, PI-103; alkylating agents such as thiotepa and CYTOXAN® cyclophosphamide; alkyl sulfonates such as busulfan, improsulfan and piposulfan; aziridines such as benzodopa, carboquone, meturedopa, and uredopa; ethylenimines and methylmelamines including altretamine, triethylenemelamine, trietylenephosphoramide, triethylenethiophosphoramide and trimethylol melamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomycins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, ADRIAMYCIN® doxonubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulene Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylomithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Phanmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003).

In addition, the methods of treatment can further include the use of radiation or radiation therapy. Further, the methods of treatment can further include the use of surgical treatments.

In some embodiments of any of the aspects, the level of M. schaedleri is quantified using a standard detection method for bacteria, including but not limited to quantitative 16S sequencing, serially diluted plates assays, direct counting by optical microscopy chambers, and the like. In some embodiments of any of the aspects, the level of XCL1 polypeptide is quantified using a standard detection method for polypeptides, including but not limited to ELISA (enzyme linked immunosorbent assay), western blot, immunoprecipitation, or immunofluorescence using detection reagents such as an antibody or protein binding agents. In some embodiments of any of the aspects, the level of NKTs and/or CD103+ cDC1s is quantified using a standard detection method for immune cells, including but not limited to flow cytometry on blood or tissue samples or laser capture microdissection, immunohistochemistry, or immunofluorescence on tissue samples.

In some embodiments of any of the aspects, the stratification method results in higher treatment efficacy compared to a method of treating without first detecting the level of M. schaedleri, obtaining results from an assay detecting the level of M. schaedleri, detecting the level of XCL1 polypeptide, NKTs, and/or CD103+ cDC1s or obtaining results from an assay detecting the level of XCL1 polypeptide, NKTs, and/or CD103+ cDC1s.

In some embodiments of any of the aspects, the stratification method results in treatment efficacy that is increased by at least 5%, at least 10%, at least 15%, at least 20%, at least 30%, at least 40%, at least 50%, at least 60%, at least 70%, at least 80%, at least 90%, at least 100%, at least 150%, at least 200%, at least 300%, or at least 400%, or more, as compared to a method of treating without first detecting the level of M. schaedleri, obtaining results from an assay detecting the level of M. schaedleri, detecting the level of XCL1 polypeptide, NKTs, and/or CD103+ cDC1s or obtaining results from an assay detecting the level of XCL1 polypeptide, NKTs, and/or CD103+ cDC1s.

In some embodiments of any of the aspects, the stratification method results in higher treatment efficacy compared to a method of treating without first stratifying the subject. In some embodiments of any of the aspects, the stratification method results in a treatment efficacy that is increased by at least 5%, at least 10%, at least 15%, at least 20%, at least 30%, at least 40%, at least 50%, at least 60%, at least 70%, at least 80%, at least 90%, at least 100%, at least 150%, at least 200%, at least 300%, or at least 400%, or more, as compared to compared to a method of treating without first stratifying the subject.

In some embodiments of any of the aspects, the stratification method results in lower treatment complications compared to a method of treating without first detecting the level of M. schaedleri, obtaining results from an assay detecting the level of M. schaedleri, detecting the level of XCL1 polypeptide, NKTs, and/or CD103+ cDC1s or obtaining results from an assay detecting the level of XCL1 polypeptide, NKTs, and/or CD103+ cDC1s.

In some embodiments of any of the aspects, the stratification method results in treatment complications that are decreased by at least 5%, at least 10%, at least 15%, at least 20%, at least 30%, at least 40%, at least 50%, at least 60%, at least 70%, at least 80%, at least 90%, at least 100%, at least 150%, at least 200%, at least 300%, or at least 400%, or more, as compared to a method of treating without first: detecting the level of M. schaedleri; obtaining results from an assay detecting the level of M. schaedleri; detecting the level of XCL1 polypeptide, NKTs, and/or CD103+ cDC1s; or obtaining results from an assay detecting the level of XCL1 polypeptide, NKTs, and/or CD103+ cDC1s.

In some embodiments of any of the aspects, the stratification method results in lower treatment complications compared to a method of treating without first stratifying the subject. In some embodiments of any of the aspects, the stratification method results in treatment complications that are decreased by at least 5%, at least 10%, at least 15%, at least 20%, at least 30%, at least 40%, at least 50%, at least 60%, at least 70%, at least 80%, at least 90%, at least 100%, at least 150%, at least 200%, at least 300%, or at least 400%, or more, as compared to a method of treating without first stratifying the subject.

Non-limiting examples of cancer treatment (e.g., chemotherapy, radiation) complications, which can be decreased using the treatment or stratification methods as described herein, include: anemia; appetite loss; bleeding and bruising (e.g., thrombocytopenia); constipation; delirium; diarrhea; edema; fatigue; fertility issues in boys and men; fertility issues in girls and women; flu-like symptoms; hair loss (e.g., alopecia); infection and neutropenia; lymphedema; memory or concentration problems; mouth and throat problems; nausea and vomiting; nerve problems (e.g., peripheral neuropathy); immunotherapy and organ-related inflammation; pain; sexual health issues in men; sexual health issues in women; skin and nail changes; sleep problems; or urinary and bladder problems.

### Administration

In some embodiments, the methods described herein relate to treating a subject having or diagnosed as having cancer with a Mucispirillum composition as described herein, a diet high in sulfur amino acids (SAA), and/or an XCL1 polypeptide (or XCR1 agonist). Subjects having cancer can be identified by a physician using current methods of diagnosing cancer.

Symptoms and/or complications of cancer which characterize these conditions and aid in diagnosis are well known in the art and include but are not limited to, fatigue or extreme tiredness that does not get better with rest; weight loss or gain of 10 pounds or more for no known reason; eating problems such as not feeling hungry; trouble swallowing, belly pain, or nausea and vomiting; swelling or lumps anywhere in the body; thickening or lump in the breast or other part of the body; pain, especially new or with no known reason; that does not go away or gets worse; skin changes such as a lump that bleeds or turns scaly, a new mole or a change in a mole, a sore that does not heal, or a yellowish color to the skin or eyes (e.g., jaundice); cough or hoarseness that does not go away; unusual bleeding or bruising for no known reason; change in bowel habits, such as constipation or diarrhea, that does not go away or a change in how stools appear; bladder changes such as pain when passing urine, blood in the urine or needing to pass urine more or less often; fever or nights sweats; headaches; vision or hearing problems; mouth changes such as sores, bleeding, pain, or numbness.

Symptoms and/or complications of colorectal cancer which characterize these conditions and aid in diagnosis are well known in the art and include but are not limited to, a persistent change in bowel habits, including diarrhea or constipation or a change in the consistency of stool; rectal bleeding or blood in stool; persistent abdominal discomfort, such as cramps, gas or pain; a feeling that the bowel doesn't empty completely; weakness or fatigue; or unexplained weight loss.

Tests that may aid in a diagnosis of, e.g. colorectal cancer include, but are not limited to, colonoscopy, proctoscopy, colon or rectum biopsy, stool tests (e.g., Cologuard®), genetic testing (e.g., for changes in the KRAS, NRAS, or BRAF genes; microsatellite instability (MSI); changes in any of the mismatch repair (MMR) genes (MLH1, MSH2, MSH6, and PMS2); changes in the EPCAM gene), CT-guided needle biopsy, ultrasound, MRI, PET scan, A family history of colorectal cancer, or exposure to risk factors for colorectal cancer (e.g. lack of regular physical activity; a diet low in fruit and vegetables; a low-fiber and high-fat diet, or a diet high in processed meats; overweight and obesity; alcohol consumption; or tobacco use) can also aid in determining if a subject is likely to have colorectal cancer or in making a diagnosis of colorectal cancer.

The compositions and methods described herein can be administered to a subject having or diagnosed as having cancer (e.g., colorectal cancer, which is also referred to herein as colon cancer). In some embodiments, the methods described herein comprise administering an effective amount of compositions described herein, e.g. a Mucispirillum composition as described herein, a diet high in sulfur amino acids (SAA), and/or an XCL1 polypeptide (or XCR1 agonist) to a subject in order to alleviate a symptom of a cancer (e.g., colorectal cancer). As used herein, “alleviating a symptom of a cancer” is ameliorating any condition or symptom associated with the cancer (e.g., colorectal cancer). As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection. or intratumoral administration. Administration can be local or systemic.

The term “effective amount” as used herein refers to the amount of a Mucispirillum composition as described herein, a diet high in sulfur amino acids (SAA), and/or an XCL1 polypeptide (or XCR1 agonist) needed to alleviate at least one or more symptom of the disease or disorder, and relates to a sufficient amount of pharmacological composition to provide the desired effect. The term “therapeutically effective amount” therefore refers to an amount of a Mucispirillum composition as described herein, a diet high in sulfur amino acids (SAA), and/or an XCL1 polypeptide (or XCR1 agonist) that is sufficient to provide a particular anti-cancer effect when administered to atypical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of a Mucispirillum composition as described herein, a diet high in sulfur amino acids (SAA), and/or an XCL1 polypeptide (or XCR1 agonist), which achieves a half-maximal inhibition of symptoms) as determined in cell culture, or in an appropriate animal model. Levels in plasma or in the gut can be measured, for example, by high performance liquid chromatography. The effects of any particular dosage can be monitored by a suitable bioassay, e.g., assay for the level of Mucispirillum bacteria, XCL1 polypeptide, NKTs, and/or CD103+ cDC1s, among others. The dosage can be determined by a physician and adjusted, as necessary, to suit observed effects of the treatment.

Pharmaceutical compositions comprising a Mucispirillum composition as described herein, sulfur amino acids (SAA), and/or an XCL1 polypeptide (or XCR1 agonist) can also be formulated to be suitable for oral administration, for example as discrete dosage forms, such as, but not limited to, tablets (including without limitation scored or coated tablets), pills, caplets. capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia PA. (2005).

In certain embodiments, an effective dose of a composition comprising a Mucispirillum composition as described herein, a diet high in sulfur amino acids (SAA), and/or an XCL1 polypeptide (or XCR1 agonist) as described herein can be administered to a patient once. In certain embodiments, an effective dose of a composition comprising a Mucispirillum composition as described herein, a diet high in sulfur amino acids (SAA), and/or an XCL1 polypeptide (or XCR1 agonist) can be administered to a patient repeatedly. For systemic administration, subjects can be administered a therapeutic amount of a composition comprising a Mucispirillum composition as described herein, sulfur amino acids (SAA), and/or an XCL1 polypeptide (or XCR1 agonist), such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. cancer (e.g., colorectal cancer) by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the Mucispirillum composition as described herein, a diet high in sulfur amino acids (SAA), and/or the XCL1 polypeptide (or XCR1 agonist). The desired dose or amount can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising a Mucispirillum composition as described herein, a diet high in sulfur amino acids (SAA), and/or an XCL1 polypeptide (or XCR1 agonist) can be administered over a period of time, such as over a 5 minute, 10 minute, 15 minute, 20 minute, or 25 minute period.

The dosage ranges for the administration of the compositions described herein, according to the methods described herein depend upon, for example, the form of the Mucispirillum composition as described herein, the diet high in sulfur amino acids (SAA), and/or the XCL1 polypeptide (or XCR1 agonist), its potency, and the extent to which symptoms, markers, or indicators of a condition described herein are desired to be reduced, for example the percentage reduction desired for tumor and/or cancer symptoms, or the extent to which, for example, the level of Mucispirillum bacteria, XCL1 polypeptide, NKTs, and/or CD103+ cDC1s are desired to be increased. The dosage should not be so large as to cause adverse side effects, such as septicemia or autoimmunity. Generally, the dosage will vary with the age, condition, and sex of the patient and can be determined by one of skill in the art. The dosage can also be adjusted by the individual physician in the event of any complication.

The efficacy of the Mucispirillum composition as described herein, the diet high in sulfur amino acids (SAA), and/or the XCL1 polypeptide (or XCR1 agonist) in, e.g. the treatment of a condition described herein, or to induce a response as described herein can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. the level of Mucispirillum bacteria, XCL1 polypeptide, NKTs, and/or CD103+ cDC1s, e.g., cancer indicators such as tumor size, tumor growth, and/or tumor metastatic activity. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or organ damage; e.g., symptoms and/or complications of cancer, as described further herein; e.g., symptoms and/or complications of colorectal cancer, as described further herein); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g. increased levels of Mucispirillum bacteria, XCL1 polypeptide, NKTs, and/or CD103+ cDC1s; e.g., decreases in cancer indicators such as tumor size, tumor growth, and/or tumor metastatic activity). It is well within the ability of one skilled in the art to monitor efficacy of administration and/or treatment by measuring any one of such parameters, or any combination of parameters. Efficacy can be assessed in animal models of a condition described herein, for example treatment of cancer (e.g., colorectal cancer). When using an experimental animal model, efficacy of treatment is evidenced when a statistically significant change in a marker is observed, e.g. increased levels of Mucispirillum bacteria, XCL1 polypeptide, NKTs, and/or CD103+ cDC1s, e.g., decreases in cancer indicators such as tumor size, tumor growth, and/or tumor metastatic activity.

In vitro and animal model assays are provided herein which allow the assessment of a given dose of a Mucispirillum composition as described herein, a diet high in sulfur amino acids (SAA), and/or an XCL1 polypeptide (or XCR1 agonist). By way of non-limiting example, the effects of a dose of a Mucispirillum composition as described herein, a diet high in sulfur amino acids (SAA), and/or an XCL1 polypeptide (or XCR1 agonist) can be assessed by a clinical trial in human volunteers. The efficacy of a given dosage combination can also be assessed in an animal model, including but not limited to the murine models described further herein (see e.g., Example 1).

### Cancer

As used herein, the term “cancer” relates generally to a class of diseases or conditions in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord.

In some embodiments of any of the aspects, the cancer is a primary cancer. In some embodiments of any of the aspects, the cancer is a malignant cancer. As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor. As used herein, the term “benign” or “non-malignant” refers to tumors that may grow larger but do not spread to other parts of the body. Benign tumors are self-limited and typically do not invade or metastasize.

A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancer cells form tumors, but some, e.g., leukemia, do not necessarily form tumors. For those cancer cells that form tumors, the terms cancer (cell) and tumor (cell) are used interchangeably.

As used herein the term “neoplasm” refers to any new and abnormal growth of tissue, e.g., an abnormal mass of tissue, the growth of which exceeds and is uncoordinated with that of the normal tissues. Thus, a neoplasm can be a benign neoplasm, premalignant neoplasm, or a malignant neoplasm.

A subject that has a cancer or a tumor is a subject having objectively measurable cancer cells present in the subject's body. Included in this definition are malignant, actively proliferative cancers, as well as potentially dormant tumors or micrometastases. Cancers which migrate from their original location and seed other vital organs can eventually lead to the death of the subject through the functional deterioration of the affected organs.

Examples of cancer include but are not limited to, carcinoma, lymphoma, blastoma, sarcoma, leukemia, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma (GBM); hepatic carcinoma; hepatoma; intra-epithelial neoplasm.; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

A “cancer cell” is a cancerous, pre-cancerous, or transformed cell, either in vivo, ex vivo, or in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is associated with, e.g., morphological changes, immortalization of cells, aberrant growth control, foci formation, anchorage independence, malignancy, loss of contact inhibition and density limitation of growth, growth factor or serum independence, tumor specific markers, invasiveness or metastasis, and tumor growth in suitable animal hosts such as nude mice.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

As used herein, the term “colonized” or “colonization” refers broadly to the presence of microbiota in vivo such as in the gastrointestinal tract or skin of a mammalian organism without perceptible significant alteration other than the presence of bacteria. As opposed to passing transiently through the gastrointestinal tract, the colonized microbiota becomes non-transiently (e.g. semi-permanently) established and/or reproducing in the gastrointestinal tract, e.g., in the GI lumen or associated with the GI epithelial or mucus layer. “Colonized” or “colonization” can also refer to the presence of microbiota on foodstuff(s) or environmental surface(s). The terms “colonization” and “colonized” stand in contrast to the terms “infection” or “infected” which are commonly understood to require perceptible deleterious alteration as part of their definition. “Colonization” and “colonized” may also refer to the presence of bacteria in or on a human or animal without perceptible damage, alteration, or disease. “Colonization” and “colonized” can be associated with a benefit to the human or animal.

As used herein, the term “isolated” refers to a bacterium or other entity or substance that has been (1) separated from at least some of the components with which it was associated when initially produced (whether in nature, such as human stool, or in an experimental setting, such as a Petri plate consisting of artificial growth medium), and/or (2) produced, prepared, purified, and/or manufactured by the hand of man. Isolated bacteria, proteins, metabolites, or combinations thereof may be separated from at least about 10%, about 20%, about 30%, about 40%, about 50%, about 60%, about 70%, about 80%, about 90%, or more of the other components with which they were initially associated. In some embodiments, isolated bacteria, proteins, metabolites, or combinations thereof are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components (such as other bacterial species). The terms “purify,” “purifying” and “purified” refer to a bacterium or other material that has been separated from at least some of the components with which it was associated either when initially produced or generated (e.g., whether in nature or in an experimental setting), or during any time after its initial production, as recognized by those skilled in the art of bacterial cultivation or of relevant skill (e.g., chemistry). A bacterium or a bacterial population can be considered purified if it is isolated at or after production, such as from a material or environment containing the bacterium or bacterial population, and a purified bacterium or bacterial population can contain other materials up to about 10%, about 20%, about 30%, about 40%, about 50%, about 60%, about 70%, about 80%, about 90%, or above about 90% and still be considered “isolated.” In some embodiments, purified bacteria and bacterial populations are more than about 80%, about 85%, about 90%, about 91%, about 92%, about 93%, about 94%, about 95%, about 96%, about 97%, about 98%, about 99%, or more than about 99% pure. In the instance of bacterial compositions provided herein, the one or more bacterial types present in the composition can be independently purified from one or more other bacteria produced and/or present in the material or environment containing the bacterial type. In some embodiments, a bacterium or population of bacteria is “isolated” if it comprises a single strain of bacteria. In some embodiments, such isolated bacteria can be admixed or administered with other isolated bacteria, e.g., in a defined consortium of isolated bacteria.

As used herein, “probiotic” is understood to mean live microorganisms which when administered in adequate amounts confer a health benefit on the host.

As used herein, “prebiotic” is understood to mean an ingredient that allows or promotes specific changes, in the composition and/or activity of the microbiota, e.g., gastrointestinal microbiota, that may or may not confer benefits upon the host.

As used herein, “medical food” is understood to mean a food which is formulated to be consumed or administered enterally under the supervision of a physician and which is intended for the specific dietary management of a disease or condition for which distinctive nutritional requirements, based on recognized scientific principles, are established by medical evaluation.

As used herein, “supplement” (also referred to as a dietary supplement) is understood to mean a product taken orally that comprises one or more ingredients (e.g., vitamins, minerals, amino acids, an isolated microbe or product thereof as described herein) that are intended to supplement one's diet and are not considered food. As non-limiting examples, a supplement can be in the form of a capsule, an enteric capsule, a tablet, a caplet, a pill, a troche, a lozenge, a powder, or a granule.

The term “gut” is understood to refer to the human gastrointestinal tract, also known as the alimentary canal. The gut includes the mouth, pharynx, esophagus, stomach, small intestine (duodenum, jejunum, ileum). large intestine (cecum and colon) and rectum.

As used herein, “bacterium” is understood as a single bacterial cell of a given species.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal, e.g., for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of cancer (e.g., colorectal cancer). A subject can be male or female.

A subject can be one who has been previously diagnosed with or identified as suffering from or having a condition in need of treatment (e.g. cancer) or one or more complications related to such a condition, and optionally, have already undergone treatment for cancer or the one or more complications related to cancer. Alternatively, a subject can also be one who has not been previously diagnosed as having cancer or one or more complications related to cancer. For example, a subject can be one who exhibits one or more risk factors for cancer or one or more complications related to cancer or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested confirm that a desired activity, e.g. activity and specificity of a native or reference polypeptide (e.g., XCL1) is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a polypeptide which retains at least 50% of the wild-type reference polypeptide's activity. A functional fragment can comprise conservative substitutions of the sequences disclosed herein.

In some embodiments, the polypeptide described herein can be a variant of a polypeptide sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a protein or fragment thereof that retains activity of the native or reference polypeptide. A wide variety of, for example, PCR-based, site-specific mutagenesis approaches are known in the art and can be applied by the ordinarily skilled artisan to generate and test artificial variants.

A variant amino acid or DNA sequence can be at least 80%, at least 85%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

A variant amino acid sequence can be at least 50%, at least 60%, at least 70%, at least 80%, at least 90%, at least 91%, at least 92%, at least 93%, at least 94%, at least 95%, at least 96%, at least 97%, at least 98%, at least 99%, or more, similar to a native or reference sequence. As used herein, “similarity” refers to an identical amino acid or a conservatively substituted amino acid, as described herein. Accordingly, the percentage of “sequence similarity” is the percentage of amino acids which is either identical or conservatively changed: e.g., “sequence similarity”=(% sequence identity)+(% conservative changes). It should be understood that a sequence that has a specified percent similarity to a reference sequence necessarily encompasses a sequence with the same specified percent identity to that reference sequence. The skilled person will be aware of various computer programs, using different mathematical algorithms, that are available to determine the identity or similarity between two sequences. For instance, use can be made of a computer program employing the Needleman and Wunsch algorithm (Needleman et al. (1970)); the GAP program in the Accelrys GCG software package (Accelerys Inc., San Diego U.S.A.); the algorithm of E Meyers and W. Miller (Meyers et al. (1989)) which has been incorporated into the ALIGN program (version 2.0); or more preferably the BLAST (Basic Local Alignment Tool using default parameters); see e.g., U.S. Pat. No. 10,023,890, the content of which is incorporated by reference herein in its entirety.

In some embodiments, sequencing comprises 16S rRNA gene sequencing, which can also be referred to as “16S ribosomal RNA sequencing”, “16S rDNA sequencing” or “16s rRNA sequencing”. Sequencing of the 16S rRNA gene can be used for genetic studies as it is highly conserved between different species of bacteria, but it is not present in eukaryotic species. In addition to highly conserved regions, the 16S rRNA gene also comprises nine hypervariable regions (V1-V9) that vary by species. 16S rRNA gene sequencing typically comprises using a plurality of universal primers that bind to conserved regions of the 16S rRNA gene, PCR amplifying the bacterial 16S rRNA gene regions (including hypervariable regions), and sequencing the amplified 16S rRNA genes with a next-generation sequencing technology as described herein (see also e.g., U.S. Pat. Nos. 5,654,418; 6,344,316; and 8,889,358; and US Patent Application Numbers US 2013/0157265 and US 2018/0195111, which are incorporated by reference in their entireties).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. A wide variety of, site-specific mutagenesis approaches, e.g., Kunkel's method, cassette mutagenesis, PCR site-directed mutagenesis (e.g., traditional PCR, primer extension, or inverse PCR), whole plasmid mutagenesis, in vivo site-directed mutagenesis, CRISPR/Cas-guided mutagenesis, are known in the art and can be applied by the ordinarily skilled artisan to introduce mutations into specific nucleic acid loci. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); Braman, Jeff, ed. (2002) In Vitro Mutagenesis Protocols, Methods in Molecular Biology, Vol. 182 (2nd ed.); Khudyakov and Fields (2002), Artificial DNA: Methods and Applications, CRC Press; Hsu et al. (2014), Cell 157 (6): 1262-78; Cerchione et al. (2020) PLOS ONE 15 (4): e0231716; and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

In some embodiments, the methods described herein relate to measuring, detecting, or determining the level of at least one marker. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. cancer (e.g., colorectal cancer). The term “treating” includes reducing or alleviating at least one adverse effect or symptom of a condition, disease or disorder associated with cancer (e.g., colorectal cancer). Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in or within nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in cell biology, immunology, and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (cd.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Some embodiments of the technology described herein can be defined according to any of the following numbered paragraphs:

1. A composition comprising Mucispirillum schaedleri (M. schaedleri) bacteria formulated for delivery to the intestine.

2. The composition of paragraph 1, wherein the M. schaedleri bacteria are living or inactivated.

3. The composition of paragraph 1, wherein the M. schaedleri bacteria are in dried viable form.

4. The composition of any one of paragraphs 1-3, wherein the M. schaedleri bacteria are encapsulated.

5. The composition of any one of paragraphs 1-4, wherein the M. schaedleri bacteria are comprised in an enteric capsule.

6. The composition of any one of paragraphs 1-5, wherein the M. schaedleri bacteria are maintained in an anaerobic state in the formulation.

7. The composition of any one of paragraphs 1-6, wherein the M. schaedleri bacteria are in admixture with a prebiotic.

8. The composition of any one of paragraphs 1-7, wherein the M. schaedleri bacteria are in admixture with a sulfur amino acid.

9. The composition of paragraph 8, wherein the sulfur amino acid is methionine, cysteine or a derivative thereof.

10. The composition of any one of paragraphs 1-9, wherein the M. schaedleri bacteria are formulated in a food composition.

11. The composition of paragraph 10, wherein the food composition is supplemented with a sulfur amino acid and/or a prebiotic.

12. The composition of any one of paragraphs 1-11, further comprising 1 to 20 additional species of bacteria.

13. The composition of any one of paragraphs 1-11, which comprises no more than 20 species of bacteria.

14. A composition comprising live M. schaedleri bacteria, dead M. schaedleri bacteria, conditioned M. schaedleri culture medium, or an organic solvent extract of conditioned M. schaedleri culture medium or a fraction thereof that promotes XCL1 secretion by NKT cells, wherein the composition is formulated for delivery to the intestine.

15. The composition of paragraph 14, wherein the M. schaedleri bacteria, medium or solvent extract are in dried form.

16. The composition of either of paragraphs 14 or 15, wherein the M. schaedleri bacteria, medium or extract is/are encapsulated.

17. The composition of any one of paragraphs 14-16, wherein the M. schaedleri bacteria are comprised in an enteric capsule.

18. The composition of any one of paragraphs 14-17, wherein the M. schaedleri bacteria are maintained an anaerobic state in the formulation.

19. The composition of any one of paragraphs 14-18, wherein the M. schaedleri bacteria, medium or extract is/are in admixture with a prebiotic and/or a sulfur amino acid or derivative thereof.

20. The composition of any one of paragraphs 14-19, wherein the conditioned M. schaedleri culture medium, or the organic solvent extract of conditioned M. schaedleri culture medium or a fraction thereof comprises at least one metabolite selected from FIG. 4H, FIG. 4I, or FIG. 16.

21. The composition of any one of paragraphs 14-20, wherein the conditioned M. schaedleri culture medium, or the organic solvent extract of conditioned M. schaedleri culture medium or a fraction thereof comprises at least one metabolite selected from the group consisting of succinic acid; propionic acid; nicotinic acid; aconitic acid (cis and/or trans); pentadecanoic acid; itaconic acid; 16-hydroxyhexadecanoic acid; C₈H₁₅NO₃S; crotonic acid; myristic acid; 17-hydroxyheptadecanoic acid (C₁₇H₃₄O₃); and 15-hydroxpentadecanoic acid (C₁₅H₃₀O₃).

22. The composition of any one of paragraphs 14-21, wherein the conditioned M. schaedleri culture medium, or the organic solvent extract of conditioned M. schaedleri culture medium or a fraction thereof comprises at least one metabolite selected from the group consisting of: succinic acid; nicotinic acid; aconitic acid (cis and/or trans); pentadecanoic acid; itaconic acid; 16-hydroxyhexadecanoic acid; and crotonic acid.

23. A food composition comprising the composition of any one of paragraphs 14-22.

24. The food composition of paragraph 23, further comprising 1 to 20 additional species of bacteria.

25. A method of treating cancer or promoting anti-tumor immune activity, the method comprising administering to a subject in need thereof a composition of any one of paragraphs 1-24.

26. The method of paragraph 25, wherein the cancer is colon cancer.

27. The method of paragraph 25 or 26, further comprising administering an immune checkpoint inhibitor.

28. A method of promoting responsiveness to immune checkpoint inhibitor tumor therapy, the method comprising administering to a subject in need thereof a composition of any one of paragraphs 1-24.

29. The method of paragraph 28, further comprising administering an immune checkpoint inhibitor.

30. The method of paragraph 28 or 29, wherein the subject has colon cancer.

31. The method of any one of paragraphs 28-30, wherein the subject's cancer has been determined to be resistant to immune checkpoint inhibitor therapy.

32. The method of any one of paragraphs 28-31, wherein the composition promotes XCL1 secretion by NKT cells.

33. A method of increasing CD103+ conventional dendritic cells (cDC1), the method comprising administering to a subject in need thereof a composition of any one of paragraphs 1-24.

34. The method of paragraph 33, wherein the cDC1s are associated with a tumor.

35. The method of paragraph 34, wherein the tumor is a colon cancer.

36. The method of any one of paragraphs 33-35, further comprising administering a sulfur amino acid.

37. The method of any one of paragraphs 33-36, further comprising administering an immune checkpoint inhibitor.

38. A method of increasing XCL1 secretion by NKT cells, the method comprising administering to a subject in need thereof a composition of any one of paragraphs 1-24.

39. The method of paragraph 38, wherein the subject has cancer.

40. The method of paragraph 38 or 39, wherein the subject has colon cancer.

41. A method of increasing CD8+ T cell infiltration in a colorectal tumor, the method comprising administering to a subject in need thereof a composition of any one of paragraphs 1-24.

42. The method of paragraph 41, wherein the cDC1s are associated with a tumor.

43. The method of paragraph 42, wherein the tumor is a colon cancer.

44. The method of any one of paragraphs 41-43, further comprising administering a sulfur amino acid.

45. The method of any one of paragraphs 41-44, further comprising administering an immune checkpoint inhibitor.

46. A method of increasing CD8+ T cell infiltration in a colorectal tumor, the method comprising administering a diet high in sulfur amino acids (SAA) or a supplement comprising SAAs to a subject in need thereof.

47. The method of paragraph 46, wherein the diet high in sulfur amino acids comprises greater than 0.04 grams of SAA per kilogram body weight per day.

48. The method of paragraph 46 or 47, further comprising administering a composition of any one of paragraphs 13 to 20 to the subject.

49. A method of establishing or maintaining a tumor-suppressive gut environment in a subject in need thereof, the method comprising administering a diet high in sulfur amino acids (SAA) or a supplement comprising SAAs.

50. The method of paragraph 49, wherein the diet high in sulfur amino acids or a supplement comprising SAAs comprises greater than 0.04 grams of SAA per kilogram body weight per day.

51. The method of paragraphs 49 or 50, further comprising administering a composition of any one of paragraphs 14 to 21 to the subject.

52. A method of treating cancer, the method comprising administering to a subject in need thereof an XCL1 polypeptide.

53. The method of paragraph 52, wherein the cancer is colon cancer.

54. The method of paragraph 52 or 53, wherein the XCL1 polypeptide is administered to the gut.

55. A method of treating cancer, the method comprising administering to a subject in need thereof a microorganism engineered to express XCL1 polypeptide.

56. A method of treating cancer, the method comprising administering to a subject in need thereof an agonist of the XCL1 receptor, XCR1.

57. The method of paragraph 56, wherein the XCR1 agonist comprises SEQ ID NOs: 9-11 or an amino acid sequence that is at least 95% identical and maintains its function.

58. A method of treating cancer in a subject in need thereof, the method comprising:
detecting the level of M. schaedleri in a sample from the subject;
administering a cancer immunotherapeutic agent if the level of M. schaedleri is at or above a pre-determined threshold; and
administering a cancer immunotherapeutic agent and a composition of any one of paragraphs 1-24 if the level of M. schaedleri is below a pre-determined threshold.

59. A method of treating cancer in a subject in need thereof, the method comprising:
obtaining results from an assay detecting the level of M. schaedleri in a sample from the subject;
administering a cancer immunotherapeutic agent if the level of M. schaedleri is at or above a pre-determined threshold; and
administering a cancer immunotherapeutic agent and a composition of any one of paragraphs 1-24 if the level of M. schaedleri is below a pre-determined threshold.

60. A method of treating cancer in a subject in need thereof, the method comprising:
detecting the level of XCL1 polypeptide, NKTs, and/or CD1103+ cDC1s in a sample from the subject;
administering a cancer immunotherapeutic agent if the level of XCL1 polypeptide, NKTs, and/or CD103+ cDC1s is at or above a pre-determined threshold; and
administering a cancer immunotherapeutic agent and a composition of any one of paragraphs 1-24 if the level of XCL1 polypeptide, NKTs, and/or CD103+ cDC1s is below a pre-determined threshold.

61. A method of treating cancer in a subject in need thereof, the method comprising:
obtaining results from an assay detecting the level of XCL1 polypeptide, NKTs, and/or CD103+ cDC1s in a sample from the subject;
administering a cancer immunotherapeutic agent if the level of XCL1 polypeptide, NKTs, and/or CD103+ cDC1s is at or above a pre-determined threshold; and
administering a cancer immunotherapeutic agent and a composition of any one of paragraphs 1-24 if the level of XCL1 polypeptide, NKTs, and/or CD103+ cDC1s is below a pre-determined threshold.

62. A method of stratifying a subject for cancer treatment, the method comprising:
detecting the level of M. schaedleri in a sample from the subject; and
classifying the subject as high risk if the level of M. schaedleri is below a pre-determined threshold; or
classifying the subject as low risk if the level of M. schaedleri is at or above a pre-determined threshold.

63. A method of stratifying a subject for cancer treatment, the method comprising:
obtaining results from an assay detecting the level of M. schaedleri in a sample from the subject; and
classifying the subject as high risk if the level of M. schaedleri is below a pre-determined threshold; or
classifying the subject as low risk if the level of M. schaedleri is at or above a pre-determined threshold.

64. A method of stratifying a subject for cancer treatment, the method comprising:
detecting the level of XCL1 polypeptide, NKTs, and/or CD103+ cDC1s in a sample from the subject; and
classifying the subject as high risk if the level of XCL1 polypeptide, NKTs, and/or CD103+ cDC1s is below a pre-determined threshold; or
classifying the subject as low risk if the level of XCL1 polypeptide, NKTs, and/or CD103+ cDC1s is at or above a pre-determined threshold.

65. A method of stratifying a subject for cancer treatment, the method comprising:
obtaining results from an assay detecting the level of XCL1 polypeptide, NKTs, and/or CD103+ cDC1s in a sample from the subject; and
classifying the subject as high risk if the level of XCL1 polypeptide, NKTs, and/or CD103+ cDC1s is below a pre-determined threshold; or
classifying the subject as low risk if the level of XCL1 polypeptide, NKTs, and/or CD103+ cDC1s is at or above a pre-determined threshold.

66. The method of any one of paragraphs 58-65, wherein the subject has colon cancer.

67. The method of any one of paragraphs 62-65, further comprising administering the composition of any one of paragraphs 1-24.

68. The method of any one of paragraphs 58-67, further comprising administering a sulfur amino acid.

69. The method of any one of paragraphs 58-61, wherein the cancer immunotherapeutic agent is selected from the group consisting of an immune checkpoint inhibitor; chemotherapy; a dendritic cell vaccine; chimeric antigen receptor T cells (CAR-T); and NKT cell-based therapies.

70. The method of any one of paragraphs 58-61, wherein the cancer immunotherapeutic agent comprises an immune checkpoint inhibitor.

71. The method of any one of paragraphs 62-65, further comprising administering an immune checkpoint inhibitor.

72. The method of any one of paragraphs 58-71, further comprising administering a diet high in sulfur amino acids or a supplement comprising SAAs.

73. The method of any one of paragraphs 58-61 or 66-69, wherein the method results in higher treatment efficacy compared to a method of treating without first: detecting the level of M. schaedleri; obtaining results from an assay detecting the level of M. schaedleri; detecting the level of XCL1 polypeptide, NKTs, and/or CD103+ cDC1s; or obtaining results from an assay detecting the level of XCL1 polypeptide, NKTs, and/or CD103+ cDC1s.

74. The method of any one of paragraphs 62-72, wherein the method results in higher treatment efficacy compared to a method of treating without first stratifying the subject.

75. The method of any one of paragraphs 58-61 or 66-72, wherein the method results in lower treatment complications compared to a method of treating without first detecting the level of M. schaedleri, obtaining results from an assay detecting the level of M. schaedleri, detecting the level of XCL1 polypeptide, NKTs, and/or CD103+ cDC1s or obtaining results from an assay detecting the level of XCL1 polypeptide, NKTs, and/or CD103+ cDC1s.

76. The method of any one of paragraphs 62-72, wherein the method results in lower treatment complications compared to a method of treating without first stratifying the subject.

77. An enteric delivery formulation comprising at least one metabolite selected from FIG. 4H, FIG. 4I, or FIG. 16.

78. An enteric delivery formulation comprising at least one metabolite selected from the group consisting of: succinic acid; propionic acid; nicotinic acid; aconitic acid (cis and/or trans); pentadecanoic acid; itaconic acid; 16-hydroxyhexadecanoic acid; C₈H₁₅NO₃S; crotonic acid; myristic acid; 17-hydroxyheptadecanoic acid (C₁₇H₃₄O₃); and 15-hydroxpentadecanoic acid (C₁₅H₃₀O₃).

79. An enteric delivery formulation comprising at least one metabolite selected from the group consisting of: succinic acid; nicotinic acid; aconitic acid (cis and/or trans); pentadecanoic acid; itaconic acid; 16-hydroxyhexadecanoic acid; and crotonic acid.

80. The enteric delivery formulation of any one of paragraphs 77-79, formulated for delivery to the intestine.

The technology described herein is further illustrated by the following examples which in no way should be construed as being further limiting.

### EXAMPLES

### Example 1: Diet-Microbial Interaction Enhances Anti-Tumor Immunity in Colon Cancer

Immune checkpoint inhibitors (ICI) have transformed cancer treatment, yet response to ICI is not universal, especially for colorectal cancer (CRC). While certain gut microbiota members have been associated with ICI treatment response in patients, mechanisms governing microbial modulation of anti-tumor immunity remain elusive. It was contemplated herein that microbial metabolic capabilities, rather than a specific taxonomic configuration, could better differentiate ICI responders from ICI non-responders. As the etiopathogenesis of CRC is associated with both the microbiota and dietary factors, biochemistry that would be influenced by dietary factors was examined. Microbial metabolic features were examined in ICI-responsive patients. As described herein, gut microbial sulfur amino acid (Saa) metabolic pathways and gene abundance were positively associated with ICI-responsiveness, and a diet enriched in Saa slowed tumor growth at a magnitude comparable to α-PD-1 treatment in a heterotopic mismatch repair defective CRC model. In genetically engineered mouse model of mismatch repair proficient and ICI-resistant CRC, a high Saa diet blunted neoplastic progression and promoted an expansion of a niche for Mucispirillum schaedleri, a mucus-dwelling immunomodulatory species present in the human and mouse colon. The high Saa diet, studied here in mice and achievable in humans via dietary supplementation, induced a M. schaedleri bloom that turned ‘cold’ CRC tumors ‘warm’ by increasing NKT cell secretion of XCL1, expanding and activating cDC1s in tumor-draining lymph nodes, ultimately leading to enhanced tumoral CD8⁺ T cell numbers and function and reduced tumor growth. Together, described herein are a diet-microbiota-host interaction and substantiate microbiota-targeted, diet-based cancer therapeutics. Described herein in multiple aspects is a diet, based on microbiome analyses of immunotherapy-responsive patients, that enhanced anti-tumor immunity in CRC models.

As further described herein, gut microbiome sulfur amino acid (Saa) metabolic gene abundance correlated with improved anti-tumor immunity and ICI response, prompting testing of the effects of dietary Saa in mouse models of CRC. A high Saa diet slowed tumor growth in a heterotopic CRC model, and such a diet augmented CD8⁺ T cell infiltration and decreased neoplastic progression in a genetically-driven CRC model. The high Saa diet increased the abundance of Mucispirillum schaedleri, which increased CD103′ conventional dendritic cells (cDC1) abundance and activation state in the tumor draining lymph nodes (TDLN), enhancing anti-tumor immune responses. M. schaedleri conditioned medium (CDM) induced XCL1 production by Natural Killer T (NKT) cells in vitro, indicating a direct causal effect of M. schaedleri on this cDC1 activation pathway. Thus, described herein is a diet-microbiota-host interaction and signature regulating anti-tumor immunity, which provided a framework for identifying and evaluating microbiota-targeted, diet-based cancer adjunctive therapies.

Saa Biosynthesis Genes were Enriched in ICI-Responsive Patients.

To quantify microbial pathways enriched in ICI responders' microbiomes, a set of analyses was performed on the microbial metabolic potential in stool metagenomics datasets from ICI responders vs. non-responders in eight ICI-microbiome studies; see e.g., Andrews et al. 2021, supra; Frankel et al. 2017, supra; Gopalakrishnan et al., 2018, supra; Lee et al. 2021, supra; Matson et al. 2018, supra; Routy et al. 2018, supra; Baruch et al., Science 371, 602-609 (2021); Davar et al., Science 371, 595-602 (2021); the contents of each of which are incorporated herein by reference in their entireties. The initial testing focused on a cohort of ICI-treated renal cell cancer (RCC) patients as this patient population had less reported antibiotic (Abx) usage as compared to non-small cell lung cancer (NSCLC) and melanoma patients, and Abx can act as an additional modifier of the microbiota and ICI-response. Functional profiling was used from HUMAnN 2 (see e.g., Franzosa et al., Nat Methods 15, 962-968 (2018), the contents of which are incorporated herein by reference in their entirety; see e.g., supplemental methods) with stool metagenomes from the RCC cohort (see e.g., Routy et al., 2018 supra), selecting those that met Response Evaluation Criteria in Solid Tumors (RECIST) criteria for a partial or complete response and excluding those with stable disease. Profiles from these samples (n=16) were compared with those from ICI non-responder (n=32) RCC patients by least-square linear model-based differential abundance analysis.

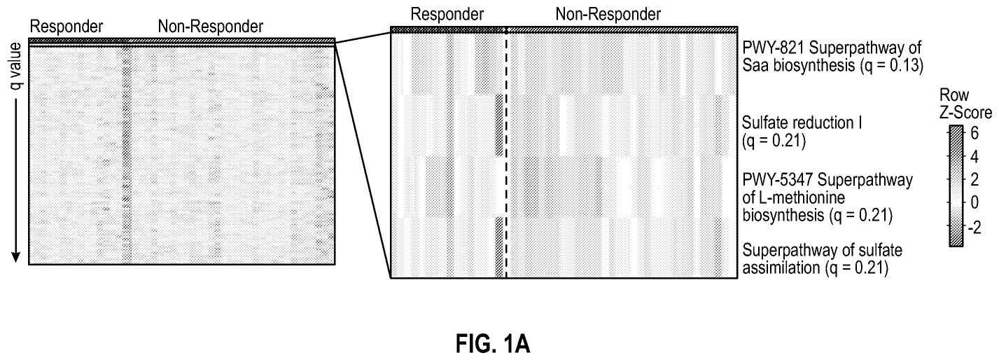

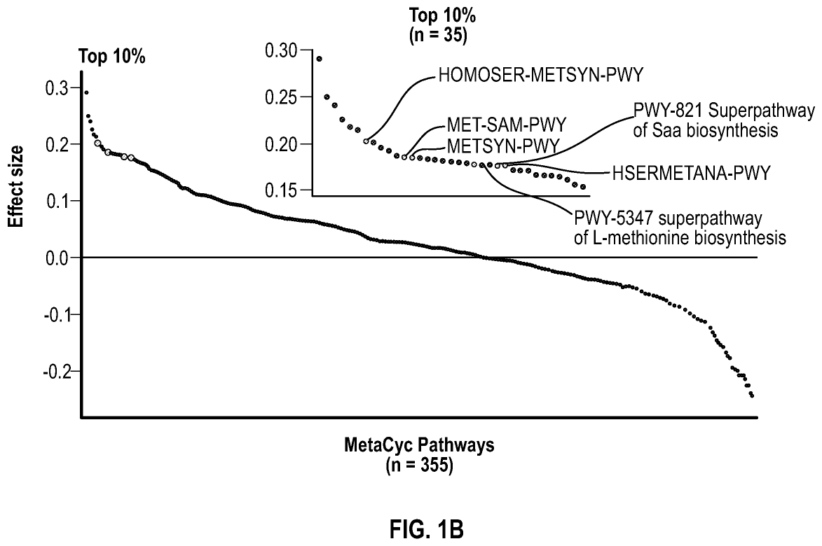

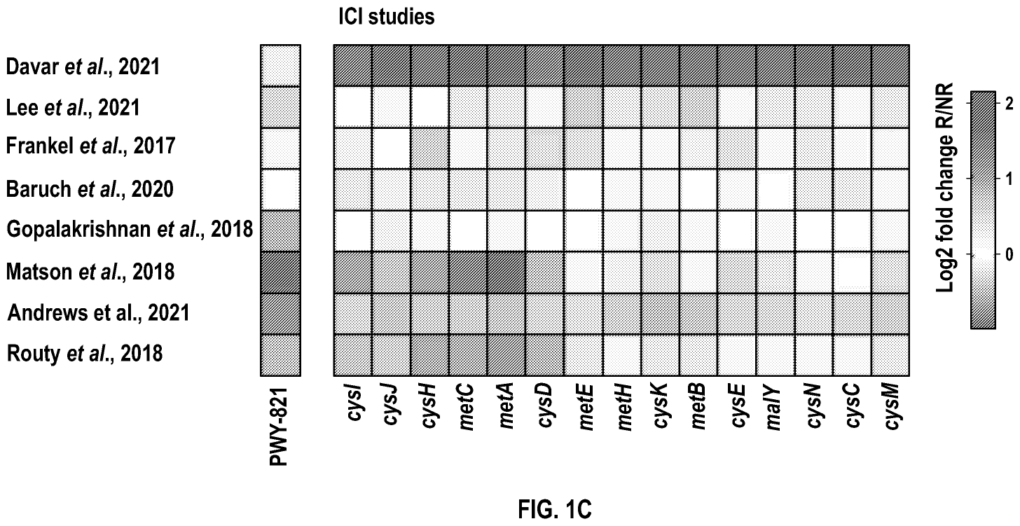

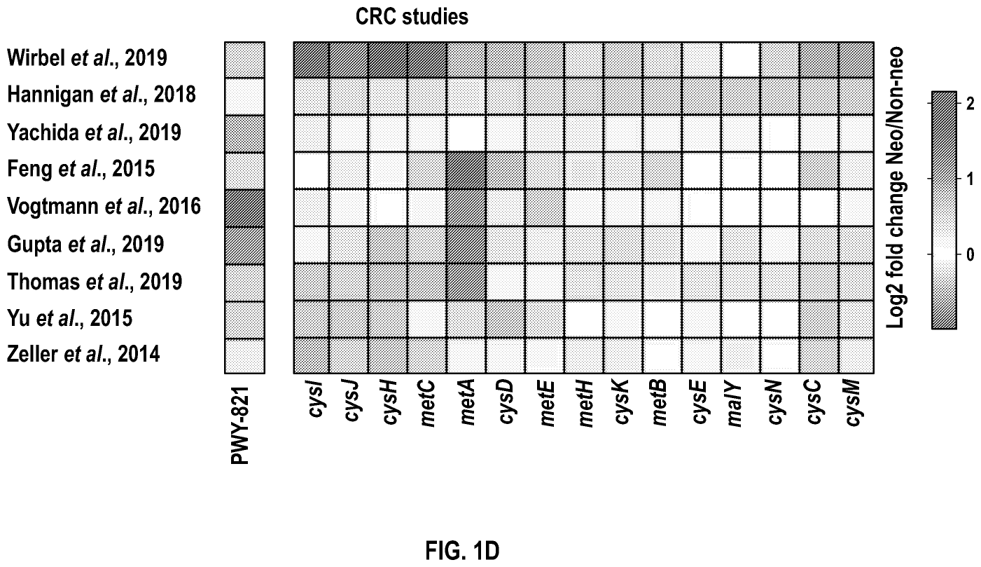

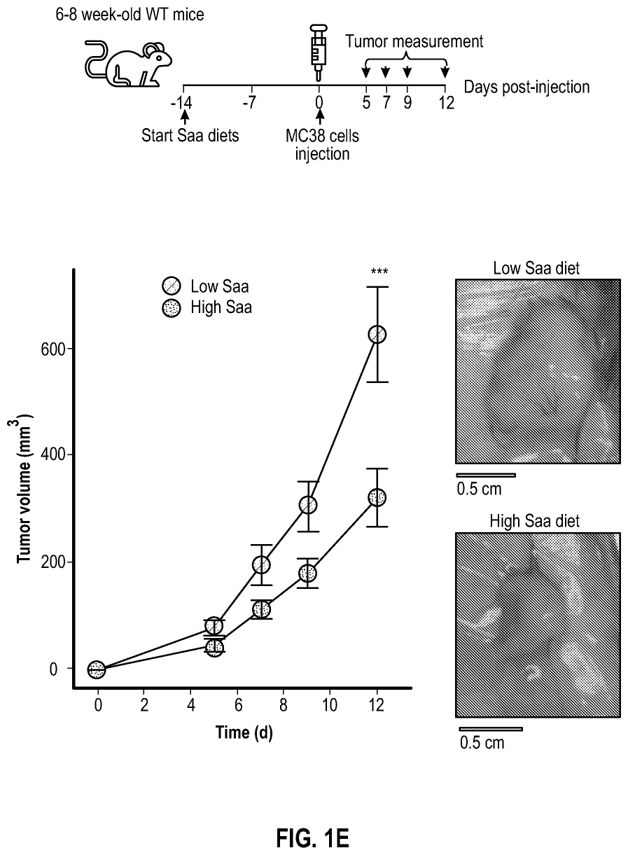

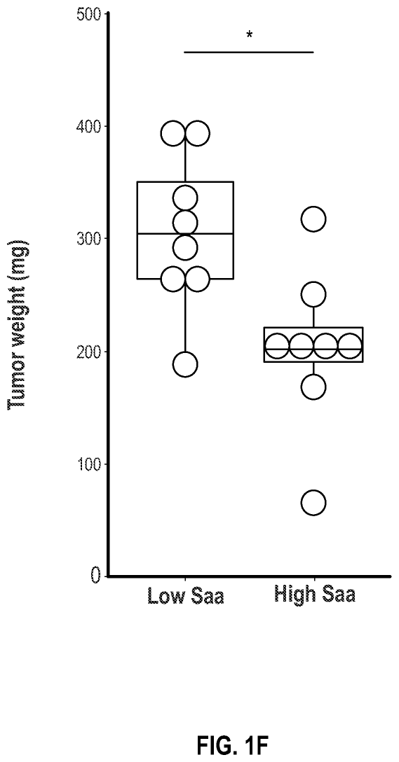

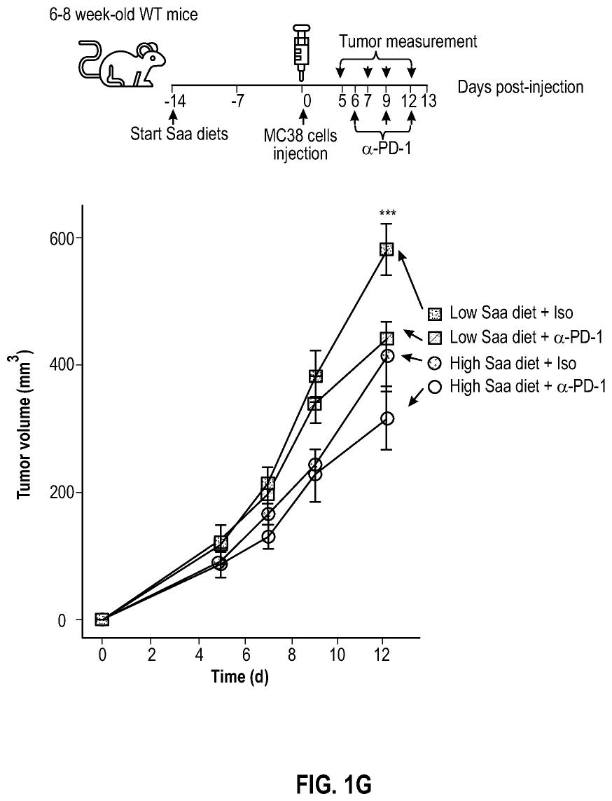

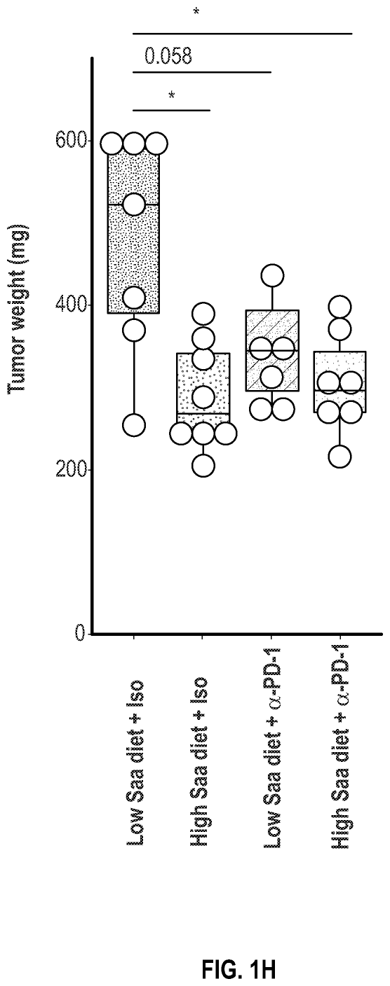

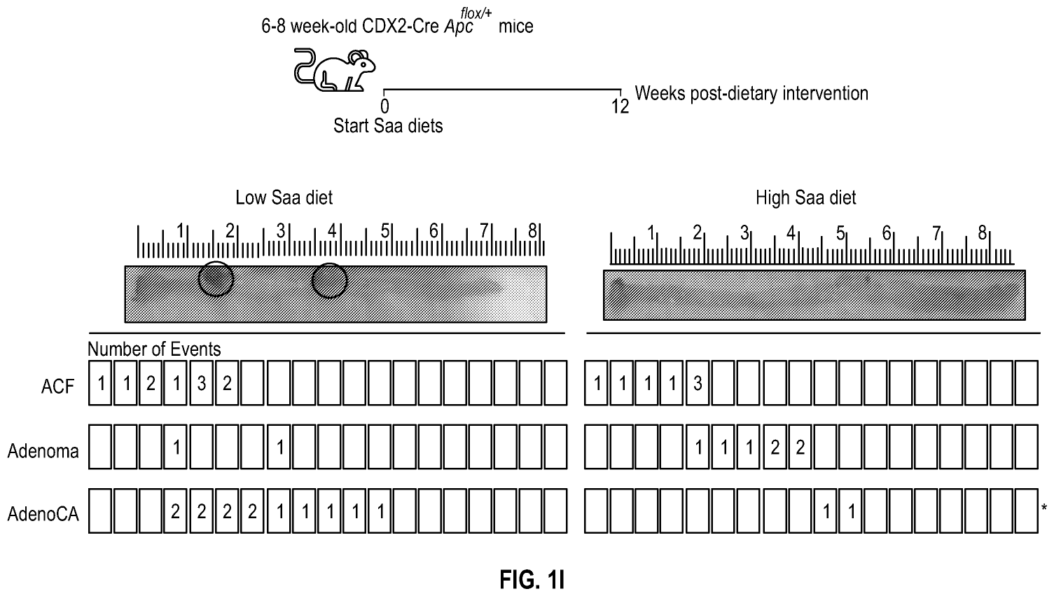

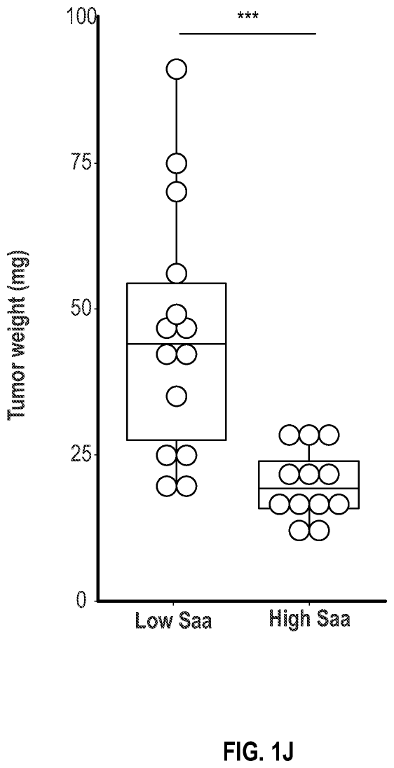

The MetaCyc superpathway of Saa biosynthesis (PWY-821) was the most enriched metabolic pathway in the microbiomes of ICI responders vs. non-responders, followed by three other sulfur-related pathways: sulfate reduction 1, superpathway of L-methionine biosynthesis (PWY-5347), and superpathway of sulfate assimilation (see e.g., FIG. 1A). Based on these results, an unbiased meta-analysis was performed on the microbiomes of responders (R) vs. non-responders (NR) with melanoma, RCC, or non-small cell lung cancer (NSCLC) from the eight ICI-microbiome studies (R, n=203, NR, n=134). Six Saa pathways were among the top 10% of pathways enriched in ICI responders vs. non-responders (ranked by effect size, Cohen's D test), including the superpathway of Saa biosynthesis PWY-821 (see e.g., FIG. 1B, Table 1). A targeted analysis of the superpathway of Saa biosynthesis abundance within each study independently revealed its enrichment in responder vs. non-responder microbiomes in 7 out of the 8 studies (see e.g., FIG. 1C). All the analyzed ICI-microbiome cohorts were controlled for common co-variates such as age, sex, and TNM stage (tumor (T), node (N), and metastasis (M)), but not all have available survival or dietary pattern data. In support of this approach, microbial metabolic pathways related to inosine, produced by lactic-acid bacteria and reported to enhance responses to ICI therapy in preclinical cancer models, were also enriched in the top 10% of pathways in ICI R vs. NR (see e.g., Table 1); see e.g., Mager et al., Science 369, 1481 (2020), the contents of which are incorporated herein by reference in their entirety.

To extend the MetaCyc pathways analysis, a set of 15 bacterial gene homologs was examined, based on E. coli Saa biosynthesis genes, encompassing the enzyme repertoire of the superpathway of Saa biosynthesis (n=11 genes) and also including genes from the superpathway of methionine biosynthesis enriched in ICI-responders (see e.g., FIG. 1B) that are prevalent in human gut metagenomes. A bacterial pangenome database was generated of the 15 Saa metabolism gene homologs (criteria: e-value 10⁻⁹, coverage 80%) and stool metagenomic sequences were aligned against this reference. Consistent with the HUMAnN MetaCyc analysis, Saa metabolic genes were enriched in ICI responders vs. non-responders (see e.g., log 2-fold change shown in FIG. 1C).

The enrichment of microbiome Saa metabolic gene carriage across ICI responders with different cancer types indicated a role for microbial Saa metabolism in anti-tumor immunity. This provided an opportunity to enhance anti-tumor immunity in immunologically “cold” tumors, like mismatch repair proficient CRC. Stool metagenomes were examined from nine CRC cohorts (containing a total of 812 patients (610 with CRC and 202 with adenomas), and 639 healthy controls) to profile Saa metabolism-related gene carriage in CRC patients vs. controls. See e.g., Feng et al., Nat Commun 6, 6528 (2015); Gupta et al., mSystems 4, c00438-19 (2019); Hannigan et al. mBio 9, c02248-18 (2018); Vogtmann et al., PLoS One 11, e0155362 (2016); Wirbel et al., Nat Med 25, 679-689 (2019); Yachida et al., Nat Med 25, 968-976 (2019); Yu et al., Gut 66, 70-78 (2017); Zeller et al., Mol Syst Biol 10, 766 (2014); Thomas et al., Nat Med 25, 667-678 (2019); the contents of each of which are incorporated herein by reference in their entireties. All the CRC cohorts selected for analysis were controlled for common co-variates such as age, sex, and TNM stage, but lacked survival data. There are no publicly available CRC ICI microbiome profiling studies to date. In these histopathologically-, genetically-, and geographically-diverse CRC cohorts, the Saa superpathway and 15 Saa metabolism gene homologs were present and enriched compared to controls in some cohorts (see e.g., FIG. 1D). That these Saa gene abundances in some CRC cohorts shared a pattern similar to those observed in many ICI responders led to the consideration that a proportion of CRC patients can respond to targeting Saa pathways.

### Dietary Saa and the Microbiota Modulated Neoplastic Progression in Mouse Models of CRC.

Since Saa metabolic genes were enriched in the microbiomes of ICI responders (see e.g., FIG. 1A), and diet can affect the microbiome by changing nutritional niche availability, there is increasing interest in microbiome-directed foods for regulating immune system function in cancer patients. As described herein, Saa pathways can be targeted via dietary Saa supplementation, which can then in turn influence CRC anti-tumor immunity. Two isocaloric mouse diets were formulated to represent edge cases of human Saa consumption, i.e., diets with low vs. high amounts of methionine and cysteine (see e.g., Table 2). The low Saa diet had sufficient methionine to avoid methionine restriction, and the high diet was well below the threshold for hyperhomocysteinemia, thus representing physiologically relevant human Saa consumption which can be achieved through dietary modification or supplementation.

A mismatch-repair deficient (dMMR) heterotopic colon adenocarcinoma (MC38 cell) mouse model was used that responded to the anti-PD-1 ICI treatment and was examined in the microbiome meta-analyses (see e.g., FIG. 1A-1C). Wild-type (WT) C57BL/6 mice raised in the vivarium were placed on high or low Saa diets 2 weeks prior to MC38 cell flank-injection and monitored for 12 days after injection. Mice fed the high Saa diet had slower tumor growth with a ˜50% reduction in tumor volume and 30% reduction in tumor weight (see e.g., FIG. 1E-1F). Since MC38 tumors responded to anti-PD-1 antibody (Ab) treatment, it was tested whether the diet could exert an additive or synergistic effect in combination with anti-PD-1 Ab. While the high Saa diet was neither additive nor synergistic with the anti-PD-1 Ab, of the diet reduced terminal tumor volume and weight similar to the level observed with anti-PD-1 Ab treatment, indicating that the high Saa diet had comparable efficacy as ICI therapy in this model (see e.g., FIG. 1G-1H).

Next, it was tested whether the Saa dietary intervention could affect tumor progression in a mismatch-repair proficient (pMMR), genetically-driven colonic tumor model that does not respond to ICI. Ape^{flox/+} mice were bred to CDX2-Cre mice for colon-specific heterozygous deletion of the tumor suppressor Apc (cAPC mice), because their pMMR status and Apc inactivation reflect two attributes observed in 85% of CRC patients; see e.g., Hinoi et al., Cancer Research 67, 9721 (2007), the contents of which are incorporated herein by reference in their entirety. At 6-8 weeks of age, cAPC mice were randomized to a dietary intervention of either low or high Saa for 12 weeks. Concordant with the findings in the MC38 CRC model, there was a 4-fold decrease in occurrence of advanced neoplastic lesions (i.e., adenocarcinomas) in cAPC mice fed the high Saa diet (see e.g., FIG. 1I), and these tumors were on average half the weight of those from low-Saa diet fed cAPC mice (see e.g., FIG. 1J). These data support that dietary Saa supplementation is a modulator of tumor progression in both MMR deficient (dMMR) and pMMR mouse CRC models.

Dietary Saa Modulated the Abundance of Mucispirillum schaedleri and Mucus Layer Thickness.

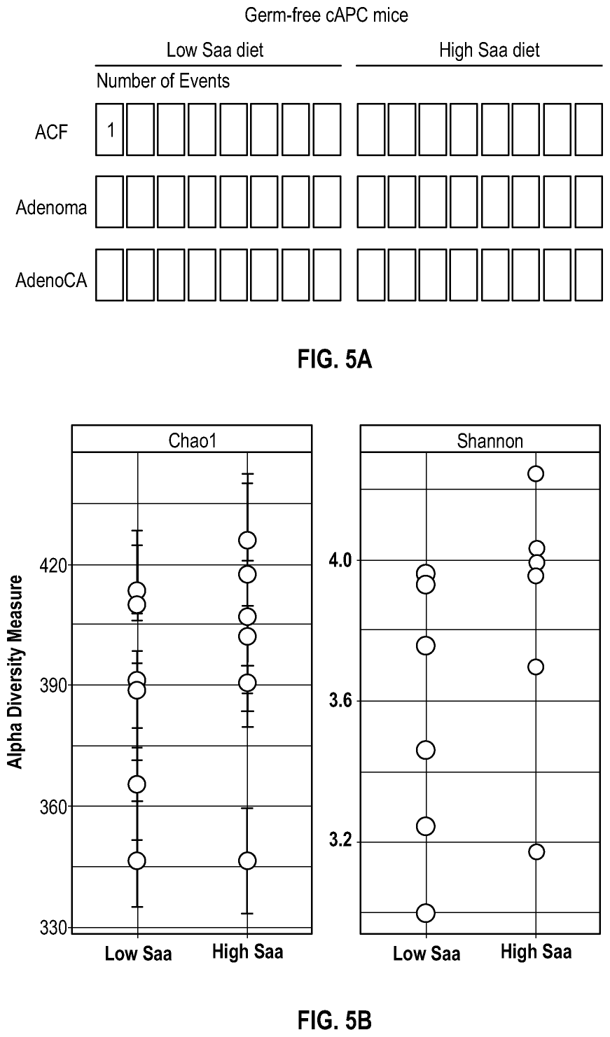

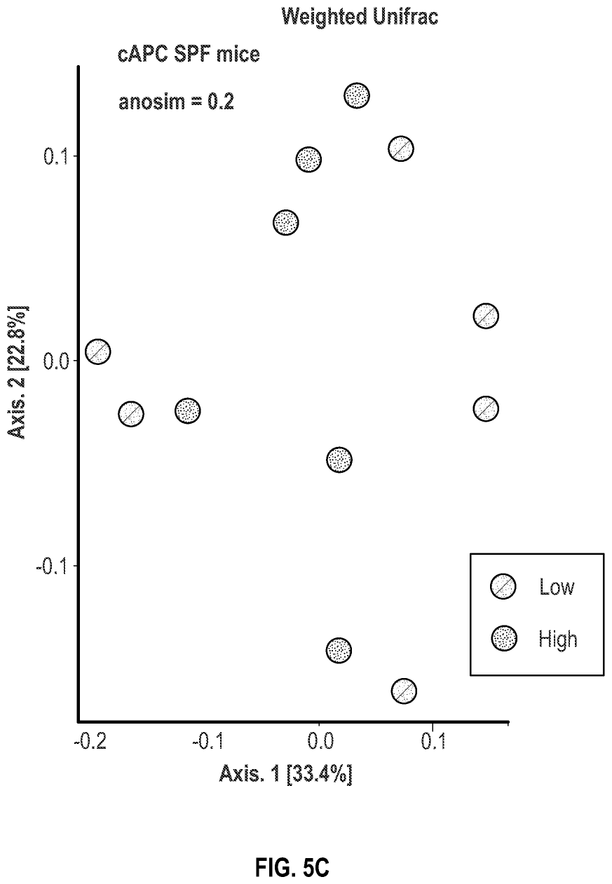

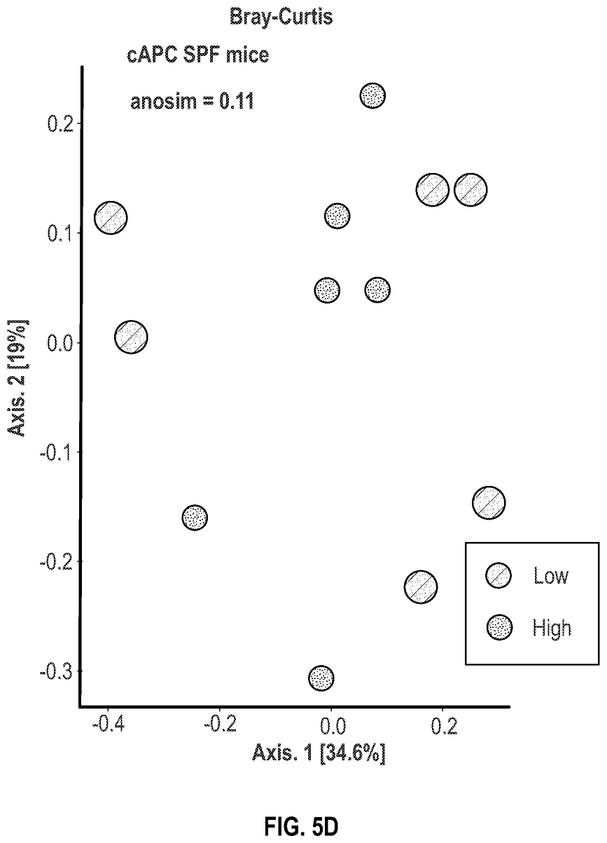

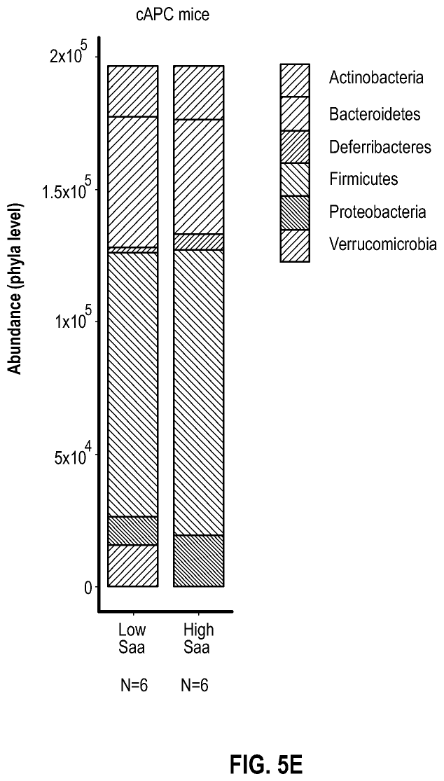

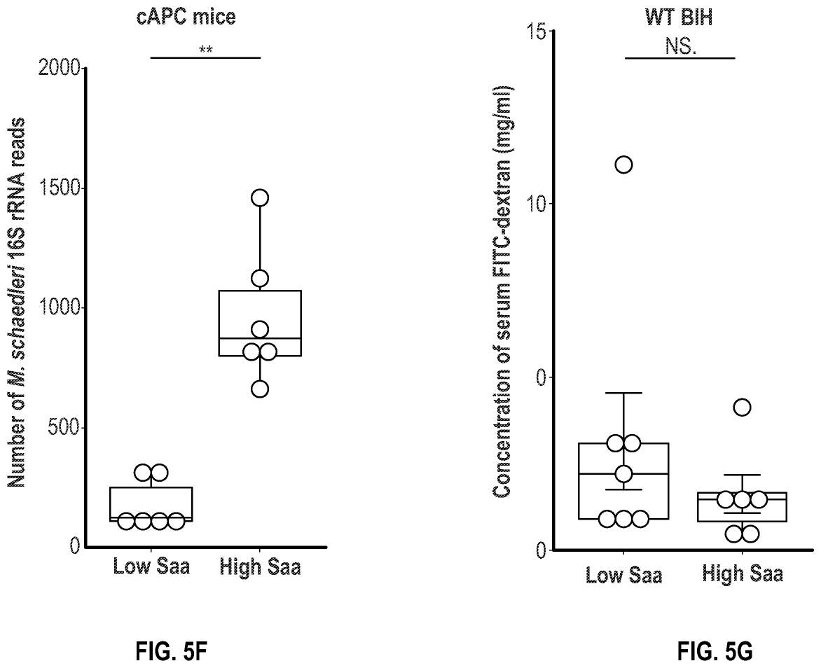

To determine if the effect on tumor growth and progression was dependent on the gut microbiota, cAPC mice were axenically re-derived, and the dietary intervention was repeated in germ-free (GF) cAPC mice starting at two months of age. Over our experimental time course of >4 mos., there were no colonic tumors in GF mice on either Saa diet (see e.g., FIG. 5A), supporting the microbiota dependency of CRC. In sum, the data point to dietary Saa supplementation as a modulator of tumor progression in both dMMR and pMMR mouse CRC models.

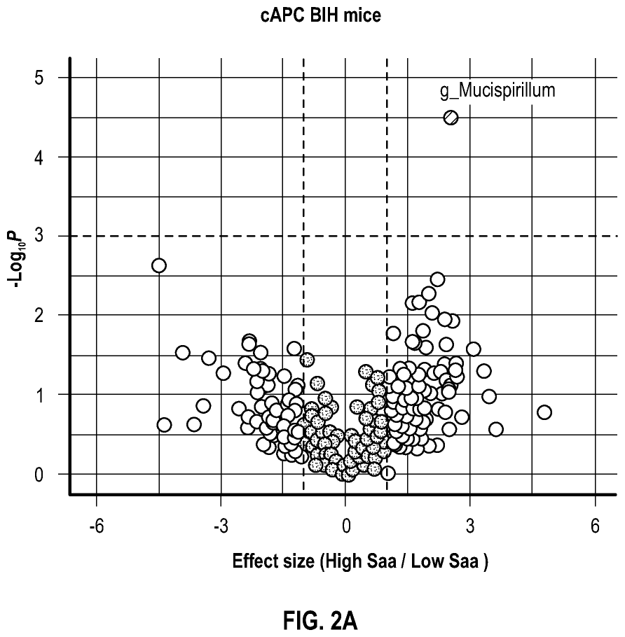

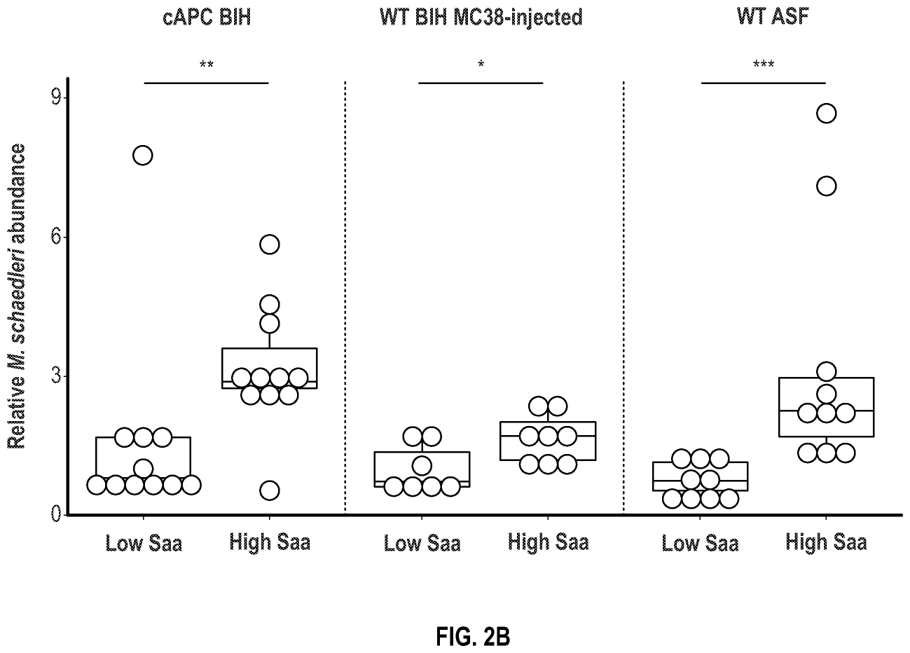

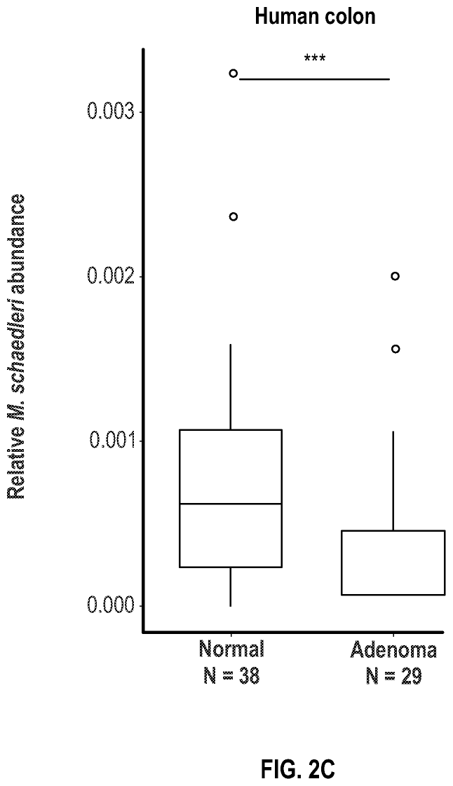

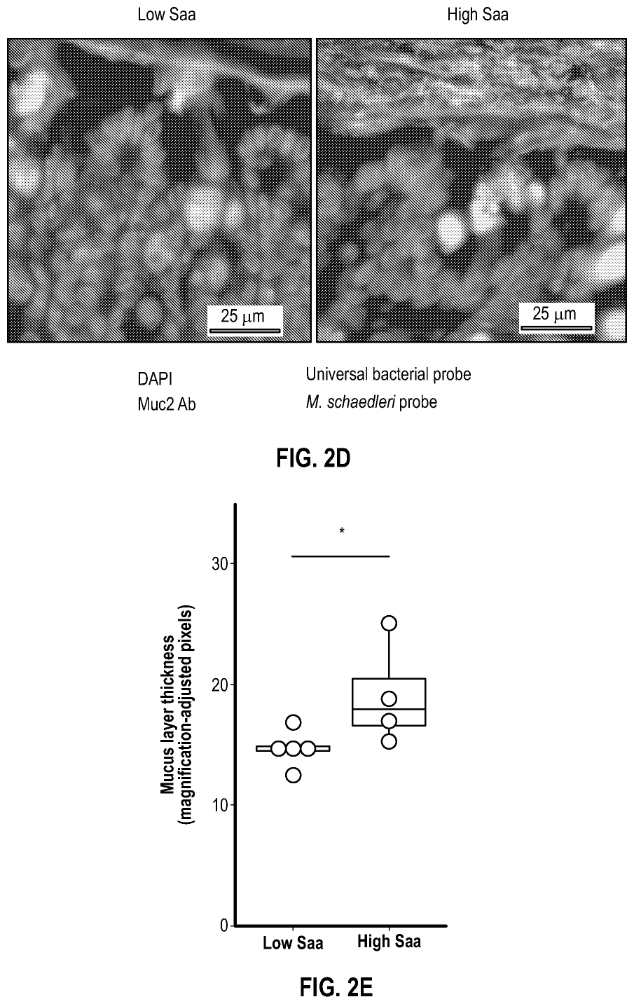

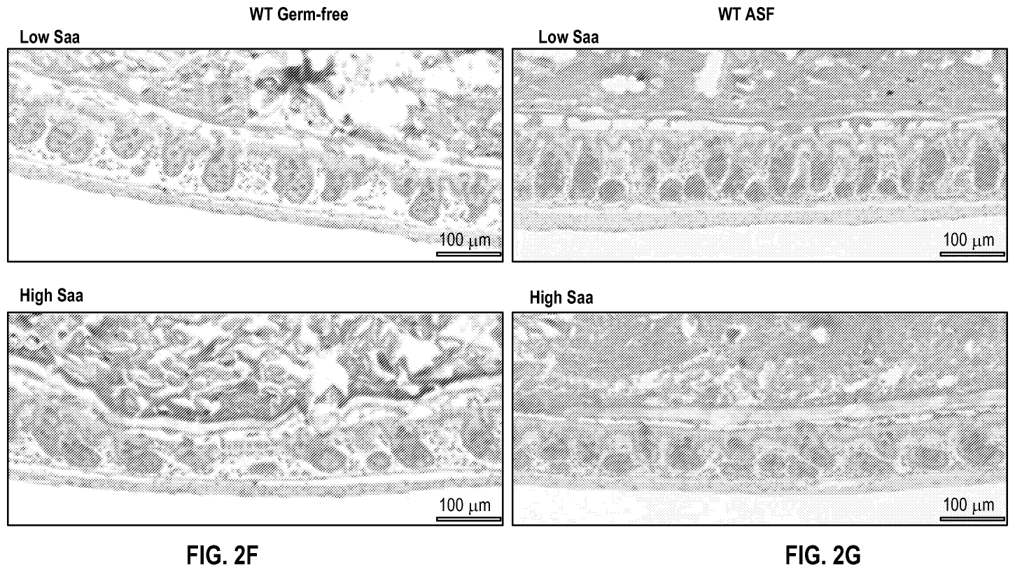

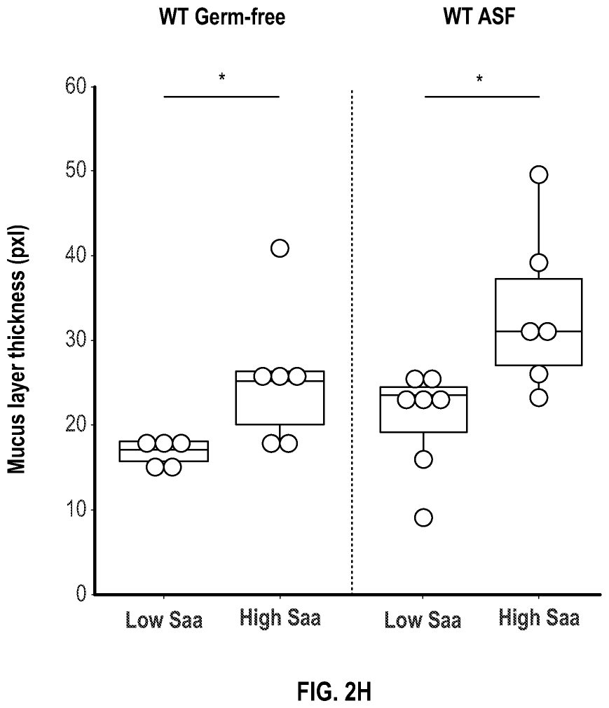

Since tumor development in cAPC mice was microbiota-dependent (see e.g., FIG. 5A), it was tested if gut microbiota composition was affected by dietary Saa by preforming a 16S rRNA gene amplicon survey on cecal contents from conventionally-reared cAPC mice fed the low vs. high Saa diets. As expected with this subtle dietary change, there was no statistically significant α-diversity differences (see e.g., FIG. 5B) or β-diversity separation (see e.g., FIG. 5C-5D) or. Only one taxon significantly changed in abundance; Mucispirillum schaedleri was the was enriched in cecal contents of mice fed the high Saa diet (see e.g., FIG. 2A and FIG. 5E-5F). To analyze housing-independent changes within specific operational taxonomic units (OTUs), MaAsLin 2 can be used. While Akkermansia muciniphila appeared to be increased in the cecal contents of mice on the low Saa diet (see e.g., FIG. 26A), this was not statistically significant and attributable to cage effects (see e.g., FIG. 28F).

M. schaedleri is a Gram-negative anaerobic Deferribacteraceae family member that inhabits the outer mucus layer of the mouse colon; M. schaedleri has been detected in up to 42% of human colonic mucosal biopsies; see e.g., Herp et al., Cell Host Microbe 25, 681-694.c8 (2019); Loy et al., mSystems 2, e00171-16 (2017); Robertson et al., Int J Syst Evol Microbiol 55, 1199-1204 (2005); Zmora et al., Cell 174, 1388-1405.e21 (2018); the contents of each of which are incorporated herein by reference in their entireties. Quantitative (q)-PCR analysis of cecal DNA from cAPC mice confirmed that M. schaedleri increased in mice fed the high Saa diet vs. low Saa diet (see e.g., FIG. 2B, left panel). The effects were detectable in WT mice from the vivarium (see e.g., FIG. 2B, middle panel) and observed in altered Schaedler flora (ASF) mice, which harbor M. schaedleri as part of their 8-member community (see e.g., FIG. 2B, right panel).

To assess the human relevance of M. schaedleri, human microbiome datasets were searched for M. schaedleri-specific reads (see e.g., Methods). The analyses did not detect reliable signals for M. schaedleri in human stool metagenomes. Since M. schaedleri is mucus-associated, it was contemplated that searching for its presence in human mucosal tissue microbiome samples can be more fruitful than in fecal metagenomic samples. Tissue associated levels of M. schaedleri were readily detectable in colonic biopsy samples and were enriched in normal colonic biopsy samples compared to adenomas (see e.g., FIG. 2C). Such samples and profiling were not available from ICI-responsive patients who did not experience ICI-induced colitis.

Given these human mucosal data (see e.g., FIG. 2C), M. schaedleri's localization was examined in the mouse colon using bacterial-directed fluorescence in situ hybridization (FISH). Since M. schaedleri localizes to the mucus layer, it is contemplated that dietary Saa can influence the abundance of M. schaedleri by modulating mucus layer thickness, creating a more favorable niche for M. schaedleri. Dietary amino acid supplementation (including L-cysteine) can increase colonic mucin synthesis in rats; see e.g., Faure et al., J Nutr 136, 1558-1564 (2006), the contents of which are incorporated herein by reference in their entirety. M. schaedleri bloomed in the colonic mucus in mice fed the high Saa diet (see e.g., FIG. 2D), and the mucus layer also appeared thicker (see e.g., FIG. 2E).

To assess the effect of dietary Saa and its microbiota-dependence on mucus layer thickness. GF mice were first fed low and high Saa diets, and the distal colon mucus layer was measured by Alcian blue staining. In the absence of a microbiota, the high Saa diet increased the inner mucus layer thickness by 20% (see e.g., FIG. 2F, FIG. 2H). This analysis was next extended to examine mice with an altered Schaedler flora (ASF) microbiome (comprised of a defined consortium of 8 bacterial species including M. schaedleri); see e.g., Wymore et al., ILAR J 56, 169-178 (2015), the contents of which are incorporated herein by reference in their entirety. The high Saa diet induced a ˜50% increase in mucus layer thickness (see e.g., FIG. 2G-2H), indicating that the microbiota amplifies the high Saa diet effect on mucus thickness.

In these mice, an increase in M. schaedleri abundance in the mucus layer was observed in response to the high Saa diet, as assessed by qPCR (see e.g., FIG. 28E). qPCR was also performed to quantify M. schaedleri levels in the context of a more complete conventionally-reared mouse microbiota in the dMMR heterotopic CRC mice described above, and these results again confirmed increased M. schaedleri abundance in mice fed the high Saa diet (see e.g., FIG. 28F).

It was also investigated if this mucus thickness difference altered colonic barrier integrity, as reduced colonic barrier integrity has been implicated in CRC-promoting smoldering inflammation. However, FITC-dextran intestinal permeability testing did not reveal a difference between WT mice fed the low vs. high Saa diet (see e.g., FIG. 5G). Collectively, these data indicate that dietary Saa levels modulated the abundance of M. schaedleri, a member of the mouse and human microbiome, by affecting the colonic mucus layer and its habitability as an ecological niche for this species.

High Saa Diet LED to Increased Numbers of CD8⁺ T Cells in cAPC Tumors

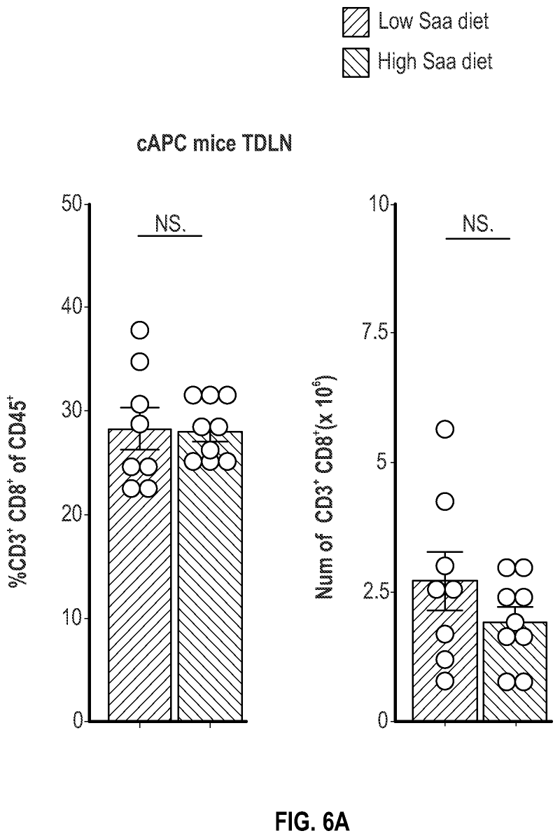

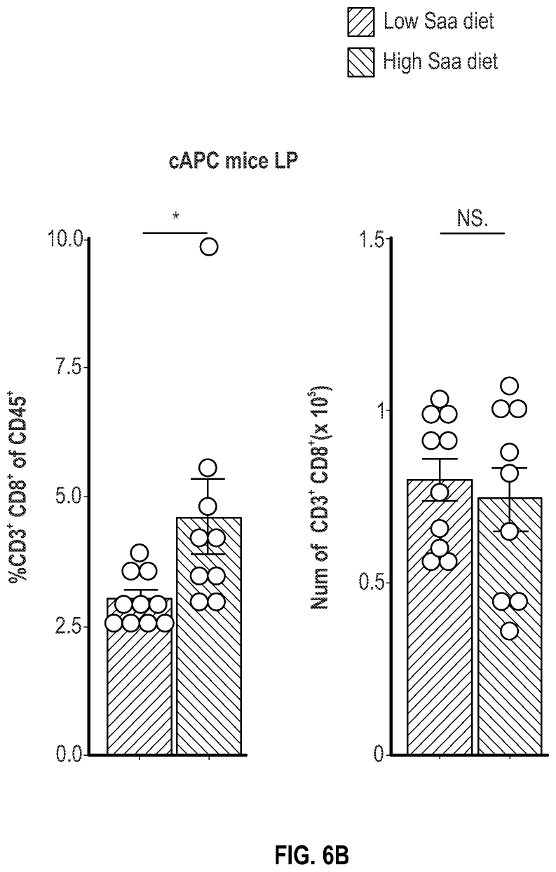

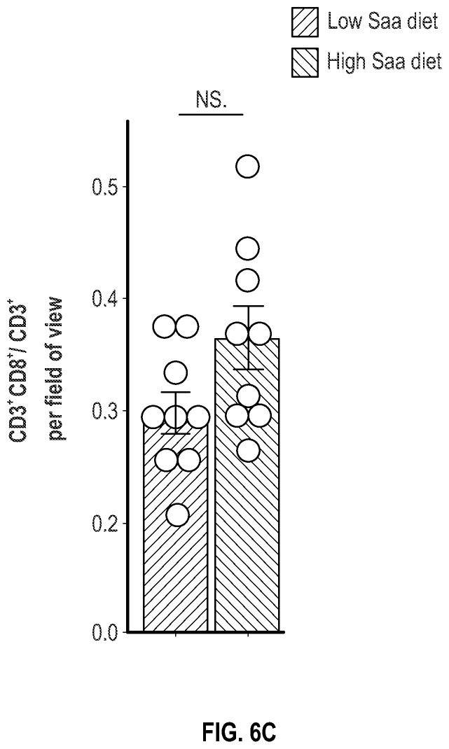

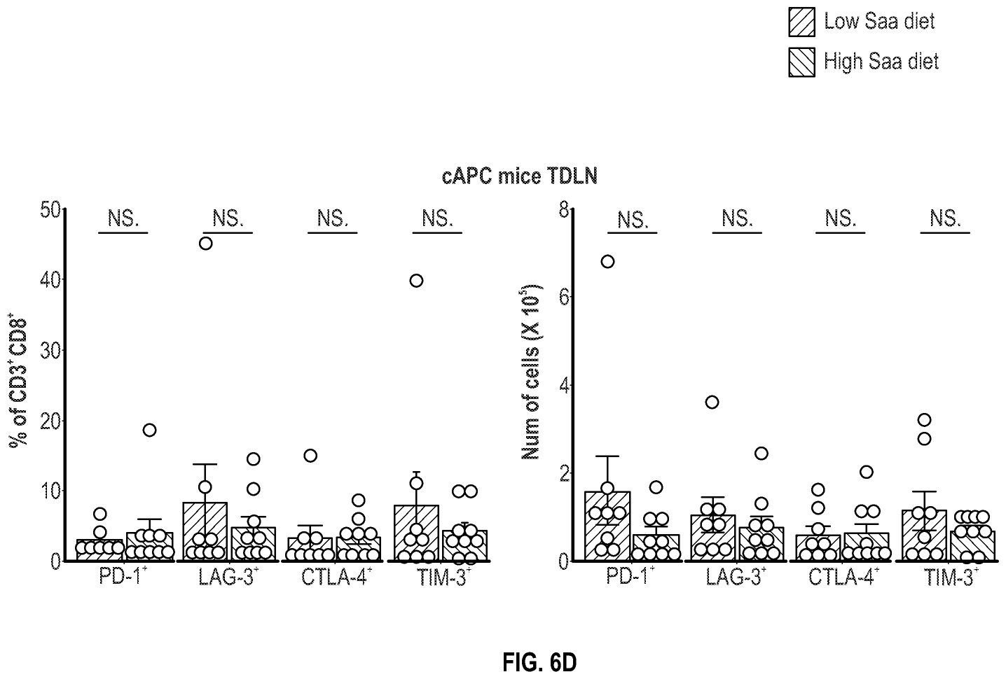

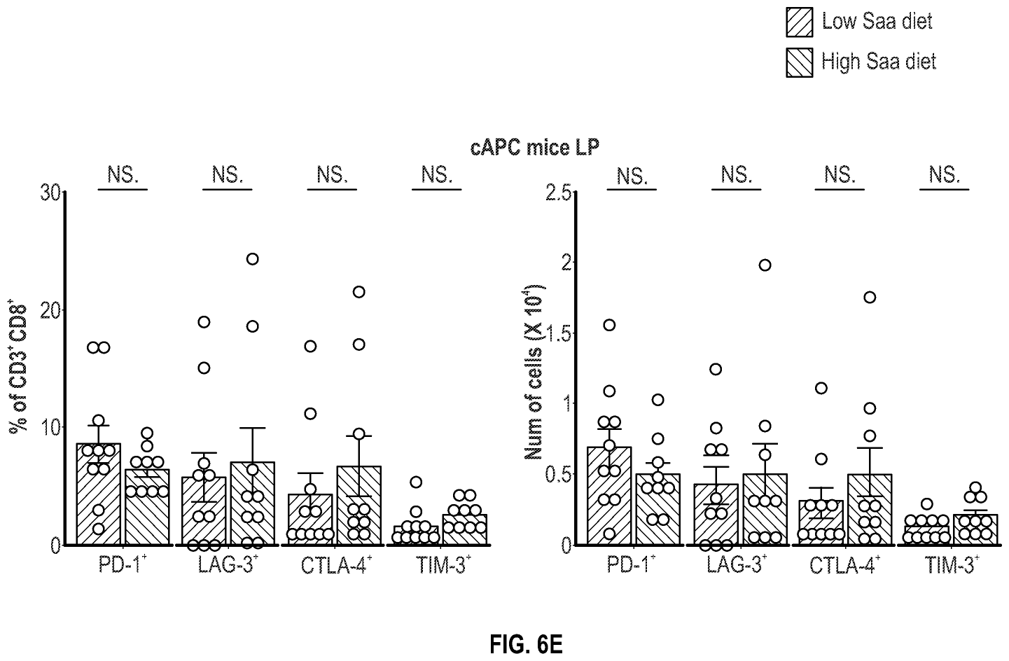

To identify the mechanistic links uniting dietary Saa, microbial activities, and anti-tumor immunity, intratumoral cytotoxic CD8⁺ T cells were assessed, as a critical classifier of “warm” tumors, in tumors from cAPC mice fed the high or low Saa diets. As used herein, the term “warm tumors” refers to tumors with a “T cell inflamed” phenotype, and the term “cold tumors” conversely refers to tumors with lack or paucity of tumor T cell infiltration; see e.g., Bonaventura et al., Front Immunol 10, 168 (2019), the contents of which are incorporated herein by reference in their entirety. There were higher frequencies and numbers of CD8⁺ T-cells in tumors from high Saa-fed cAPC mice compared with tumors from low Saa-fed mice (see e.g., FIG. 3A). CD8⁻ T cell frequency and number remained relatively unchanged in the adjacent normal colon lamina propria (LP) and the tumor draining lymph nodes (TDLN) (see e.g., FIG. 6A-6B). Utilizing immunofluorescence microscopy, a 2-fold increased ratio of CD8⁺ T cells was also observed in tumors from high Saa diet-fed cAPC mice (see e.g., FIG. 3B-3C), while no differences were observed in the CD3⁺CD8⁺/CD3⁺ cell ratio in the surrounding colonic LP (see e.g., FIG. 6C). To gain functional insight into the tumor infiltrating CD8⁺ T-cells enriched during Saa feeding, the expression of several immune co-inhibitory receptors, IFNγ, and Granzyme B (GZMB) was profiled. The proportions of IFNγ⁺ and GZMB⁺ CD8⁺ T cells were significantly increased in tumors from mice fed the high Saa diet (see e.g., FIG. 3E), while the proportions of PD-1⁺ and LAG-3⁺ CD8⁺ T-cells were significantly decreased (see e.g., FIG. 3D), indicating that these CD8⁺ T-cells were more capable of mounting anti-tumor immune responses. The expression of the immune checkpoint markers TIM-3 and CTLA-4 did not differ between the two groups. No differences were observed in the expression of the immune checkpoint markers in the LP or TDLN (see e.g., FIG. 6D-6E). In sum, the tumor microenvironment of mice fed high Saa diet was immunologically warmer than that in mice fed low Saa diet.

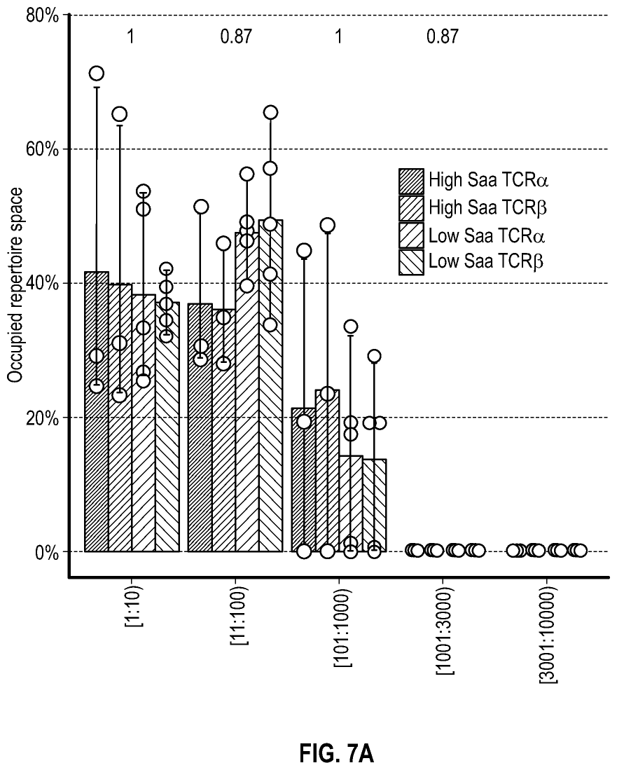

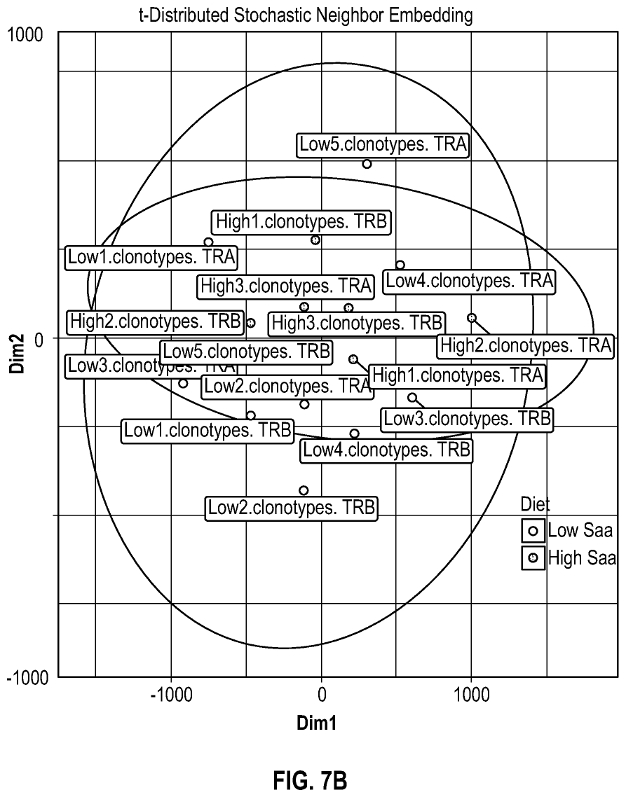

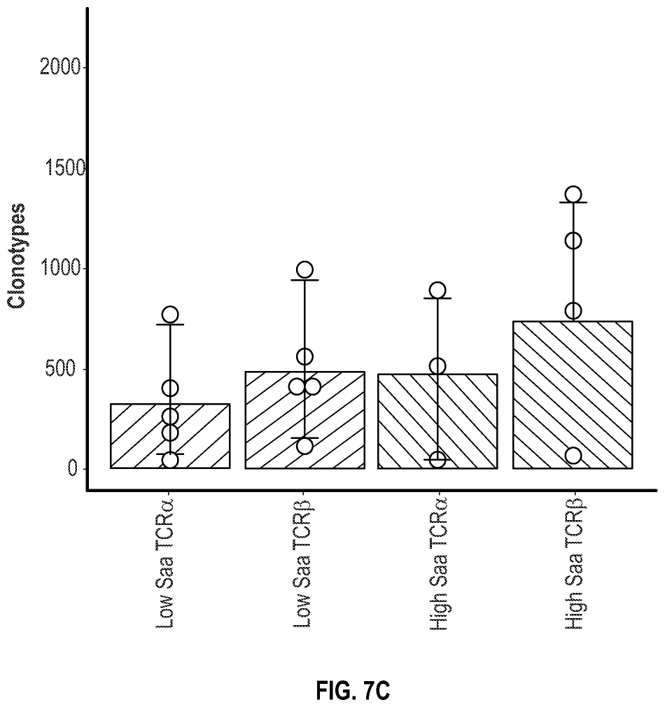

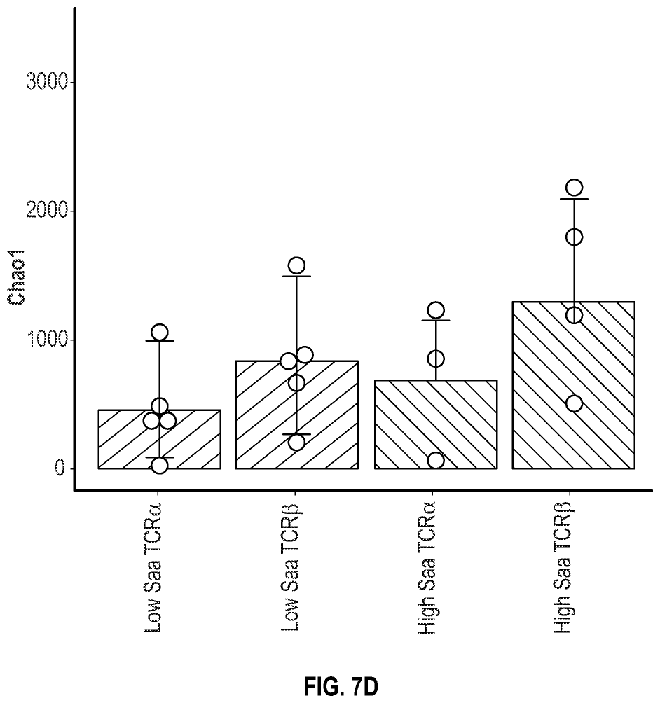

As tumor neoantigen diversity can contribute to tumor infiltrating T-cell frequencies and numbers, a TCR-Seq analysis of intratumoral CD8⁺ T-cells from cAPC mice was performed. The TCR-Seq analysis did not reveal any differences in the CD8⁺ T-cell clonotypic composition or abundance in tumors from low vs. high Saa diet-fed cAPC mice (see e.g., FIG. 7). Thus, dietary Saa did not influence TCR diversity in cAPC colonic tumors, therefore prompting examination of antigen presenting cells as a mechanism through which the diet affects tumoral infiltration and activation of CD8⁺ T cells.

Mucispirillum schaedleri Abundance Correlated with CD103⁺CD11b⁻ Dendritic Cell (cDC1) Frequency in Tumor Draining Lymph Nodes (TDLN).

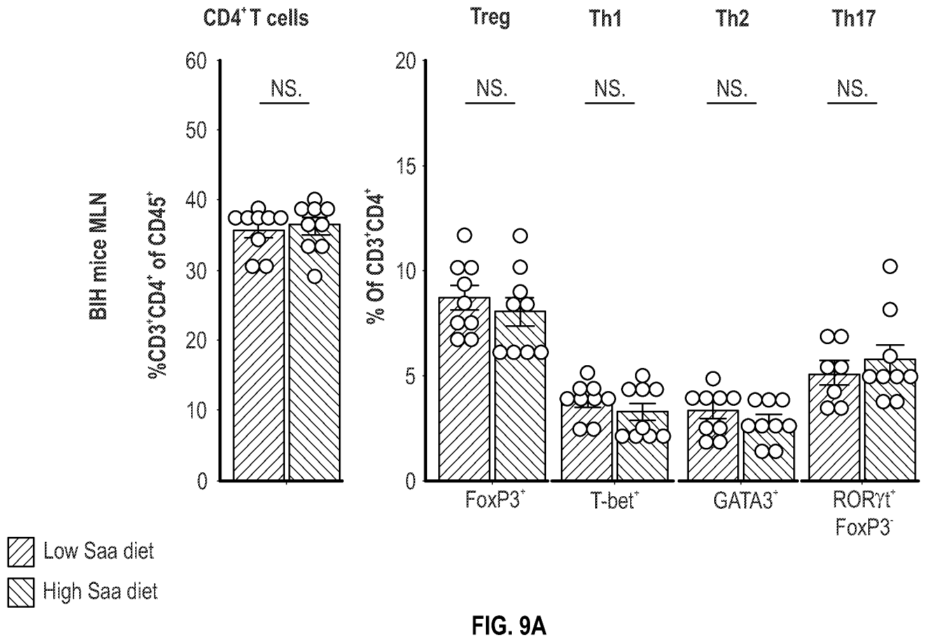

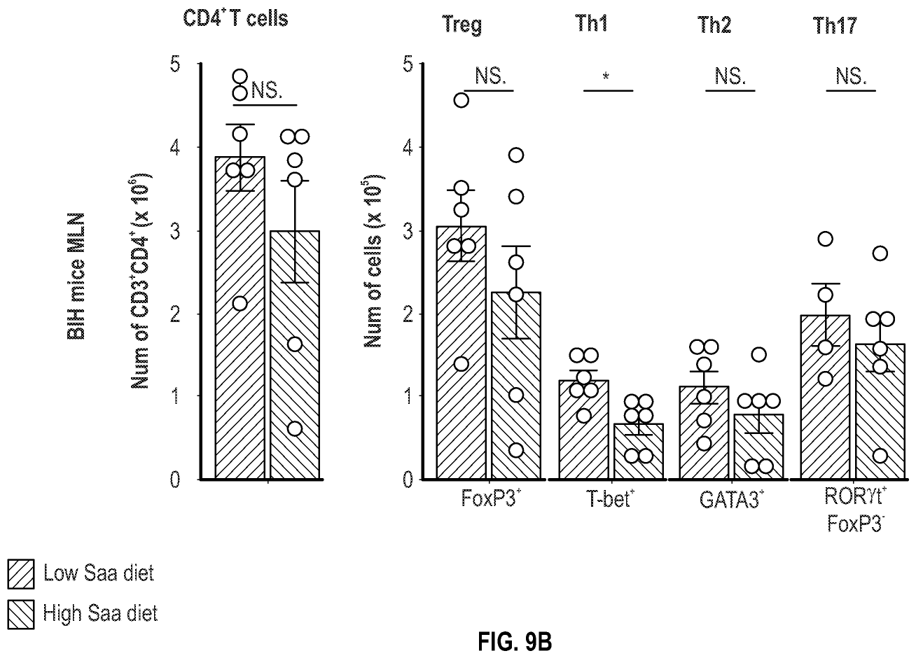

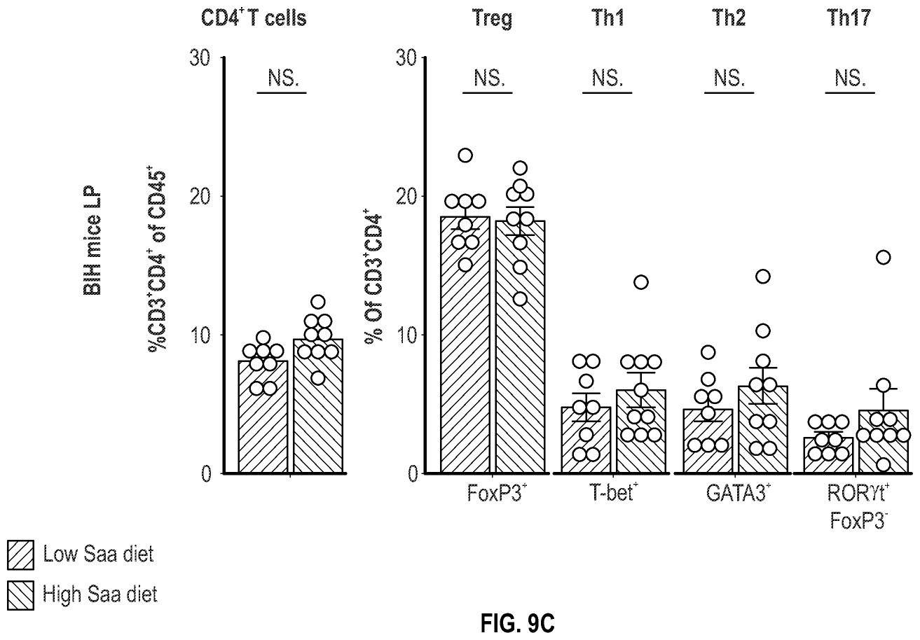

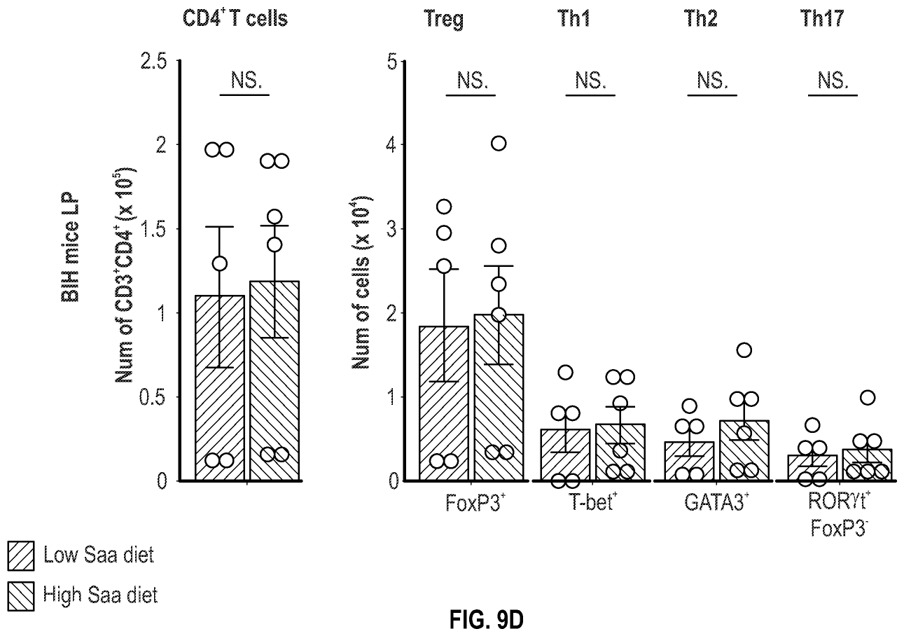

M. schaedleri is considered a mucus-dwelling bacterium, increasing its interaction with host cells, similar to other mucosal-associated microorganisms such as segmented filamentous bacterium (SFB). Whereas SFB has been extensively studied for its ability to induce a Th17 immune response, the immunomodulatory effects and underlying mechanisms of M. schaedleri remain less well-characterized. M. schaedleri induced a T_{H}1-type colitis in Nod2^{-/-}×Cybb^{-/-} mice and to play a role in peripheral regulatory T cell (Treg) development; see e.g., Caruso et al., Sci Immunol 4, eaaw4341 (2019); Campbell et al., Immunity 48, 1245-1257.e9 (2018); the contents of each of which are incorporated herein by reference in their entireties. To address what immune modulating effects can be induced by M. schaedleri in the diet-CRC model, immune cell populations in the colonic LP and TDLN of cAPC mice, and colon LP and MLN of WT bred in-house (BIH) mice fed the Saa diets were profiled (see e.g., FIG. 8 for gating strategies). Analysis of the major subsets of CD4⁺ T cells, including Th1 cells, Th2 cells, Th17 cells, and Tregs, did not reveal any differences between mice fed low or high Saa diets in either colonic LP or MLN (see e.g., FIG. 9).

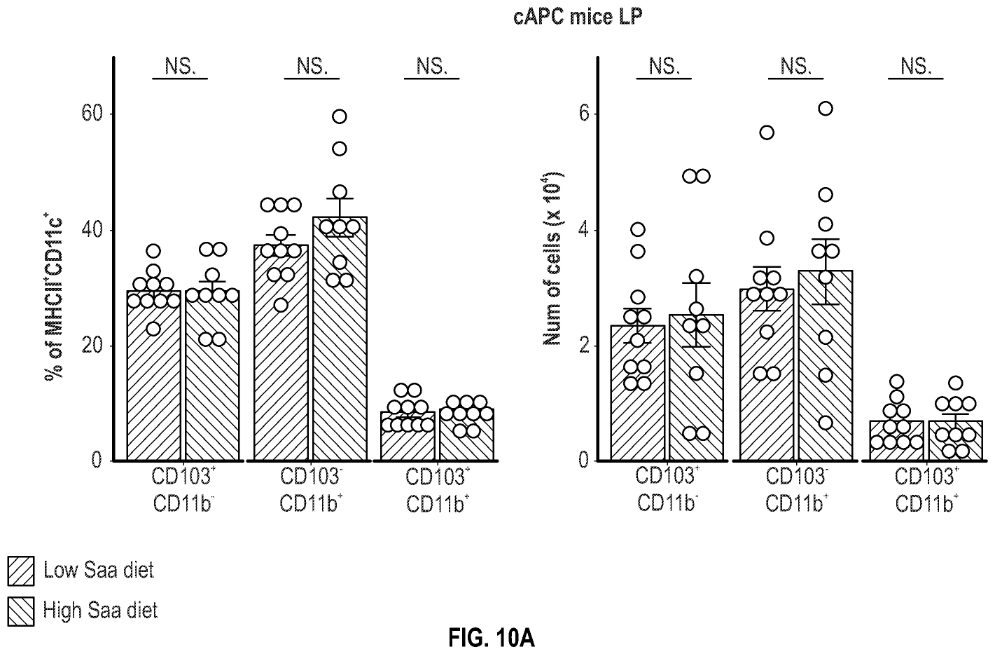

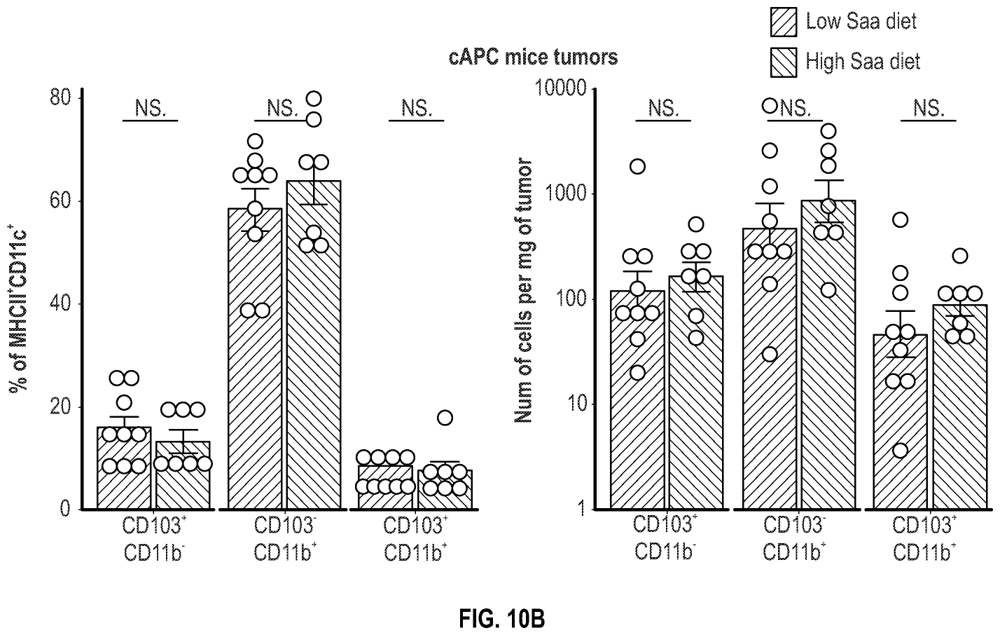

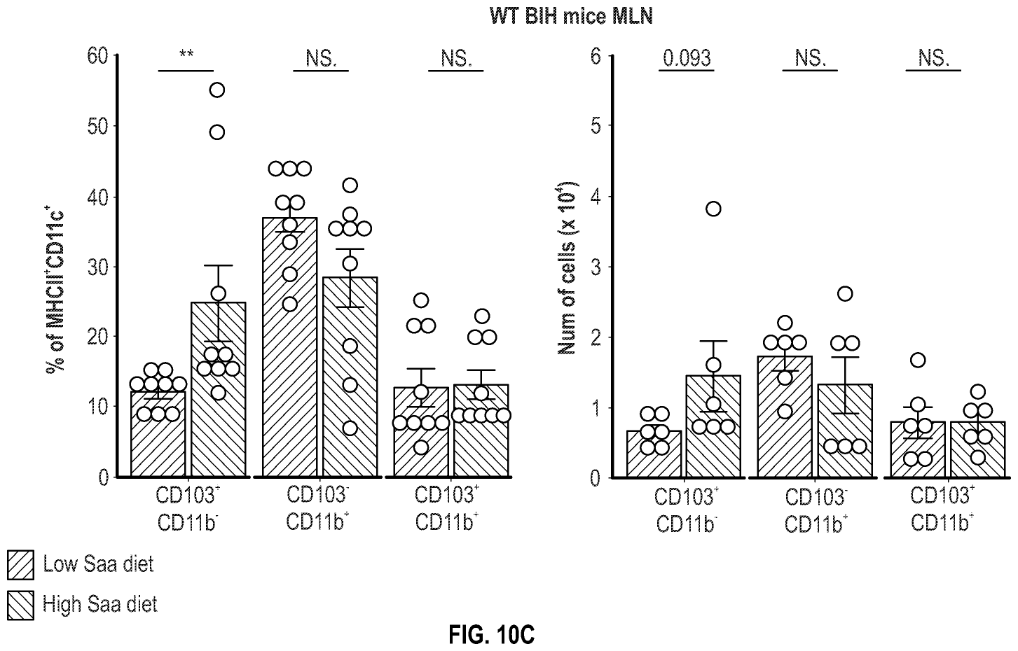

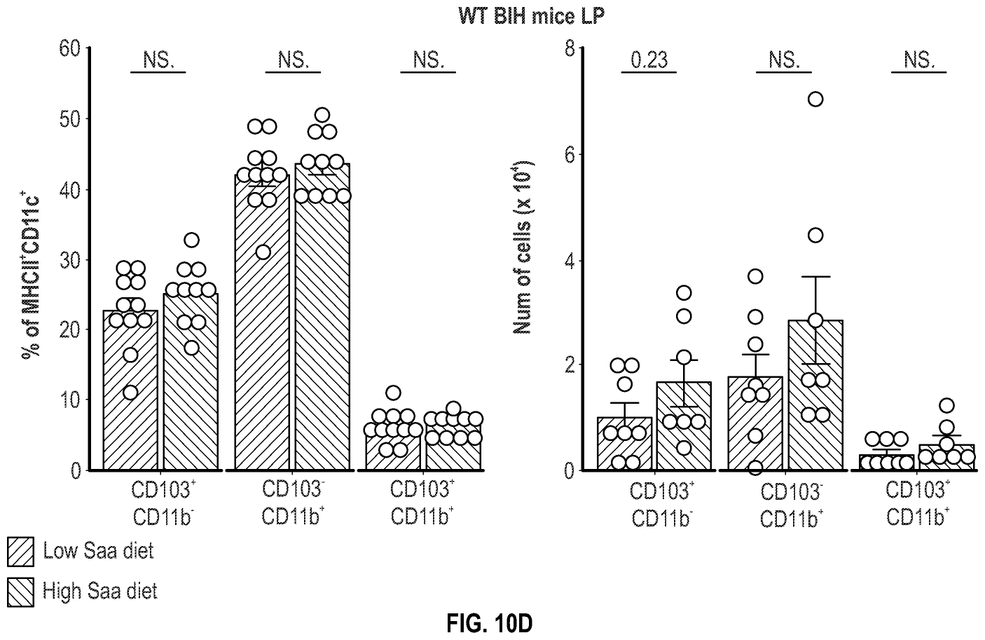

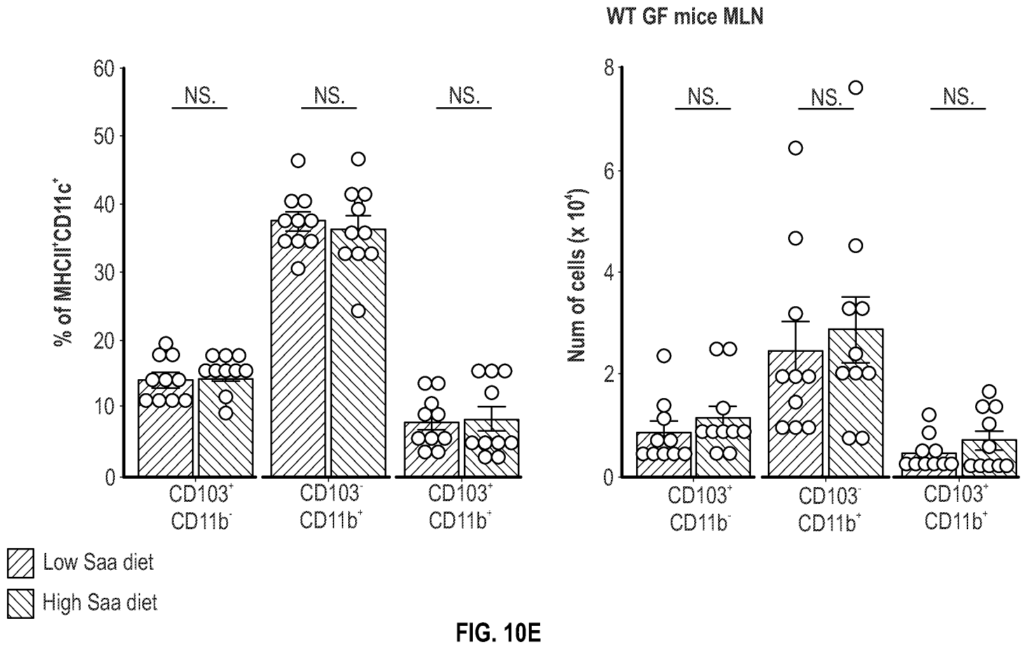

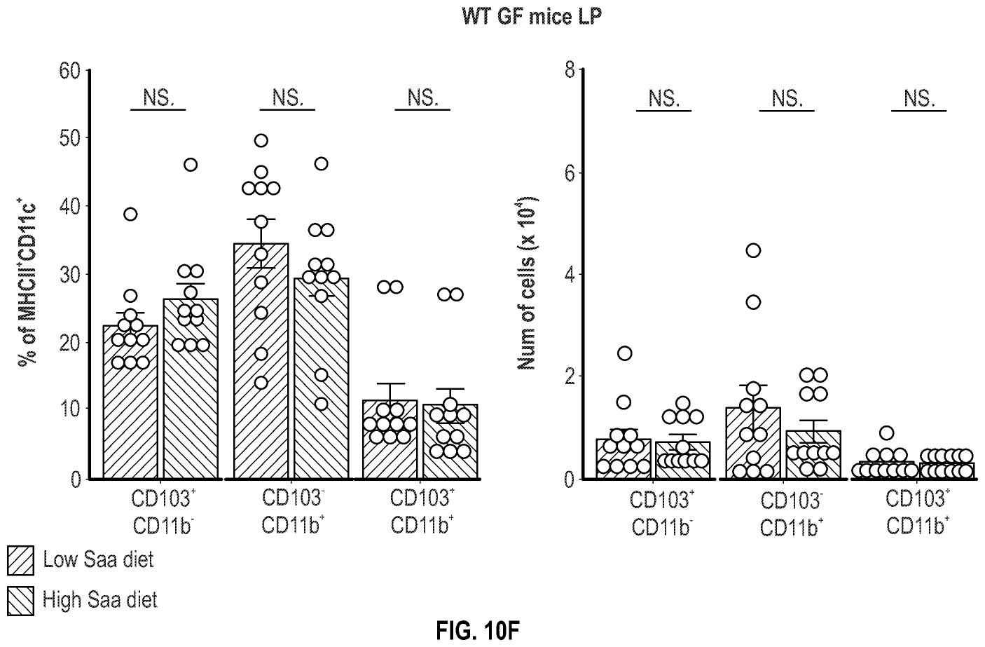

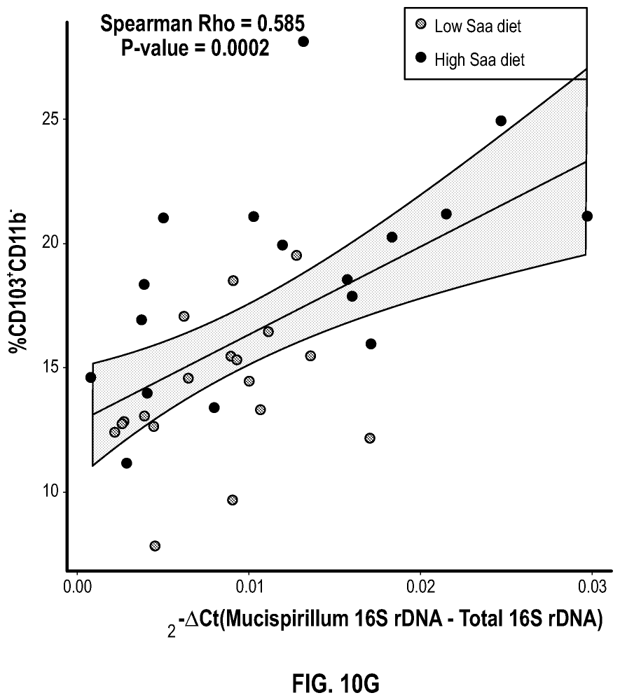

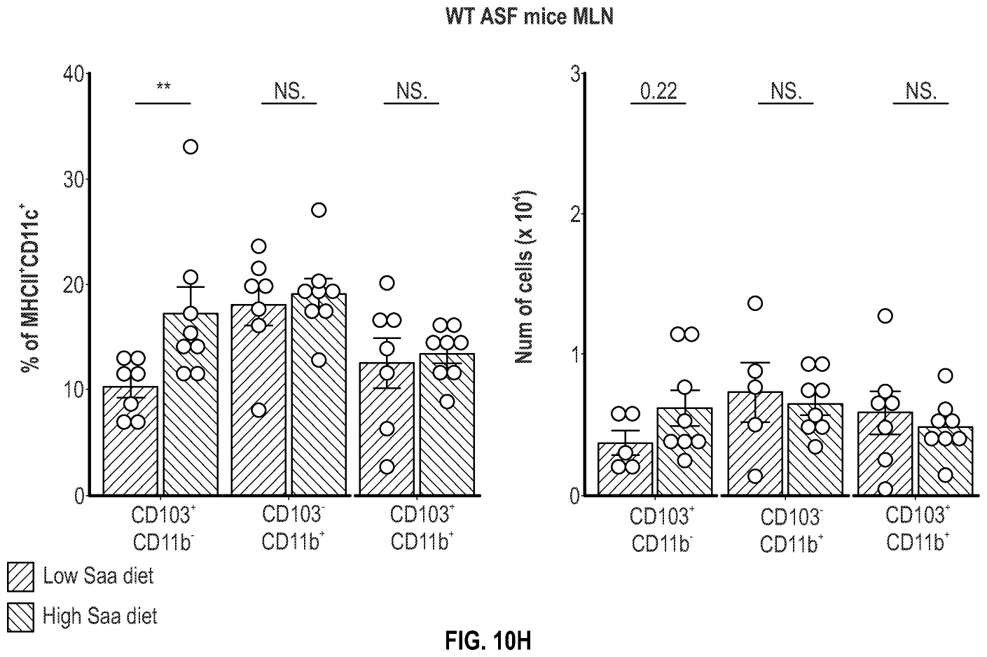

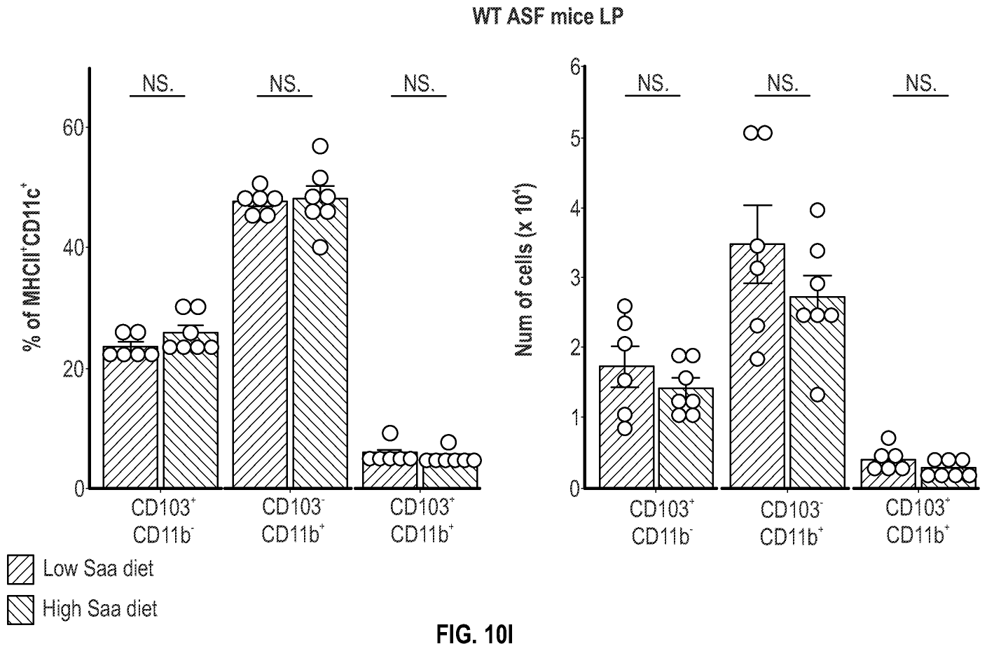

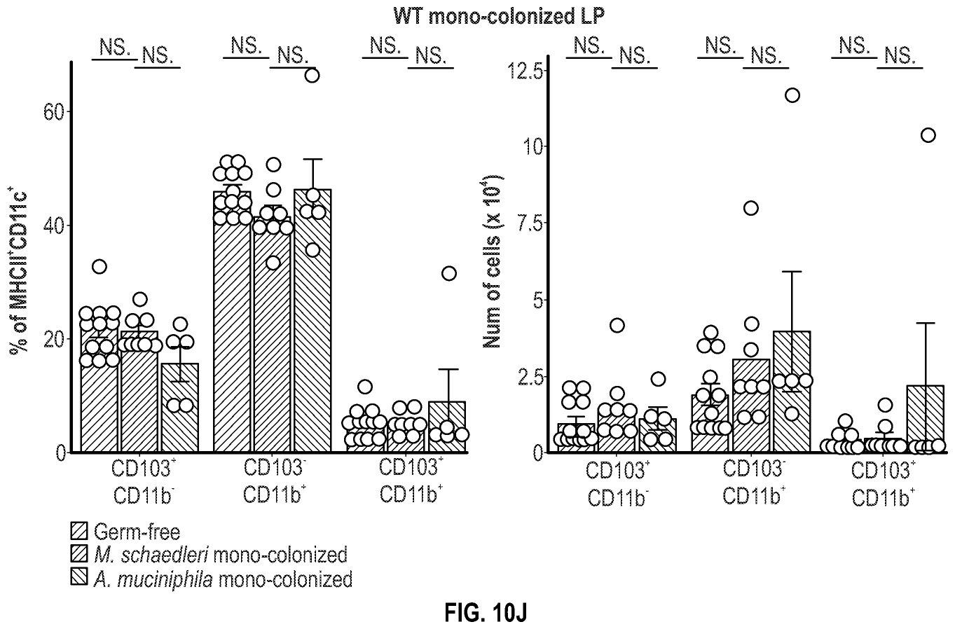

In contrast, analysis of myeloid cells revealed a significant increase in the frequency and number of CD103⁺CD11b⁻ dendritic cells (termed cDC1) in the TDLN of cAPC mice fed the high Saa diet, while other CD11c⁺ cell populations remained unchanged (see e.g., FIG. 3G). LP and tumor CD11c⁺ cell frequencies and numbers were unchanged between the low and high Saa-fed cAPC mice (see e.g., FIG. 10A-10B). cDC1 are initiators of anti-tumor immunity as they cross-present tumor-derived antigens in the TDLN to elicit CD8+ T cell anti-tumor responses. Similar results were obtained for myeloid cell populations in the LP and MLN from WT BIH mice fed the Saa diets (see e.g., FIG. 10C-10D). To determine whether the increase in cDC1 was is dependent on microbial factors, germ-free (GF) mice were fed low and high Saa diets and their immune cell populations were analyzed. There was no increase in cDC1 in GF mice fed the high Saa diet in the MLN or LP (see e.g., FIG. 10E-10F). Utilizing data from all the conventionally-reared mice (WT and cAPC), there was a significant positive correlation (Spearman ρ=0.585, P value=0.0002) between M. schaedleri cecal abundance and MLN/TDLN cDC1 population frequency (see e.g., FIG. 10G).

Next, it was assessed if M. schaedleri contributed to the cDC1 increase using three approaches. First, ASF mice, which harbor M. schaedleri, were fed the low or high Saa diets, and an increase in cDC1 was only observed in the MLN of the high Saa diet fed group (see e.g., FIG. 10H-10I); such data demonstrates that the 8-species ASF consortium, which includes M. schaedleri, was sufficient for a cDC1 increase in the presence of the high Saa diet. Second, GF mice were monocolonized with either M. schaedleri or A. muciniphila, and both groups were fed the high Saa diet. A. muciniphila was chosen as it is also a mucus-dwelling bacterium with immunomodulatory properties relevant for ICI response. cDC1 frequencies and numbers only increased in the MLN of mice monocolonized with M. schaedleri, but not in those colonized with A. muciniphila, demonstrating that it was not just the presence of gut colonization per se that was required for this observed cDC1 effect (see e.g., FIG. 3H and FIG. 10J). Third, since many bacteria secrete bioactive molecules, it was tested if abiotic M. schaedleri conditioned media (“CDM” or “CM”) affected cDC1 frequencies in the MLN of C57BL/6 BIH mice. M. schaedleri CDM gavage over a two-week period increased cDC1 frequency in MLN, while the abiotic medium control had no effect (see e.g., FIG. 31). These results indicate that M. schaedleri, and specific factors secreted by it, were sufficient for increasing MLN/TDLN cDC1 frequency and number.

Depletion of cDC1 or XCL1 Abolished the Anti-Tumor Effect of the High Saa Diet.

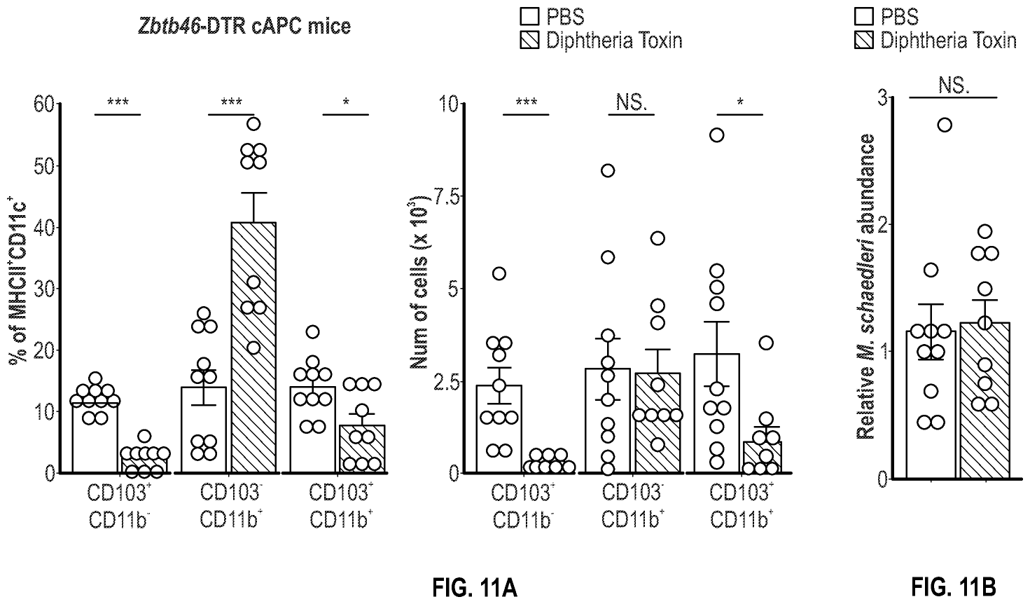

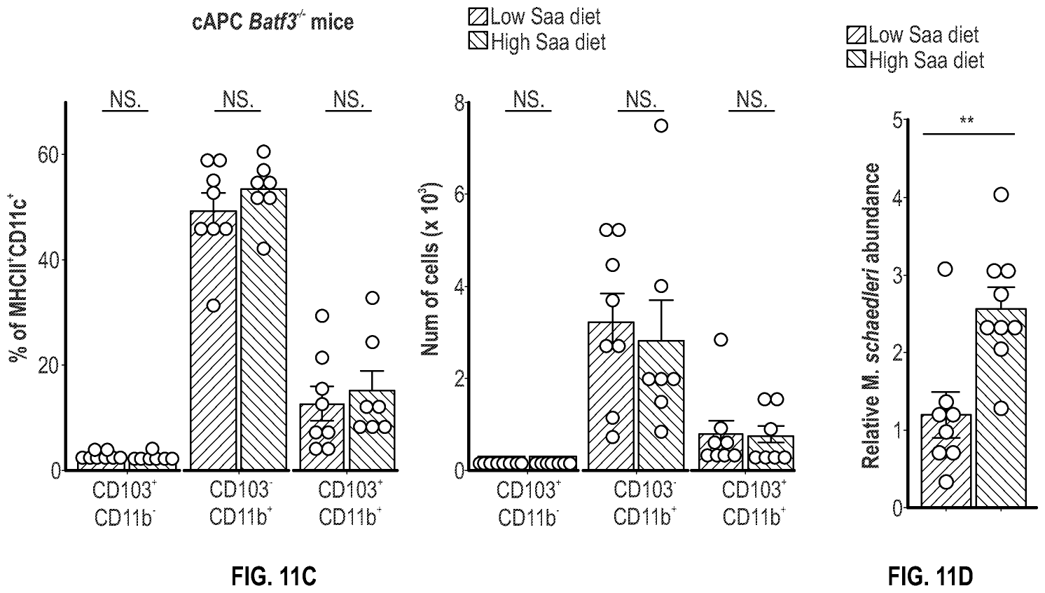

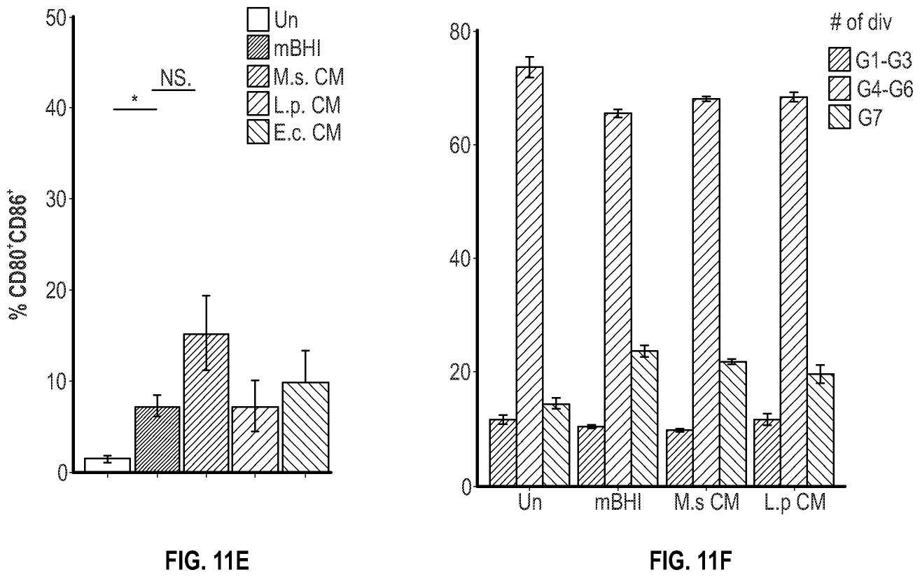

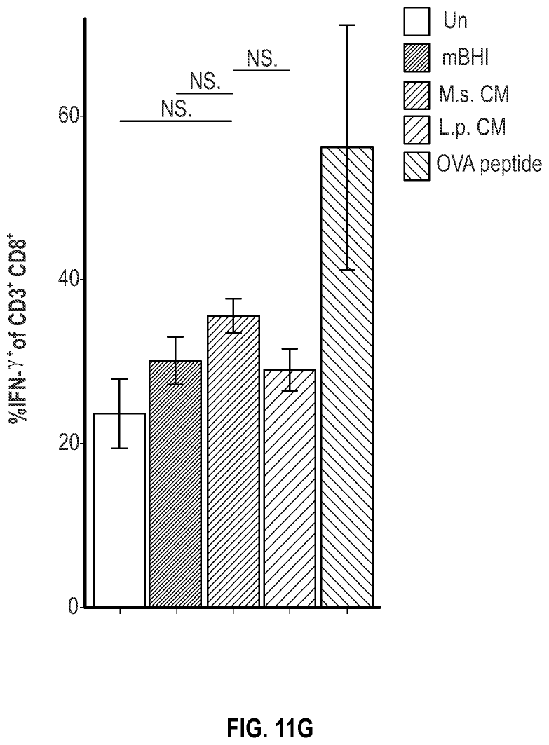

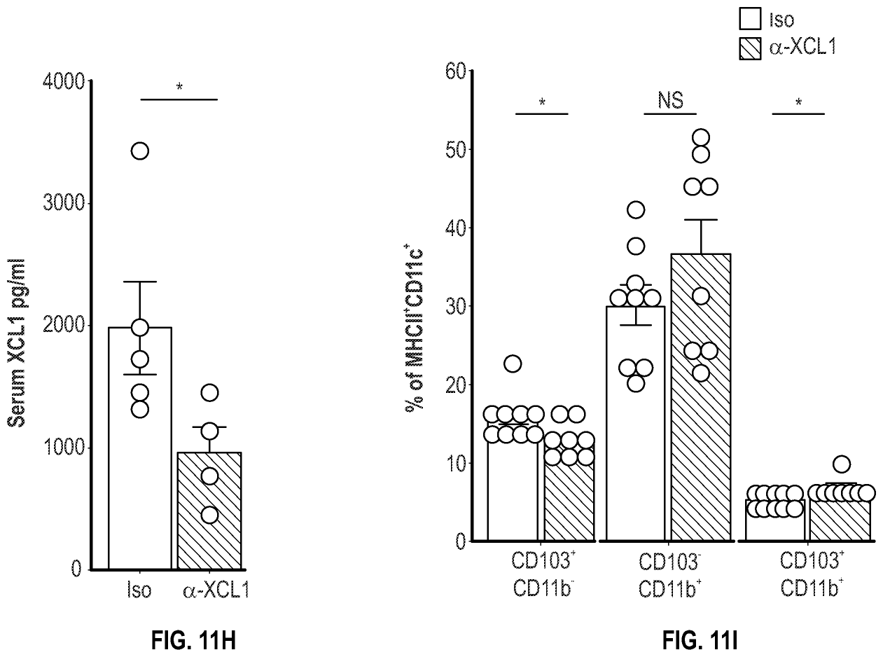

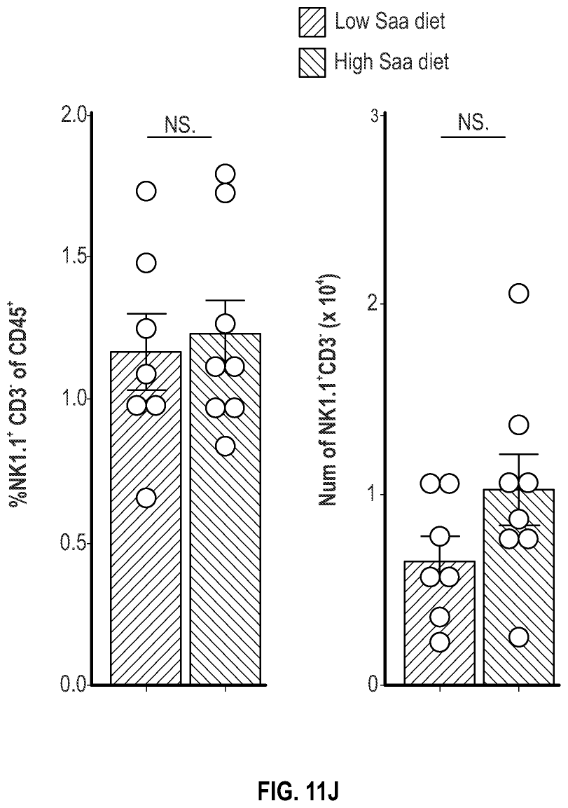

The role of cDC1 cells in CD8⁺ T cell-mediated anti-tumor immunity was tested in the context of CRC. To determine whether cDC1 cells mediated the high Saa diet-dependent and M schaedleri-dependent effects on anti-tumor immunity and tumor growth as described above, three approaches were used to deplete cDC1. First, bone marrow chimeric mice were generated with hematopoietic cells from Zbtb46-DTR mice, which express the diphtheria toxin receptor (DTR) under the regulation of the cDC1 specific promoter upstream of Zbtb46 and permit diphtheria toxin-mediated cDC1 depletion; see e.g., Meredith et al., J Exp Med 209, 1153-1165 (2012), the contents of which are incorporated herein by reference in their entirety. These cAPC Zbtb46-DTR mice were fed a high Saa diet (which drives M. schaedleri expansion and the subsequent MLN-TDLN cDC1 increases) and received diphtheria toxin (DT) injections starting 4 weeks post-irradiation and engraftment to deplete cDC1 or PBS injections as a control. The expected decrease in TDLN cDC1 numbers was observed in DT-treated mice, while the frequencies and numbers of other CD11c+ cells were unchanged (see e.g., FIG. 11A). There was no difference in M. schaedleri cecal abundance between the DT and PBS treated groups (see e.g., FIG. 11B). cDC1 depletion increased the frequency of neoplastic lesions even in the presence of the high Saa diet (see e.g., FIG. 4A). This finding further supports cDC1's role as a mediator of anti-tumor immunity and regulation of neoplastic progression and substantiates cDC1 function in mediating the antitumor effects of a high Saa diet-M. schaedleri bloom.

As a second approach for depleting cDC1 in cAPC mice, the cAPC line was crossed to Batf3 knock-out (KO) mice, generating cAPC Batf3^{-/-} mice. Like Zbtb46, Batf3 is a transcription factor crucial for the development of cDC1. cAPC Batf3^{-/-} mice were fed the high or low Saa diets and, as expected, there were diminished frequencies and numbers of cDC1, but not cDC2 in the TDLN of both groups (see e.g., FIG. 11C). Higher M. schaedleri abundance was also detected in the cecal contents of the high Saa diet-fed group as compared to the mice receiving the low Saa diet (see e.g., FIG. 11D). Supporting findings in FIG. 4A, Batf3 deficiency led to loss of the protective effect of high Saa diet as there was no difference in tumor numbers or neoplastic progression between the two diet groups (see e.g., FIG. 4B). As the data supported that cDC1 were a critical link connecting the Saa diet, M. schaedleri, and anti-tumor immunity (see e.g., FIG. 3F-3I and FIG. 4A-4B), it was examined if M. schaedleri acted directly on cDC1s. The in vitro testing of the abiotic M. schaedleri CM on cDC1 did not support that it directly enhanced cDC1 activation of CD8⁺ T cells via increased expression of co-stimulatory molecules or cross-presentation (see e.g., FIG. 11E-11G).

To determine what drives the observed effects on cDC1, further testing focused on a factor known to activate and recruit cDC1, lymphotactin (XCL1). In a third approach to perturb cDC1s an α-XCL1 antibody (Ab) treatment was used to deplete this cDC1 specific chemokine and cytokine in the setting of the high Saa diet; see e.g., Matsumoto et al., J Immunol 199, 82-90 (2017); Lei et al., Microbes Infect 14, 262-267 (2012); the contents of each of which are incorporated herein by reference in their entireties. cAPC mice treated with α-XCL1 Ab had reduced serum levels of XCL1 and lower cDC1 in their TDLN (see e.g., FIG. 11H-11I). Reduction of XCL1 levels led to a significant increase in adenocarcinomas in cAPC mice fed high Saa diet (see e.g., FIG. 4C) even though the α-XCL1 Ab only reduced serum levels by two-fold on average (see e.g., FIG. 11H). Collectively, these experiments affirm the role of cDC1 in the immunoregulation of tumor growth in cAPC mice and link diet-microbial interactions and anti-tumor immunity.

Dietary Saa and M. schaedleri Induced XCL1 Secretion from Natural Killer T (NKT) Cells.

The observations of increased MLN/TDLN cDC1s in mice harboring M. schaedleri fed a high Saa diet led to an investigation of the factors driving cDC1 accumulation and activation. Tests focused on XCL1, since XCL1 played a role in the protective effect of the high Saa diet in cAPC mice (see e.g., FIG. 4C). Furthermore. cDC1 selectively express the XCL1 receptor XCR1. Both NK and NKT cells can secrete XCL1, thereby directing the role of cDC1s in orchestrating CD8⁺ T cell-mediated anti-tumor immunity; see e.g., Barry et al., Nat Med 24, 1178-1191 (2018); Böttcher et al., Cell 172, 1022-1037.c14 (2018); the contents of each of which are incorporated herein by reference in their entireties. To determine if XCL1 secretion by these cell types was affected by dietary Saa, NK and NKT cells from the MLN of BIH C57BL6 WT mice fed the two Saa diets were sorted; NKT cells from high Saa diet-fed mice secreted higher XCL1 levels when cultured ex vivo as compared to NKT from low Saa diet-fed mice (see e.g., FIG. 4D). NK cells secreted less XCL1 than NKT cells regardless of diet (see e.g., FIG. 4D). TDLN NKT, but not NK, cell frequency increased in cAPC mice fed the high Saa vs. low Saa diet (see e.g., FIG. 4E, FIG. 11J), indicating that high Saa diet induced both secretion of XCL1 from NKT cells and their increased frequencies and numbers in the TDLN.

To test if M. schaedleri could directly influence NKT cell XCL1 production, an in vitro system was employed using the NKT cell line GW1 (see e.g., Donado et al., Cell Rep 31, 107466 (2020); the contents of which are incorporated herein by reference in their entirety) and abiotic bacterially conditioned medium (CDM). While CDM from two immunoregulatory gut bacteria, Lactobacillus plantarum and A. muciniphila had no effect, M. schaedleri CDM dramatically stimulated XCL1 production at a magnitude comparable to IL-12 treatment (see e.g., FIG. 4F). The XCL1-stimulatory activity of M. schaedleri CDM was heat-labile and primarily found in the organic phase of the supernatant (see e.g., FIG. 4F). As GW1 cells are a mouse cell line, the findings were next extended to human NKT cells. NKT cells were expanded from healthy human donor's peripheral blood mononuclear cells; the NKT cell were treated them with M. schaedleri CDM. There was a ˜4-fold increase in XCL1 in treated versus untreated NKT cells (see e.g., FIG. 4G). Taken together, these data indicate that NKT cell-derived XCL1 production contributed to the accumulation of activated cDC1s in the MLN/TDLN, and that diet and microbial factors triggered this NKT XCL1 production in mouse and human NKT cells.

To hone in on the M. schaedleri activities driving the NKT XCL1 secretion, untargeted LC-MS/MS metabolomics were performed on the abiotic M. schaedleri CM samples (n=4 independent samples) along with the medium control without M. schaedleri (n=2 independent samples). Of the 4,263 features detected, relatively few were enriched in the CM (n=36) and had a 2 or higher fold change and a p-value below 0.05, one-way ANOVA with post-hoc Tukey's HSD test (see e.g., FIG. 4H and FIG. 4I herein, and Table 3 of U.S. Provisional Application No. 63/389,382). Consonant with the extraction results (see e.g., FIG. 4F), many of the known or predicted metabolic features enriched in the M. schaedleri abiotic CM are lipids and lipid components (e.g., fatty acids) (see e.g., FIG. 4H and FIG. 4I herein, and Table 3 of U.S. Provisional Application No. 63/389,382). Without wishing to be bound by theory, it is contemplated herein that several of the M. schaedleri metabolites can work in concert to enhance NKT secretion of XCL1 which acts on cDC1 to promote their anti-tumor immunity via the CD8⁺ T cell compartment.

An Activated Transcriptional Signature in TDLN cDC1s of cAPC Mice Fed High Saa Diet Correlated with Better Survival in CRC Patients.

In studies described herein of ICI-responsive microbiomes, high Saa diet, and M. schaedleri, cDC1s emerged as a convergence point for understanding diet-microbiome-immunostimulatory interactions in CRC. Thus, to define the dietary Saa-M. schaedleri effects on cDC1s, single cell RNA sequencing (scRNA-Seq) was performed on FACS-sorted DCs from the TDLN of cAPC mice fed low and high Saa diets. TDLN are a pivotal immunoregulatory site where cDC1 cross-presentation events critical for anti-tumor CD8⁺ T cells occur. Additionally, they are readily available for study in mice and have not been profiled to date in human CRC scRNA-seq studies and, as such, represent an under-utilized cell population of potential use for prognosis.

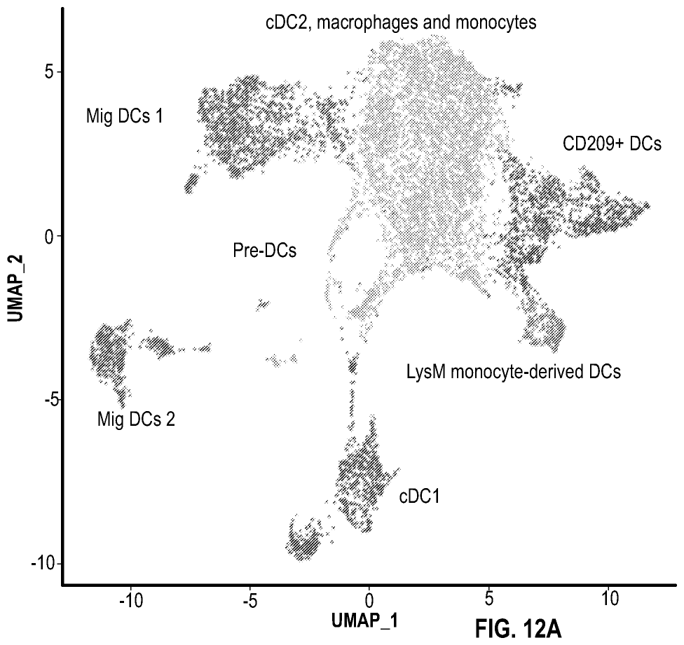

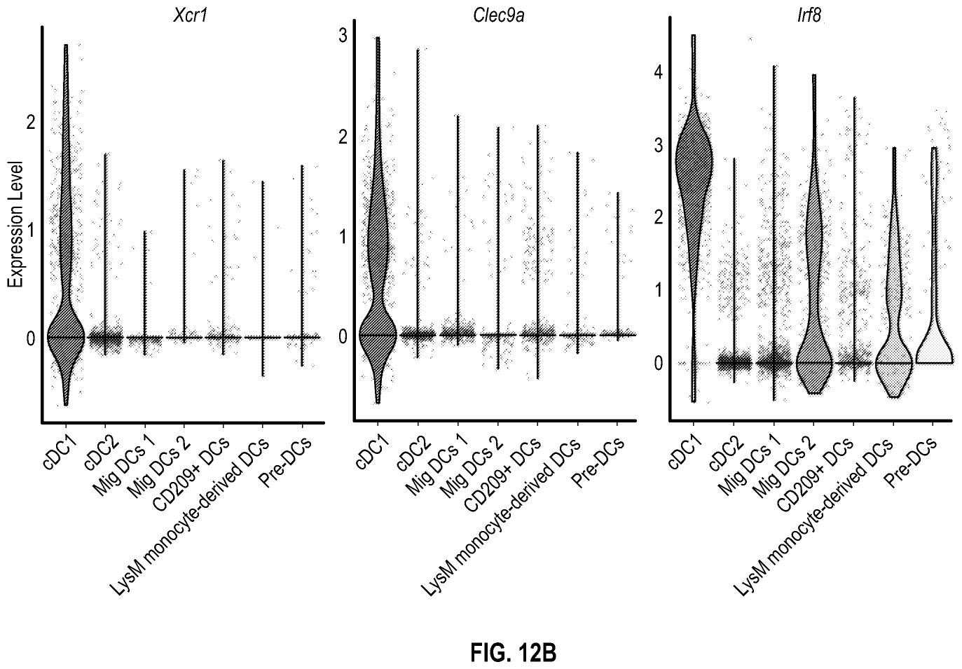

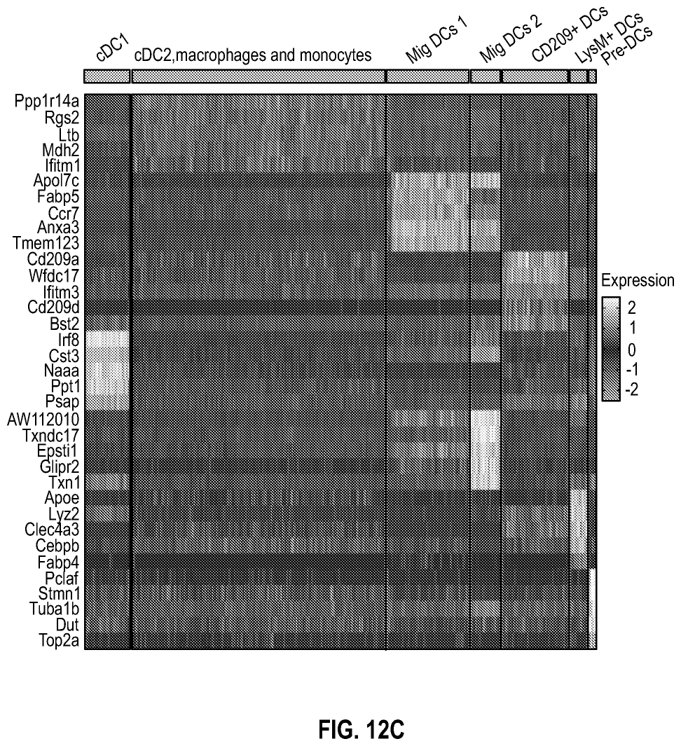

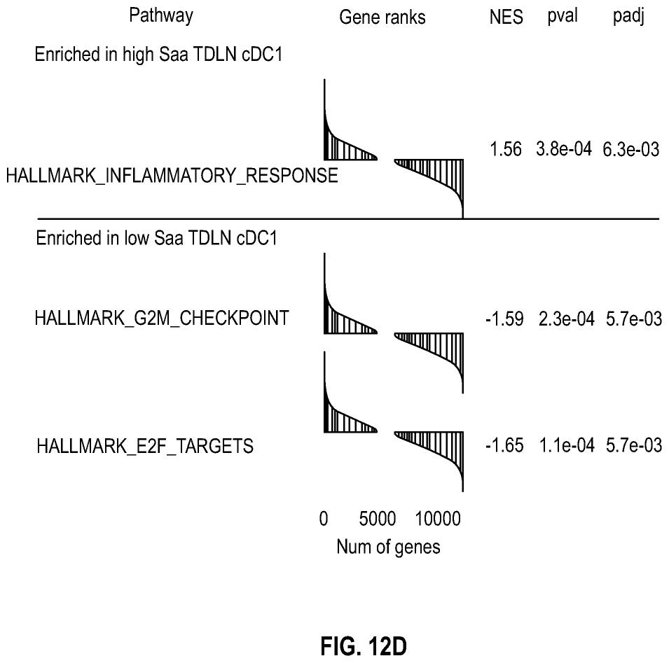

Sequencing of ˜12,500 CD11c⁺MHCII⁺ CD64⁻ cells revealed substantial cellular heterogeneity with 6 DC clusters as defined by manually-curated inspection of known DC markers (see e.g., FIG. 12A, Table 4). The largest cluster included CD11b⁺ cells, representing cDC2s, macrophages and monocytes, followed by clusters of migratory DCs (expressing CCR7), DC-SIGN⁺ (CD209) DCs, and a cDC1 cluster identified by the expression of Xcr1, Clec9a and Irf8 genes (see e.g., FIG. 12B-12C). Fast gene set enrichment analysis (fgsea) of the Hallmark Gene Set Pathways revealed enrichment of the inflammatory response pathway (M5932) in cDC1s isolated from TDLN of high Saa diet-M. schaedleri (Ms) expanded mice (see e.g., FIG. 12D), while cell cycle-related pathways were enriched in cDC1s from low Saa diet fed mice (see e.g., FIG. 12D).

Differentially expressed genes were identified in cDC1 from mice on the low vs. high Saa diets (see e.g., Table 5). cDC1 from high Saa diet-Ms expanded (HSME) mice expressed higher levels of Cxcl9 and Ccl4 transcripts (see e.g., FIG. 4J), both of which play critical roles in anti-tumor immune responses. Other transcripts expressed at higher levels in cDC1 from HSME mice included genes encoding the calcium binding proteins S100A6 and S100A4, which function in effective DC antigen presentation, co-stimulatory molecule expression, and T cell activation (see e.g., FIG. 4J). Genes expressed more highly in cDC1 from mice fed the low Saa diet (or decreased with the high Saa diet) included the thioesterase Ppt1, which balances viral resistance vs. T cell cross-priming (see e.g., FIG. 4J, Table 5). Decreased Ppt1 expression enhances tumor clearance in mice. Overall, the scRNA-Seq analysis showed that cDC1s from HSME mice displayed higher transcription of activation and immunostimulatory genes, consistent with the findings that the high Saa diet restricted tumor growth and reduced neoplastic progression.

Leveraging the cDC1 differentially abundant genes, a cDC1 gene expression signature was generated that is associated with the M. schaedleri-dependent beneficial anti-tumor effects of high Saa diet (see e.g., Table 5). The signature's association with survival data from the Tissue Cancer Gene Atlas (TCGA) dataset of colon and rectal adenocarcinoma tumor (TCGA COAD-READ, N=359) bulk RNA-Seq transcriptomes was examined. Patients with a transcriptome score more similar to the HSME mouse cDC1 transcriptional state (HSME-DC; “high activation”) had a statistically significant longer survival time (Cox model, P=0.005 for univariate analysis and P=0.017 for disease stage adjusted multivariate analysis) compared to patients with a transcriptional score resembling the low-Saa-M. schaedleri depleted (LSMD; “low activation”) mouse cDC1 state (see e.g., FIG. 4K). For patients with tumor gene expression more similar to LSMD-DC, there was an increased hazard ratio (HR) of 1.611 (1.152-2.253, 95% confidence interval (CI)) with the univariate analysis and a HR of 1.521 (1.077-2.147, 95% CI) in multivariate analysis including stage. Thus, the gene expression signature detected in cDC1s from HSME cAPC mice correlated with improved survival in CRC patients.

### Discussion

In the search for microbial bioactivities underlying anti-tumor immunity, an enrichment of Saa pathways and genes in the gut microbiome was identified as a shared feature of ICI responsiveness across cohorts. Targeting these pathways via dietary Saa supplementation slowed tumor progression in both MMR-deficient and -proficient CRC models. Methionine-dependence has been reported in CRC tumor cells, and methionine restriction limits tumor growth in mouse models. Such results raised the question of why reduced tumor growth was observed in high Saa diets herein. It is contemplated herein that both very low Saa diet or high Saa diet can be tumor-suppressive for different underlying reasons, with low Saa diet restricting cancer cell growth due to metabolic dependency, and high Saa diet enhancing anti-tumor immunity, as described herein. Low Saa diet was not sufficiently methionine restrictive for it to affect tumor growth herein. Dietary sulfur takes many different forms in organic and inorganic compounds; for example, total dietary sulfur intake exhibits a correlation with human colonic adenomas and CRC. Dietary Saa modulation also has pleiotropic effects on the host and microbiome, from altering redox potential to protein post-translational modifications all within a dose range that is non-toxic for the host. Given the anti-tumor immune effects observed herein, Saa supplementation can be clinically investigated in CRC patient cohorts. In addition, further investigation in ICI-refractory patients can also be performed, given that this pathway was enriched in the microbiota of ICI non-responder melanoma patients who received a fecal microbiota transplant and became ICI responsive. See e.g., Gao et al., Nature 572, 397-401 (2019); Hoffman (Humana Press, 2019); Komninou et al., Nutr Cancer 54, 202-208 (2006); Tan et al., Clin Cancer Res 5, 2157-2163 (1999); Nguyen et al., Gastroenterology 158, 1313-1325 (2020); Nguyen et al., Gastroenterology 161, 1423-1432.e4 (2021); Yan et al., Journal of Biological Chemistry 285, 41525-41532 (2010); Lobel et al., Science 369, 1518-1524 (2020); Baruch et al. 2021, supra; the contents of each of which are incorporated herein by reference in their entireties

Described herein is the identification of common functional features across the diverse gut microbial taxa associated with ICI response in patients that can be targeted to improve anti-tumor immunity. Also described herein is a dietary intervention to target Saa pathways, which attenuated tumor growth in a malignancy, CRC, that is generally recalcitrant to ICI-therapy and viewed as immunologically cold. The dietary intervention initiated a mucus-M. schaedleri-anti-tumor immune cascade of effects (schematized in FIG. 13), indicating that food can be functionalized to improve anti-tumor immunity in CRC. The M. schaedleri data indicated that mucus was a link between diet, the microbiota, and immune function, and that the colonic mucus layer is a site of microbial activity involved with anti-tumor immunity and immune function. Additionally, M. schaedleri's immunomodulatory effects focused attention on NKT cells as a link in the chain connecting diet, gut microbial factors, and enhanced cDC1 cell function that converged on CD8⁺ T cells. These findings confirm the role of cDC1s in anti-tumor immunity. The cDC1 depletion data described herein, in conjunction with the data on depleting XCL1, which is a cDC1 specific chemokine and activator, demonstrate the role of cDC1 in linking Saa, M. schaedleri, and enhanced immunity against CRC.

Collectively, the data described herein highlight the utility of mouse models in exploring diet-microbe interactions not only for identifying biogeographies such as the mucus layer or tumor-draining lymph nodes, but also for identifying CRC prognostic signatures that lie at the interface of diet, microbiome, and host immunity. Altogether, the experiments herein elucidate how diet-microbiota interactions enhanced anti-tumor immunity in a multi-step fashion that tuned innate immune cell responses to control tumor progression in CRC. Overall, the data described herein provide an examination of patient stool metagenomic profiles from ICI-responsive patients in order to determine the mechanisms by which microbiota-diet interactions enhance anti-tumor immunity for immunologically cold tumors.

### Materials and Methods

Meta-Analysis of Microbial Metabolic Pathways in Microbiomes of Anti PD-1 Treatment Responders Vs. Non-Responders.

Fecal metagenomic shotgun reads for the ICI and CRC datasets were obtained from the sequence read archive (SRA) of the National Center for Biotechnology Information (NCBI). Any metagenomic samples with known use of antibiotics as described in available clinical metadata were excluded from meta-analysis. Sequence raw reads were quality trimmed using TRIMMOMATIC (v0.39), which was configured to perform sliding window scan with the following parameters: “ILLUMINACLIP:${adapter_library_FASTA}::2:36:7:1: keepBothReads LEADING:3 TRAILING:3 SLIDINGWINDOW:4:15 MINLEN:36.” To build an expanded collection of bacterial Saa biosynthesis reference genes from the MetaCyc pathway database, 72,380 complete and draft-level bacterial GENBANK genomic assemblies with sufficient taxonomic coverage over representative human gut microbiome genera were accessed. Incorporating a priority list of 15 Saa protein sequences for custom BLAST+ search (options: e-value 1e-09, coverage 80), protein-coding regions of each assembly were annotated iteratively via PROKKA, a fast hierarchical genome annotation pipeline, and 484,023 bacterial Saa gene homologs were detected. See e.g., Bolger et al., Bioinformatics 30, 2114-2120 (2014); Xiao et al., Nature Biotechnology 33, 1103-1108 (2015); Seemann, Bioinformatics 30, 2068-2069 (2014); the contents of each of which are incorporated herein by reference in their entireties.

120,861 of these Saa utilization gene homologs were found to have nonzero abundance when reads were mapped using BWA-MEM v0.7.17 with default parameters, and quantified using htseq-count (default union method) from HTSeq. Gene read count matrix was constructed by aggregating total gene abundance across bacterial genomes (reads-per-kilobases; RPK). Together with pathway relative abundance profiles generated using HUMAnN 2, these data were used to fit linear mixed-effects models (LMM) with study effect as random covariate as implemented in the lmerTest R/CRAN package. Effect sizes were derived from fitted LMMs for pathways that are present in at least 20% of samples using the emmeans R/CRAN package. Interquartile range-guided rank-based combinatorial mean fold changes were computed to contrast biological groups of interest. See e.g., Li, arXiv 1303.3997v2 (2013); Anders et al., Bioinformatics 31, 166-169 (2015); Kuznetsova et al., Journal of Statistical Software 82, (2017); Russell, emmeans: Estimated Marginal Means, aka Least-Squares Means (2021); Xiao et al., Bioinformatics 30, 801-807 (2014); the contents of each of which are incorporated herein by reference in their entireties.

### Mice and Dietary Interventions.

Mice (WT C57BL/6, CDX2-Cre APC^{flox/+} (cAPC), Zbtb46-DTR, cAPC Batf3^{-/-} and Ptprc^{a} (Ly5.1, CD45.1)) were housed in a barrier facility with constant ambient temperature of 24° C. and 12 h of day/night cycles. Born in-house (BIH) mice were conventionally-reared, specific pathogen-free C57BL6/J mice bred in the barrier facility vivarium. All mouse strains were purchased from JACKSON LABORATORY and then bred at in house. For gnotobiotic experiments, mice were housed at in semi-rigid isolators (PLASTIC CONCEPTS INC.) and experiments were conducted in individual ventilated ISOCAGEP system (TECNIPLAST). Routine surveillance, including 16S rRNA gene amplicon sequencing and qPCR analyses (using universal 16S rDNA primers) and Sanger sequencing, were performed on fecal samples and cage swabs to validate the gnotobiotic status (germ-free, monocolonized, or ASF) of the mice. For re-derivation of cAPC mice, pregnant female mice were euthanized and their uteri were removed under aseptic conditions, using chemical sterilant (MB-10, QUIP LABS) in a semi-rigid isolator. The sterilized pups were introduced to germ-free foster dams. After 3 weeks, the germ-free status of the mice and their genotypes were evaluated using PCR.

Sulfur amino acid (Saa) diets were formulated to represent edge cases of Saa consumption, with the following considerations. Human dietary cysteine and methionine consumption typically ranges between 0.03-0.06 g/kg body weight/day. Across human diets with a range in their protein consumption (44 g-140 g/day), low levels of cysteine are between 0.01-0.04/g/kg/d. Very high levels of methionine or cysteine are in the range >=6 g/kg/day; such levels raise concerns for contributing to homocysteinemia in humans. See e.g., Elshorbagy et al., J Nutr Biochem 23, 332-340 (2012); Paul et al., Nature 509, 96-100 (2014); O'Keefe et al., J Nutr 137, 175S-182S (2007); O'Keefe et al., Nat Commun 6, 6342 (2015); Nimni et al., Nutr Metab (Lond) 4, 24 (2007); David et al., Nature 505, 559-563 (2014); the contents of each of which are incorporated herein by reference in their entireties. Given these data and with veterinary approval, the two isocaloric diets employed herein were formulated and manufactured by RESEARCH DIETS, INC (see e.g., Table 2 for the diet formulations). For gnotobiotic experiments, the same formulations, irradiated, were ordered from TEST DIET.

At 6-8 weeks of age, WT, cAPC, cAPC Batf3^{-/-} or cAPC Zbtb46-DTR mice were transitioned to Saa diets. After 12 weeks on the Saa diets, mice were sacrificed and tissues (normal, neoplastic, and adjacent normal) were collected either for histology, flow cytometry or immunofluorescence and cecal contents were frozen for microbial analysis. For gnotobiotic experiments, GF, ASF, M. schaedleri- or A. muciniphila-monocolonized mice were transferred to Saa diets at 6-8 weeks of age. After 4 weeks, mice were sacrificed and tissues were analyzed by flow-cytometry. GF cAPC mice transitioned to the Saa diets on weaning and were maintained on the diets for 12 weeks. In bacterial CDM feeding experiments, WT bred in-house mice fed low Saa-diet were gavaged every two days with 100 μl of 5 to 7-day-old filtered culture supernatant of M. schaedleri or sterile mBHI medium.

To generate cAPC Zbtb46-DTR mice, cAPC mice were irradiated at 10 weeks of age with one dose of 1000 rad and then injected with 10⁶ bone-marrow cells from Zbtb46-DTR mice. To deplete Zbtb46 expressing cells in cAPC Zbtb46-DTR bone chimera mice, mice were injected with 400 ng (˜20 ng/g body weight) of diphtheria toxin (DT) 4 weeks after irradiation, followed by twice weekly injections of 100 ng DT (˜4 ng/g body weight) to maintain Zbtb46 expressing cells depletion for the rest of the experiment. For XCL1 depletion, cAPC mice were fed high Saa diet for 8 weeks and then injected i.p. with 300 μg of anti-XCL1 antibody or InVivoMAb rat IgG1 Isotype control, anti-trinitrophenol (BIOXCELL) every 2-3 days for 4 weeks. Animal studies and experiments were approved and carried out in accordance with guidelines for animal use and care.

### MC38 Colon Carcinoma Flank Tumor Model.

MC38 mouse colon carcinoma cells (KERAFAST) were grown in RPMI GLUTAMAX medium supplemented with 10% fetal bovine serum (FBS), 500 U/ml of Penicillin/Streptomycin, 1 mM sodium pyruvate, and 50 μM β-mercaptoethanol. At ˜80% confluence, cells were harvested, washed in PBS and resuspended on ice in a 1:1 solution of CULTREX reduced growth factor basement membrane extract (R&D SYSTEMS) and PBS at a concentration of 3×10⁶ cells/ml. WT bred in-house C57BL/6J mice were put on low or high Saa diets two weeks prior to MC38 engraftment. On the day of engraftment, the mice were shaved on their left flank and injected subcutaneously with 150 μl (0.5×10⁶ cells) of the MC38 cell solution. Mice were monitored every other day and tumor volume was calculate using Volume=(Width²×Length)/2 (Formula 1). 12 days post-engraftment, mice were sacrificed, and tumors were excised and weighed. For anti-PD-1 experiments, mice were treated as above with the addition of i.p. injections of either INVIVOMAB anti-mouse PD-1 (BIOXCELL) or INVIVOMAB rat IgG2a isotype control anti-trinitrophenol (BIOXCELL), 250 g per mouse, at days 6, 9 and 12. Isotype or anti-PD-1 treated mice were sacrificed at day 13 post-engraftment and tumor weight was measured. See e.g., Corbett et al., Cancer Res 35, 2434-2439 (1975); Faustino-Rocha et al., Lab Anim (NY) 42, 217-224 (2013); the contents of each of which are incorporated herein by reference in their entireties.

### Histopathology.

After sacrifice, colons were opened using blunt scissors and the luminal contents were removed. Cecal contents were flash frozen in liquid N₂. Tissues were fixed in 4% paraformaldehyde, processed, and paraffin-embedded using standard protocols by a rodent histopathology core. Five hematoxylin and eosin (H&E) stained slides (five level sections obtained 50 μm apart) for each sample were blindly evaluated a board-certified GI pathologist for neoplastic lesions—aberrant crypt foci (ACF), adenoma, or adenocarcinoma (adenoCA).

### Cecal DNA Extraction and Real-Time Quantitative PCR (RT-qPCR) Analysis.

Mouse cecal contents were collected into 1.5 ml tubes and immediately frozen in liquid N₂. Thawed cecal contents were resuspended in 300 μl of Tris-EDTA solution (100 mM Tris and 15 mM EDTA) in 2 ml tubes with ˜300 μl of zirconium beads (20 micron), and 500 μl of TE-saturated phenol (SIGMA-ALDRICH) were added. The tubes were placed in a bead-beater for 2 min and centrifuged for 15 min at max speed. The aqueous phase was moved to a clean tube and 1:1 volume of phenol:chloroform:isoamyl alcohol (25:24:1) was added. Tubes were vortexed for 1 min and then centrifuged for 2 min at max speed. The procedure was repeated 3 times. The aqueous phase was moved to a clean tube and 2 volumes of 100% ethanol (EtOH) and 1/10 volume of sodium acetate (NaOAc) pH 5.2 were added. After 1 hour at −20° C., the tubes were centrifuged at 14,000 revolutions per minute (rpm) for 20 min at 4° C., the liquid was discarded, and 1 ml of cold 70% EtOH was added to wash the pellet, followed by another 20 min of centrifugation at 4° C. at 14,000 rpm. Finally, the liquid was aspirated off and the pellet was air-dried for 10 min at room temperature, followed by resuspension in 100 μl of sterile, molecular biology-grade H₂O. DNA concentration was measured by spectrophotometry at 260 nm. For RT-PCR analysis, 50 ng of cecal DNA were mixed with 10 μl 2×SYBR green (KAPPA SYBR FAST) and 0.29 μM of each forward/reverse primer set (see e.g., Table 6) in a 20 μl reaction. Real-time PCR reactions were performed on an APPLIED BIOSYSTEMS STRATAGENE MX3005P machine. ΔΔCt were calculated using 2^{-((XaCt-XbCt)-(Yact-Ybct))} (Formula 2), where X and Y are genes and a and b are biological samples, b being the reference sample or the mean of biological repeats. Raw data were extracted and analyzed using the LIBREOFFICE Calc program and RStudio.

Bacterial 16S rRNA Gene Amplicon Library Generation and Sequencing.

The procedures in this section were performed in a biological safety cabinet to minimize potential contamination and were based on the EARTH MICROBIOME PROJECT protocol. 50 ng of cecal DNA was used in a PCR reaction using the THERMO FISHER PLATINUM HOT START PCR MASTER MIX (cat. no. 13000014) according to the reagent protocol. Forward (10 μM) and reverse (1.3 μM) primers were used (see e.g., Table 6) to amplify the V4 region of the 16S rRNA gene. For each sample, the reverse primer contains a unique 12 bp GOLAY barcode. Each sample was amplified in triplicate in a 25 μl reaction volume in 96-well plates. Sterile, molecular biology-grade water and E. coli genomic DNA served as negative and positive controls, respectively. The PCR reaction started with 3 min of 94° C., then 35 cycles of 45 seconds 94° C., 60 seconds 50° C. and 90 seconds 72° C., followed by 10 min of 72° C. After amplification, the triplicate reactions were pooled and amplicons were purified using AMPURE magnetic beads. DNA concentration was determined by the dsDNA broad range assay kit (THERMO FISHER) using a QUBIT machine and a sample of several libraries was run on an agarose gel to visualize the specific amplicon. The libraries were pooled so that the final DNA concentration was 50 ng/μl and each library has an equal abundance. The pooled amplicon library was analyzed on an AGILENT 4200 TAPESTATION system. DNA sequencing was performed on an ILLUMINA MISEQ machine at a bio-polymer core using the MISEQ V2 kit with 250 bp paired-end reads. See e.g., Thompson et al., Nature 551, 457-463 (2017); Walters et al., mSystems 1, e00009-15 (2016); the contents of each of which are incorporated herein by reference in their entireties.

Analysis of Bacterial 16S rRNA Gene Amplicon Surveys in Mice.

16S rRNA gene amplicon survey analysis was conducted according to the standard operating protocol (SOP) of Comeau et al. mSystems 2, e00127-16 (2017), using the microbiome-helper wrapper. Briefly, fastq files were obtained for each library (median paired-end read count: 79,584 250-bps) and quality of reads was evaluated using FastQC (v0.11.5). Based on the sequence quality report, reads were trimmed using fastx-toolkit to keep only high-confidence base calls and stitched using PEAR. Chimeric reads were filtered using VSEARCH. Operational taxonomic units (OTUs) were picked using QIIME (v1.9) with SortMeRNA filtering and OTUs with fewer than 0.1% of the reads were excluded as low-confidence. Finally, OTU read count data was rarefied to the lowest library size. Analyses of α-diversity and β-diversity (weighted Unifrac PCoA) were performed and visualized using the phyloseq R/Bioconductor (v1.30) and metacoder R/CRAN packages. Differential OTU abundances between the two diets were analyzed using MaAsLin 2 with cage as a random covariate. For measuring the abundance of M. schaedleri in human mucosal-associated bacterial amplicon samples, 16S rRNA gene sequences were analyzed using mothur software suite following 454 and ILLUMINA MISEQ SOPs. See e.g., Comeau et al. mSystems 2, e00127-16 (2017); Andrews, FastQC, available on the worldwide web at: bioinformatics.babraham.ac.uk/projects/fastqc/ (2010); Zhang et al., Bioinformatics 30, 614-620 (2014); Rognes et al., PeerJ 4, e2584 (2016); Caporaso et al., Nat Methods 7, 335-336 (2010); Kopylova et al., Bioinformatics 28, 3211-3217 (2012); McMurdie et al., PLoS One 8, e61217 (2013); Schloss et al., Appl Environ Microbiol 75, 7537-7541 (2009); the contents of each of which are incorporated herein by reference in their entireties.

### Alcian BLUE Staining.

Unopened colons were fixed overnight in methanol-Carnoy's solution (6:3:1 methanol:chloroform:glacial acetic acid) followed by routine paraffin embedding and sectioning. Sections were deparaffinized, hydrated and stained with 1% Alcian Blue solution to visualize the mucus layer, then counterstained with Nuclear Fast Red (AMRESCO 1B1369). HISTOMOUNT (NATIONAL DIAGNOSTIC HS-103) was used as mounting medium and images were acquired on NIKON ECLIPSE NI-U microscope.

### Immunofluorescence Staining and FISH.

To detect CD3 and CD8 positive T-cells in mouse colon tumor tissues, immunofluorescence staining of mouse colon tissues was performed on formalin-fixed paraffin embedded 5 micrometer sections. Colon sections were deparaffinized, pretreated with 3% hydrogen peroxide in methanol, then with 1 mM EDTA pH 8.0 (at >90° C. for 15 min) for antigen retrieval. After blocking in Tris-buffered saline (TBS) 1% BSA with 10% donkey serum, the sections were stained overnight (0/N) at 4° C. with anti-CD3 (1:3400, ABCAM ab5690) and anti-CD8 (1:2500, EBIOSCIENCE 14-0808-80). After washing, Donkey anti-Rat ALEXA FLUOR 594 IgG (JACKSON IMMUNORESEARCH 712-585-153) was applied to detect CD8 and then Donkey anti-rabbit-HRP IgG (JACKSON IMMUNORESEARCH 711-035-152) followed by TSA Fluorescein reagent (1:2200, NEL741E001KT, PERKINELMER) to reveal CD3 positive cells. 4′,6-diamidino-2-phenylindole (DAPI) was used as a nuclear counterstain and PROLONG GOLD antifade as the mounting medium (P36934, LIFE TECHNOLOGIES). Images were acquired on a NIKON ECLIPSE TI laser scanning microscope with 20× and 60× objectives.

For Mucispirillum spp. detection and mucus layer and goblet cell visualization, unopened distal colons were fixed overnight in methanol-Carnoy's fixative followed by routine paraffin embedding and sectioning. Colon sections were deparaffinized, pretreated with 3% hydrogen peroxide in methanol, and blocked with TRUEBLACK LIPOFUSCIN AUTOFLUORESCENCE QUENCHER (BIOTIUM 23007) according to manufacturer recommendations. Fluorescence in situ hybridization was performed at 50° C. for 90 min in 5% formamide-0.1% SDS-TBS buffer with 2.5 ng/μl of each Mucispirillum genus specific probes MCS487 (5′-Cy5-GCCGGGGCTGCTTATACAGGT-3′, SEQ ID NO: 1) and MCS547 (5′-Cy5-CAGTCACTCCGAACAACGCT-3′, SEQ ID NO: 2), and 5 ng/μl of a eubacterial 16S RNA sequence specific probe EUB338 (5′-Cy3-GCTGCCTCCCGTAGGAGT-3′, SEQ ID NO: 3). After washing, all the subsequent steps were performed at 4° C. The tissue sections were blocked with 3% Donkey serum in 1% BSA-TBS for 1 h, stained O/N using an anti-Muc2 antiserum, and a Donkey anti-rabbit Alexa Fluor 488 IgG (INVITROGEN A21206). DAPI was used as nuclear counterstain and PROLONG GOLD antifade as mounting medium (P36934, LIFE TECHNOLOGIES). Images were acquired on a NIKON ECLIPSE NI-U equipped with a 40× objective. See e.g., Berry et al., ISME J 6, 2091-2106 (2012); Johansson et al., PNAS 105, 15064-15069 (2008); the contents of each of which are incorporated herein by reference in their entireties.

### Gut Barrier Leakage Assay.

Mice were gavaged with 10 mg of FITC-dextran (SIGMA 46944) in 100 μl PBS with ad libitum access to food and water. Serum was collected 3 h later. Serum was diluted 1:1 in PBS and fluorescence levels were measured with 485 nm excitation and 530 nm emission wavelengths. A standard curve was generated by diluting FITC-dextran at various concentrations in a 1:1 mix of control serum (from PBS alone gavaged mice) and PBS.

### Cell Suspension Preparation and Flow Cytometry Analysis.

Colons were removed and placed in 10 ml of phosphate-buffered solution (PBS) with 1 mM dithiothreitol (DTT) in 50 ml conical tubes and incubated on ice for 10 min. Thereafter, the colons were transferred into a new tube with 10 ml of PBS with 5 mM ethylenediaminetetraacetic acid (EDTA) and 3% FBS and placed on a rotating wheel at 37° C. for 15 min. Next, epithelial cell suspension was passed through a 100 μm cell-strainer to a new tube and the colonic tissues were collected and moved to a new 50 ml tube with 10 ml of PBS with 5 mM EDTA and 3% FBS, and this procedure was repeated. The final filtrate was stored as the epithelial cell fraction. The tissues were moved to a new 50 ml conical tube with 20 ml of PBS and were centrifuged for 5 min at 1500 rpm at 4° C. to wash the colons off EDTA and DTT. Next, the tissues were transferred to 60 mm petri dishes and manually minced with a blade for 1 min in 2 ml of digestion medium (RPMI with GLUTAMAX, 10% FBS, penicillin-streptomycin, 0.5 mg/ml of dispase enzyme (STEM CELL TECHNOLOGY), 1 mg/ml of collagenase D (ROCHE) and 50 μg/ml of DNAse I). 8 ml of digestion medium was added and the tissue suspension was moved to a 50 ml conical tube and put on a rotating wheel for 30 min at 37° C. The lamina propria (LP) cell suspension was passed through a 40 μm cell-strainer to a new tube containing 5 ml PBS with 5 mM EDTA, the remaining cell suspension was moved back to the digestion tube and 10 ml of fresh digestion medium were added and the procedure was repeated.

Tumor tissue was processed similarly, with just one round of 1 h digestion in 5 ml digestion media. For interferon-gamma (IFNγ) and Granzyme B (GZMB) measurements in CD8⁺ T cells, tumor cells were enriched for CD8+ T cells using magnetic assisted cell sorting (MACS) in a two-step process. First, dead cells were depleted using DEAD CELL REMOVAL KIT (MILTENYI BIOTECH) and then CD8⁺ T cells were isolated using the mouse CD8α⁺ T CELL ISOLATION KIT (MILTENYI BIOTECH) and incubated overnight in a 96-well plate precoated with 2 μg/ml anti-CD3 antibody (BIOLEGEND) and 5 μg/ml anti-CD28 (BIOLEGEND) in RPMI with GLUTAMAX, 10% FBS, penicillin-streptomycin (50 U/ml), 1 mM sodium pyruvate, 55 μM 2-mercaptoethanol and 10 mM HEPES (4-(2-hydroxyethyl)-1-piperazineethanesulfonic acid). Following overnight stimulation, the cells were treated with brefeldin A and monensin for 2 hr and then fixed, stained for IFNγ and GZMB, and analyzed.

Mesenteric lymph nodes (MLN) or tumor draining lymph nodes (TDLN) were removed surgically and placed in a 1 ml of RPMI medium and kept on ice, before being crushed through a 70 μm cell-strainer using a 1 ml syringe plunge. 1-2 million cells of LP, tumor or MLN/TDLN cells were taken for staining and placed in 100 μl of PBS with LIVE/DEAD FIXABLE YELLOW DEAD CELL STAIN KIT (THERMO FISHER) in a 96-well round-bottom plate and left covered for 15 min at room-temp. After 15 min, 100 μl of FACS buffer (PBS with 1 mM EDTA and 2% FBS) were added, the plate was centrifuged for 5 min at 1500 rpm and the liquid was discarded. The cells were resuspended in 100 μl of FACS buffer containing 1 μl of anti-CD16/CD32 antibody (BIOLEGEND, cat no 101302) to block Fc receptors and placed in 4° C. for 10 min. 100 μl of antibody stain mix in FACS buffer (see e.g., Table 7 for antibody listing) were added to each well and the plate was incubated at 4° C. for 30 min before centrifugation for 5 min at 1500 rpm, liquid removal and resuspension in 250 μl of FACS buffer. For intracellular protein staining BIOLEGEND FOXP3 FIXATION/PERMEABILIZATION KIT (Cat 4421403) was used according to the manufacturer's instructions. The samples were analyzed on either an LSR-II BD machine or a BD FACSYMPHONY machine. Data were analyzed using FLOWJO (TREE STAR INC.) and FLOWLOGIC (INIVAI TECHNOLOGIES PTY. LTD.) software.

TCR-Seq Analysis on Tumor CD8⁺ T-Cells from Mouse Tumors.

Tumors were surgically removed from cAPC mice fed low or high Saa diet and digested to single cell suspension as described above. CD8⁺ T-cells were sorted by gating on live CD45⁺CD3⁺CD8⁺ CD4⁻ cells on a MOFLO ASTRIOS cell sorter (BECKMAN COULTER) machine. Between 1000-3000 cells were collected from each of 8 cAPC mice tumors (5 fed low Saa and 3 fed high Saa diet). The cells were processed using the TAKARA SMARTER MOUSE TCR a/b profiling kit, according to the manufacturer instructions, to produce amplicon libraries for high-throughput sequencing. The concentration and distribution of amplicons in the samples was evaluated using AGILENT 4200 TAPESTATION analysis. The libraries were sequenced on an ILLUMINA MISEQ machine using the MISEQ REAGENT KIT v3 (ILLUMINA, Cat. No. MS-102-3003) with paired-end, 2×300 base-pair reads. Fastq read quality was analyzed using FastQC v0.11.5 and trimmed accordingly using the fastx-toolkit. Processed reads were analyzed using MiXCR and the immunarch (0.6.5) R/Bioconductor package to generate abundance tables for clonotypes and statistical analyses. See e.g., Bolotin et al., Nature Methods 12, 380-381 (2015); ImmunoMind, T. immunarch: An R Package for Painless Analysis of Large-Scale Immune Repertoire Data. Zenodo (2019); the contents of each of which are incorporated herein by reference in their entireties.

### Bacterial Strains and Cultures.

Mucispirillum schaedleri was cultured in modified brain-heart infusion (mBHI) medium composed of 37 g BHI, 5 g yeast extract, 2 mg vitamin K, 5 mg hemin, 0.5 g L-cysteine and 150 ml fetal bovine serum in 1 μL tap water. The medium was pH adjusted to 7.2 and filtered through 0.2 μm. For agar plates the same medium was used with the addition of 12 g of agar per 1 μL. M. schaedleri was grown in mBHI under anaerobic conditions (Coy anaerobic chamber with 80% N2, 10% CO₂ and 10% H₂ gas mix atmosphere) at 37° C. for 5 days, at which point a bacterial pellet was observed. For CM assays, the cultures were centrifuged at max rpm for 5 min and the supernatant was filtered through a 0.2 μm filter and kept at −20° C.

Lactobacillus plantarum WCSF-1 and Akkermansia muciniphila ATCC BAA-835 were plated on LB or BHI plates, respectively. To obtain bacterial conditioned media, A. muciniphila and L. plantarum were grown in mBHI under the same conditions as M. schaedleri, except for a shorter growth period (overnight) for Lactobacillus, to accommodate the faster growth. Supernatants were harvested in the same manner as for M. schaedleri cultures. To heat-treat supernatants of M. schaedleri, samples were incubated for 10 min at 100° C. To fractionate organic and aqueous fractions from bacterial supernatants and cell pellets, samples were processed using methanol:chloroform extraction. Briefly, 150 ml of aqueous sample (supernatant sample, or resuspended cell pellet) was mixed with 160 ml of methanol (SIGMA-ALDRICH), followed by addition of 320 ml of chloroform (SIGMA-ALDRICH) and a short vortex. The samples were then centrifuged at max rpm for 3 min and the organic and aqueous phases were separated to new 1.5 ml tubes. The fractions were dried using a SPEED-VAC MACHINE (EPPENDORF) and resuspended in 150 ml cell culture medium. For mono-colonization experiments, a 5 ml Mucispirillum schaedleri culture (O.D._{600 nm}˜0.8) was centrifuged at 4,000 rpm for 5 min and the bacterial pellet was resuspended in 1 ml mBHI in the anaerobic chamber. Thereafter, each germ-free mouse received 100 μl of the bacterial suspension or sterile mBHI medium.

In Vitro cDC1 Immunological Assays.

Given ex vivo colonic and lymph node sorted cDC1 viability and cell number issues for use in in vitro, sorted splenic cDC1s were employed. Splenic cDC1 cells were isolated from WT C57BL6/J mice using magnetic associated cell separation (MACS) kit (CD11c⁺ Dendritic Cell Isolation Kit, mouse, MILTENYI BIOTECH) according to the manufacturer instructions and plated in a 96-well plate, at a density of 5×10⁴ cells per well. The cells were incubated overnight with sterile mBHI medium, M. schaedleri CM, L. plantarum CM or E. coli CM. Cells were washed, stained with antibodies and analyzed using flow cytometry. For CD8 T cell activation experiments, Naive CD8 T cells (CD44⁻/CD62L⁺) were isolated from OT-I (ovalbumin transgenic TCR) mouse spleens. For cross-presentation assays, splenic cDC1 were treated with bacterial CM or sterile medium in the presence of 0.7 mg/ml of ovalbumin protein (OVA, ENDOFIT) for 12 hours and then washed with PBS, before the naive OT-I CD8 T cells were added at a 1:1 ratio of DC:T cells. As control, 1 mg of the OT-I specific OVA peptide (SIGMA-ALDRICH) was incubated with untreated splenic cDC. For CD8 T cell activation assay, after overnight incubation with the loaded DCs, the cells were treated with Brefeldin A and monensin (both 1:1000, BIOLEGEND) for 2 h, fixed (BD CYTOFIX) and IFNg expression was measured using flow cytometry. For CD8 T cell proliferation assay, OT-1 CD8 T cells were stained with carboxyfluorescein succinimidyl ester (CFSE) (CELLTRACE, THERMO FISHER) and the co-culture was incubated for 3 days and then the cells were analyzed using flow cytometry to measure the number of cell divisions.

Measurements of XCL1 Secretion from Mouse NK and NKT Cells and Mouse Serum.

NK and NKT cells were sorted from the MLN of WT BIH mice fed Saa diets using a BD FACSARIA IIU cell sorter. Sorted NK and NKT cells were plated in a 96-well plate and incubated overnight with IL-2 (10 ng/ml) supplementation. The following day, cell media was collected and XCL1 was measured using R&D SYSTEMS MOUSE XCL1/LYMPHOTACTIN DUOSET ELISA KIT. For in vitro XCL1 secretion assays, 6×10⁵ cells per well of GW1 NKT cell line, were plated in a 24-well plate (0.5 ml volume) and were stimulated overnight with 50 ml (10% v/v) of M. schaedleri, A. muciniphila, or L. plantarum conditioned media or their fractions (see above). Sterile mBHI medium was used as control. The next day, cell culture supernatants were collected and XCL1 concentration was determined using R&D SYSTEMS MOUSE XCL1/LYMPHOTACTIN DUOSET ELISA KIT. For measurements of mouse serum XCL1, mouse blood was collected into serum separator tubes (BD) tubes, inverted 5 times and allowed to clot for 30 min at room temperature. Then, samples were centrifuged for 15 min at 1300 g at 4° C. and the serum layer was carefully removed into a new 1.5 ml tube without disturbing the buffy coat layer. Serum XCL1 was measured using the ONESTEP MOUSE XCL1 ELISA KIT (ABCAM).

Measurements of XCL1 Secretion from Human NKT Cells.

Briefly, to generate human NKT cells the buffy coats from human donors' peripheral blood were obtained from RESEARCH BLOOD COMPONENTS LLC and separated using differential centrifugation on a FICOLL density gradient. Magnetically Isolated CD14^{neg} cells were cultured with 10 U/ml rhIL-2 for 5 days. Then, Vα24⁺ hNKT cells were magnetically isolated and incubated for 2 days with mitomycin C-treated human dendritic cells loaded with α-galactosylceramide (α-GalCer)+20 U/ml rhIL-2. Then, the cells were harvested and further expanded for 10 days in complete RPMI medium+20 U/ml rhIL-2, with medium replacement every 2 days. Finally, the purity of Vα24Jα18⁺ CD3⁺ human NKT cells was estimated using flow cytometry (˜99%) and cells were frozen in complete RPMI medium+10% dimethyl sulfoxide (DMSO) at 5×10⁶ cells per vial. For assaying the effect of M. schaedleri supernatant on human NKT cells, frozen cells were thawed and cultured in complete RPMI medium+rhIL-2 at cell density of 200,000 cells per well in a 96-well plate. The cells were treated with either sterile mBHI or M. schaedleri CDM at 20% volume per volume (v/v) for 16 h and then the culture supernatant was collected. As a positive control for activation, anti-human CD3 was used to stimulate the NKT cells. XCL1 concentration was measured using ONESTEP HUMAN XCL1 ELISA (ABCAM) according to the manufacturer's instructions. See e.g., Li et al., Bio Protoc 3, e418 (2013); the contents of which are incorporated herein by reference in their entirety.

Metabolomics of M. schaedleri Conditioned Medium

Metabolomics studies were carried out at a Center for Mass Spectrometry.

### Sample Preparation.

All solvents were HPLC-MS grade from SIGMA ALDRICH.

Proteins were precipitated from the samples by adding 300 ul of acetonitrile to 100 ul of sample and centrifuging 10 min at 6000 relative centrifugal force (rcf). Supernatants were dried under nitrogen flow and resuspended in 50 ul acetonitrile 30% in water. 25 ul of each sample was pooled to create the pooled sample.

### Instrument Parameters.

Samples were analyzed by LC-MS on a VANQUISH LC coupled to an ID-X MS (THERMOFISHER SCIENTIFIC). Five μL of sample or standard was injected on a ZIC-pHILIC peek-coated column (150 mm×2.1 mm, 5 micron particles, maintained at 40° C., SIGMA ALDRICH). Buffer A was 20 mM Ammonium Carbonate. 0.1% Ammonium hydroxide in water, and Buffer B was Acetonitrile 97% in water. The liquid chromatography (LC) program was as follow: starting at 93% B, to 40% B in 19 min, then to 0% B in 9 min, maintained at 0% B for 5 min, then back to 93% B in 3 min and re-equilibrated at 93% B for 9 min. The flow rate was maintained at 0.15 mL min⁻¹, except for the first 30 seconds where the flow rate was uniformly ramped from 0.05 to 0.15 mL min⁻¹. Data was acquired on the ID-X in switching polarities at 120,000 resolution, with an AGC (Automatic Gain Control) target of 1e5, and a m/z range of 65 to 1000. MS1 data was acquired in switching polarities for all samples. MS2 and MS3 data were acquired on the pooled samples using the AQUIRX DEEPSCAN function, with 5 reinjections, separately in positive and negative ion mode.

### Data Analysis.

Data were analyzed in COMPOUND DISCOVERER 3.2(CD, THERMOFISHER SCIENTIFIC). Identification was based on MS2/MS3 matching with a local mzvault library and corresponding retention time built with pure standards, or on mzcloud match. Each match was manually inspected.

Single Cell Analysis of TDLN cDC1 from cAPC Mice.

TDLN were surgically removed from cAPC mice and enzymatically digested into a single-cell solution; see e.g., Fletcher et al., Front Immunol 2, 35 (2011), the contents of which are incorporated herein by reference in their entirety. TDLN CD45⁺ CD11c⁺ MHCII^{high} CD64⁻ cells were sorted by flow cytometry using a SONY SH800S sorter. Following sorting, the cells were spun down and approximately 9,000 single cells per sample were loaded to the CHROMIUM CONTROLLER (10×GENOMICS). scRNA-Seq libraries were generated using the 10×GENOMICS CHROMIUM SINGLE CELL 3′ KIT v3 and the 10×CHROMIUM CONTROLLER (10×GENOMICS) according to the standard v3 protocol.

The resulting 3′ scRNA-Seq libraries were pooled together and sequenced on a HISEQ 3000 (ILLUMINA, R2 read length 98 base pairs). Reads were mapped using CELL RANGER 3.0.2 (10× GENOMICS) to the mouse transcriptome reference dataset, and transcript-per-million (TPM) was calculated for each gene in each filtered sample. Cells with either <300 detected genes or more than 3,500 detected genes or >0.15 mitochondrial fraction were excluded from further analysis. Finally, the resulting expression matrix was filtered to remove genes detected in <3 cells. Cell expression was normalized followed by selection of highly variable features, data scaling, and cell clustering. All the above steps were performed using the Seurat v3 R/CRAN package. Genes were identified that were differentially expressed (had a P value lower than 0.05 and a |log 2FC|>0.2) using the MAST test implemented in Seurat v3. To evaluate enrichment of gene sets signatures in cDC1, a fast gene set enrichment analysis (fgsea) was performed using the package fgsa v1.16.0 R/Bioconductor with the Hallmark Gene Set Pathways as the reference. See e.g., Liberzon et al., Cell Syst 1, 417-425 (2015); Stuart et al., Cell 177, 1888-1902.e21 (2019); Finak et al., Genome Biol 16, 278 (2015); Korotkevich et al., Fast gene set enrichment analysis (2016); the contents of each of which are incorporated herein by reference in their entireties.

Survival Analysis of cDC1 Gene Signature in Human CRC Bulk Transcriptomic Datasets.

Normalized read count data were obtained for 29 DE-cDC1 genes (FPKM) from the COAD and READ projects under The Cancer Genome Atlas (TCGA) Research Network using the XenaHost (“gdcHub”) function implemented in the UCSCXenaTools (v1.3.6) R/CRAN package; see e.g., Wang et al., Journal of Open Source Software 4, 1627 (2019); the contents of which are incorporated herein by reference in their entirety. Genes and transcriptional signature scores of cDC1 (see e.g., Table 5) were determined from the TDLN of cAPC mice fed low vs. high Saa diets. Survival metadata were curated using clinical and molecular phenotype variables available through the latest “gdcHub” filter as well as the legacy “tcgaHub” host. To evaluate the prognostic significance of cDC1 transcriptional signature found in mouse TDLN for CRC patients, activation scores of DE-cDC1 genes for patient j were computed as follows using Formula 3:

$${log}_{10} [ \frac{{\sum }_{i ∈ H} {g}_{ij}}{{\sum }_{i ∈ L} {g}_{ij}} ]$$

where gᵢⱼ is the normalized expression of scRNA-Seq cDC1 gene I from either high-Saa or low-Saa diet DEG set, which are denoted by H and L, respectively. These were used to dichotomize individuals into distinct survival outcomes by identifying a cut-point that maximized hazard ratio through univariate Cox regression analysis. To test whether risk stratification by transcriptional state score was an independent prognosticator, multivariate Cox model regression analysis was performed by fitting metadata variables from univariate test to adjust for survival covariates.

### Other Quantitative Analyses.

Unless otherwise stated, data were analyzed and visualized using R v4.0.2 and the R packages in Table 8. For two-condition analysis, Wilcoxon rank sum-test (Mann-Whitney U test) was performed, unless otherwise stated (see e.g., Brief Description of the Drawings). For multiple condition analysis, one-way analysis of variance (ANOVA) test with Tukey test for post-hoc analysis was performed, unless otherwise stated. For most experiments, three independent biological repeats were performed, unless otherwise stated. Error-bars typically represent standard error of the mean (SEM).

### Data Availability.

All sequencing data were deposited under the bioproject accession PRJNA688219.

TABLE 1
Table 1: MetaCyc pathways enrichment analysis in microbiomes of ICI treated patients. Table 1 shows the MetaCyc pathways enrichment analysis for stool
metagenomic samples from ICI responder vs. non-responder patients. The pathways, their coefficients and p values are listed below in Table 1.

| | | cohen_d_{—} | cohen_d_{—} | cohen_d_eff_{—} | cohen_d_eff_{—} | | | lmm.adj |
|---|---|---|---|---|---|---|---|---|
| feature | contrast | eff_size | eff_size_SE | size_lower_95CI | size_upper_95CI | lmm.coef | lmm.pvalue | Pvalue |
| PWY-7234 | R - NR | 0.291035757 | 0.114044689 | 0.026296615 | 0.555774899 | 0.261077375 | 0.010655674 | 0.049917185 |
| PWY-6588 | R - NR | 0.249901535 | 0.113952804 | −0.0161455 | 0.51594857 | 0.45019482 | 0.028268328 | 0.109923638 |
| PWY-7003 | R - NR | 0.240884335 | 0.113920931 | −0.025007554 | 0.506776224 | 0.374016748 | 0.034426328 | 0.129675328 |
| COA-PWY | R - NR | 0.225640777 | 0.113831112 | −0.038839889 | 0.490121442 | 0.145482172 | 0.047308133 | 0.166773114 |
| DAPLYSINESYN-PWY | R - NR | 0.217819136 | 0.113749858 | −0.045031735 | 0.480670007 | 0.228477257 | 0.055228189 | 0.184446296 |
| PWY-6415 | R - NR | 0.214365901 | 0.11340894 | −0.042409995 | 0.471141796 | 0.457407775 | 0.057833356 | 0.190413598 |
| HOMOSER-METSYN-PWY | R - NR | 0.202710424 | 0.113857944 | −0.064583429 | 0.470004278 | 0.173028666 | 0.075074978 | 0.228831156 |
| PWY0-1479 | R - NR | 0.201102512 | 0.113675133 | −0.0609398 | 0.463144824 | 0.583839957 | 0.076474927 | 0.231053564 |
| PWY-5088 | R - NR | 0.195278342 | 0.113464053 | −0.062861093 | 0.453417777 | 0.587478153 | 0.084359659 | 0.242317045 |
| ALL-CHORISMATE-PWY | R - NR | 0.192096554 | 0.112247146 | −0.082698369 | 0.466891477 | 0.485757733 | 0.085295048 | 0.243571567 |
| PRPP-PWY | R - NR | 0.186673929 | 0.113562575 | −0.073604869 | 0.446952727 | 0.126140017 | 0.099556772 | 0.273585144 |
| MET-SAM-PWY | R - NR | 0.185371891 | 0.11380835 | −0.081628149 | 0.45237193 | 0.153267385 | 0.103406313 | 0.281090523 |
| METSYN-PWY | R - NR | 0.184837764 | 0.113807945 | −0.082191679 | 0.451867207 | 0.143684553 | 0.104406185 | 0.281916893 |
| PWY-1861 | R - NR | 0.18414466 | 0.113746507 | −0.080894702 | 0.449184021 | 0.357001566 | 0.105324484 | 0.283847451 |
| PWY-5104 | R - NR | 0.183753547 | 0.113775667 | −0.082243849 | 0.449750943 | 0.206162129 | 0.10625342 | 0.284732356 |
| PWY-4242 | R - NR | 0.182803858 | 0.113670421 | −0.080171562 | 0.445779279 | 0.133869018 | 0.107428125 | 0.286201646 |
| PWY-6122 | R - NR | 0.181377679 | 0.11378684 | −0.085180221 | 0.44793558 | 0.092094589 | 0.11094483 | 0.292776904 |
| PWY-6277 | R - NR | 0.181377679 | 0.11378684 | −0.085180221 | 0.44793558 | 0.092094589 | 0.11094483 | 0.292776904 |
| PANTOSYN-PWY | R - NR | 0.179977954 | 0.113475096 | −0.078899571 | 0.438855479 | 0.091202967 | 0.111838318 | 0.294239322 |
| PWY-5103 | R - NR | 0.178965162 | 0.113801785 | −0.088321592 | 0.446251917 | 0.121127628 | 0.115899298 | 0.297878012 |
| HSERMETANA-PWY | R - NR | 0.177868387 | 0.113681011 | −0.085675551 | 0.441412325 | 0.143784475 | 0.117358816 | 0.297878012 |
| PWY-6590 | R - NR | 0.177492193 | 0.113356705 | −0.079616044 | 0.43460043 | 0.408316825 | 0.116201002 | 0.297878012 |
| CENTFERM-PWY | R - NR | 0.177275907 | 0.113348947 | −0.079734548 | 0.434286361 | 0.411756731 | 0.116603227 | 0.297878012 |
| PWY-821 | R - NR | 0.176406078 | 0.113486871 | −0.082818601 | 0.435630758 | 0.359091677 | 0.119223294 | 0.300224086 |
| PWY-5347 | R - NR | 0.176004806 | 0.113790353 | −0.091100128 | 0.44310974 | 0.125701297 | 0.121997325 | 0.304121903 |
| OANTIGEN-PWY | R - NR | 0.171416501 | 0.113764964 | −0.095132802 | 0.437965805 | 0.212300175 | 0.131888851 | 0.319393373 |
| PWY-6549 | R - NR | 0.170856188 | 0.113623362 | −0.091566191 | 0.433278567 | 0.4493795 | 0.13219462 | 0.319393373 |
| UDPNAGSYN-PWY | R - NR | 0.170788999 | 0.113765335 | −0.095819599 | 0.437397596 | 0.256872181 | 0.133317167 | 0.320880628 |
| PWY-5920 | R - NR | 0.16601482 | 0.113465198 | −0.093190756 | 0.425220396 | 0.384292267 | 0.142504746 | 0.33434727 |
| PWY-6121 | R - NR | 0.165951132 | 0.113745758 | −0.100326534 | 0.432228798 | 0.077205775 | 0.144563881 | 0.337476887 |
| PWY-6936 | R - NR | 0.1651397 | 0.113755562 | −0.101536451 | 0.431815851 | 0.176614142 | 0.146618584 | 0.341702076 |
| BRANCHED-CHAIN-AA-SYN-PWY | R - NR | 0.164688355 | 0.113766809 | −0.102426281 | 0.43180299 | 0.103357876 | 0.14781656 | 0.342777273 |
| PWY-7209 | R - NR | 0.161036572 | 0.113748977 | −0.105698644 | 0.427771787 | 0.396408865 | 0.15690375 | 0.355004271 |
| ARGORNPROST-PWY | R - NR | 0.155786368 | 0.113461613 | −0.103714189 | 0.415286926 | 0.350750077 | 0.168811655 | 0.371705158 |
| PWY-4702 | R - NR | 0.153412917 | 0.112216314 | −0.117479849 | 0.424305683 | 0.501842919 | 0.169223538 | 0.371745257 |
| PWY-7196 | R - NR | 0.153186542 | 0.113509459 | −0.107385118 | 0.413758202 | 0.233088286 | 0.176387959 | 0.377665017 |
| PWY-7210 | R - NR | 0.152387753 | 0.113077666 | −0.102561491 | 0.407336996 | 0.470035864 | 0.175762956 | 0.377577548 |
| PWY-7198 | R - NR | 0.14898329 | 0.113095751 | −0.10607748 | 0.40404406 | 0.468038053 | 0.185741362 | 0.390504429 |
| PWY0-41 | R - NR | 0.147564982 | 0.113408235 | −0.111212772 | 0.406342736 | 0.324016432 | 0.192103549 | 0.399072253 |
| RUMP-PWY | R - NR | 0.14698596 | 0.113696573 | −0.118862757 | 0.412834677 | 0.325475417 | 0.196025846 | 0.404212823 |
| PWY-6285 | R - NR | 0.144787106 | 0.113452607 | −0.11491511 | 0.404489323 | 0.313533163 | 0.200947251 | 0.410075589 |
| COA-PWY-1 | R - NR | 0.144290043 | 0.113158809 | −0.111259766 | 0.399839853 | 0.046095676 | 0.200421937 | 0.409647181 |
| PWY-6471 | R - NR | 0.143273242 | 0.113615552 | −0.120318801 | 0.406865285 | 0.402441222 | 0.206937542 | 0.415064381 |
| P108-PWY | R - NR | 0.141309454 | 0.113177137 | −0.114442915 | 0.397061824 | 0.364255757 | 0.2100187 | 0.418837294 |
| PWY-5100 | R - NR | 0.140241913 | 0.113713305 | −0.126600395 | 0.407084222 | 0.120914777 | 0.21754153 | 0.42592984 |
| PWY-7211 | R - NR | 0.137175496 | 0.113280956 | −0.119855086 | 0.394206078 | 0.136999762 | 0.224428949 | 0.43360959 |
| PWY-6317 | R - NR | 0.136096322 | 0.113718022 | −0.131181622 | 0.403374266 | 0.088386877 | 0.231525238 | 0.442146692 |
| PWY-7197 | R - NR | 0.132579341 | 0.112117826 | −0.141898992 | 0.407057675 | 0.085088988 | 0.234443482 | 0.445889782 |
| PWY-7392 | R - NR | 0.13236797 | 0.113609488 | −0.131566765 | 0.396302706 | 0.320848645 | 0.243655275 | 0.457181133 |
| PWY-5505 | R - NR | 0.127819946 | 0.112109527 | −0.14663807 | 0.402277962 | 0.293756898 | 0.251632726 | 0.463429136 |
| PWY-6859 | R - NR | 0.123578746 | 0.113497478 | −0.137813519 | 0.384971012 | 0.345482426 | 0.275528674 | 0.487559643 |
| PYRIDNUCSYN-PWY | R - NR | 0.123257716 | 0.113569541 | −0.139957782 | 0.386473215 | 0.076967883 | 0.277362455 | 0.489454678 |
| PWY0-166 | R - NR | 0.123086734 | 0.11289018 | −0.131497335 | 0.377670803 | 0.058199045 | 0.273052257 | 0.487084858 |
| HISTSYN-PWY | R - NR | 0.122990994 | 0.113689812 | −0.144018283 | 0.39000027 | 0.067568397 | 0.279445483 | 0.490699237 |
| PWY-6527 | R - NR | 0.121735754 | 0.113681093 | −0.145024244 | 0.388495753 | 0.085392324 | 0.28431165 | 0.494357501 |
| GLUTORN-PWY | R - NR | 0.120498115 | 0.113444017 | −0.139804289 | 0.380800518 | 0.198756075 | 0.28726888 | 0.496936006 |
| SER-GLYSYN-PWY | R - NR | 0.118015795 | 0.113612921 | −0.146666372 | 0.382697963 | 0.124284469 | 0.298707801 | 0.512280209 |
| PWY0-781 | R - NR | 0.117689962 | 0.113639264 | −0.147838217 | 0.383218141 | 0.104224717 | 0.30027564 | 0.513706854 |
| PWY-5177 | R - NR | 0.115749678 | 0.11349429 | −0.14583075 | 0.377330107 | 0.17981547 | 0.307115363 | 0.522845179 |
| PWY-7115 | R - NR | 0.114128272 | 0.113638022 | −0.151525536 | 0.37978208 | 0.32719181 | 0.31515324 | 0.528790772 |
| PWY-6737 | R - NR | 0.111985784 | 0.113588883 | −0.152229595 | 0.376201164 | 0.073560388 | 0.323913248 | 0.536397265 |
| P4-PWY | R - NR | 0.1109486 | 0.11362602 | −0.154456214 | 0.376353414 | 0.107429351 | 0.328741632 | 0.542462551 |
| ARGSYN-PWY | R - NR | 0.109989375 | 0.113497224 | −0.151848427 | 0.371827178 | 0.132394366 | 0.331869232 | 0.545688396 |
| ARGSYNBSUB-PWY | R - NR | 0.109027264 | 0.113409677 | −0.150882726 | 0.368937254 | 0.172066499 | 0.33542403 | 0.548821256 |
| PWY-6147 | R - NR | 0.1089353 | 0.113543927 | −0.154131386 | 0.372001987 | 0.149673733 | 0.336909767 | 0.55008893 |
| PWY-5121 | R - NR | 0.106719133 | 0.113469149 | −0.15455251 | 0.367990777 | 0.182762726 | 0.346238607 | 0.559568591 |
| PWY-7400 | R - NR | 0.106356192 | 0.113500759 | −0.155683822 | 0.368396205 | 0.123128898 | 0.348137061 | 0.559907071 |
| PWY66-422 | R - NR | 0.106205938 | 0.113666637 | −0.160826384 | 0.373238259 | 0.072925762 | 0.350236184 | 0.560699213 |
| ANAGLYCOLYSIS-PWY | R - NR | 0.105675369 | 0.113367373 | −0.153483109 | 0.364833846 | 0.04190283 | 0.350179593 | 0.560699213 |
| TEICHOICACID-PWY | R - NR | 0.105275299 | 0.113651577 | −0.161253236 | 0.371803835 | 0.176870989 | 0.354341841 | 0.563395456 |
| UNMAPPED | R - NR | 0.102638113 | 0.113505403 | −0.159634499 | 0.364910725 | 0.033048963 | 0.365303329 | 0.574587458 |
| PWY-7560 | R - NR | 0.100422706 | 0.113482376 | −0.161347194 | 0.362192606 | 0.156342387 | 0.375572656 | 0.584123337 |
| NAGLIPASYN-PWY | R - NR | 0.099020227 | 0.112561805 | −0.157692152 | 0.355732607 | 0.192175239 | 0.3762146 | 0.584123337 |
| PWY-6270 | R - NR | 0.097122401 | 0.113465115 | −0.164327904 | 0.358572705 | 0.157227117 | 0.391345218 | 0.596277941 |
| PWY-5188 | R - NR | 0.096589003 | 0.113658058 | −0.170574189 | 0.363752196 | 0.05358873 | 0.395562985 | 0.600876961 |
| PWY-7184 | R - NR | 0.096045596 | 0.112753279 | −0.1587473 | 0.350838492 | 0.049786846 | 0.39168114 | 0.596277941 |
| THISYNARA-PWY | R - NR | 0.092957002 | 0.113494371 | −0.169324396 | 0.355238399 | 0.057062722 | 0.412226746 | 0.619913533 |
| PWY0-1061 | R - NR | 0.092487166 | 0.113478743 | −0.169417741 | 0.354392073 | 0.251030955 | 0.414472017 | 0.620526851 |
| P164-PWY | R - NR | 0.091968328 | 0.113443194 | −0.169107482 | 0.353044138 | 0.129861097 | 0.416821447 | 0.622948203 |
| PWY-1042 | R - NR | 0.091833739 | 0.113606998 | −0.173726591 | 0.357394069 | 0.039459296 | 0.418817 | 0.623979224 |
| PWY-3001 | R - NR | 0.091735474 | 0.113642918 | −0.175075355 | 0.358546303 | 0.043355516 | 0.419627835 | 0.624177691 |
| TRNA-CHARGING-PWY | R - NR | 0.091169082 | 0.113504613 | −0.171425724 | 0.353763888 | 0.059388029 | 0.421363273 | 0.624177691 |
| PWY-6595 | R - NR | 0.091102132 | 0.113552317 | −0.172799634 | 0.355003898 | 0.130933582 | 0.422084293 | 0.624183976 |
| PWY-6386 | R - NR | 0.091069069 | 0.113427154 | −0.169667028 | 0.351805165 | 0.031134903 | 0.421274377 | 0.624177691 |
| PWY-6113 | R - NR | 0.088297823 | 0.112986282 | −0.166473692 | 0.343069338 | 0.206592398 | 0.432401194 | 0.630892814 |
| PWY-6284 | R - NR | 0.087359882 | 0.113043078 | −0.167768474 | 0.342488239 | 0.192589932 | 0.437684611 | 0.635143156 |
| CRNFORCAT-PWY | R - NR | 0.08456739 | 0.113182287 | −0.172003042 | 0.341137821 | 0.237882158 | 0.453436938 | 0.650077568 |
| THRESYN-PWY | R - NR | 0.082983386 | 0.113616226 | −0.183211662 | 0.349178435 | 0.03664688 | 0.465168024 | 0.660604844 |
| PWY-6387 | R - NR | 0.082722217 | 0.113433614 | −0.178354405 | 0.34379884 | 0.028135189 | 0.46516295 | 0.660604844 |
| PWY-7219 | R - NR | 0.082048215 | 0.113540351 | −0.181783399 | 0.345879829 | 0.02907499 | 0.469612018 | 0.662989489 |
| PWY-7383 | R - NR | 0.080435092 | 0.113180695 | −0.176163179 | 0.337033363 | 0.082874697 | 0.475808514 | 0.669585368 |
| PWY-5367 | R - NR | 0.079995669 | 0.113428091 | −0.181015023 | 0.341006361 | 0.229056469 | 0.479971995 | 0.673407945 |
| PWY-7187 | R - NR | 0.078815173 | 0.113505927 | −0.184148695 | 0.341779041 | 0.034903332 | 0.487048884 | 0.680600843 |
| PWY-5198 | R - NR | 0.078521696 | 0.11326946 | −0.17941285 | 0.336456243 | 0.240332867 | 0.486985349 | 0.680600843 |
| PWY-7117 | R - NR | 0.075515909 | 0.113577286 | −0.189623821 | 0.340655639 | 0.221520943 | 0.50600731 | 0.700085436 |
| PWY-7208 | R - NR | 0.075469742 | 0.112038238 | −0.198813749 | 0.349753233 | 0.040843947 | 0.498211402 | 0.691354987 |
| METHANOGENESIS-PWY | R - NR | 0.074962368 | 0.113324968 | −0.184005379 | 0.333930114 | 0.214833942 | 0.507334454 | 0.700258597 |
| PWY-5863 | R - NR | 0.074942569 | 0.113352698 | −0.184560595 | 0.334445733 | 0.170277718 | 0.507637321 | 0.700258597 |
| PWY-5676 | R - NR | 0.074584405 | 0.113399772 | −0.185905286 | 0.335074095 | 0.105445388 | 0.509993673 | 0.702814578 |
| P124-PWY | R - NR | 0.072332835 | 0.113578574 | −0.192940973 | 0.337606644 | 0.108577232 | 0.52412473 | 0.716628915 |
| PWY-7221 | R - NR | 0.071636231 | 0.113437551 | −0.189769691 | 0.333042153 | 0.02609129 | 0.527128615 | 0.718990617 |
| PWY-7409 | R - NR | 0.071598122 | 0.112034423 | −0.202676028 | 0.345872272 | 0.24447264 | 0.520494946 | 0.713763207 |
| PWY-7242 | R - NR | 0.071084377 | 0.113313142 | −0.187724672 | 0.329893425 | 0.072129157 | 0.529481111 | 0.720478288 |
| POLYAMINSYN3-PWY | R - NR | 0.070226428 | 0.113242167 | −0.187381315 | 0.327834171 | 0.224427239 | 0.533999622 | 0.722348325 |
| GLUCUROCAT-PWY | R - NR | 0.06909277 | 0.1132595 | −0.188808669 | 0.326994209 | 0.064126623 | 0.540736735 | 0.729446476 |
| PWY-5971 | R - NR | 0.068383374 | 0.112477234 | −0.189065471 | 0.325832219 | 0.168731753 | 0.540814544 | 0.729446476 |
| COBALSYN-PWY | R - NR | 0.068323396 | 0.113422112 | −0.192786634 | 0.329433427 | 0.083087895 | 0.546315924 | 0.733787968 |
| PEPTIDOGLYCANSYN-PWY | R - NR | 0.068102046 | 0.113350568 | −0.191473006 | 0.327677099 | 0.023072527 | 0.547147496 | 0.733787968 |
| PWY-621 | R - NR | 0.068099796 | 0.113611482 | −0.198417799 | 0.334617392 | 0.066549255 | 0.548950605 | 0.735446301 |
| PWY0-1296 | R - NR | 0.067837245 | 0.113603013 | −0.198388128 | 0.334062618 | 0.039790997 | 0.550429367 | 0.736720419 |
| PWY-7286 | R - NR | 0.066661982 | 0.113360175 | −0.193130588 | 0.326454551 | 0.125963425 | 0.555718819 | 0.7405969 |
| PWY-6167 | R - NR | 0.066582885 | 0.113370844 | −0.193431621 | 0.326597391 | 0.116798838 | 0.556256598 | 0.7405969 |
| ARG + POLYAMINE-SYN | R - NR | 0.065992581 | 0.113430639 | −0.195359711 | 0.327344873 | 0.094870022 | 0.56015014 | 0.742611202 |
| PWY-6892 | R - NR | 0.064988612 | 0.112931916 | −0.189594295 | 0.31957152 | 0.168202349 | 0.563123614 | 0.744122197 |
| PWY-5705 | R - NR | 0.064929669 | 0.11273085 | −0.189733984 | 0.319593322 | 0.191980178 | 0.562476428 | 0.744122197 |
| PWY-2941 | R - NR | 0.064905985 | 0.113591731 | −0.201012418 | 0.330824388 | 0.082769564 | 0.567714008 | 0.74837465 |
| PWY0-1298 | R - NR | 0.064671117 | 0.113526364 | −0.19920405 | 0.328546284 | 0.066191224 | 0.568663876 | 0.748808009 |
| PWY-7328 | R - NR | 0.063089133 | 0.113039316 | −0.192143885 | 0.318322152 | 0.147305098 | 0.575180872 | 0.75182818 |
| THISYN-PWY | R - NR | 0.063013362 | 0.112758637 | −0.191527599 | 0.317554323 | 0.136641548 | 0.574193014 | 0.751240344 |
| PWY-2723 | R - NR | 0.062298609 | 0.112379678 | −0.197040868 | 0.321638086 | 0.20530109 | 0.577081779 | 0.753607262 |
| PWY-6165 | R - NR | 0.062073378 | 0.113114726 | −0.193896611 | 0.318043366 | 0.098584921 | 0.581782486 | 0.757619729 |
| PWY-5840 | R - NR | 0.061288468 | 0.11314294 | −0.195018721 | 0.317595656 | 0.115680834 | 0.58673142 | 0.760517235 |
| GALACT-GLUCUROCAT-PWY | R - NR | 0.060902915 | 0.11324345 | −0.19683602 | 0.31864185 | 0.052445318 | 0.589677515 | 0.762919195 |
| PWY-5686 | R - NR | 0.060379285 | 0.113491018 | −0.202600745 | 0.323359314 | 0.022289487 | 0.594380348 | 0.765456611 |
| PPGPPMET-PWY | R - NR | 0.060246054 | 0.113357161 | −0.199582933 | 0.320075041 | 0.122068291 | 0.594374077 | 0.765456611 |
| PWY-5384 | R - NR | 0.059197396 | 0.11319634 | −0.197840703 | 0.316235496 | 0.162770504 | 0.599870107 | 0.77039436 |
| DTDPRHAMSYN-PWY | R - NR | 0.059082648 | 0.113458034 | −0.20306289 | 0.321228186 | 0.038303113 | 0.602122337 | 0.77186665 |
| PWY-5692 | R - NR | 0.057906287 | 0.113234697 | −0.199725036 | 0.315537609 | 0.124197894 | 0.608069694 | 0.775695427 |
| URDEGR-PWY | R - NR | 0.057906287 | 0.113234697 | −0.199725036 | 0.315537609 | 0.124197894 | 0.608069694 | 0.775695427 |
| HEMESYN2-PWY | R - NR | 0.056854393 | 0.112021731 | −0.217388686 | 0.331097472 | 0.078641513 | 0.609828699 | 0.776752613 |
| PWY-6609 | R - NR | 0.056819982 | 0.113532714 | −0.207411817 | 0.321051782 | 0.029202279 | 0.61655646 | 0.783178178 |
| AEROBACTINSYN-PWY | R - NR | 0.056675099 | 0.112898091 | −0.19780512 | 0.311155319 | 0.20875439 | 0.613938356 | 0.780562792 |
| PWY-6612 | R - NR | 0.055964202 | 0.113488474 | −0.207030985 | 0.318959388 | 0.108253793 | 0.621609842 | 0.784599764 |
| PWY-6630 | R - NR | 0.055960064 | 0.113305343 | −0.20291129 | 0.314831418 | 0.147552142 | 0.620580211 | 0.784599764 |
| PWY0-1338 | R - NR | 0.053699784 | 0.112627882 | −0.201584373 | 0.308983941 | 0.192694763 | 0.631555028 | 0.791428024 |
| FOLSYN-PWY | R - NR | 0.052058452 | 0.113475028 | −0.210646095 | 0.314762999 | 0.098136022 | 0.646077284 | 0.803584324 |
| GLUCOSE1PMETAB-PWY | R - NR | 0.051168053 | 0.113098833 | −0.204698506 | 0.307034613 | 0.160747284 | 0.64975289 | 0.804363846 |
| PWY-6545 | R - NR | 0.050819961 | 0.112993259 | −0.204118039 | 0.305757962 | 0.023440743 | 0.651464427 | 0.804818 |
| ARO-PWY | R - NR | 0.050495242 | 0.113590585 | −0.215738113 | 0.316728597 | 0.023010446 | 0.656671661 | 0.810533722 |
| POLYISOPRENSYN-PWY | R - NR | 0.047373032 | 0.113353883 | −0.212556851 | 0.307302916 | 0.128526555 | 0.675421786 | 0.82782161 |
| PWY-7539 | R - NR | 0.047149902 | 0.113233639 | −0.210567264 | 0.304867069 | 0.062507734 | 0.676272543 | 0.828137255 |
| METHGLYUT-PWY | R - NR | 0.045773597 | 0.112978532 | −0.209082597 | 0.300629792 | 0.147986894 | 0.684043227 | 0.832540841 |
| PYRIDNUCSAL-PWY | R - NR | 0.044887864 | 0.113524595 | −0.219322461 | 0.309098188 | 0.086500055 | 0.692391661 | 0.838715533 |
| PWY-6507 | R - NR | 0.044754462 | 0.113416042 | −0.216584842 | 0.306093765 | 0.046772858 | 0.692721354 | 0.838715533 |
| GLYCOGENSYNTH-PWY | R - NR | 0.044515117 | 0.113598867 | −0.222139689 | 0.311169924 | 0.045699993 | 0.69521083 | 0.839686369 |
| PWY-6263 | R - NR | 0.043632144 | 0.112012826 | −0.230589135 | 0.317853424 | 0.096044166 | 0.695303502 | 0.839686369 |
| PWY-241 | R - NR | 0.041238927 | 0.113527847 | −0.223126221 | 0.305604076 | 0.108023644 | 0.716287347 | 0.853188683 |
| CALVIN-PWY | R - NR | 0.041024146 | 0.113553436 | −0.224140815 | 0.306189107 | 0.019873696 | 0.717825741 | 0.854292186 |
| GALACTUROCAT-PWY | R - NR | 0.036744135 | 0.113310399 | −0.222419745 | 0.295908015 | 0.034407071 | 0.745190304 | 0.873455638 |
| PWY-5189 | R - NR | 0.034184165 | 0.113350687 | −0.225811378 | 0.294179708 | 0.066986376 | 0.762541692 | 0.882691778 |
| GLYCOL-GLYOXDEG-PWY | R - NR | 0.033661754 | 0.112770083 | −0.220753101 | 0.288076609 | 0.117483982 | 0.764100685 | 0.883017017 |
| ECASYN-PWY | R - NR | 0.033086397 | 0.112087326 | −0.236267129 | 0.302439922 | 0.111453326 | 0.766581998 | 0.885151753 |
| P162-PWY | R - NR | 0.032607354 | 0.113405096 | −0.228606685 | 0.293821394 | 0.047982444 | 0.77338557 | 0.888597742 |
| PWY-7315 | R - NR | 0.032468639 | 0.112893971 | −0.222008731 | 0.286946009 | 0.107048802 | 0.772583699 | 0.888407615 |
| COMPLETE-ARO-PWY | R - NR | 0.031757598 | 0.1135718 | −0.234153888 | 0.297669085 | 0.013790845 | 0.779757871 | 0.892979481 |
| PWY66-409 | R - NR | 0.030802474 | 0.113303325 | −0.228270588 | 0.289875536 | 0.03723403 | 0.785266881 | 0.896347151 |
| PWY-5022 | R - NR | 0.029899585 | 0.11269748 | −0.22476957 | 0.28456874 | 0.078687727 | 0.789631244 | 0.896407805 |
| NAD-BIOSYNTHESIS-II | R - NR | 0.029775668 | 0.113445641 | −0.23245251 | 0.292003846 | 0.083755225 | 0.792733551 | 0.898911651 |
| PWY-5897 | R - NR | 0.02835746 | 0.113176176 | −0.228607658 | 0.285322577 | 0.055098749 | 0.801548788 | 0.901632831 |
| PWY-5898 | R - NR | 0.02835746 | 0.113176176 | −0.228607658 | 0.285322577 | 0.055098749 | 0.801548788 | 0.901632831 |
| PWY-5899 | R - NR | 0.02835746 | 0.113176176 | −0.228607658 | 0.285322577 | 0.055098749 | 0.801548788 | 0.901632831 |
| PWY-5860 | R - NR | 0.028220008 | 0.112005404 | −0.2459831 | 0.302423116 | 0.08092963 | 0.800008236 | 0.901632831 |
| PWY-5862 | R - NR | 0.028220008 | 0.112005404 | −0.2459831 | 0.302423116 | 0.08092963 | 0.800008236 | 0.901632831 |
| PWY-5791 | R - NR | 0.028117886 | 0.11326413 | −0.230252234 | 0.286488006 | 0.05954128 | 0.803460775 | 0.901632831 |
| PWY-5837 | R - NR | 0.028117886 | 0.11326413 | −0.230252234 | 0.286488006 | 0.05954128 | 0.803460775 | 0.901632831 |
| PWY-5845 | R - NR | 0.027846779 | 0.112005263 | −0.246355986 | 0.302049543 | 0.078013643 | 0.802598014 | 0.901632831 |
| PWY-5850 | R - NR | 0.027846779 | 0.112005263 | −0.246355986 | 0.302049543 | 0.078013643 | 0.802598014 | 0.901632831 |
| PWY-5896 | R - NR | 0.027846779 | 0.112005263 | −0.246355986 | 0.302049543 | 0.078013643 | 0.802598014 | 0.901632831 |
| PWY-6606 | R - NR | 0.027749367 | 0.113491189 | −0.235714269 | 0.291213003 | 0.029595127 | 0.806696892 | 0.903811285 |
| PWY0-1533 | R - NR | 0.026575109 | 0.112251734 | −0.235711182 | 0.2888614 | 0.087533095 | 0.811775895 | 0.907239784 |
| PWY-7269 | R - NR | 0.02651334 | 0.112351477 | −0.23294467 | 0.285971351 | 0.085198672 | 0.81235654 | 0.907239784 |
| PWY0-1261 | R - NR | 0.025203622 | 0.113282911 | −0.23352094 | 0.283928183 | 0.036596758 | 0.82352953 | 0.914596042 |
| PWY-7357 | R - NR | 0.02416309 | 0.113585484 | −0.242320089 | 0.290646269 | 0.013883353 | 0.83155917 | 0.919854677 |
| REDCITCYC | R - NR | 0.024151879 | 0.113157305 | −0.232566796 | 0.280870554 | 0.067795265 | 0.830446504 | 0.919854677 |
| ASPASN-PWY | R - NR | 0.024111517 | 0.113259158 | −0.23419249 | 0.282415525 | 0.013174608 | 0.830995148 | 0.919854677 |
| AST-PWY | R - NR | 0.023047038 | 0.112801072 | −0.231311235 | 0.277405311 | 0.076558683 | 0.837270872 | 0.923976393 |
| PWY490-3 | R - NR | 0.022302369 | 0.113120292 | −0.2339433 | 0.278548038 | 0.057567418 | 0.843166426 | 0.926716528 |
| PWY-7237 | R - NR | 0.02175145 | 0.113571772 | −0.244272549 | 0.287775449 | 0.027770528 | 0.848117265 | 0.928814388 |
| PWY0-162 | R - NR | 0.021390954 | 0.112533311 | −0.234711649 | 0.277493556 | 0.010523714 | 0.848365903 | 0.928814388 |
| PWY-724 | R - NR | 0.020772304 | 0.113471813 | −0.242213992 | 0.2837586 | 0.009202376 | 0.854622144 | 0.929310372 |
| NONOXIPENT-PWY | R - NR | 0.020759545 | 0.113488952 | −0.24269907 | 0.284218161 | 0.014256561 | 0.85475252 | 0.929310372 |
| ILEUSYN-PWY | R - NR | 0.019137725 | 0.113561559 | −0.246560384 | 0.284835833 | 0.007581305 | 0.866159979 | 0.936703263 |
| VALSYN-PWY | R - NR | 0.019137725 | 0.113561559 | −0.246560384 | 0.284835833 | 0.007581305 | 0.866159979 | 0.936703263 |
| PWY-6895 | R - NR | 0.018020678 | 0.112002242 | −0.256174691 | 0.292216047 | 0.0554573 | 0.871479345 | 0.939448004 |
| PWY4FS-7 | R - NR | 0.017710333 | 0.113160802 | −0.239076896 | 0.274497562 | 0.019952312 | 0.87523851 | 0.940656311 |
| PWY4FS-8 | R - NR | 0.017702164 | 0.113160766 | −0.239084585 | 0.274488912 | 0.019943067 | 0.875295522 | 0.940656311 |
| PWY-6891 | R - NR | 0.016762721 | 0.112001949 | −0.25743193 | 0.290957372 | 0.057962805 | 0.880380527 | 0.943941026 |
| LACTOSECAT-PWY | R - NR | 0.015449776 | 0.113561214 | −0.250263136 | 0.281162688 | 0.034289788 | 0.891774091 | 0.947555491 |
| PWY-5747 | R - NR | 0.015178223 | 0.113029951 | −0.240130546 | 0.270486992 | 0.044974566 | 0.892745654 | 0.947555491 |
| PWY-6823 | R - NR | 0.015031092 | 0.112579116 | −0.240514973 | 0.270577156 | 0.042610684 | 0.893170922 | 0.947555491 |
| FERMENTATION-PWY | R - NR | 0.014813682 | 0.113361987 | −0.245542462 | 0.275169825 | 0.034960187 | 0.895851162 | 0.94886815 |
| PWY-7199 | R - NR | 0.014476322 | 0.113496743 | −0.249241472 | 0.278194115 | 0.010803365 | 0.898438947 | 0.949447971 |
| PWY0-42 | R - NR | 0.014439594 | 0.112919583 | −0.240141704 | 0.269020891 | 0.041365341 | 0.897773182 | 0.949447971 |
| UBISYN-PWY | R - NR | 0.013922699 | 0.112001365 | −0.260270522 | 0.28811592 | 0.042920093 | 0.90053045 | 0.949501895 |
| TCA-GLYOX-BYPASS | R - NR | 0.013620385 | 0.112929921 | −0.241008668 | 0.268249439 | 0.038195277 | 0.903557689 | 0.951256813 |
| PWY-4981 | R - NR | 0.013536395 | 0.113571437 | −0.252537898 | 0.279610689 | 0.016268624 | 0.905127728 | 0.952191642 |
| ENTBACSYN-PWY | R - NR | 0.012623353 | 0.113216102 | −0.245000876 | 0.270247582 | 0.033480682 | 0.910972823 | 0.954743289 |
| PWY-6151 | R - NR | 0.010788985 | 0.11340556 | −0.250568989 | 0.272146959 | 0.005830766 | 0.92410052 | 0.962662697 |
| PWY-5101 | R - NR | 0.010077468 | 0.113224577 | −0.247688792 | 0.267843729 | 0.020510789 | 0.92888399 | 0.964105613 |
| PWY-5690 | R - NR | 0.009895602 | 0.113379211 | −0.250856868 | 0.270648072 | 0.011171313 | 0.930338319 | 0.964480055 |
| P185-PWY | R - NR | 0.009876098 | 0.11350835 | −0.254199097 | 0.273951294 | 0.014161708 | 0.930626529 | 0.964480055 |
| PWY-6628 | R - NR | 0.009395214 | 0.113347944 | −0.25067679 | 0.269467219 | 0.023005761 | 0.933818777 | 0.966659383 |
| KDO-NAGLIPASYN-PWY | R - NR | 0.007597496 | 0.11285139 | −0.246780331 | 0.261975323 | 0.023979221 | 0.946055812 | 0.973963063 |
| PWY-5345 | R - NR | 0.006926588 | 0.11217113 | −0.258145431 | 0.271998608 | 0.009817918 | 0.950477138 | 0.975026237 |
| PWY-7228 | R - NR | 0.006900725 | 0.113239169 | −0.251112618 | 0.264914068 | 0.003899067 | 0.95127918 | 0.975026237 |
| PWY-5154 | R - NR | 0.006205594 | 0.112899074 | −0.248296111 | 0.2607073 | 0.008301701 | 0.955955948 | 0.978245895 |
| GLYOXYLATE-BYPASS | R - NR | 0.004906243 | 0.113162862 | −0.251932016 | 0.261744502 | 0.015446596 | 0.96530813 | 0.980623895 |
| PWY-6969 | R - NR | 0.004655157 | 0.113309068 | −0.254633727 | 0.263944041 | 0.005943241 | 0.967159332 | 0.980623895 |
| PWY-4041 | R - NR | 0.003578298 | 0.112872966 | −0.250843982 | 0.258000577 | 0.012014345 | 0.974585464 | 0.983746426 |
| PWY-6629 | R - NR | 0.002894107 | 0.112911441 | −0.251655363 | 0.257443577 | 0.008247637 | 0.979454517 | 0.986637665 |
| P461-PWY | R - NR | 0.00189834 | 0.113433064 | −0.260146312 | 0.263942992 | 0.002557547 | 0.986631966 | 0.991604194 |
| GLYCOLYSIS-TCA-GLYOX-BYPASS | R - NR | 0.000934704 | 0.112903817 | −0.253584821 | 0.255454228 | 0.0024291 | 0.993363111 | 0.996933791 |
| FUCCAT-PWY | R - NR | 0.000243604 | 0.112076874 | −0.269260338 | 0.269747545 | 0.000551062 | 0.998255999 | 0.998255999 |
| PWY-7111 | R - NR | −0.000496541 | 0.113577505 | −0.266823509 | 0.265830426 | −0.000174232 | 0.996512112 | 0.997941828 |
| PWY-6123 | R - NR | −0.00061897 | 0.113487507 | −0.26411044 | 0.2628725 | −0.000335285 | 0.995645094 | 0.997941828 |
| P105-PWY | R - NR | −0.001583455 | 0.113075894 | −0.257350691 | 0.254183782 | −0.003974461 | 0.988785154 | 0.993053292 |
| PWY-7204 | R - NR | −0.001939099 | 0.112090085 | −0.270743564 | 0.266865365 | −0.006963208 | 0.986119572 | 0.991604194 |
| PWY-5675 | R - NR | −0.003154374 | 0.113264094 | −0.26160379 | 0.255295043 | −0.005916249 | 0.977727335 | 0.98620474 |
| PWY-6163 | R - NR | −0.003878413 | 0.113563228 | −0.269706134 | 0.261949307 | −0.001748144 | 0.97275451 | 0.983320273 |
| PWY-6731 | R - NR | −0.004439285 | 0.113310586 | −0.263758154 | 0.254879583 | −0.010470647 | 0.9686822 | 0.980623895 |
| PWY-7399 | R - NR | −0.004465941 | 0.113463088 | −0.267284689 | 0.258352807 | −0.010241025 | 0.968573608 | 0.980623895 |
| POLYAMSYN-PWY | R - NR | −0.004685272 | 0.113324441 | −0.26427974 | 0.254909197 | −0.004633447 | 0.966955241 | 0.980623895 |
| PWY0-1319 | R - NR | −0.005034959 | 0.113403611 | −0.266359696 | 0.256289779 | −0.001595673 | 0.964536678 | 0.980623895 |
| PWY-5667 | R - NR | −0.005132145 | 0.113403161 | −0.266446128 | 0.256181838 | −0.001626817 | 0.963852347 | 0.980623895 |
| PWYG-321 | R - NR | −0.005268861 | 0.112482077 | −0.262051842 | 0.251514121 | −0.010356293 | 0.962418117 | 0.980623895 |
| PWY-6531 | R - NR | −0.00588461 | 0.112971252 | −0.260750844 | 0.248981624 | −0.01483342 | 0.958274611 | 0.978752503 |
| TCA | R - NR | −0.006397425 | 0.113341906 | −0.266348389 | 0.253553539 | −0.00791418 | 0.954903522 | 0.978022976 |
| NONMEVIPP-PWY | R - NR | −0.00708108 | 0.113304459 | −0.266276947 | 0.252114787 | −0.00306789 | 0.95005997 | 0.975026237 |
| PWY0-321 | R - NR | −0.007866151 | 0.112391759 | −0.26630287 | 0.250570568 | −0.022126013 | 0.943871132 | 0.97243107 |
| PWY4LZ-257 | R - NR | −0.009243748 | 0.113288819 | −0.268137406 | 0.249649909 | −0.008620047 | 0.934820938 | 0.966674096 |
| PWY-7254 | R - NR | −0.010442226 | 0.113054918 | −0.265989074 | 0.245104623 | −0.032783002 | 0.926122229 | 0.963387952 |
| PWY-5723 | R - NR | −0.011259157 | 0.112955289 | −0.266024388 | 0.243506073 | −0.037096331 | 0.920244072 | 0.959418016 |
| PWY-6124 | R - NR | −0.011715493 | 0.113454599 | −0.274291169 | 0.250860183 | −0.007202279 | 0.917672175 | 0.958531655 |
| PWY-5913 | R - NR | −0.012025528 | 0.113392995 | −0.273085734 | 0.249034679 | −0.033900376 | 0.915414619 | 0.957256659 |
| PWY-5083 | R - NR | −0.012409596 | 0.113117555 | −0.2686445 | 0.243825308 | −0.03588439 | 0.912338891 | 0.955457683 |
| DENOVOPURINE 2-PWY | R - NR | −0.012698669 | 0.113410552 | −0.274169043 | 0.248771704 | −0.005683935 | 0.910725074 | 0.954743289 |
| PWY-5861 | R - NR | −0.013585261 | 0.112668557 | −0.268382176 | 0.241211654 | −0.036924126 | 0.903486868 | 0.951256813 |
| PWY0-1415 | R - NR | −0.01405256 | 0.112752079 | −0.268481055 | 0.240375936 | −0.035987366 | 0.900281354 | 0.949501895 |
| PWY-5838 | R - NR | −0.015904121 | 0.112502993 | −0.272399432 | 0.24059119 | −0.042121077 | 0.886920959 | 0.947204199 |
| FUC-RHAMCAT-PWY | R - NR | −0.016338153 | 0.113324182 | −0.275898404 | 0.243222099 | −0.049771773 | 0.885133499 | 0.946855451 |
| PWY-5941 | R - NR | −0.017455772 | 0.113445476 | −0.279769099 | 0.244857555 | −0.050622972 | 0.877579609 | 0.942385488 |
| PWY-561 | R - NR | −0.019761746 | 0.113097767 | −0.275747753 | 0.236224261 | −0.053571673 | 0.86079104 | 0.932969171 |
| PWY66-399 | R - NR | −0.020885077 | 0.112344936 | −0.280455463 | 0.238685309 | −0.020058034 | 0.851656167 | 0.928814388 |
| KETOGLUCONMET-PWY | R - NR | −0.021024521 | 0.113230672 | −0.278858757 | 0.236809715 | −0.063788382 | 0.852300309 | 0.928814388 |
| P125-PWY | R - NR | −0.021137653 | 0.113486293 | −0.284519133 | 0.242243827 | −0.035627223 | 0.852130352 | 0.928814388 |
| ARGDEG-PWY | R - NR | −0.021235914 | 0.113000121 | −0.276292013 | 0.233820184 | −0.086506245 | 0.850306345 | 0.928814388 |
| ORNARGDEG-PWY | R - NR | −0.021235914 | 0.113000121 | −0.276292013 | 0.233820184 | −0.086506245 | 0.850306345 | 0.928814388 |
| PWY-5030 | R - NR | −0.022609869 | 0.112968636 | −0.277446266 | 0.232226529 | −0.039183688 | 0.840674387 | 0.926267912 |
| ORNDEG-PWY | R - NR | −0.02497778 | 0.113148086 | −0.281569375 | 0.231613814 | −0.097020213 | 0.824715765 | 0.91518538 |
| PWY-5659 | R - NR | −0.025742157 | 0.113388432 | −0.286627154 | 0.235142839 | −0.016063408 | 0.820127387 | 0.911542861 |
| TRPSYN-PWY | R - NR | −0.025913247 | 0.113530914 | −0.290563711 | 0.238737218 | −0.019899207 | 0.819383103 | 0.911441284 |
| GOLPDLCAT-PWY | R - NR | −0.026040811 | 0.113561111 | −0.291659566 | 0.239577943 | −0.048332803 | 0.818606764 | 0.911303862 |
| P23-PWY | R - NR | −0.026434173 | 0.113516484 | −0.290641905 | 0.237773559 | −0.059047139 | 0.815770148 | 0.909596746 |
| PWY-6803 | R - NR | −0.027728487 | 0.112780116 | −0.282115003 | 0.226658028 | −0.09569705 | 0.804775652 | 0.90238298 |
| GLCMANNANAUT-PWY | R - NR | −0.029409916 | 0.113547128 | −0.294533375 | 0.235713543 | −0.018161426 | 0.795575546 | 0.900748955 |
| HEME-BIOSYNTHESIS-II | R - NR | −0.029692173 | 0.112005975 | −0.30389668 | 0.244512335 | −0.059534865 | 0.7898149 | 0.896407805 |
| FAO-PWY | R - NR | −0.030290554 | 0.113458465 | −0.292844269 | 0.232263161 | −0.063098548 | 0.789277497 | 0.896407805 |
| PWY-7446 | R - NR | −0.030591607 | 0.113319468 | −0.289979741 | 0.228796527 | −0.1268406 | 0.786755737 | 0.896407805 |
| SULFATE-CYS-PWY | R - NR | −0.030873998 | 0.112702365 | −0.285521219 | 0.223773222 | −0.046643256 | 0.782956066 | 0.894440809 |
| PWY-5918 | R - NR | −0.032798942 | 0.112167935 | −0.298305858 | 0.232707973 | −0.063548759 | 0.768683867 | 0.886112864 |
| P122-PWY | R - NR | −0.033215445 | 0.113214308 | −0.29072657 | 0.224295679 | −0.100142468 | 0.768583843 | 0.886112864 |
| PWY0-1277 | R - NR | −0.033827877 | 0.112843848 | −0.288202728 | 0.220546975 | −0.110999759 | 0.763186946 | 0.882691778 |
| ALLANTOINDEG-PWY | R - NR | −0.034530379 | 0.112985127 | −0.289454985 | 0.220394227 | −0.080702248 | 0.758878347 | 0.882206237 |
| PWY-5910 | R - NR | −0.034837878 | 0.113560327 | −0.300321265 | 0.230645509 | −0.06428029 | 0.758975423 | 0.882206237 |
| PHOSLIPSYN-PWY | R - NR | −0.035346016 | 0.112431531 | −0.293161323 | 0.222469291 | −0.034877747 | 0.751814937 | 0.87754171 |
| HCAMHPDEG-PWY | R - NR | −0.037537349 | 0.11281813 | −0.291904486 | 0.216829789 | −0.151815626 | 0.738038528 | 0.867257395 |
| PWY-6690 | R - NR | −0.037537349 | 0.11281813 | −0.291904486 | 0.216829789 | −0.151815626 | 0.738038528 | 0.867257395 |
| PWY-5464 | R - NR | −0.037577117 | 0.113439696 | −0.299576051 | 0.224421817 | −0.100716443 | 0.740153126 | 0.868464724 |
| PWY0-862 | R - NR | −0.03868406 | 0.112521294 | −0.295056413 | 0.217688293 | −0.072963758 | 0.729476331 | 0.862894413 |
| PWY0-1241 | R - NR | −0.039018773 | 0.112946494 | −0.293705945 | 0.2156684 | −0.108628799 | 0.728553851 | 0.862894413 |
| PWY-922 | R - NR | −0.039282939 | 0.113563568 | −0.304806577 | 0.226240698 | −0.086509799 | 0.729371327 | 0.862894413 |
| PWY-6125 | R - NR | −0.040178625 | 0.113178527 | −0.297112884 | 0.216755635 | −0.021917478 | 0.721751458 | 0.858232568 |
| PWY-6282 | R - NR | −0.041359038 | 0.112270282 | −0.303280154 | 0.220562078 | −0.077685045 | 0.710967626 | 0.848299834 |
| PWY-6519 | R - NR | −0.042604629 | 0.112590695 | −0.298164482 | 0.212955224 | −0.080498159 | 0.703496417 | 0.841835341 |
| BIOTIN-BIOSYNTHESIS-PWY | R - NR | −0.042793872 | 0.112612644 | −0.298137611 | 0.212549867 | −0.078534385 | 0.702307497 | 0.841563318 |
| PWY-5855 | R - NR | −0.042945182 | 0.112502785 | −0.299624496 | 0.213734132 | −0.13278491 | 0.700990481 | 0.841429674 |
| PWY-5856 | R - NR | −0.042945182 | 0.112502785 | −0.299624496 | 0.213734132 | −0.13278491 | 0.700990481 | 0.841429674 |
| PWY-5857 | R - NR | −0.042945182 | 0.112502785 | −0.299624496 | 0.213734132 | −0.13278491 | 0.700990481 | 0.841429674 |
| PWY-6708 | R - NR | −0.042945182 | 0.112502785 | −0.299624496 | 0.213734132 | −0.13278491 | 0.700990481 | 0.841429674 |
| PWY-7664 | R - NR | −0.043381468 | 0.112385025 | −0.302257809 | 0.215494874 | −0.082771595 | 0.69779087 | 0.841205574 |
| FASYN-ELONG-PWY | R - NR | −0.045122921 | 0.112395014 | −0.30380332 | 0.213557478 | −0.084780492 | 0.686318275 | 0.833416229 |
| PWY-3841 | R - NR | −0.046456809 | 0.113385683 | −0.307080578 | 0.214166959 | −0.020406559 | 0.681508713 | 0.830813232 |
| PWY-5173 | R - NR | −0.046841421 | 0.113427045 | −0.308415649 | 0.214732806 | −0.153486012 | 0.679225366 | 0.830235599 |
| FASYN-INITIAL-PWY | R - NR | −0.047495292 | 0.11238597 | −0.306412314 | 0.211421729 | −0.093427395 | 0.670747067 | 0.822814504 |
| PWY-7388 | R - NR | −0.047841746 | 0.112361044 | −0.307349292 | 0.211665801 | −0.094507209 | 0.66842103 | 0.822814504 |
| PWY-6353 | R - NR | −0.051098429 | 0.113208295 | −0.30838632 | 0.206189463 | −0.046753608 | 0.650755335 | 0.804654072 |
| GALACTARDEG-PWY | R - NR | −0.051422213 | 0.113091852 | −0.307212325 | 0.204367898 | −0.153301272 | 0.648094847 | 0.803584324 |
| GLUCARGALACTSUPER-PWY | R - NR | −0.051422213 | 0.113091852 | −0.307212325 | 0.204367898 | −0.153301272 | 0.648094847 | 0.803584324 |
| PENTOSE-P-PWY | R - NR | −0.051422516 | 0.113444659 | −0.313360939 | 0.210515908 | −0.049299669 | 0.649944425 | 0.804363846 |
| P161-PWY | R - NR | −0.051451696 | 0.113137214 | −0.307762335 | 0.204858942 | −0.059667222 | 0.648136285 | 0.803584324 |
| 3-HYDROXYPHENYLACETATE- | R - NR | −0.051738133 | 0.11262714 | −0.307013449 | 0.203537184 | −0.157750246 | 0.644063528 | 0.802779183 |
| DEGRADATION-PWY | | | | | | | | |
| PWY-5136 | R - NR | −0.05307212 | 0.113468032 | −0.31557688 | 0.209432639 | −0.114627865 | 0.639628195 | 0.798390079 |
| PWY-5005 | R - NR | −0.054871936 | 0.112775214 | −0.309331643 | 0.199587771 | −0.130223768 | 0.624712974 | 0.786296275 |
| PWY-7229 | R - NR | −0.054999496 | 0.113049255 | −0.310356749 | 0.200357756 | −0.021913859 | 0.625206924 | 0.786296275 |
| PWY-841 | R - NR | −0.05524316 | 0.113479456 | −0.318010231 | 0.207523911 | −0.025511757 | 0.626057176 | 0.786656901 |
| PWY66-398 | R - NR | −0.059636638 | 0.113152746 | −0.316080012 | 0.196806736 | −0.154608411 | 0.596914335 | 0.767304246 |
| GLUDEG-I-PWY | R - NR | −0.063350311 | 0.112026964 | −0.337606201 | 0.210905579 | −0.1604214 | 0.569629098 | 0.748808009 |
| PWY0-1297 | R - NR | −0.064291453 | 0.113326219 | −0.323447117 | 0.19486421 | −0.073005202 | 0.569651938 | 0.748808009 |
| PWY-7013 | R - NR | −0.064657266 | 0.112566045 | −0.320716091 | 0.19140156 | −0.267689656 | 0.563422036 | 0.744122197 |
| PWY-6126 | R - NR | −0.066004995 | 0.113006643 | −0.32098037 | 0.188970379 | −0.030333589 | 0.557451913 | 0.741145591 |
| PWY-6897 | R - NR | −0.066235823 | 0.113080722 | −0.321821471 | 0.189349825 | −0.04002433 | 0.556508702 | 0.7405969 |
| GLUCARDEG-PWY | R - NR | −0.066489934 | 0.113143036 | −0.322754443 | 0.189774575 | −0.196455737 | 0.555373026 | 0.7405969 |
| PWY-7279 | R - NR | −0.067442515 | 0.113579738 | −0.332890742 | 0.198005713 | −0.173831962 | 0.55258133 | 0.738892276 |
| PWY-5656 | R - NR | −0.068684137 | 0.113082433 | −0.324270053 | 0.18690178 | −0.21611513 | 0.542018461 | 0.730364644 |
| SO4ASSIM-PWY | R - NR | −0.07061311 | 0.112396779 | −0.329741754 | 0.188515533 | −0.128748765 | 0.527397129 | 0.718990617 |
| PWY-6608 | R - NR | −0.071100771 | 0.113334723 | −0.330316501 | 0.188114958 | −0.065087251 | 0.529529475 | 0.720478288 |
| LPSSYN-PWY | R - NR | −0.072690633 | 0.112035479 | −0.346967369 | 0.201586103 | −0.197948008 | 0.51415504 | 0.706551315 |
| PWY-5097 | R - NR | −0.073873824 | 0.11342684 | −0.334984451 | 0.187236803 | −0.028706954 | 0.514223593 | 0.706551315 |
| HEXITOLDEGSUPER-PWY | R - NR | −0.073934629 | 0.113410798 | −0.334680223 | 0.186810966 | −0.076722252 | 0.51376554 | 0.706551315 |
| RIBOSYN2-PWY | R - NR | −0.079546055 | 0.112042472 | −0.353839911 | 0.194747802 | −0.091519718 | 0.47531215 | 0.669561817 |
| PWY-7456 | R - NR | −0.080106347 | 0.113005958 | −0.335019286 | 0.174806592 | −0.2385884 | 0.476452194 | 0.6696761 |
| PWY-5138 | R - NR | −0.081196356 | 0.112879498 | −0.335652324 | 0.173259612 | −0.246541078 | 0.469696708 | 0.662989489 |
| UNINTEGRATED | R - NR | −0.084134712 | 0.113466149 | −0.34594777 | 0.177678346 | −0.014943796 | 0.457812535 | 0.654822028 |
| SALVADEHYPOX-PWY | R - NR | −0.084789315 | 0.113427377 | −0.345677253 | 0.176098624 | −0.075751037 | 0.45402982 | 0.650077568 |
| P42-PWY | R - NR | −0.085291533 | 0.113397398 | −0.345512255 | 0.174929188 | −0.066418641 | 0.451135439 | 0.649262962 |
| PWY-7220 | R - NR | −0.087743763 | 0.113080657 | −0.343174366 | 0.16768684 | −0.050432397 | 0.435928331 | 0.633477635 |
| PWY-7222 | R - NR | −0.087743763 | 0.113080657 | −0.343174366 | 0.16768684 | −0.050432397 | 0.435928331 | 0.633477635 |
| PWY-2942 | R - NR | −0.088761576 | 0.113427358 | −0.349557123 | 0.17203397 | −0.035084474 | 0.433146835 | 0.631182653 |
| GLYCOCAT-PWY | R - NR | −0.090405999 | 0.112054839 | −0.36473013 | 0.183918132 | −0.195962579 | 0.4172325 | 0.622948203 |
| GLUCONEO-PWY | R - NR | −0.091173657 | 0.112094871 | −0.362973651 | 0.180626337 | −0.149546101 | 0.41342371 | 0.619913533 |
| PWY0-1586 | R - NR | −0.098590191 | 0.112065201 | −0.372939692 | 0.175759309 | −0.072096792 | 0.376378813 | 0.584123337 |
| 1CMET2-PWY | R - NR | −0.100263643 | 0.113503358 | −0.362557743 | 0.162030457 | −0.044471219 | 0.376496776 | 0.584123337 |
| PANTO-PWY | R - NR | −0.102791148 | 0.113607325 | −0.367938886 | 0.162356591 | −0.040840455 | 0.365436012 | 0.574587458 |
| PWY-5973 | R - NR | −0.103318011 | 0.112990719 | −0.358063071 | 0.151427049 | −0.047855784 | 0.358266446 | 0.567695754 |
| PWY-6901 | R - NR | −0.108682908 | 0.113166513 | −0.364760971 | 0.147395154 | −0.114799304 | 0.335087328 | 0.548821256 |
| HISDEG-PWY | R - NR | −0.109018486 | 0.113231459 | −0.365896252 | 0.147859279 | −0.150017602 | 0.334083536 | 0.548038328 |
| PWY-6168 | R - NR | −0.111627605 | 0.113623942 | −0.376935492 | 0.153680283 | −0.052945013 | 0.325767371 | 0.538190828 |
| PWY66-400 | R - NR | −0.112609216 | 0.113237545 | −0.369510005 | 0.144291573 | −0.102434655 | 0.318436281 | 0.529841535 |
| ANAEROFRUCAT-PWY | R - NR | −0.112704269 | 0.113229199 | −0.369490852 | 0.144082314 | −0.055112529 | 0.317964351 | 0.52968763 |
| PWY66-389 | R - NR | −0.114205178 | 0.113448092 | −0.374782652 | 0.146372295 | −0.393648879 | 0.313253968 | 0.527506079 |
| PWY-1269 | R - NR | −0.114367268 | 0.113613138 | −0.379215146 | 0.150480609 | −0.096723154 | 0.313922488 | 0.527994931 |
| PWY-6385 | R - NR | −0.124054897 | 0.113573579 | −0.387347708 | 0.139237915 | −0.053165813 | 0.274293554 | 0.487167687 |
| PWY-6700 | R - NR | −0.124376392 | 0.113625543 | −0.389156789 | 0.140404006 | −0.054309795 | 0.273484751 | 0.487084858 |
| PWY-7235 | R - NR | −0.130903633 | 0.113180246 | −0.386829972 | 0.125022707 | −0.375182919 | 0.245635687 | 0.458470064 |
| PWY-7663 | R - NR | −0.137002421 | 0.113388865 | −0.395744233 | 0.121739391 | −0.067906283 | 0.225812444 | 0.43360959 |
| P441-PWY | R - NR | −0.145903611 | 0.113437984 | −0.405284099 | 0.113476877 | −0.114984712 | 0.19738284 | 0.405812142 |
| PWY-5989 | R - NR | −0.15036401 | 0.112151517 | −0.424924821 | 0.1241968 | −0.235652879 | 0.177633043 | 0.379748434 |
| PWY-7323 | R - NR | −0.153628748 | 0.113649248 | −0.417644966 | 0.110387469 | −0.190609312 | 0.176156488 | 0.377665017 |
| PWY-7282 | R - NR | −0.156075987 | 0.113129699 | −0.411286691 | 0.099134717 | −0.118814101 | 0.165834124 | 0.370309518 |
| PWY-7371 | R - NR | −0.157791401 | 0.113333867 | −0.415090223 | 0.099507422 | −0.649384311 | 0.162523097 | 0.365349829 |
| PWY-4984 | R - NR | −0.167414344 | 0.113555988 | −0.428412601 | 0.093583914 | −0.211184478 | 0.139746266 | 0.329536802 |
| PWY-6703 | R - NR | −0.173665753 | 0.113736237 | −0.439099728 | 0.091768222 | −0.091457689 | 0.126673577 | 0.311880623 |
| PWY-7039 | R - NR | −0.177036219 | 0.112785022 | −0.433067706 | 0.078995268 | −0.535328966 | 0.114383885 | 0.29625214 |
| ARGININE-SYN4-PWY | R - NR | −0.194302801 | 0.113794925 | −0.460129794 | 0.071524192 | −0.2872599 | 0.087669723 | 0.24875393 |
| CITRULBIO-PWY | R - NR | −0.195422786 | 0.113698075 | −0.45837088 | 0.067525309 | −0.218212663 | 0.08531984 | 0.243571567 |
| COLANSYN-PWY | R - NR | −0.197251726 | 0.113345982 | −0.453734596 | 0.059231145 | −0.210150487 | 0.080695438 | 0.236732345 |
| PWY-5695 | R - NR | −0.199633501 | 0.11385399 | −0.467063538 | 0.067796537 | −0.071249374 | 0.079606453 | 0.23594609 |
| PWY-6305 | R - NR | −0.207362366 | 0.113636562 | −0.46818715 | 0.053462417 | −0.193651595 | 0.067539888 | 0.215264117 |
| PWY-5484 | R - NR | −0.207952101 | 0.113416612 | −0.465002437 | 0.049098234 | −0.163595115 | 0.065803246 | 0.211737411 |
| GLYCOLYSIS | R - NR | −0.207984528 | 0.113401428 | −0.464844097 | 0.04887504 | −0.164923426 | 0.065697226 | 0.211737411 |
| PYRIDOXSYN-PWY | R - NR | −0.213758334 | 0.113872727 | −0.480566295 | 0.053049627 | −0.217247911 | 0.060508692 | 0.198286701 |
| GLYCOLYSIS-E-D | R - NR | −0.225423621 | 0.112340174 | −0.500446288 | 0.049599047 | −0.247202758 | 0.04363129 | 0.158205925 |
| PWY0-845 | R - NR | −0.226152623 | 0.113888488 | −0.492358284 | 0.040053037 | −0.228835838 | 0.047031033 | 0.166773114 |
| PWY-3781 | R - NR | −0.239016928 | 0.113980911 | −0.507211396 | 0.02917754 | −0.462748271 | 0.036069097 | 0.13499319 |
| RHAMCAT-PWY | R - NR | −0.243834984 | 0.113439537 | −0.500185869 | 0.012515901 | −0.155010838 | 0.03093024 | 0.118622567 |

TABLE 2
Nutritional compositions of the low Saa
and high Saa diets (RESEARCH DIETS, INC).

| | Product # | | | |
|---|---|---|---|---|
| | A15121501 | | A15121502 | |
| | High SAA | | Low SAA | |
| | gm % | kcal % | gm % | kcal % |
| Protein | 17 | 18 | 17 | 18 |
| Carbohydrate | 69 | 71 | 69 | 71 |
| Fat | 5 | 12 | 5 | 12 |
| Total | | 100 | | 100 |
| kcal/gm | | 3.9 | | 3.9 |
| Ingredient (gm) | gm | kcal | gm | kcal |
| L-Arginine | 9.2 | 37 | 10.4 | 41.6 |
| L-Histidine-HCl—H2O | 5.5 | 22 | 6.3 | 25.2 |
| L-Isoleucine | 7.4 | 30 | 8.3 | 33.2 |
| L-Leucine | 11 | 44 | 12.5 | 50 |
| L-Lysine-HCl | 12.9 | 52 | 14.6 | 58.4 |
| L-Methionine | 15 | 60 | 3 | 12 |
| L-Phenylalanine | 7.4 | 30 | 8.3 | 33.2 |
| L-Threonine | 7.4 | 30 | 8.3 | 33.2 |
| L-Tryptophan | 1.8 | 7 | 2.1 | 8.4 |
| L-Valine | 7.4 | 30 | 8.3 | 33.2 |
| L-Alanine | 8.9 | 36 | 10.5 | 42 |
| L-Asparagine-H2O | 4.6 | 18 | 5.2 | 20.8 |
| L-Aspartate | 9.2 | 37 | 10.4 | 41.6 |
| L-Cystine | 8 | 32 | 0.3 | 1.2 |
| L-Glutamic Acid | 27.6 | 110 | 31.3 | 125.2 |
| L-Glutamine | 4.6 | 18 | 5.2 | 20.8 |
| Glycine | 9.2 | 37 | 10.4 | 41.6 |
| L-Proline | 4.6 | 18 | 5.2 | 20.8 |
| L-Serine | 4.6 | 18 | 5.2 | 20.8 |
| L-Tyrosine | 3.7 | 15 | 4.2 | 16.8 |
| L-Amino Acids, total | 170 | 680 | 170 | 680 |
| Corn Starch | 550.5 | 2202 | 550.5 | 2202 |
| Maltodextrin 10 | 125 | 500 | 125 | 500 |
| Sucrose | 0 | 0 | 0 | 0 |
| Cellulose | 50 | 0 | 50 | 0 |
| Corn Oil | 50 | 450 | 50 | 450 |
| Mineral Mix S10001 | 35 | 0 | 35 | 0 |
| Sodium Bicarbonate | 7.5 | 0 | 7.5 | 0 |
| Vitamin Mix V10001 | 10 | 40 | 10 | 40 |
| Choline Bitartrate | 2 | 0 | 2 | 0 |
| FD&C Yellow Dye #5 | 0.05 | 0 | 0 | 0 |
| FD&C Red Dye #40 | 0 | 0 | 0.05 | 0 |
| FD&C Blue Dye #1 | 0 | 0 | 0 | 0 |
| Total | 1000.05 | 3872 | 1000.05 | 3872 |

Table 3 of U.S. Provisional Application No. 63/389,382: Metabolites identified in mBHI GC and M. schaedleri CM. Table 3 of U.S. Provisional Application No. 63/399,382 was submitted as a large data table, the contents of which are incorporated herein by reference in their entirety.

TABLE 4
Marker genes for scRNA-Seq clusters. Table 4 shows lists of top 20 marker
genes for cell clusters of: sorted MHCII+CD11c+CD64− MLN cells,
or subset clusters of dendritic cells from the MHCII+CD11c+CD64− MLN cells.

| | | | | | | |
|---|---|---|---|---|---|---|
| gene | cluster | p_val | avg_log2FC | pct. 1 | pct. 2 | p_val_adj |

MHCII+CD11c+CD64− cells

| | | | | | | |
|---|---|---|---|---|---|---|
| Ifitm3 | DCs_Macrophages_monocytes | 0 | 2.121747977 | 0.928 | 0.319 | 0 |
| Il1b | DCs_Macrophages_monocytes | 0 | 2.079870124 | 0.554 | 0.216 | 0 |
| S100a4 | DCs_Macrophages_monocytes | 0 | 1.775680035 | 0.973 | 0.43 | 0 |
| Ifitm2 | DCs_Macrophages_monocytes | 0 | 1.669565705 | 0.721 | 0.277 | 0 |
| Mdh2 | DCs_Macrophages_monocytes | 0 | 1.654112385 | 0.9 | 0.526 | 0 |
| Pigs2 | DCs_Macrophages_monocytes | 0 | 1.599490747 | 0.535 | 0.134 | 0 |
| Gm2a | DCs_Macrophages_monocytes | 0 | 1.566716192 | 0.968 | 0.471 | 0 |
| Ms4a6c | DCs_Macrophages_monocytes | 0 | 1.516409721 | 0.833 | 0.268 | 0 |
| Ppp1r14a | DCs_Macrophages_monocytes | 0 | 1.492282437 | 0.598 | 0.136 | 0 |
| Atf3 | DCs_Macrophages_monocytes | 0 | 1.488954058 | 0.636 | 0.265 | 0 |
| Rgs2 | DCs_Macrophages_monocytes | 0 | 1.432001611 | 0.852 | 0.473 | 0 |
| Ifitm6 | DCs_Macrophages_monocytes | 0 | 1.39469028 | 0.568 | 0.096 | 0 |
| Cfp | DCs_Macrophages_monocytes | 0 | 1.371763601 | 0.67 | 0.207 | 0 |
| Cyp4f16 | DCs_Macrophages_monocytes | 0 | 1.340217806 | 0.634 | 0.152 | 0 |
| Napsa | DCs_Macrophages_monocytes | 0 | 1.326677176 | 0.968 | 0.62 | 0 |
| Cd300a | DCs_Macrophages_monocytes | 0 | 1.31624184 | 0.659 | 0.181 | 0 |
| Rasgef1b | DCs_Macrophages_monocytes | 0 | 1.282495121 | 0.515 | 0.124 | 0 |
| Pmaip1 | DCs_Macrophages_monocytes | 0 | 1.273071734 | 0.809 | 0.359 | 0 |
| S100a6 | DCs_Macrophages_monocytes | 0 | 1.264229483 | 0.943 | 0.556 | 0 |
| Ifitm1 | DCs_Macrophages_monocytes | 2.44E−290 | 1.648182985 | 0.335 | 0.079 | 3.75E−286 |
| Apol7c | Mig DCs 1 | 0 | 3.378703057 | 0.728 | 0.102 | 0 |
| Fabp5 | Mig DCs 1 | 0 | 3.302080699 | 0.9 | 0.312 | 0 |
| Anxa3 | Mig DCs 1 | 0 | 2.921775852 | 0.918 | 0.13 | 0 |
| Ccr7 | Mig DCs 1 | 0 | 2.899975662 | 0.895 | 0.247 | 0 |
| Tmem123 | Mig DCs 1 | 0 | 2.725664586 | 0.895 | 0.391 | 0 |
| Il4i1 | Mig DCs 1 | 0 | 2.495224982 | 0.917 | 0.254 | 0 |
| Cacnb3 | Mig DCs 1 | 0 | 2.427084687 | 0.744 | 0.056 | 0 |
| Birc2 | Mig DCs 1 | 0 | 2.36701866 | 0.903 | 0.34 | 0 |
| Tspan3 | Mig DCs 1 | 0 | 2.328204331 | 0.795 | 0.217 | 0 |
| Fscnl | Mig DCs 1 | 0 | 2.254895358 | 0.863 | 0.129 | 0 |
| Cxcl16 | Mig DCs 1 | 0 | 2.232564779 | 0.767 | 0.168 | 0 |
| Cd63 | Mig DCs 1 | 0 | 2.211638956 | 0.734 | 0.109 | 0 |
| Etv3 | Mig DCs 1 | 0 | 2.200057777 | 0.863 | 0.32 | 0 |
| Socs2 | Mig DCs 1 | 0 | 2.182484265 | 0.637 | 0.043 | 0 |
| Tmcc3 | Mig DCs 1 | 0 | 2.176249507 | 0.708 | 0.057 | 0 |
| Swap70 | Mig DCs 1 | 0 | 2.077127296 | 0.818 | 0.259 | 0 |
| Marcks | Mig DCs 1 | 0 | 2.075550455 | 0.961 | 0.556 | 0 |
| Gadd45b | Mig DCs 1 | 0 | 2.07390139 | 0.927 | 0.453 | 0 |
| Slc4a8 | Mig DCs 1 | 0 | 1.968831191 | 0.586 | 0.058 | 0 |
| Samsn1 | Mig DCs 1 | 0 | 1.951727693 | 0.913 | 0.483 | 0 |
| Xcl1 | NK/NKT cells | 0 | 4.467181739 | 0.948 | 0.05 | 0 |
| Nkg7 | NK/NKT cells | 0 | 3.746363809 | 0.969 | 0.047 | 0 |
| Ncr1 | NK/NKT cells | 0 | 3.25954971 | 0.877 | 0.03 | 0 |
| Ms4a4b | NK/NKT cells | 0 | 3.233950898 | 0.961 | 0.116 | 0 |
| Ctla2a | NK/NKT cells | 0 | 3.188544398 | 0.663 | 0.036 | 0 |
| Klrk1 | NK/NKT cells | 0 | 3.094465143 | 0.961 | 0.187 | 0 |
| Ctsw | NK/NKT cells | 0 | 2.887884086 | 0.885 | 0.063 | 0 |
| Il2rb | NK/NKT cells | 0 | 2.752767478 | 0.837 | 0.045 | 0 |
| Ifng | NK/NKT cells | 0 | 2.735151705 | 0.475 | 0.024 | 0 |
| Klrc1 | NK/NKT cells | 0 | 2.685523118 | 0.658 | 0.024 | 0 |
| Cd160 | NK/NKT cells | 0 | 2.680675378 | 0.757 | 0.033 | 0 |
| Klrel | NK/NKT cells | 0 | 2.604682799 | 0.698 | 0.056 | 0 |
| Ccl5 | NK/NKT cells | 0 | 2.569078355 | 0.982 | 0.618 | 0 |
| Txk | NK/NKT cells | 0 | 2.55101218 | 0.73 | 0.049 | 0 |
| Gimap4 | NK/NKT cells | 0 | 2.452140448 | 0.786 | 0.064 | 0 |
| Trbc1 | NK/NKT cells | 0 | 2.434602802 | 0.807 | 0.048 | 0 |
| Hcst | NK/NKT cells | 0 | 2.427363483 | 0.876 | 0.208 | 0 |
| AW112010 | NK/NKT cells | 0 | 2.426492504 | 0.995 | 0.449 | 0 |
| Gimap6 | NK/NKT cells | 0 | 2.325635916 | 0.838 | 0.106 | 0 |
| Cd7 | NK/NKT cells | 0 | 2.194158212 | 0.917 | 0.454 | 0 |
| Ly6d | pDCs | 0 | 4.902807554 | 0.994 | 0.064 | 0 |
| Siglech | pDCs | 0 | 4.26422793 | 0.988 | 0.021 | 0 |
| Ctsl | pDCs | 0 | 4.091982291 | 0.995 | 0.075 | 0 |
| Bst2 | pDCs | 0 | 4.085827844 | 0.999 | 0.456 | 0 |
| Cox6a2 | pDCs | 0 | 3.838392279 | 0.913 | 0.015 | 0 |
| Ly6c2 | pDCs | 0 | 3.601858764 | 0.941 | 0.057 | 0 |
| Irf8 | pDCs | 0 | 3.303008142 | 0.995 | 0.246 | 0 |
| Ly6a | pDCs | 0 | 3.271406128 | 0.923 | 0.098 | 0 |
| Ccr9 | pDCs | 0 | 3.100094655 | 0.917 | 0.054 | 0 |
| Gm21762 | pDCs | 0 | 3.058841073 | 0.902 | 0.007 | 0 |
| Tcf4 | pDCs | 0 | 2.897318455 | 0.987 | 0.277 | 0 |
| Iglc3 | pDCs | 0 | 2.808413201 | 0.881 | 0.05 | 0 |
| Lgals1 | pDCs | 0 | 2.769032618 | 0.947 | 0.243 | 0 |
| Smim5 | pDCs | 0 | 2.635104929 | 0.874 | 0.04 | 0 |
| Atp1b1 | pDCs | 0 | 2.510717508 | 0.893 | 0.065 | 0 |
| Pltp | pDCs | 0 | 2.448670342 | 0.845 | 0.071 | 0 |
| Klk1 | pDCs | 0 | 2.408720941 | 0.561 | 0.001 | 0 |
| Lair1 | pDCs | 0 | 2.393620774 | 0.922 | 0.137 | 0 |
| Cd8b1 | pDCs | 0 | 2.333547819 | 0.527 | 0.018 | 0 |
| Ctsb | pDCs | 0 | 2.317154548 | 0.984 | 0.468 | 0 |
| Cst3 | cDC1 | 0 | 2.909189861 | 0.994 | 0.881 | 0 |
| Naaa | cDC1 | 0 | 2.607818878 | 0.828 | 0.083 | 0 |
| Ppt1 | cDC1 | 0 | 2.39328544 | 0.869 | 0.2 | 0 |
| Aif1 | cDC1 | 0 | 2.026319957 | 0.879 | 0.188 | 0 |
| Ifi205 | cDC1 | 0 | 1.917342252 | 0.728 | 0.011 | 0 |
| Psap | cDC1 | 0 | 1.719027235 | 0.963 | 0.596 | 0 |
| Gatm | cDC1 | 0 | 1.22319606 | 0.589 | 0.028 | 0 |
| Xcr1 | cDC1 | 0 | 1.177838324 | 0.426 | 0.007 | 0 |
| Rab7b | cDC1 | 0 | 1.171616949 | 0.52 | 0.006 | 0 |
| Irf8 | cDC1 | 7.50979e−322 | 1.644333548 | 0.885 | 0.257 | 1.15403e−317 |
| Plbd1 | cDC1 | 3.30529e−321 | 1.370009933 | 0.964 | 0.532 | 5.07925e−317 |
| Tmsb10 | cDC1 | 0.00E+00 | 1.216139713 | 0.999 | 0.953 | 4.68E−305 |
| BC028528 | cDC1 | 1.85E−294 | 1.302883304 | 0.662 | 0.128 | 2.85E−290 |
| Atpif1 | cDC1 | 6.66E−275 | 1.293830402 | 0.977 | 0.673 | 1.02E−270 |
| Cd24a | cDC1 | 2.49E−246 | 1.221662779 | 0.667 | 0.115 | 3.83E−242 |
| Anxa1 | cDC1 | 1.17E−243 | 1.196122646 | 0.631 | 0.115 | 1.79E−239 |
| Lgals3 | cDC1 | 1.36E−214 | 1.425746004 | 0.834 | 0.344 | 2.10E−210 |
| Vim | cDC1 | 6.33E−180 | 1.250781565 | 0.905 | 0.545 | 9.73E−176 |
| Cxcl9 | cDC1 | 8.55E−136 | 1.468135459 | 0.271 | 0.017 | 1.31E−131 |
| Stmn1 | cDC1 | 2.57E−95 | 1.477847905 | 0.388 | 0.126 | 3.95E−91 |
| Igkc | B-cells | 0 | 5.69720454 | 0.971 | 0.085 | 0 |
| Cd79a | B-cells | 0 | 4.688539705 | 0.988 | 0.006 | 0 |
| Ebf1 | B-cells | 0 | 3.95337434 | 0.932 | 0.006 | 0 |
| Iglc2 | B-cells | 0 | 3.658239736 | 0.867 | 0.021 | 0 |
| Iglc1 | B-cells | 0 | 3.416022381 | 0.541 | 0.008 | 0 |
| Cd79b | B-cells | 0 | 3.294017428 | 0.922 | 0.134 | 0 |
| Ms4a1 | B-cells | 0 | 3.04307811 | 0.852 | 0.045 | 0 |
| Fcmr | B-cells | 0 | 2.819793439 | 0.719 | 0.003 | 0 |
| Gm8369 | B-cells | 0 | 2.466265905 | 0.619 | 0.025 | 0 |
| Ighd | B-cells | 0 | 2.425488496 | 0,562 | 0.011 | 0 |
| Bank1 | B-cells | 0 | 2.375613329 | 0.674 | 0.013 | 0 |
| Gm31243 | B-cells | 0 | 2.097050013 | 0.588 | 0.008 | 0 |
| Vpreb3 | B-cells | 0 | 1.990852138 | 0.467 | 0.001 | 0 |
| Ralgps2 | B-cells | 2.91498 − 321 | 2.069773147 | 0.609 | 0.04 | 4.47946e−317 |
| Ighm | B-cells | 0.00E+00 | 2.908251271 | 0.873 | 0.469 | 1.94E−305 |
| Mzb1 | B-cells | 6.93E−299 | 2.544091621 | 0.666 | 0.051 | 1.07E−294 |
| Iglc3 | B-cells | 1.39E−252 | 2.338612498 | 0.82 | 0.084 | 2.14E−248 |
| Capg | B-cells | 2.84E−187 | 1.695187694 | 0.537 | 0.07 | 4.36E−183 |
| Cd72 | B-cells | 5.73E−137 | 1.80085941 | 0.613 | 0.264 | 8.81E−133 |
| Apoe | B-cells | 1.70E−96 | 2.158589021 | 0.445 | 0.104 | 2.61E−92 |
| Txndc17 | Mig DCs 2 | 0 | 3.845551494 | 0.92 | 0.45 | 0 |
| Epsti1 | Mig DCs 2 | 0 | 3.270726162 | 0.948 | 0.584 | 0 |
| Glipr2 | Mig DCs 2 | 0 | 3.057495156 | 0.753 | 0.104 | 0 |
| Fscn1 | Mig DCs 2 | 0 | 3.056888143 | 0.942 | 0.179 | 0 |
| Crip1 | Mig DCs 2 | 0 | 2.955498341 | 1 | 0.674 | 0 |
| Txn1 | Mig DCs 2 | 0 | 2.921358744 | 0.954 | 0.576 | 0 |
| Cd63 | Mig DCs 2 | 0 | 2.551504175 | 0.891 | 0.148 | 0 |
| Ftl1 | Mig DCs 2 | 0 | 2.520092251 | 1 | 0.974 | 0 |
| Psme2 | Mig DCs 2 | 0 | 2.189900753 | 0.986 | 0.709 | 0 |
| AW112010 | Mig DCs 2 | 9.88131e−324 | 2.824011793 | 0.98 | 0.489 | 1.51846e−319 |
| Zmynd15 | Mig DCs 2 | 1.59761e−319 | 2.821806221 | 0.769 | 0.073 | 2.45504e−315 |
| Serpinb6b | Mig DCs 2 | 3.18349e−316 | 2.514748732 | 0.889 | 0.184 | 4.89207e−312 |
| Aldh1a2 | Mig DCs 2 | 1.02375e−311 | 2.507277868 | 0.616 | 0.049 | 1.57E−307 |
| Apol7c | Mig DCs 2 | 2.11E−297 | 2.837806664 | 0.901 | 0.141 | 3.25E−293 |
| Nudt17 | Mig DCs 2 | 1.04E−294 | 2.363269683 | 0.765 | 0.084 | 1.59E−290 |
| Ccl22 | Mig DCs 2 | 5.68E−284 | 2.732543673 | 0.668 | 0.042 | 8.73E−280 |
| Cst3 | Mig DCs 2 | 1.01E−278 | 2.170529414 | 1 | 0.885 | 1.55E−274 |
| Pla2g16 | Mig DCs 2 | 1.78E−260 | 2.270091601 | 0.767 | 0.243 | 2.73E−256 |
| Bcl2l14 | Mig DCs 2 | 2.14E−260 | 2.302095577 | 0.565 | 0.063 | 3.28E−256 |
| Cd81 | Mig DCs 2 | 3.59E−229 | 2.255038528 | 0.66 | 0.12 | 5.51E−225 |
| Emb | ILC2 | 1.59E−199 | 2.294446917 | 0.94 | 0.338 | 2.44E−195 |
| Il7r | ILC2 | 2.73E−175 | 2.249761263 | 0.898 | 0.246 | 4.19E−171 |
| Odc1 | ILC2 | 2.54E−173 | 2.887566298 | 0.817 | 0.346 | 3.90E−169 |
| Ccdc184 | ILC2 | 1.58E−162 | 1.77822243 | 0.453 | 0.014 | 2.44E−158 |
| Rora | ILC2 | 2.64E−151 | 1.939405549 | 0.586 | 0.073 | 4.05E−147 |
| Upp1 | ILC2 | 3.49E−148 | 1.552981038 | 0.327 | 0.005 | 5.37E−144 |
| Klrb1b | ILC2 | 1.41E−145 | 1.590919454 | 0.702 | 0.065 | 2.17E−141 |
| Il18r1 | ILC2 | 1.46E−145 | 1.657681287 | 0.668 | 0.059 | 2.25E−141 |
| Dgat1 | ILC2 | 3.76E−140 | 1.886893541 | 0.565 | 0.09 | 5.78E−136 |
| Rasl11a | ILC2 | 1.18E−128 | 2.092545163 | 0.319 | 0.013 | 1.81E−124 |
| Gem | ILC2 | 6.65E−122 | 2.086123903 | 0.694 | 0.15 | 1.02E−117 |
| Cxcr6 | ILC2 | 1.20E−120 | 1.671761677 | 0.597 | 0.05 | 1.84E−116 |
| S100a10 | ILC2 | 2.93E−119 | 1.494228281 | 0.948 | 0.39 | 4.51E−115 |
| Ncoa7 | ILC2 | 1.02E−117 | 1.922370936 | 0.584 | 0.168 | 1.57E−113 |
| Pxdc1 | ILC2 | 3.02E−115 | 1.942967185 | 0.39 | 0.049 | 4.64E−111 |
| Vps37b | ILC2 | 4.06E−104 | 1.676435958 | 0.801 | 0.258 | 6.25E−100 |
| Tnfaip3 | ILC2 | 2.62E−100 | 1.427890962 | 0.846 | 0.339 | 4.02E−96 |
| Nrgn | ILC2 | 1.92E−97 | 1.6667647 | 0.537 | 0.072 | 2.95E−93 |
| Rgcc | ILC2 | 7.57E−85 | 1.813205824 | 0.301 | 0.03 | 1.16E−80 |
| Cebpb | ILC2 | 3.70E−84 | 1.55758279 | 0.749 | 0.289 | 5.68E−80 |
| Lyz2 | DCs_Macrophages | 3.70E−288 | 5.304924057 | 0.826 | 0.132 | 5.69E−284 |
| Clec4a3 | DCs_Macrophages | 9.18E−232 | 2.417914504 | 0.748 | 0.081 | 1.41E−227 |
| Lst1 | DCs_Macrophages | 1.54E−226 | 2.223476769 | 0.933 | 0.443 | 2.36E−222 |
| Gngt2 | DCs_Macrophages | 1.72E−221 | 2.381008755 | 0.936 | 0.486 | 2.64E−217 |
| Apoe | DCs_Macrophages | 4.52E−208 | 4.435396916 | 0.728 | 0.103 | 6.94E−204 |
| Ly6i | DCs_Macrophages | 1.38E−204 | 2.333078673 | 0.564 | 0.028 | 2.12E−200 |
| Csf1r | DCs_Macrophages | 1.88E−199 | 2.062058809 | 0.681 | 0.078 | 2.89E−195 |
| Adgre4 | DCs_Macrophages | 6.60E−191 | 1.796252429 | 0.607 | 0.032 | 1.01E−186 |
| Tgfbi | DCs_Macrophages | 1.09E−190 | 2.001768492 | 0.644 | 0.066 | 1.68E−186 |
| Ace | DCs_Macrophages | 2.17E−188 | 1.756458713 | 0.463 | 0.006 | 3.33E−184 |
| Pla2g7 | DCs_Macrophages | 6.20E−180 | 2.126869965 | 0.651 | 0.055 | 9.53E−176 |
| Fcgr4 | DCs_Macrophages | 1.30E−150 | 1.778368299 | 0.393 | 0.008 | 1.99E−146 |
| Msrb1 | DCs_Macrophages | 5.70E−145 | 1.976033631 | 0.658 | 0.175 | 8.76E−141 |
| Cyp4f18 | DCs_Macrophages | 3.62E−130 | 1.649911956 | 0.527 | 0.054 | 5.56E−126 |
| Gsr | DCs_Macrophages | 1.58E−129 | 1.740667051 | 0.705 | 0.185 | 2.43E−125 |
| Cebpb | DCs_Macrophages | 9.24E−118 | 2.30348161 | 0.748 | 0.292 | 1.42E−113 |
| Clec4a1 | DCs_Macrophages | 3.01E−115 | 1.654722376 | 0.621 | 0.133 | 4.62E−111 |
| Plin2 | DCs_Macrophages | 1.58E−112 | 1.782320548 | 0.664 | 0.185 | 2.42E−108 |
| Fabp4 | DCs_Macrophages | 9.36E−110 | 2.34735662 | 0.312 | 0.007 | 1.44E−105 |
| Ctsb | DCs_Macrophages | 8.48E−77 | 1.606543145 | 0.839 | 0.501 | 1.30E−72 |
| Cd3g | T-cells | 0 | 3.900417574 | 0.968 | 0.017 | 0 |
| Cd3d | T-cells | 0 | 3.470139173 | 0.957 | 0.027 | 0 |
| Trac | T-cells | 0 | 2.874510163 | 0.801 | 0.012 | 0 |
| Cd3e | T-cells | 1.02E−307 | 2.331350271 | 0.682 | 0.003 | 1.56E−303 |
| Trbc2 | T-cells | 1.62E−263 | 3.348522905 | 0.942 | 0.096 | 2.49E−259 |
| Lat | T-cells | 6.82E−244 | 2.370724649 | 0.711 | 0.035 | 1.05E−239 |
| Gzmk | T-cells | 1.18E−161 | 1.884726054 | 0.357 | 0.001 | 1.82E−157 |
| Lck | T-cells | 1.83E−119 | 1.778150819 | 0.671 | 0.089 | 2.81E−115 |
| Trbc1 | T-cells | 5.78E−117 | 2.65951297 | 0.643 | 0.121 | 8.87E−113 |
| Ms4a4b | T-cells | 1.79E−112 | 2.08839864 | 0.834 | 0.196 | 2.75E−108 |
| Cd2 | T-cells | 2.16E−112 | 1.861158652 | 0.668 | 0.092 | 3.31E−108 |
| Ptprcap | T-cells | 1.85E−106 | 1.704876292 | 0.892 | 0.336 | 2.84E−102 |
| Ctla4 | T-cells | 7.24E−97 | 2.043527918 | 0.329 | 0.016 | 1.11E−92 |
| Hcst | T-cells | 9.41E−96 | 1.916418956 | 0.776 | 0.271 | 1.45E−91 |
| Gzmb | T-cells | 2.55E−87 | 2.55664367 | 0.336 | 0.018 | 3.92E−83 |
| Cd8a | T-cells | 6.18E−76 | 1.701552888 | 0.495 | 0.094 | 9.50E−72 |
| Cd8b1 | T-cells | 7.40E−76 | 1.847779219 | 0.469 | 0.049 | 1.14E−71 |
| Nkg7 | T-cells | 6.92E−63 | 2.0505357 | 0.567 | 0.14 | 1.06E−58 |
| Ctla2a | T-cells | 1.94E−56 | 1.860788452 | 0.531 | 0.096 | 2.98E−52 |
| Ccl5 | T-cells | 7.89E−24 | 2.528328833 | 0.664 | 0.659 | 1.21E−19 |

Dendritic cells

| | | | | | | |
|---|---|---|---|---|---|---|
| Irf8 | cDC1 | 0 | 3.099873558 | 0.969 | 0.142 | 0 |
| Cst3 | cDC1 | 0 | 2.658072405 | 1 | 0.965 | 0 |
| Naaa | cDC1 | 0 | 2.645264727 | 0.903 | 0.107 | 0 |
| Ppt1 | cDC1 | 0 | 2.492888999 | 0.936 | 0.212 | 0 |
| Psap | cDC1 | 0 | 2.038340043 | 0.994 | 0.619 | 0 |
| Ifi205 | cDC1 | 0 | 2.008321033 | 0.809 | 0.019 | 0 |
| Aif1 | cDC1 | 0 | 1.926446163 | 0.965 | 0.243 | 0 |
| Mpeg1 | cDC1 | 0 | 1.653466398 | 0.83 | 0.13 | 0 |
| Tmsb10 | cDC1 | 0 | 1.553169203 | 0.999 | 0.945 | 0 |
| Rab7b | cDC1 | 0 | 1.280861209 | 0.583 | 0.007 | 0 |
| Clec9a | cDC1 | 0 | 1.275599343 | 0.584 | 0.028 | 0 |
| Gatm | cDC1 | 0 | 1.245328725 | 0.619 | 0.043 | 0 |
| Xcr1 | cDC1 | 1.95E−307 | 1.248629277 | 0.466 | 0.012 | 2.99E−303 |
| BC028528 | cDC1 | 4.81E−277 | 1.3747698 | 0.727 | 0.144 | 7.39E−273 |
| Cd24a | cDC1 | 1.63E−271 | 1.377948873 | 0.727 | 0.119 | 2.50E−267 |
| Anxa1 | cDC1 | 4.34E−230 | 1.267131197 | 0.681 | 0.135 | 6.67E−226 |
| Fuca1 | cDC1 | 9.07E−178 | 1.181773572 | 0.814 | 0.35 | 1.39E−173 |
| Lgals3 | cDC1 | 1.79E−167 | 1.287796182 | 0.886 | 0.445 | 2.75E−163 |
| Cxcl9 | cDC1 | 1.00E−117 | 1.556930186 | 0.303 | 0.022 | 1.54E−113 |
| Lgals1 | cDC1 | 6.30E−115 | 1.156645643 | 0.63 | 0.203 | 9.68E−111 |
| Rgs2 | cDC2, macrophages | 0 | 1.539334766 | 0.889 | 0.518 | 0 |
| | and monocytes | | | | | |
| Ppp1r14a | cDC2, macrophages | 0 | 1.527100437 | 0.707 | 0.203 | 0 |
| | and monocytes | | | | | |
| Ltb | cDC2, macrophages | 0 | 1.467276536 | 0.889 | 0.427 | 0 |
| | and monocytes | | | | | |
| Mdh2 | cDC2, macrophages | 0 | 1.417470286 | 0.913 | 0.663 | 0 |
| | and monocytes | | | | | |
| Nr4a1 | cDC2, macrophages | 0 | 1.353851558 | 0.854 | 0.448 | 0 |
| | and monocytes | | | | | |
| Stk17b | cDC2, macrophages | 0 | 1.335456018 | 0.842 | 0.47 | 0 |
| | and monocytes | | | | | |
| Runx3 | cDC2, macrophages | 0 | 1.297417143 | 0.734 | 0.334 | 0 |
| | and monocytes | | | | | |
| Pglyrp1 | cDC2, macrophages | 0 | 1.272328389 | 0.629 | 0.172 | 0 |
| | and monocytes | | | | | |
| Rasgef1b | cDC2, macrophages | 0 | 1.271439349 | 0.57 | 0.196 | 0 |
| | and monocytes | | | | | |
| Cyp4f16 | cDC2, macrophages | 0 | 1.197171198 | 0.669 | 0.266 | 0 |
| | and monocytes | | | | | |
| Stap1 | cDC2, macrophages | 0 | 1.195834046 | 0.705 | 0.332 | 0 |
| | and monocytes | | | | | |
| Btg2 | cDC2, macrophages | 0 | 1.110605596 | 0.902 | 0.648 | 0 |
| | and monocytes | | | | | |
| Gsn | cDC2, macrophages | 0 | 1.101974159 | 0.77 | 0.419 | 0 |
| | and monocytes | | | | | |
| Ffar2 | cDC2, macrophages | 0 | 1.083410092 | 0.509 | 0.142 | 0 |
| | and monocytes | | | | | |
| Gm6377 | cDC2, macrophages | 0.00E+00 | 1.136495595 | 0.675 | 0.32 | 1.61E−305 |
| | and monocytes | | | | | |
| Fosb | cDC2, macrophages | 5.26E−298 | 1.092962191 | 0.776 | 0.437 | 8.09E−294 |
| | and monocytes | | | | | |
| Ptgs2 | cDC2, macrophages | 8.40E−282 | 1.359445209 | 0.598 | 0.235 | 1.29E−277 |
| | and monocytes | | | | | |
| Nr4a2 | cDC2, macrophages | 5.96E−172 | 1.177165516 | 0.457 | 0.206 | 9.16E−168 |
| | and monocytes | | | | | |
| Ifitm1 | cDC2, macrophages | 5.71E−142 | 1.414553831 | 0.361 | 0.149 | 8.77E−138 |
| | and monocytes | | | | | |
| Ccl3 | cDC2, macrophages | 1.21E−83 | 1.300218079 | 0.346 | 0.191 | 1.86E−79 |
| | and monocytes | | | | | |
| Apol7c | Mig DCs 1 | 0 | 3.085936313 | 0.714 | 0.115 | 0 |
| Fabp5 | Mig DCs 1 | 0 | 2.996084388 | 0.902 | 0.397 | 0 |
| Ccr7 | Mig DCs 1 | 0 | 2.732892843 | 0.883 | 0.273 | 0 |
| Anxa3 | Mig DCs 1 | 0 | 2.72107705 | 0.911 | 0.159 | 0 |
| Tmem123 | Mig DCs 1 | 0 | 2.603415519 | 0.888 | 0.408 | 0 |
| Cacnb3 | Mig DCs 1 | 0 | 2.358208257 | 0.724 | 0.061 | 0 |
| Birc2 | Mig DCs 1 | 0 | 2.35670024 | 0.891 | 0.352 | 0 |
| Il4i1 | Mig DCs 1 | 0 | 2.225059713 | 0.909 | 0.319 | 0 |
| Socs2 | Mig DCs 1 | 0 | 2.220812879 | 0.622 | 0.03 | 0 |
| Tmcc3 | Mig DCs 1 | 0 | 2.163465356 | 0.692 | 0.059 | 0 |
| Tspan3 | Mig DCs 1 | 0 | 2.152330892 | 0.78 | 0.251 | 0 |
| Gadd45b | Mig DCs 1 | 0 | 2.114438073 | 0.915 | 0.442 | 0 |
| Cd63 | Mig DCs 1 | 0 | 2.086668066 | 0.718 | 0.099 | 0 |
| Id2 | Mig DCs 1 | 0 | 2.067156882 | 0.872 | 0.491 | 0 |
| Etv3 | Mig DCs 1 | 0 | 2.012719199 | 0.852 | 0.382 | 0 |
| Cxcl16 | Mig DCs 1 | 0 | 2.006275428 | 0.751 | 0.218 | 0 |
| Fscn1 | Mig DCs 1 | 0 | 2.003678952 | 0.853 | 0.156 | 0 |
| Swap70 | Mig DCs 1 | 0 | 2.0011343 | 0.806 | 0.288 | 0 |
| Mxd1 | Mig DCs 1 | 0 | 1.951624156 | 0.727 | 0.255 | 0 |
| Ankrd33b | Mig DCs 1 | 0 | 1.933841051 | 0.568 | 0.025 | 0 |
| AW112010 | Mig DCs 2 | 0 | 4.017231627 | 0.98 | 0.41 | 0 |
| Txndc17 | Mig DCs 2 | 0 | 3.895765872 | 0.92 | 0.462 | 0 |
| Epsti1 | Mig DCs 2 | 0 | 3.320899472 | 0.944 | 0.56 | 0 |
| Glipr2 | Mig DCs 2 | 0 | 3.160734693 | 0.75 | 0.074 | 0 |
| Txn1 | Mig DCs 2 | 0 | 2.995895475 | 0.954 | 0.551 | 0 |
| Fscn1 | Mig DCs 2 | 0 | 2.825492286 | 0.936 | 0.225 | 0 |
| Crip1 | Mig DCs 2 | 0 | 2.744841548 | 0.998 | 0.647 | 0 |
| Serpinb6b | Mig DCs 2 | 0 | 2.727775702 | 0.885 | 0.136 | 0 |
| Ftl1 | Mig DCs 2 | 0 | 2.336661189 | 1 | 0.985 | 0 |
| Pla2g16 | Mig DCs 2 | 7.75E−307 | 2.535031637 | 0.765 | 0.174 | 1.19E−302 |
| Cd63 | Mig DCs 2 | 1.29E−283 | 2.388178205 | 0.889 | 0.155 | 1.99E−279 |
| Aldh1a2 | Mig DCs 2 | 5.59E−279 | 2.467659147 | 0.616 | 0.06 | 8.59E−275 |
| Zmynd15 | Mig DCs 2 | 4.93E−278 | 2.676613189 | 0.767 | 0.087 | 7.57E−274 |
| Ccl22 | Mig DCs 2 | 3.74E−256 | 2.654266499 | 0.666 | 0.049 | 5.75E−252 |
| Apol7c | Mig DCs 2 | 1.25E−252 | 2.493747328 | 0.903 | 0.168 | 1.92E−248 |
| Nudt17 | Mig DCs 2 | 4.17E−250 | 2.222671578 | 0.761 | 0.104 | 6.41E−246 |
| Tagln2 | Mig DCs 2 | 9.14E−246 | 2.327981614 | 0.887 | 0.593 | 1.40E−241 |
| Cd81 | Mig DCs 2 | 1.46E−231 | 2.315514188 | 0.656 | 0.109 | 2.25E−227 |
| Bcl2l14 | Mig DCs 2 | 3.04E−231 | 2.26056625 | 0.563 | 0.076 | 4.66E−227 |
| Ccl5 | Mig DCs 2 | 4.70E−160 | 2.545747118 | 0.918 | 0.665 | 7.22E−156 |
| Cd209a | CD209+ DCs | 0 | 1.96785916 | 0.731 | 0.12 | 0 |
| Wfdc17 | CD209+ DCs | 0 | 1.926080975 | 0.803 | 0.189 | 0 |
| Ifitm3 | CD209+ DCs | 0 | 1.629016209 | 0.984 | 0.746 | 0 |
| S100a6 | CD209+ DCs | 6.96E−271 | 1.160639828 | 0.991 | 0.779 | 1.07E−266 |
| Cybb | CD209+ DCs | 3.60E−263 | 1.254860784 | 0.916 | 0.473 | 5.52E−259 |
| H2-DMb1 | CD209+ DCs | 5.40E−262 | 1.176612835 | 0.941 | 0.608 | 8.30E−258 |
| Fxyd5 | CD209+ DCs | 1.80E−258 | 1.208059775 | 0.913 | 0.524 | 2.76E−254 |
| Ccr2 | CD209+ DCs | 1.10E−257 | 1.421550761 | 0.588 | 0.093 | 1.68E−253 |
| Cd209d | CD209+ DCs | 3.52E−253 | 1.761382964 | 0.289 | 0.005 | 5.41E−249 |
| Ifitm2 | CD209+ DCs | 4.35E−251 | 1.276715823 | 0.941 | 0.581 | 6.69E−247 |
| Cd300a | CD209+ DCs | 1.38E−237 | 1.07931427 | 0.892 | 0.444 | 2.11E−233 |
| Plac8 | CD209+ DCs | 3.88E−232 | 1.436496716 | 0.942 | 0.64 | 5.97E−228 |
| Clec10a | CD209+ DCs | 7.25E−229 | 1.169548596 | 0.347 | 0.017 | 1.11E−224 |
| Ccl9 | CD209+ DCs | 2.56E−199 | 1.138607648 | 0.431 | 0.05 | 3.93E−195 |
| Lgals3 | CD209+ DCs | 4.86E−199 | 1.160391766 | 0.856 | 0.43 | 7.47E−195 |
| Trappc5 | CD209+ DCs | 1.77E−196 | 1.006136677 | 0.714 | 0.275 | 2.71E−192 |
| Bst2 | CD209+ DCs | 6.41E−191 | 1.55197217 | 0.762 | 0.435 | 9.85E−187 |
| Vim | CD209+ DCs | 1.90E−159 | 1.164076733 | 0.888 | 0.619 | 2.93E−155 |
| Tcf4 | CD209+ DCs | 1.52E−154 | 1.298966571 | 0.572 | 0.243 | 2.34E−150 |
| Klrd1 | CD209+ DCs | 5.42E−150 | 1.101310995 | 0.868 | 0.55 | 8.33E−146 |
| Apoe | LysM monocyte- | 3.76E−235 | 4.846580991 | 0.721 | 0.078 | 5.79E−231 |
| | derived DCs | | | | | |
| Lyz2 | LysM monocyte- | 4.12E−234 | 4.976099244 | 0.795 | 0.167 | 6.33E−230 |
| | derived DCs | | | | | |
| Clec4a3 | LysM monocyte- | 5.68E−194 | 2.308116568 | 0.741 | 0.109 | 8.73E−190 |
| | derived DCs | | | | | |
| Gngt2 | LysM monocyte- | 7.37E−172 | 2.161563686 | 0.923 | 0.552 | 1.13E−167 |
| | derived DCs | | | | | |
| Ly6i | LysM monocyte- | 1.12E−168 | 2.257901748 | 0.542 | 0.037 | 1.72E−164 |
| | derived DCs | | | | | |
| Lst1 | LysM monocyte- | 5.26E−162 | 1.881989233 | 0.916 | 0.581 | 8.08E−158 |
| | derived DCs | | | | | |
| Csf1r | LysM monocyte- | 1.48E−161 | 1.916038988 | 0.673 | 0.105 | 2.28E−157 |
| | derived DCs | | | | | |
| Cebpb | LysM monocyte- | 3.30E−161 | 2.672655849 | 0.758 | 0.236 | 5.07E−157 |
| | derived DCs | | | | | |
| Ace | LysM monocyte- | 9.88E−156 | 1.706715038 | 0.441 | 0.008 | 1.52E−151 |
| | derived DCs | | | | | |
| Adgre4 | LysM monocyte- | 1.05E−152 | 1.69819561 | 0.582 | 0.045 | 1.62E−148 |
| | derived DCs | | | | | |
| Tgfbi | LysM monocyte- | 9.60E−152 | 1.893156487 | 0.616 | 0.089 | 1.47E−147 |
| | derived DCs | | | | | |
| Pla2g7 | LysM monocytc- | 3.16E−140 | 1.972045709 | 0.616 | 0.072 | 4.86E−136 |
| | derived DCs | | | | | |
| Cyp4f18 | LysM monocyte- | 5.43E−136 | 1.66653837 | 0.519 | 0.049 | 8.34E−132 |
| | derived DCs | | | | | |
| Pltp | LysM monocyte- | 3.76E−130 | 1.8895308 | 0.542 | 0.064 | 5.77E−126 |
| | derived DCs | | | | | |
| Fcgr4 | LysM monocyte- | 7.26E−127 | 1.753890858 | 0.374 | 0.011 | 1.12E−122 |
| | derived DCs | | | | | |
| Msrb1 | LysM monocyte- | 4.65E−121 | 1.900764213 | 0.636 | 0.197 | 7.15E−117 |
| | derived DCs | | | | | |
| Gsr | LysM monocyte- | 2.39E−112 | 1.677712727 | 0.694 | 0.196 | 3.67E−108 |
| | derived DCs | | | | | |
| Ctsb | LysM monocyte- | 1.30E−111 | 1.90532387 | 0.838 | 0.514 | 2.00E−107 |
| | derived DCs | | | | | |
| Plin2 | LysM monocyte- | 2.02E−98 | 1.723555113 | 0.646 | 0.2 | 3.10E−94 |
| | derived DCs | | | | | |
| Fabp4 | LysM monocyte- | 5.31E−97 | 2.334623945 | 0.303 | 0.009 | 8.16E−93 |
| | derived DCs | | | | | |
| Pclaf | Pre-DCs | 6.11E−173 | 3.051375136 | 0.852 | 0.028 | 9.40E−169 |
| Stmn1 | Pre-DCs | 5.48E−156 | 3.265647128 | 0.961 | 0.161 | 8.43E−152 |
| Ptma | Pre-DCs | 1.24E−137 | 1.702014346 | 1 | 0.953 | 1.90E−133 |
| Rrm2 | Pre-DCs | 3.18E−130 | 1.969930973 | 0.625 | 0.004 | 4.89E−126 |
| Tuba1b | Pre-DCs | 7.65E−128 | 2.519090859 | 0.938 | 0.349 | 1.18E−123 |
| Dut | Pre-DCs | 6.56E−115 | 2.150914829 | 0.875 | 0.169 | 1.01E−110 |
| Spc24 | Pre-DCs | 6.53E−112 | 1.795928388 | 0.695 | 0.027 | 1.00E−107 |
| Tyms | Pre-DCs | 1.76E−109 | 1.76166811 | 0.734 | 0.047 | 2.71E−105 |
| Top2a | Pre-DCs | 1.84E−104 | 2.191855497 | 0.711 | 0.039 | 2.82E−100 |
| Mcm7 | Pre-DCs | 3.39E−98 | 1.788607662 | 0.781 | 0.086 | 5.21E−94 |
| Ranbp1 | Pre-DCs | 5.92E−93 | 1.735535013 | 0.977 | 0.504 | 9.10E−89 |
| Tubb5 | Pre-DCs | 8.43E−88 | 2.003935494 | 0.969 | 0.672 | 1.30E−83 |
| Hmgb2 | Pre-DCs | 5.50E−85 | 2.149965872 | 1 | 0.76 | 8.45E−81 |
| Ran | Pre-DCs | 1.02E−75 | 1.498443587 | 0.977 | 0.63 | 1.56E−71 |
| Hmgb1 | Pre-DCs | 1.33E−71 | 1.489329422 | 0.977 | 0.704 | 2.05E−67 |
| Smc2 | Pre-DCs | 1.07E−64 | 1.463940677 | 0.664 | 0.068 | 1.65E−60 |
| Mcm3 | Pre-DCs | 1.62E−62 | 1.499524011 | 0.625 | 0.083 | 2.48E−58 |
| Cks1b | Pre-DCs | 2.63E−59 | 1.534421754 | 0.812 | 0.154 | 4.04E−55 |
| Birc5 | Pre-DCs | 3.76E−52 | 1.657693196 | 0.562 | 0.042 | 5.78E−48 |
| H2afx | Pre-DCs | 3.14E−51 | 1.601774228 | 0.594 | 0.122 | 4.82E−47 |

TABLE 5
cDC1 differentially expressed genes. Table 5 contains lists
of differentially expressed genes identified by MAST DE
analysis on cDC1 cells from cAPC mice fed Saa diets.

| | | | | | |
|---|---|---|---|---|---|
| Gene | p_val | avg_logFC | pct. 1 | pct. 2 | p_val_adj |

High; Cdc1

| | | | | | |
|---|---|---|---|---|---|
| S100a6 | 1.65E−07 | 1.2442738 | 0.731 | 0.725 | 2.53E−03 |
| Lgals3 | 9.24E−07 | 1.209401 | 0.875 | 0.901 | 1.42E−02 |
| Rhob | 1.56E−05 | 1.209944 | 0.306 | 0.249 | 2.40E−01 |
| Gpr141 | 3.98E−05 | 1.2114041 | 0.252 | 0.169 | 6.12E−01 |
| Plac8 | 4.14E−05 | 1.2012551 | 0.937 | 0.92 | 6.37E−01 |
| S100a4 | 1.51E−04 | 1.3673024 | 0.422 | 0.358 | 1.00E+00 |
| S100a10 | 1.90E−04 | 1.2546132 | 0.512 | 0.514 | 1.00E+00 |
| Anxa2 | 1.91E−04 | 1.260274 | 0.656 | 0.652 | 1.00E+00 |
| Acadl | 1.92E−04 | 1.2042345 | 0.726 | 0.681 | 1.00E+00 |
| Cxc19 | 3.13E−04 | 1.2077842 | 0.274 | 0.345 | 1.00E+00 |
| Ifitm3 | 3.66E−04 | 1.2395585 | 0.777 | 0.751 | 1.00E+00 |
| Tagln2 | 7.54E−04 | 1.2143097 | 0.81 | 0.808 | 1.00E+00 |
| Ccl4 | 2.98E−02 | 1.3340763 | 0.403 | 0.367 | 1.00E+00 |
| Atf3 | 0.01048253 | 0.24545128 | 0.291 | 0.31 | 1.00E+00 |
| Tmem176b | 0.00030372 | 0.22181101 | 0.219 | 0.188 | 1.00E+00 |

Low; cDC1

| | | | | | |
|---|---|---|---|---|---|
| Srsf5 | 7.63E−19 | 0.6099925 | 0.879 | 0.67 | 1.17E−14 |
| Naaa | 1.82E−08 | 0.6699308 | 0.942 | 0.875 | 2.80E−04 |
| Tlr3 | 5.24E−08 | 0.7454253 | 0.492 | 0.287 | 8.06E−04 |
| Ckb | 6.2443E−07 | 0.20867344 | 0.949 | 0.895 | 9.60E−03 |
| Cadm1 | 2.79E−06 | 0.7258138 | 0.329 | 0.171 | 4.29E−02 |
| Ppt1 | 3.98E−06 | 0.7611276 | 0.971 | 0.912 | 6.11E−02 |
| Trim35 | 4.10E−05 | 0.6924714 | 0.53 | 0.368 | 6.29E−01 |
| Tuba1b | 5.26E−05 | 0.7868961 | 0.556 | 0.394 | 8.09E−01 |
| Kcnq1ot1 | 9.55E−05 | 0.7260299 | 0.409 | 0.263 | 1.00E+00 |
| Gpr65 | 3.41E−04 | 0.7916811 | 0.482 | 0.339 | 1.00E+00 |
| Kctd12 | 2.67E−03 | 0.7636942 | 0.617 | 0.505 | 1.00E+00 |
| Malat1 | 5.40E−03 | 0.7227994 | 0.77 | 0.799 | 1.00E+00 |
| Msmo1 | 5.69E−03 | 0.7641981 | 0.227 | 0.14 | 1.00E+00 |
| mt-Nd2 | 0.00018879 | 0.21870681 | 0.965 | 0.921 | 1.00E+00 |

TABLE 6
List of PCR and RT-PCR primers used.
RT-qPCR Primers

| Primer | | SEQ ID | | |
|---|---|---|---|---|
| Name | Sequence | NO: | Source | Notes |
| M.schaedleri_F | TCTCTTCGGGGATGATTAAAC | 12 | Gomes-Neto, J. C. et | RT-PCR primer |
| M.schaedleri_R | AACTTTTCCTATATAAACATG | 13 | al. Journal of | RT-PCR primer |
| | CAC | | Microbiological | |
| | | | Methods 135, 52-62 | |
| | | | (2017). | |
| MCS487 | 5′-Cy5-GCCGGGGCTGCTTA | 14 | Berry, D et al., | M. schaedleri |
| | TACAGGT-3′ | | ISME, 2012 | specific probe |
| MCS547 | 5′-Cy5-CAGTCACTCCGAAC | 15 | | M. schaedleri |
| | AACGCT-3′ | | | specific probe |
| EUB338 | 5′-Cy3-GCTGCCTCCCGTAG | 16 | Amann et al., AEM | Universal 16S |
| | GAGT-3′ | | 1990 | rRNA probe |
| A.muciniphilla_F | CGGGATAGCCCTGGGAAA | 17 | Brugiroux et al., | RT-PCR primer |
| (YL44) | | | Nature Microbiology | |
| A.muciniphilla_F | GCGCATTGCTGCTTTAATCTT | 18 | 2016 | RT-PCR primer |
| (YL44) | T | | | |

16S rDNA library primers
List of reverse primers

| | | Reverse | | | | | |
|---|---|---|---|---|---|---|---|
| | | complement | | Golay | | | |
| | | of 3′ | | barcode | Reverse | Reverse | |
| | Primer | Illumina | Golay | SEQ ID | primer | primer | Reverse |
| Sample | Name | adapter | barcode | NO: | pad | linker | primer |
| Low_CAPC_1 | 806rcbc0 | CAAGCAGAAG | TTCCC | 20 | AGTCA | CC | GGACTAC |
| | | ACGGCATACG | TTGTC | | GTCAG | | HVGGGTW |
| | | AGA (SEQ | TCC | | (SEQ ID | | TCTAAT |
| | | ID NO: 19) | | | NO: 32) | | (SEQ ID |
| | | | | | | | NO: 33) |
| Low_cAPC_2 | 806rcbc1 | | TACGA | 21 | | | |
| | | | GACTG | | | | |
| | | | ATT | | | | |
| Low_CAPC_3 | 806rcbc2 | | TGCTG | 22 | | | |
| | | | TACGG | | | | |
| | | | ATT | | | | |
| Low_cAPC_4 | 806rcbc3 | | TATCA | 23 | | | |
| | | | CCAGG | | | | |
| | | | TGT | | | | |
| Low_CAPC_5 | 806rcbc4 | | TTGGT | 24 | | | |
| | | | CAACG | | | | |
| | | | ATA | | | | |
| Low_CAPC_6 | 806rcbc5 | | TATCG | 25 | | | |
| | | | CACAG | | | | |
| | | | TAA | | | | |
| High_cAPC_1 | 806rcbc6 | | TGTCG | 26 | | | |
| | | | TGTAG | | | | |
| | | | CCT | | | | |
| High_cAPC_2 | 806rcbc7 | | TAGCG | 27 | | | |
| | | | GAGGT | | | | |
| | | | TAG | | | | |
| High_cAPC_3 | 806rcbc8 | | TATCC | 28 | | | |
| | | | TTTGG | | | | |
| | | | TTC | | | | |
| High_cAPC_4 | 806rcbc9 | | TTACA | 29 | | | |
| | | | GCGCA | | | | |
| | | | TAC | | | | |
| High_cAPC_5 | 806rcbc10 | | TACCG | 30 | | | |
| | | | GTATG | | | | |
| | | | TAC | | | | |
| High_cAPC_6 | 806rcbc11 | | TAATT | 31 | | | |
| | | | GTGTC | | | | |
| | | | GGA | | | | |

Forward Primer

| | | | Forward | |
|---|---|---|---|---|
| Primer | | Forward primer | primer | |
| Name | 5′ Illumina adapter | pad | linker | Forward primer |
| 515F | AATGATACGGCGACCA | TATGGTAATT | GT | GTGCCAGCMGCCGC |
| | CCGAGATCTACAC (SEQ | (SEQ ID NO: 35) | | GGTAA (SEQ ID |
| | ID NO: 34) | | | NO: 36) |

Sequencing Primers

| Primer | | |
|---|---|---|
| Name | Sequence | SEQ ID NO: |
| Read 1 | TATGGTAATTGTGTGCCAGCMGCCGCGGTAA | 37 |
| Read 2 | AGTCAGTCAGCCGGACTACHVGGGTWTCTAAT | 38 |
| Index | ATTAGAWACCCBDGTAGTCCGGCTGACTGACT | 39 |

TABLE 7
List of conjugated primary antibodies used.

| | | | Stock | | |
|---|---|---|---|---|---|
| Target | Conjugate | Clone | (mg/ml) | Application | Company |
| CD3ε | FITC | 145-2C11 | 0.2 | FC 1:100 | BIOLEGEND #100305 |
| CD3ε | Pacific | 145-2C11 | 0.2 | FC 1:100 | BIOLEGEND #100333 |
| | Blue | | | | |
| CD4 | APC/Cy7 | GK1.5 | 0.2 | FC 1:100 | BIOLEGEND #100413 |
| CD8α | Pacific | 53-6.7 | 0.2 | FC 1:100 | BIOLEGEND #100728 |
| | Blue | | | | |
| CD45 | APC | 30-F11 | 0.2 | FC 1:100 | BIOLEGEND #103111 |
| CD45 | Pacific | 30-F11 | 0.2 | FC 1:100 | BIOLEGEND #103125 |
| | Blue | | | | |
| CD45.2 | Pacific | 104 | 0.2 | FC 1:100 | BIOLEGEND #109819 |
| | Blue | | | | |
| CD45.1 | APC | A20 | 0.2 | FC 1:100 | BIOLEGEND #110714 |
| CD103 | PE | 2E7 | 0.2 | FC 1:100 | EBIOSCIENCES #12-1031-81 |
| CD11c | FITC | N418 | 0.2 | FC 1:100 | BIOLEGEND #117305 |
| CD11b | PerCP | M1/70 | 0.2 | FC 1:100 | BIOLEGEND #101229 |
| NK1.1 | PE/cy7 | PK136 | 0.2 | FC 1:100 | EBIOSCIENCES #25-5941-82 |
| I-A/I-E | APC/Cy7 | M5/114.15.2 | 0.2 | FC 1:100 | BIOLEGEND #107627 |
| (MHCII) | | | | | |
| PE-labeled | PE | CD1d PBS-57 | 1.3 | FC 1:500 | NIH TETRAMER CORE |
| tetramer | | | | | FACILITY |
| PE-labeled | PE | | 1.3 | FC 1:500 | NIH TETRAMER CORE |
| tetramer | | | | | FACILITY |
| (unloaded) | | | | | |
| T-bet | PerCP/Cy5 | 4B10 | 0.2 | FC 1:100 | BIOLEGEND #644805 |
| RORγt | APC | B2D | 0.2 | FC 1:100 | EBIOSCIENCES |
| | | | | | #17-6981-80 |
| FOXP3 | PE | FJK-16s | 0.2 | FC 1:100 | EBIOSCIENCES |
| | | | | | #12-5773-80 |
| GATA3 | FITC | L50-823 | 0.2 | FC 1:100 | BD #560077 |
| | (Alexa | | | | |
| | Fluor 488) | | | | |
| LAG-3 | PerCP- | C9B7W | 0.2 | FC 1:100 | EBIOSCIENCES |
| | eFluor710 | | | | #46-2231-80 |
| PD-1 | FITC | 29F.1A12 | 0.2 | FC 1:100 | BIOLEGEND #135213 |
| CTLA-4 | PE | UC10-4B9 | 0.2 | FC 1:100 | BIOLEGEND #106305 |
| TIM-3 | PE/Cy7 | B8.2C12 | 0.2 | FC 1:100 | BIOLEGEND #134010 |
| CD64 | PE | X54-5/7.1 | 0.2 | FC 1:100 | BIOLEGEND #139303 |
| IFNγ | PE/Cy7 | XMG1.2 | 0.2 | FC 1:100 | BIOLEGEND #505826 |
| GZMB | Alexa | GB11 | 0.2 | FC 1:100 | BIOLEGEND #515406 |
| | Fluor 647 | | | | |
| CD80 | APC | 16-10A1 | 0.2 | FC 1:100 | BIOLEGEND #104713 |
| CD86 | APC/Cy7 | GL-1 | 0.2 | FC 1:100 | BIOLEGEND #105029 |

FC (flow cytometry)

TABLE 8
List of R packages used for analysis. Table 8 contains details
on the R environment used in the analysis of data in Example 1. R version
4.0.3 (2020 Oct. 10). Platform: x86_64-apple-darwin17.0 (64-
bit). Running under: MAC OS 11. RSTUDIO 1.4.1103.

| Package | Version |
|---|---|
| BiocManager R/CRAN | 1.30.12 |
| BiocParallel R/Bioconductor | 1.24.1 |
| broom R/CRAN | 0.7.4 |
| data.table R/CRAN | 1.13.6 |
| dplyr R/CRAN | 1.0.3 |
| ggpbur R/CRAN | 0.2.4 |
| ggplot2 R/CRAN | 3.2.1 |
| ggsiginif R/CRAN | 0.6.0 |
| ggthemes R/CRAN | 4.2.0 |
| magrittr R/CRAN | 1.5.0 |
| matrixTests R/CRAN | 0.1.8 |
| pandr R/CRAN | 0.6.3 |
| phyloseq R/CRAN | 1.30.0 |
| PMCMR R/CRAN | 4.3.0 |
| PMCMRplus R/CRAN | 1.4.2 |
| RcolorBrewer R/CRAN | 1.1-2 |
| reshape2 R/CRAN | 1.4.3 |
| scales R/CRAN | 1.1.0 |
| tidyverse R/CRAN | 1.3.0 |
| vegan R/CRAN | 2.5-6 |
| viridis R/CRAN | 0.5.1 |
| yingtools2 R/Github | 0.0.0.95 |
| biomaRt R/Bioconductor | 2.46.2 |
| Seurat R/CRAN | 3.2.2 |
| DESeq2 R/Bioconductor | 1.3.0 |
| fgsea R/Bioconductor | 1.16.0 |
| metacoder R/CRAN | 0.3.4 |
| MAST R/Bioconductor | 1.16.0 |
| UCSCXenaTools R/CRAN | 1.4.0 |
| Maaslin2 R/Bioconductor | 1.4.0 |
| gplots R/CRAN | 3.1.1 |
| cowplot R/CRAN | 1.1.1 |
| emmeans R/CRAN | 1.7.1-1 |
| ImerTest R/CRAN | 3.1-3 |

### Example 2: In Vitro Metabolomics Data on M. schaedleri Conditioned Media

The samples were prepared for analysis as follows. Samples were cultured for 5 days to generate the conditioned medium for metabolomics. 100 ul of each sample was mixed with 300 ul acetonitrile. Samples were centrifuged 10 min at maximum speed. The supernatants were transferred to new microcentrifuge tube and dried under N₂ flow. The samples were resuspended in 50 ul acetonitrile 30% in water and were centrifuged again. 25 ul of each supernatant was transferred to microinserts. The rest of the supernatants were combined to form the pool sample (used for tandem mass spectrometry (MSMS) data acquisition). See e.g., FIG. 15 of U.S. Provisional Application No. 63/389,382, filed Jul. 15, 2022, the contents of which are incorporated herein by reference in their entirety, for the instrument parameters for mass spectrometry. FIG. 15 of U.S. Provisional Application No. 63/389,382 is a schematic showing instrument parameters for the in vitro metabolomics analysis of M. schaedleri conditioned media; see e.g., FIGS. 16-19 and Table 9 of U.S. Provisional Application No. 63/389,382 for results of the analysis.

COMPOUND DISCOVERER (CD) was used to extract the metabolomics data. The data went through the following steps. 1. Peaks were extracted from each file from the MS1 data. This was based on some threshold and criteria. 2. The different retention time were aligned between the files. 3. If a peak was found in one file but not another, the software searched in that file to see if a small peak was present. If a peak was present (but was so below threshold to be detected in the first place), this peak was integrated. If no peak was detected, then the local noise was integrated to add a low value in the data table. Hence there was always be a value for each peak in each file. If no peak was found, the data was tagged as a gap. 4. Various adducts of one likely compound were joined into one compound. 5. Peaks found in the blanks were marked as background. 6. Area was normalized by median centering. 7. Each compound's mass, isotopes, and MSMS data, was analyzed to calculate the most likely formula.

8. The software then attempted to identify (ID) each compound using available MSMS data as follows. A. First the MSMS data, if present was searched against the online database (mzCloud) and local database (mzvault). B. If a good match was found, the compound was assigned this ID. C. If selected, the software then searched a mass list (based on accurate mz and retention time). D. Finally, the software searched the mass and calculated formula through some of CHEMSPIDER databases for candidates fitting the mass.

9. All of the IDs that have been assigned were then manually checked. If necessary, the compounds were tagged (e.g., poor integration, poor match, or ID based on mass list only). Compounds with ID have a tag when the integration did not appear optimal.

For compounds without ID, the following procedure can be followed. (1) Best candidate formula was provided (if possible). Note that other formulae can be possible. (2) COMPOUND DISCOVER can have a candidate from CHEMSPIDER. If MS2 data is present, some additional analyses can be done to try to rank candidates. In all cases for those, ID can be confirmed with more MS2 or a standard. (3) Integration was not checked manually for compounds without ID. For such compounds, CD can check that the peaks were well integrated.

There were several levels of information produced by COMPOUNDDISCOVERER (CD), as detailed below.

1. Mass and retention time of each feature. Mass was reported as “molecular weight” of a monoisotopic compound (i.e., not the ion actually measured). This was done by CD by assuming either a proton adduct or loss of a proton (depending on polarity) and calculating the original mass by adding or removing a proton and electron. In some cases, CD can also detect other adducts. All detected adducts and charge states of the same compound were grouped into one feature. The “Ref” column lists the mass and rt, as a unique identifier for referencing a particular feature (line).

2. Name: IF a name is present, it is a high confidence ID, from Mzcloud match or mzVault (mzVault has precedence). These were manually curated for integration and library match.

3. Tags: Tags were added by manual curation. “Poor match” indicates that the name is to be taken with some caution, because the MSMS match was not entirely convincing. This means the compound is probably related to the name, but might not be it exactly. The “Bad” integration tag indicates that visually the integration seemed non-optimal, so the integration can be checked to confirm the significance.

4. Area for each feature. Note that CD performed Gap filling, and the data in samples where a peak was not originally detected can be reanalyzed. If no small peak was found to integrate, CD integrated the local noise to fill in a value. This means that there was a value for each compound in each file, even if the compound was not detected in some files. If need be, access to which value was a gap fill can be found in CD. When normalization was performed, then normalized and not-normalized data was presented.

5. Predicted formula: CD calculated predicted the formula based on the accurate mass. The best fit is provided in column at the beginning of the table (see e.g., Table 9 of U.S. Provisional Application No. 63/389,382). This is the formula CD considered the best fit, based on mass accuracy, isotopic pattern fit, and fragments masses, if present. All predicted formulae were provided in a column at the end of the table (see e.g., Table 9 of U.S. Provisional Application No. 63/389,382). Note that other formulae are possible (depending on element assumptions, etc.). If a library match was found, the formula from the identified compound was used.

6. Mzvault hits: CD searched the local msms database using mzvault. These are the highest confidence IDs because they also used the retention time information.

7. Mzcloud hits: CD searched mzcloud, an online msms database, using MSMS spectra collected during the msms runs. If a good hit was found, the name of the will appeared in the “name” column (see e.g., Table 9 of U.S. Provisional Application No. 63/389,382). These identifications have 95% confidence.

8. Mzcloud hits were manually curated, and matches were kept in the “name” column.

9. Tags about ID level: Compounds with a name have a tag indicating the level of confidence in the ID.

Level 1 ID were based on MSMS and retention time match with a local database, based on standards run on the instrument. This can be considered a definitive ID. Note that isomers can still be all identified as the same compound.

Level 2 ID are based on MSMS match with mzCloud (an online database). If the compound had this tag assigned, it indicates that the manual curation of the library match was convincing. This is a very strong candidate for this compound and can be used as an almost certain ID.

Masslist hit are based on accurate mass and retention time from a local database. Usually this happens only when the MSMS data acquired was not intense or good enough to yield a mzvault match (which would have been a level 1 ID). These were considered as very strong, to the same level as Level 2 IDs.

Poor library match: this is when there was an mzcloud match but the msms data match was not entirely convincing, either because there are some fragments discrepancies, or too few fragments. The compound is probably related to the name but might not be the named compound exactly.

FISHhit: no msms libraries or masslist match were found, but the MSMS data was FISHscored for a strong candidate from CHEMSPIDER. This indicates that the fragment observed fit well the theoretical fragments one could expect from the proposed candidate structure. This is to be taken a good candidate.

Note that some compounds had have several peaks in HILIC chromatography, so they appeared several times in the list. See e.g., FIG. 15-19 and Table 9 of U.S. Provisional Application No. 63/389,382.

FIG. 16A-16B of U.S. Provisional Application No. 63/389,382 is a series of graphs showing an overview of results from the media metabolomics analysis, showing the overall intensities. BHI1 and BHI2 are un-conditioned media controls, and Ssup1, Ssup2, Ssup3, and Ssup4 are M. schaedleri conditioned media. FIG. 16A of U.S. Provisional Application No. 63/389,382 shows normalized data. This plot represents for each file the spread of areas of all compounds. Very little biomass difference was observed between the samples (see e.g., the not normalized plot in FIG. 16B of U.S. Provisional Application No. 63/389,382), as expected for media. The median centering corrects the minor differences. FIG. 16B of U.S. Provisional Application No. 63/389,382 shows not normalized data.

FIG. 17A-17B of U.S. Provisional Application No. 63/389,382 is a series of PCA plots from the media metabolomics analysis. FIG. 17A of U.S. Provisional Application No. 63/389,382 is a PCA plot showing PC 1 vs PC 2, which together account for over 72% of the variance. Note that most of the variance seems to be related to MSsup1 (over 50% is explained by PCI which separate that samples from the others). FIG. 17B of U.S. Provisional Application No. 63/389,382 is a PCA plot showing PC3 vs PC2 which combined separate the samples by their group. The data indicate overall that the inter-sample variance was much higher than the inter-group variance.

FIG. 18 of U.S. Provisional Application No. 63/389,382 is a volcano plot from the media metabolomics analysis. In line with the PCA results (see e.g., FIG. 17A-17B of U.S. Provisional Application No. 63/389,382), there were relatively few compounds that significantly changed between the two groups (compounds that are in the shaded area have a 2 or higher fold change, and a p-value below 0.05).

FIG. 19 of U.S. Provisional Application No. 63/389,382 is a heatmap showing clustering from the media metabolomics analysis. Similar to the PCA (see e.g., FIG. 17A-17B of U.S. Provisional Application No. 63/389,382), Mssup 1 was quite different from the other samples. mBHI1 and MSsup2 were relatively close together, as well as mBHI2 and MSsup3 clustering together. MSsup4 was in between those samples and MSsup1.

Table 9 of U.S. Provisional Application No. 63/389,382 was submitted as a large data table, the contents of which are incorporated herein by reference in their entirety.

### Example 3: CAPC Cecal Content Metabolomics Analysis

CDX2-Cre Ape^{flox/+} (cAPC) mice were fed low Saa diet (“low”) or high Saa diet (“high”) for 12 weeks before sacrifice and cecal content harvest. Cecal contents were processed for hydrophilic interaction liquid chromatography/positive ion mode MS detection to measure polar metabolites (HILIC-POS analysis); see e.g., schematic in FIG. 20 of U.S. Provisional Application No. 63/389,382. FIG. 20 of U.S. Provisional Application No. 63/389,382 is a schematic showing the experimental setup of the cAPC cecal content metabolomics analysis. CDX2-Cre Apc^{flox/+} (cAPC) mice were fed low Saa diet (“low”) or high Saa diet (“high”) for 12 weeks before sacrifice and cecal content harvest. Cecal contents were processed for hydrophilic interaction liquid chromatography/positive ion mode MS detection to measure polar metabolites (HILIC-POS analysis).

See e.g., FIGS. 21-25 and Table 10 of U.S. Provisional Application No. 63/389,382 for results of the analysis.

Greater than 33,000 LC-MS peaks were recorded. Most peaks were noise or introduced a high level of noise. MetaboAnalystR analysis was performed by computing the standard deviation (SD) per group, for all the metabolites, and then the top 5,000 metabolites with the lowest SD per group were selected. Thus, the most informative signals were kept. Then normalization and statistical analyses were performed. See e.g., FIG. 21-23 of U.S. Provisional Application No. 63/389,382 for the MetaboAnalystR analysis. Similar analysis was performed using the MetaboDiffR package; see g., FIG. 24-25 of U.S. Provisional Application No. 63/389,382.

In the analyses described in this Example, the term “identification” refers to running a sample against a reference standard. e.g., hypotaurine.

In the analyses described in this Example, the term “annotation” refers to prediction using physio-chemical and intensity data, which can require validation follow-up.

Overall, 104 metabolite abundances were significantly changed on Saa diets (e.g., between “high Saa” and “low Saa” diets). Only hypotaurine (derived from cysteine) was identified in the platform as being increased on the high Saa diet, confirming the experimental setup.

Annotation analysis was performed using xMSannotator R package using the Human Metabolome Database (HMDB), Kyoto Encyclopedia of Genes and Genomes (KEGG) database, LIPIDMAPS database, and Toxin and Toxin Target Database (T3DB). Positive mode adducts were +H and +Na.˜1000 predictions were for HMDB; ˜500 for KEGG; 28 for T3DB; and >2000 for LIPIDMAPS. See e.g., FIGS. 20-25 and Table 10 of U.S. Provisional Application No. 63/389,382.

FIG. 21A-21B of U.S. Provisional Application No. 63/389,382 is a series of graphs showing results from the cAPC cecal content metabolomics analysis using MetaboAnalystR. FIG. 21A of U.S. Provisional Application No. 63/389,382 shows metabolite normalization. FIG. 21B of U.S. Provisional Application No. 63/389,382 shows sample normalization.

FIG. 22A-22B of U.S. Provisional Application No. 63/389,382 is a series of graphs showing results from the cAPC cecal content metabolomics analysis using MetaboAnalystR. FIG. 22A of U.S. Provisional Application No. 63/389,382 shows an Orthogonal Projections to Latent Structures Discriminant Analysis (OPLS-DA) plot. OPLS-DA is suitable for diagnosing differences between two groups or systems. It shows which variables have the largest discriminatory power, and it shows how the variables are correlated. OPLS-DA can also quantify how much of the variation in the X block was actually relevant to the analysis question. FIG. 22B of U.S. Provisional Application No. 63/389,382 shows a Principal Component Analysis (PCA) plot.

FIG. 23A-23B of U.S. Provisional Application No. 63/389,382 is a series of graphs showing results from the cAPC cecal content metabolomics analysis using MetaboAnalystR. FIG. 23A of U.S. Provisional Application No. 63/389,382 shows a heatmap of Euclidean distance clustering. Note that one “low” sample clustered with the “high” (as in the PCA plot; see e.g., FIG. 22B of U.S. Provisional Application No. 63/389,382). FIG. 23B of U.S. Provisional Application No. 63/389,382 shows a volcano plot of −log₁₀(FDR adjust p value) vs log 2 fold change.

FIG. 24A-24B of U.S. Provisional Application No. 63/389,382 is a series of graphs showing the MetaboDiff analysis from the cAPC cecal content metabolomics analysis. FIG. 24A of U.S. Provisional Application No. 63/389,382 shows an analysis of all >33,000 features and how many had missing values, per sample. Overall ˜40% of features were found in most samples. FIG. 24B of U.S. Provisional Application No. 63/389,382 shows a volcano plot of the MetaboDiff analysis.

FIG. 25 of U.S. Provisional Application No. 63/389,382 shows a MetaboDiff PCA plot, which is very similar to the MetaboAnalyst plot, with the one “high” invading the “low” space (see e.g., FIG. 22B and FIG. 23A of U.S. Provisional Application No. 63/389,382).

Table 10 of U.S. Provisional Application No. 63/389,382 was submitted as a large data table, the contents of which are incorporated herein by reference in their entirety.

### Example 4

FIG. 26A-26D of U.S. Provisional Application No. 63/389,382 is a series of schematics, images, and graphs showing that high Saa diet-fed mice have higher Mucispirillum schaedleri abundance and thicker mucus (see also FIG. 2 herein). FIG. 26A of U.S. Provisional Application No. 63/389,382 shows enrichment of microbial taxa from 16S rRNA amplicon profiling in the cecal contents of cAPC mice fed low versus high Saa diet. Inset shows taxa q values based on Microbiome Multivariable Associations with Linear Models (MaAsLin 2) regression models correcting for cage effects; see e.g., Mallick et al., PLoS Comput Biol 17, e1009442 (2021), the contents of which are incorporated herein by reference in their entirety. FIG. 26B of U.S. Provisional Application No. 63/389,382 shows 16S rRNA amplicon-based abundance of M. schaedleri in cAPC mice cecal samples. Each symbol represents data from an individual mouse, and fill shade indicates cage affiliation. FIG. 26C of U.S. Provisional Application No. 63/389,382 shows reverse transcription quantitative polymerase chain reaction (RT-qPCR) analysis of M. schaedleri 16S rRNA in cecal contents DNA from cAPC mice. Each symbol represents data from an individual mouse, fill color represents cage affiliation. FIG. 26D of U.S. Provisional Application No. 63/389,382 shows the relative abundance of M. schaedleri-specific reads in 16S ribosomal RNA (rRNA) amplicon sequence data from tissue biopsies of healthy controls (“normal”), colonic adenoma (“adenoma”) or CRC patients (“adenoCA”); see e.g., Sanapareddy et al., ISME J. 2012, 6(10):1858-68; Visnovska et al., Sci Data. 2019, 6(1):116; the contents of each of which are incorporated herein by reference in their entireties.

FIG. 27A-27G of U.S. Provisional Application No. 63/389,382 is a series of schematics, images, and graphs showing that dietary Saa and M. schaedleri promoted CD8⁺ T cells in tumors, and M. schaedleri monocolonization was sufficient for increased cDC1 in the tumor-draining lymph nodes (TDLN) (see also FIG. 3 herein). FIG. 27A of U.S. Provisional Application No. 63/389,382 shows flow cytometry data representing relative frequencies of CD8⁺ T-cells and their immune checkpoint receptor expression in tumors from cAPC mice fed Saa diets. FIG. 27B-27D of U.S. Provisional Application No. 63/389,382 show representative data of cDC1 (CD103⁺CD11b⁺) and CD103⁻CD11b⁺ cells from the TDLN of cAPC mice fed low or high Saa diets; FIG. 27B of U.S. Provisional Application No. 63/389,382 shows representative flow cytometry plots, FIG. 27C of U.S. Provisional Application No. 63/389,382 shows frequencies, and FIG. 27D of U.S. Provisional Application No. 63/389,382 shows numbers of the cDC1 (CD103⁺CD11b⁺) and CD103⁻CD11b⁺ cells. FIG. 27E-27F of U.S. Provisional Application No. 63/389,382 show representative data of cDC1 (CD103⁺CD11b⁻), CD103⁻CD11b⁺ and CD103⁺CD11b⁺ cells from the MLN of GF, M. schaedleri-, or A. muciniphila-monocolonized mice fed high Saa diet; FIG. 27E of U.S. Provisional Application No. 63/389,382 shows frequencies, and FIG. 27F of U.S. Provisional Application No. 63/389,382 shows numbers of the cDC1 (CD103⁻CD11b⁻), CD103⁻CD11b⁺ and CD103⁺CD11b⁺ cells. Each symbol represents data from an individual mouse. FIG. 27G of U.S. Provisional Application No. 63/389,382 shows frequencies of cDC1 and CD103⁻CD11b⁺ cells from the mesentery lymph nodes (MLN) of WT mice fed low Saa diet and gavaged with brain-heart infusion media (mBHI) or M. schaedleri conditioned media (CDM) three times per week. Each symbol represents data from an individual mouse. Each column represents an individual mouse. * P value<0.05, ** P value<0.01, *** P value<0.001. Error bars represent standard error of the mean (SEM). One-Way ANOVA with FDR correction was performed for FIG. 27E and FIG. 27F of U.S. Provisional Application No. 63/389,382; Mann-Whitney test was performed for FIG. 27A, FIG. 27C, FIG. 27D, and FIG. 27G of U.S. Provisional Application No. 63/389,382.

FIG. 28A-28F of U.S. Provisional Application No. 63/389,382 is a series of graphs showing dietary Saa effects on tumor growth in GF cAPC mice, the cecal microbiome, mucus layer thickness and M. schaedleri detection in human stool samples (see also FIG. 5 herein). FIG. 28A of U.S. Provisional Application No. 63/389,382 shows 16S rRNA amplicon abundance of A. muciniphila in cecal samples from cAPC mice fed Saa diets. Each symbol represents data from an individual mouse, fill shade represents cage affiliation. FIG. 28B of U.S. Provisional Application No. 63/389,382 shows phylogenetic tree, reconstructed from the cecal microbiomes of cAPC mice, highlighting in red the M. schaedleri branch, the only taxon significantly altered based on MaAsLin 2 regression models correcting for cage effects. FIG. 28C of U.S. Provisional Application No. 63/389,382 shows representative images of Alcian Blue staining for mucus thickness measurements in proximal colon tissue sections of ASF mice. Lines illustrate the measured mucus thickness. FIG. 28D of U.S. Provisional Application No. 63/389,382 shows quantification of mucus thickness measurements from images as in FIG. 28C of U.S. Provisional Application No. 63/389,382. Each symbol represents data from an individual mouse (average of 7 field of views per mouse). FIG. 28E and FIG. 28F of U.S. Provisional Application No. 63/389,382 show RT-qPCR analysis of M. schaedleri 16S rRNA in cecal contents DNA from ASF mice (FIG. 28E of U.S. Provisional Application No. 63/389,382) or WT specific pathogen-free (SPF) bred in-house mice (FIG. 28F of U.S. Provisional Application No. 63/389,382) fed Saa diets. Error bars represent standard error of the mean (SEM). * P value<0.05, *** P value<0.001. Mann-Whitney test was performed for FIG. 28A, FIG. 28D, FIG. 28E and FIG. 28F of U.S. Provisional Application No. 63/389,382.

FIG. 29A-29P of U.S. Provisional Application No. 63/389,382 is a series of graphs showing the effects of dietary Saa on CD8⁺ T-cells and their expression of immune checkpoint blockade receptors in different tissues from cAPC mice (see also FIG. 6 herein). FIG. 29A-29E of U.S. Provisional Application No. 63/389,382 show flow cytometry analysis of frequencies and numbers of CD8⁺ T-cells from tumors (FIG. 29A of U.S. Provisional Application No. 63/389,382) and PD-1⁺ (FIG. 29B of U.S. Provisional Application No. 63/389,382), LAG-3⁺ (FIG. 29C of U.S. Provisional Application No. 63/389,382), TIM3⁺ (FIG. 29D of U.S. Provisional Application No. 63/389,382), and CTLA-4⁺ (FIG. 29E of U.S. Provisional Application No. 63/389,382) CD8⁺ T-cells from tumors in low or high Saa diet-fed cAPC mice. FIG. 29F-29J of U.S. Provisional Application No. 63/389,382 show flow cytometry analysis of frequencies and numbers of TDLN CD8⁺ T-cells (FIG. 29F of U.S. Provisional Application No. 63/389,382) and PD-1V (FIG. 29G of U.S. Provisional Application No. 63/389,382), LAG-3⁺ (FIG. 29H of U.S. Provisional Application No. 63/389,382), TIM3⁺ (FIG. 29I of U.S. Provisional Application No. 63/389,382), and CTLA-4⁺ (FIG. 29J of U.S. Provisional Application No. 63/389,382) TDLN CD8⁺ T-cells from low or high Saa diet-fed cAPC mice. FIG. 29K-29O of U.S. Provisional Application No. 63/389,382 show flow cytometry analysis of frequencies and numbers of colonic lamina propria (LP) CD8⁺ T-cells (FIG. 29K of U.S. Provisional Application No. 63/389,382) and PD-1⁺ (FIG. 29L of U.S. Provisional Application No. 63/389,382), LAG-3⁺ (FIG. 29M of U.S. Provisional Application No. 63/389,382), TIM3⁺ (FIG. 29N of U.S. Provisional Application No. 63/389,382), and CTLA-4⁺ (FIG. 29O of U.S. Provisional Application No. 63/389,382) colonic LP CD8⁺ T-cells from low or high Saa diet-fed cAPC mice. Each symbol represents data from an individual mouse. FIG. 29P of U.S. Provisional Application No. 63/389,382 shows quantification of CD3⁺CD8⁺/CD3⁺ ratio in images of healthy colonic tissue from cAPC mice. Each symbol represents data from an individual mouse. Error bars represent standard error of the mean (SEM). * P value<0.05, ** P value<0.01. Mann-Whitney test was performed for FIG. 29A-29P of U.S. Provisional Application No. 63/389,382.

FIG. 30A-30E of U.S. Provisional Application No. 63/389,382 is a series of plots showing that flow cytometry gating schemes for FIG. 26, FIG. 27, FIG. 29, FIG. 31, FIG. 32, and FIG. 33 of U.S. Provisional Application No. 63/389,382 (see also FIG. 8 herein). FIG. 30A of U.S. Provisional Application No. 63/389,382 shows the gating scheme of single live cells, serving as the starting cell population for subsequent gating. FIG. 30B of U.S. Provisional Application No. 63/389,382 shows gating of CD8⁺ T-cells and their expression of immune-checkpoint receptors, and their expression of IFNγ and GZMB. FIG. 30C of U.S. Provisional Application No. 63/389,382 shows the gating scheme of CD4⁺ T-cell populations. FIG. 30D of U.S. Provisional Application No. 63/389,382 shows the gating scheme of CD103/CD11b expressing dendritic cells. FIG. 30E of U.S. Provisional Application No. 63/389,382 shows the gating scheme of NK and NKT cells. Polygons indicate the gates.

FIG. 31A-31L of U.S. Provisional Application No. 63/389,382 is a series of graphs showing CD4⁺ T cell profiling in the MLN and colonic LP of WT BIH (M. schaedleri-harboring) mice fed Saa diets (see also FIG. 9 herein). FIG. 31A-31F of U.S. Provisional Application No. 63/389,382 show-frequencies and numbers of total CD4⁺ T-cells (FIG. 31A of U.S. Provisional Application No. 63/389,382) and Th1 cells (FIG. 31B of U.S. Provisional Application No. 63/389,382), Th2 cells (FIG. 31C of U.S. Provisional Application No. 63/389,382), Th17 cells (FIG. 31D of U.S. Provisional Application No. 63/389,382), Foxp3⁺ Tregs (FIG. 31E of U.S. Provisional Application No. 63/389,382), and Foxp3⁺Rorγt⁺ Tregs (FIG. 31F of U.S. Provisional Application No. 63/389,382) in the MLN of WT BIH mice fed Saa diets. FIG. 31G-31L of U.S. Provisional Application No. 63/389,382 show frequencies and numbers of total CD4⁺ T-cells (FIG. 31G of U.S. Provisional Application No. 63/389,382) and Th1 cells (FIG. 31H of U.S. Provisional Application No. 63/389,382), Th2 cells (FIG. 31I of U.S. Provisional Application No. 63/389,382), Th17 cells (FIG. 31J of U.S. Provisional Application No. 63/389,382), Foxp3⁺ Tregs (FIG. 31K of U.S. Provisional Application No. 63/389,382). and Foxp3⁺Rorγt⁺ Tregs (FIG. 31L of U.S. Provisional Application No. 63/389,382) in the colonic LP of WT BIH mice fed Saa diets. Each symbol represents data from an individual mouse. Error bars represent standard error of the mean (SEM). * P value<0.05. Mann-Whitney test was performed for FIG. 31A-31L of U.S. Provisional Application No. 63/389,382.

FIG. 32A-32I of U.S. Provisional Application No. 63/389,382 is a series of graphs showing myeloid cell profiling in cAPC, WT BIH, gnotobiotic, and cDC1-depleted mice fed Saa diets (see also FIG. 10 herein). FIG. 32A-32B of U.S. Provisional Application No. 63/389,382 show-frequencies and numbers of cDC1 (CD103⁺CD11b⁻), CD103⁻CD11b⁺, and CD103⁺CD11b⁺ cells from the LP (FIG. 32A of U.S. Provisional Application No. 63/389,382) or tumors (FIG. 32B of U.S. Provisional Application No. 63/389,382) of cAPC mice fed low or high Saa diet. FIG. 32C-32D of U.S. Provisional Application No. 63/389,382 show the frequencies and numbers of cDC1, CD103⁻CD11b⁺, and CD103⁻CD11b⁺ cells from the MLN (FIG. 32C of U.S. Provisional Application No. 63/389,382) or LP (FIG. 32D of U.S. Provisional Application No. 63/389,382) of WT BIH mice fed low or high Saa dict. FIG. 32E-32F of U.S. Provisional Application No. 63/389,382 show the frequencies and numbers of cDC1, CD103⁻CD11b⁺, and CD103⁺CD11b⁺ cells from the MLN (FIG. 32E of U.S. Provisional Application No. 63/389,382) or LP (FIG. 32F of U.S. Provisional Application No. 63/389,382) of WT GF mice fed low or high Saa diet. FIG. 32G-32I of U.S. Provisional Application No. 63/389,382 show the frequencies and numbers of cDC1, CD103⁻CD11b⁺, and CD103⁺CD11b⁺ cells from the MLN (FIG. 32G of U.S. Provisional Application No. 63/389,382) or LP (FIG. 32H of U.S. Provisional Application No. 63/389,382) of WT ASF mice fed low or high Saa diet. FIG. 32J of U.S. Provisional Application No. 63/389,382 shows the frequencies and numbers of cDC1, CD103⁻CD11b⁺, and CD103⁺CD11b⁻ cells from the LP of GF, M. schaedleri-monocolonized, or A. muciniphila-monocolonized mice fed high Saa diet. In all plots, each dot represents an individual mouse. Error bars represent standard error of the mean (SEM). * P value<0.05, ** P value<0.01, *** P value<0.001. One-Way ANOVA with FDR correction was performed for FIG. 321 of U.S. Provisional Application No. 63/389,382; and Mann-Whitney test was performed for FIG. 32A-32F and FIG. 32G-32H of U.S. Provisional Application No. 63/389,382.

FIG. 33A-33D of U.S. Provisional Application No. 63/389,382 is a series of graphs showing myeloid cell profiling in cAPC, WT bred in-house (BIH), gnotobiotic, and cDC1-depleted mice fed Saa diets (see also FIG. 11 herein). FIG. 33A of U.S. Provisional Application No. 63/389,382 shows the frequencies and numbers of cDC1 and CD103⁻CD11b⁺ cells from the TDLN of Zbtb46-DTR cAPC mice fed high Saa diet and injected with PBS or diphtheria toxin (DT). FIG. 33B of U.S. Provisional Application No. 63/389,382 shows the frequencies and numbers of cDC1 and CD103⁻CD11b⁺ cells from the TDLN of cAPC Batf3^{-/-} mice fed low or high Saa diet. FIG. 33C of U.S. Provisional Application No. 63/389,382 shows the frequencies and numbers of cDC1 and CD103⁻CD11b⁺ cells from the TDLN of cAPC mice fed high Saa diet and injected with α-XCL1 or isotype Abs. FIG. 33D of U.S. Provisional Application No. 63/389,382 shows the frequency and numbers of NKT cells in the TDLN of cAPC mice fed Saa diets. In all plots, each symbol represents an individual mouse. Error bars represent standard error of the mean (SEM). * P value<0.05, ** P value<0.01, *** P value<0.001. Mann-Whitney test was performed for FIG. 33A-33D of U.S. Provisional Application No. 63/389,382.

What is claimed herein is:

1. A composition comprising Mucispirillum schaedleri (M. schaedleri) bacteria formulated for delivery to the intestine.

2. The composition of claim 1, wherein the M. schaedleri bacteria are living or inactivated.

3. The composition of claim 1, wherein the M. schaedleri bacteria are in dried viable form.

4. The composition of claim 1, wherein the M. schaedleri bacteria are encapsulated.

5. The composition of claim 1, wherein the M. schaedleri bacteria are comprised in an enteric capsule.

6. The composition of claim 1, wherein the M. schaedleri bacteria are maintained in an anaerobic state in the formulation.

7. The composition of claim 1, wherein the M. schaedleri bacteria are in admixture with a prebiotic.

8. The composition of claim 1, wherein the M. schaedleri bacteria are in admixture with a sulfur amino acid.

9. The composition of claim 8, wherein the sulfur amino acid is methionine, cysteine or a derivative thereof.

10. The composition of claim 1, wherein the M. schaedleri bacteria are formulated in a food composition.

11. The composition of claim 10, wherein the food composition is supplemented with a sulfur amino acid and/or a prebiotic.

12. The composition of claim 1, further comprising 1 to 20 additional species of bacteria.

13. The composition of claim 1, which comprises no more than 20 species of bacteria.

14. A composition comprising live M. schaedleri bacteria, dead M. schaedleri bacteria, conditioned M. schaedleri culture medium, or an organic solvent extract of conditioned M. schaedleri culture medium or a fraction thereof that promotes XCL1 secretion by NKT cells, wherein the composition is formulated for delivery to the intestine.

15. The composition of claim 14, wherein the M. schaedleri bacteria, medium or solvent extract are in dried form.

16. The composition of claim 14, wherein the M. schaedleri bacteria, medium or extract is/are encapsulated.

17. The composition of claim 14, wherein the M. schaedleri bacteria are comprised in an enteric capsule.

18. The composition of claim 14, wherein the M. schaedleri bacteria are maintained an anaerobic state in the formulation.

19. The composition of claim 14, wherein the M. schaedleri bacteria, medium or extract is/are in admixture with a prebiotic and/or a sulfur amino acid or derivative thereof.

20. The composition of claim 14, wherein the conditioned M. schaedleri culture medium, or the organic solvent extract of conditioned M. schaedleri culture medium or a fraction thereof comprises at least one metabolite selected from FIG. 4H, FIG. 4I, or FIG. 16.

21. The composition of claim 14, wherein the conditioned M. schaedleri culture medium, or the organic solvent extract of conditioned M. schaedleri culture medium or a fraction thereof comprises at least one metabolite selected from the group consisting of succinic acid; propionic acid; nicotinic acid; aconitic acid (cis and/or trans); pentadecanoic acid; itaconic acid; 16-hydroxyhexadecanoic acid; C₈H₁₅NO₃S; crotonic acid; myristic acid; 17-hydroxyheptadecanoic acid (C₁₇H₃₄O₃); and 15-hydroxpentadecanoic acid (C₁₅H₃₀O₃).

22. The composition of claim 14, wherein the conditioned M. schaedleri culture medium, or the organic solvent extract of conditioned M. schaedleri culture medium or a fraction thereof comprises at least one metabolite selected from the group consisting of: succinic acid; nicotinic acid; aconitic acid (cis and/or trans); pentadecanoic acid; itaconic acid; 16-hydroxyhexadecanoic acid; and crotonic acid.

23. A food composition comprising the composition of claim 14.

24. The food composition of claim 23, further comprising 1 to 20 additional species of bacteria.

25. A method of treating cancer or promoting anti-tumor immune activity, the method comprising administering to a subject in need thereof a composition of claim 1.

26. The method of claim 25, wherein the cancer is colon cancer.

27. The method of claim 25, further comprising administering an immune checkpoint inhibitor.

28. A method of promoting responsiveness to immune checkpoint inhibitor tumor therapy, the method comprising administering to a subject in need thereof a composition of claim 1.

29. The method of claim 28, further comprising administering an immune checkpoint inhibitor.

30. The method of claim 28, wherein the subject has colon cancer.

31. The method of claim 28, wherein the subject's cancer has been determined to be resistant to immune checkpoint inhibitor therapy.

32. The method of claim 28, wherein the composition promotes XCL1 secretion by NKT cells.

33. A method of increasing CD103+ conventional dendritic cells (cDC1), the method comprising administering to a subject in need thereof a composition of claim 1.

34. The method of claim 33, wherein the cDC1s are associated with a tumor.

35. The method of claim 34, wherein the tumor is a colon cancer.

36. The method of claim 33, further comprising administering a sulfur amino acid.

37. The method of claim 33, further comprising administering an immune checkpoint inhibitor.

38. A method of increasing XCL1 secretion by NKT cells, the method comprising administering to a subject in need thereof a composition of claim 1.

39. The method of claim 38, wherein the subject has cancer.

40. The method of claim 38, wherein the subject has colon cancer.

41. A method of increasing CD8+ T cell infiltration in a colorectal tumor, the method comprising administering to a subject in need thereof a composition of claim 1.

42. The method of claim 41, wherein the cDC1s are associated with a tumor.

43. The method of claim 42, wherein the tumor is a colon cancer.

44. The method of claim 41, further comprising administering a sulfur amino acid.

45. The method of claim 41, further comprising administering an immune checkpoint inhibitor.

46. A method of increasing CD8+ T cell infiltration in a colorectal tumor, the method comprising administering a diet high in sulfur amino acids (SAA) or a supplement comprising SAAs to a subject in need thereof.

47. The method of claim 46, wherein the diet high in sulfur amino acids comprises greater than 0.04 grams of SAA per kilogram body weight per day.

48. The method of claim 46, further comprising administering a composition of any one of claims 13 to 20 to the subject.

49. A method of establishing or maintaining a tumor-suppressive gut environment in a subject in need thereof, the method comprising administering a diet high in sulfur amino acids (SAA) or a supplement comprising SAAs.

50. The method of claim 49, wherein the diet high in sulfur amino acids or a supplement comprising SAAs comprises greater than 0.04 grams of SAA per kilogram body weight per day.

51. The method of claim 49, further comprising administering a composition of any one of claims 14 to 21 to the subject.

52. A method of treating cancer, the method comprising administering to a subject in need thereof an XCL1 polypeptide.

53. The method of claim 52, wherein the cancer is colon cancer.

54. The method of claim 52, wherein the XCL1 polypeptide is administered to the gut.

55. A method of treating cancer, the method comprising administering to a subject in need thereof a microorganism engineered to express XCL1 polypeptide.

56. A method of treating cancer, the method comprising administering to a subject in need thereof an agonist of the XCL1 receptor, XCR1.

57. The method of claim 56, wherein the XCR1 agonist comprises SEQ ID NOs: 9-11 or an amino acid sequence that is at least 95% identical and maintains its function.

58. A method of treating cancer in a subject in need thereof, the method comprising:
detecting the level of M. schaedleri in a sample from the subject;
administering a cancer immunotherapeutic agent if the level of M. schaedleri is at or above a pre-determined threshold; and
administering a cancer immunotherapeutic agent and a composition of claim 1 if the level of M. schaedleri is below a pre-determined threshold.

59. A method of treating cancer in a subject in need thereof, the method comprising:
obtaining results from an assay detecting the level of M. schaedleri in a sample from the subject;
administering a cancer immunotherapeutic agent if the level of M. schaedleri is at or above a pre-determined threshold; and
administering a cancer immunotherapeutic agent and a composition of claim 1 if the level of M. schaedleri is below a pre-determined threshold.

60. A method of treating cancer in a subject in need thereof, the method comprising:
detecting the level of XCL1 polypeptide, NKTs, and/or CD103+ cDC1s in a sample from the subject;
administering a cancer immunotherapeutic agent if the level of XCL1 polypeptide, NKTs, and/or CD103+ cDC1s is at or above a pre-determined threshold; and
administering a cancer immunotherapeutic agent and a composition of claim 1 if the level of XCL1 polypeptide, NKTs, and/or CD103+ cDC1s is below a pre-determined threshold.

61. A method of treating cancer in a subject in need thereof, the method comprising:
obtaining results from an assay detecting the level of XCL1 polypeptide, NKTs, and/or CD103+ cDC1s in a sample from the subject;
administering a cancer immunotherapeutic agent if the level of XCL1 polypeptide, NKTs, and/or CD103+ cDC1s is at or above a pre-determined threshold; and
administering a cancer immunotherapeutic agent and a composition of claim 1 if the level of XCL1 polypeptide, NKTs, and/or CD103+ cDC1s is below a pre-determined threshold.

62. A method of stratifying a subject for cancer treatment, the method comprising:
detecting the level of M. schaedleri in a sample from the subject; and
classifying the subject as high risk if the level of M. schaedleri is below a pre-determined threshold; or
classifying the subject as low risk if the level of M. schaedleri is at or above a pre-determined threshold.

63. A method of stratifying a subject for cancer treatment, the method comprising:
obtaining results from an assay detecting the level of M. schaedleri in a sample from the subject; and
classifying the subject as high risk if the level of M. schaedleri is below a pre-determined threshold; or
classifying the subject as low risk if the level of M. schaedleri is at or above a pre-determined threshold.

64. A method of stratifying a subject for cancer treatment, the method comprising:
detecting the level of XCL1 polypeptide, NKTs, and/or CD103+ cDC1s in a sample from the subject; and
classifying the subject as high risk if the level of XCL1 polypeptide, NKTs, and/or CD103+ cDC1s is below a pre-determined threshold; or
classifying the subject as low risk if the level of XCL1 polypeptide, NKTs, and/or CD103+ cDC1s is at or above a pre-determined threshold.

65. A method of stratifying a subject for cancer treatment, the method comprising:
obtaining results from an assay detecting the level of XCL1 polypeptide, NKTs, and/or CD103+ cDC1s in a sample from the subject; and
classifying the subject as high risk if the level of XCL1 polypeptide, NKTs, and/or CD103+ cDC1s is below a pre-determined threshold; or
classifying the subject as low risk if the level of XCL1 polypeptide, NKTs, and/or CD103+ cDC1s is at or above a pre-determined threshold.

66. The method of any one of claims 58-65, wherein the subject has colon cancer.

67. The method of any one of claims 62-65, further comprising administering the composition of any one of claims 1-24.

68. The method of any one of claims 58-65, further comprising administering a sulfur amino acid.

69. The method of any one of claims 58-61, wherein the cancer immunotherapeutic agent is selected from the group consisting of: an immune checkpoint inhibitor; chemotherapy; a dendritic cell vaccine; chimeric antigen receptor T cells (CAR-T); and NKT cell-based therapies.

70. The method of any one of claims 58-61, wherein the cancer immunotherapeutic agent comprises an immune checkpoint inhibitor.

71. The method of any one of claims 62-65, further comprising administering an immune checkpoint inhibitor.

72. The method of any one of claims 58-65, further comprising administering a diet high in sulfur amino acids or a supplement comprising SAAs.

73. The method of any one of claims 58-61 or 66-65, wherein the method results in higher treatment efficacy compared to a method of treating without first: detecting the level of M. schaedleri; obtaining results from an assay detecting the level of M. schaedleri; detecting the level of XCL1 polypeptide, NKTs, and/or CD103+ cDC1s; or obtaining results from an assay detecting the level of XCL1 polypeptide, NKTs, and/or CD103+ cDC1s.

74. The method of any one of claims 62-65, wherein the method results in higher treatment efficacy compared to a method of treating without first stratifying the subject.

75. The method of any one of claims 58-61, wherein the method results in lower treatment complications compared to a method of treating without first detecting the level of M. schaedleri, obtaining results from an assay detecting the level of M. schaedleri, detecting the level of XCL1 polypeptide, NKTs, and/or CD103+ cDC1s or obtaining results from an assay detecting the level of XCL1 polypeptide, NKTs, and/or CD103+ cDC1s.

76. The method of any one of claims 62-65, wherein the method results in lower treatment complications compared to a method of treating without first stratifying the subject.

77. An enteric delivery formulation comprising at least one metabolite selected from FIG. 4H, FIG. 4I, or FIG. 16.

78. An enteric delivery formulation comprising at least one metabolite selected from the group consisting of: succinic acid; propionic acid; nicotinic acid; aconitic acid (cis and/or trans); pentadecanoic acid; itaconic acid; 16-hydroxyhexadecanoic acid; C₈H₁₅NO₃S; crotonic acid; myristic acid; 17-hydroxyheptadecanoic acid (C₁₇H₃₄O₃); and 15-hydroxpentadecanoic acid (C₁₅H₃₀O₃).

79. An enteric delivery formulation comprising at least one metabolite selected from the group consisting of: succinic acid; nicotinic acid; aconitic acid (cis and/or trans); pentadecanoic acid; itaconic acid; 16-hydroxyhexadecanoic acid; and crotonic acid.

80. The enteric delivery formulation of any one of claims 77-79, formulated for delivery to the intestine.


SEQUENCE LISTING (49 sequences)

SEQ ID NO: 1 - DNA, 21 bp, synthetic construct
  source 1..21: mol_type = other DNA
gccggggctgcttatacaggt

SEQ ID NO: 2 - DNA, 20 bp, synthetic construct
  source 1..20: mol_type = other DNA
cagtcactccgaacaacgct

SEQ ID NO: 3 - DNA, 18 bp, synthetic construct
  source 1..18: mol_type = other DNA
gctgcctcccgtaggagt

SEQ ID NO: 4 - DNA, 1556 bp, Mucispirillum schaedleri
  source 1..1556: mol_type = unassigned DNA
aaattgtatggagagtttgatcctggctcagaacgaacgctggcggcgtgcttaacacatgcaagtcagggagaaagtctcttcggggatgattaaaccggcgcacgggtgagtaacacgtgagtgacctgccttttagactggaacaacttaccgaaaggtgagctaatgccggatgagttatataagtgcatgtttatataggaaaagttggggagacctgacgctgaaagatggactcgcgtcccattagctagttggtagggtaatggcctaccaaggcgacgatgggtagccggcctgagagggtggccggccacactgggactgagacacggcccagactcctacgggaggcagcagtggggaattttgcgcaatgctcgtaagagtgacgcagcgacgccgcgtgaatgacgaaggccttcgggtcgtaaagttctttcgacagggaagaaaatgcctataagtaactgtgtatgtattgacggtacctgtataagcagccccggctaactccgtgccagcagccgcggtaatacggagggggcgagcgttgttcggagtgactgggcgtaaagagcacgtaggcggtgttgtaagtcattagtcaaagactagagctcaactttagtaaggctagtgatactataatactagagtatcagagaggattgcagaattcctggtgtagcggtgaaatgcgtagatatcaggaggaataccgttagcgaaggcggcaatctggctggaaactgacgctgaggtgcgaaagcgtgggtagcaaacaggattagataccctggtagtccacgctgtaaacgatggatgctaggtgttgggcttttaagttcagtgccgcagcaaacgcgataagcatcccgcctggggagtacgtttgcaagaatgaaactcaaaggaattgacgggggcccgcacaagcggtggagcacgtggtttaattcgatgctaaccgaagaaccttacctgggtttgacatccacagaaggcgttagagataatgctgtgcctgatttatcaggagctgtgagacaggtgctgcatggctgtcgtcagctcgtgccgtgaggtgttgggttaagtcccgcaacgagcgcaacccctatttccagttgctaacggttgaagctgagcactctggagagactgccagcgataagctggaggaaggtggggacgacgtcaagtcatcatggcccttatgtccagggctacacacgtgctacaatggcataatcagagggaagcatctccgcaaggataagcgaatctcataaattatgtctcagttcagattgcagtctgcaactcgactgcatgaagtcggaatcgctagtaatcgcagatcagcaaagctgcggtgaatacgttcccgggccttgtacacaccgcccgtcacaccacgggagtcggtcgcgcctgaagccggtggcctatcagtaatgggggagccgtctatggcgagattggtaactggggtgaagtcgtaacaaggtagccgtaccggaaggtgtggctggatcacctcctttcta

SEQ ID NO: 5 - protein, 114 aa, Homo sapiens
MRLLILALLGICSLTAYIVEGVGSEVSDKRTCVSLTTQRLPVSRIKTYTITEGSLRAVIFITKRGLKVCADPQATWVRDVVRSMDRKSNTRNNMIQTKPTGTQQSTNTAVTLTG

SEQ ID NO: 6 - protein, 114 aa, Mus musculus
MRLLLLTFLGVCCLTPWVVEGVGTEVLEESSCVNLQTQRLPVQKIKTYIIWEGAMRAVIFVTKRGLKICADPEAKWVKAAIKTVDGRASTRKNMAETVPTGAQRSTSTAVTLTG

SEQ ID NO: 7 - protein, 333 aa, Homo sapiens
MESSGNPESTTFFYYDLQSQPCENQAWVFATLATTVLYCLVFLLSLVGNSLVLWVLVKYESLESLTNIFILNLCLSDLVFACLLPVWISPYHWGWVLGDFLCKLLNMIFSISLYSSIFFLTIMTIHRYLSVVSPLSTLRVPTLRCRVLVTMAVWVASILSSILDTIFHKVLSSGCDYSELTWYLTSVYQHNLFFLLSLGIILFCYVEILRTLFRSRSKRRHRTVKLIFAIVVAYFLSWGPYNFTLFLQTLFRTQIIRSCEAKQQLEYALLICRNLAFSHCCFNPVLYVFVGVKFRTHLKHVLRQFWFCRLQAPSPASIPHSPGAFAYEGASFY

SEQ ID NO: 8 - protein, 338 aa, Mus musculus
MDSESDALSIPASRVQMESSTAFYDYHDKLSLLCENNVIFFSTISTIVLYSLVFLLSLVGNSLVLWVLVKYENLESLTNIFILNLCLSDLMFSCLLPVLISAQWSWFLGDFFCKFFNMIFGISLYSSIFFLTIMTIHRYLSVVSPISTLGIHTLRCRVLVTSCVWAASILFSIPDAVFHKVISLNCKYSEHHGFLASVYQHNIFFLLSMGIILFCYVQILRTLFRTRSRQRHRTVRLIFTVVVAYFLSWAPYNLTLFLKTGIIQQSCESLQQLDIAMIICRHLAFSHCCFNPVLYVFVGIKFRRHLKHLFQQVWLCRKTSSTVPCSPGTFTYEGPSFY

SEQ ID NO: 9 - protein, 93 aa, synthetic construct
VGTEVLEESSCVNLQTQRLPCQKIKTYIIWEGAMRAVIFVTKRGLKICADPEAKWVKACIKTVDGRASTRKNMAETVPTGAQRSTSTAVTLTG

SEQ ID NO: 10 - protein, 95 aa, synthetic construct
VGSEVSDKRCCVSLTTQRLPVSRIKTYTITEGACSLRAVIFITKRGLKVCADPQATWVRDVVRSMDRKSNTRNNMIQTKPTGTQQSTNTAVTLTG

SEQ ID NO: 11 - protein, 93 aa, synthetic construct
VGSEVSDKRTCVSLTTQRLPCSRIKTYTITEGSLRAVIFITKRGLKVCADPQATWVRDCVRSMDRKSNTRNNMIQTKPTGTQQSTNTAVTLTG

SEQ ID NO: 12 - DNA, 21 bp, synthetic construct
  source 1..21: mol_type = other DNA
tctcttcggggatgattaaac

SEQ ID NO: 13 - DNA, 24 bp, synthetic construct
  source 1..24: mol_type = other DNA
aacttttcctatataaacatgcac

SEQ ID NO: 14 - DNA, 21 bp, synthetic construct
  source 1..21: mol_type = other DNA
gccggggctgcttatacaggt

SEQ ID NO: 15 - DNA, 20 bp, synthetic construct
  source 1..20: mol_type = other DNA
cagtcactccgaacaacgct

SEQ ID NO: 16 - DNA, 18 bp, synthetic construct
  source 1..18: mol_type = other DNA
gctgcctcccgtaggagt

SEQ ID NO: 17 - DNA, 18 bp, synthetic construct
  source 1..18: mol_type = other DNA
cgggatagccctgggaaa

SEQ ID NO: 18 - DNA, 22 bp, synthetic construct
  source 1..22: mol_type = other DNA
gcgcattgctgctttaatcttt

SEQ ID NO: 19 - DNA, 23 bp, synthetic construct
  source 1..23: mol_type = other DNA
caagcagaagacggcatacgaga

SEQ ID NO: 20 - DNA, 13 bp, synthetic construct
  source 1..13: mol_type = other DNA
ttcccttgtctcc

SEQ ID NO: 21 - DNA, 13 bp, synthetic construct
  source 1..13: mol_type = other DNA
tacgagactgatt

SEQ ID NO: 22 - DNA, 13 bp, synthetic construct
  source 1..13: mol_type = other DNA
tgctgtacggatt

SEQ ID NO: 23 - DNA, 13 bp, synthetic construct
  source 1..13: mol_type = other DNA
tatcaccaggtgt

SEQ ID NO: 24 - DNA, 13 bp, synthetic construct
  source 1..13: mol_type = other DNA
ttggtcaacgata

SEQ ID NO: 25 - DNA, 13 bp, synthetic construct
  source 1..13: mol_type = other DNA
tatcgcacagtaa

SEQ ID NO: 26 - DNA, 13 bp, synthetic construct
  source 1..13: mol_type = other DNA
tgtcgtgtagcct

SEQ ID NO: 27 - DNA, 13 bp, synthetic construct
  source 1..13: mol_type = other DNA
tagcggaggttag

SEQ ID NO: 28 - DNA, 13 bp, synthetic construct
  source 1..13: mol_type = other DNA
tatcctttggttc

SEQ ID NO: 29 - DNA, 13 bp, synthetic construct
  source 1..13: mol_type = other DNA
ttacagcgcatac

SEQ ID NO: 30 - DNA, 13 bp, synthetic construct
  source 1..13: mol_type = other DNA
taccggtatgtac

SEQ ID NO: 31 - DNA, 13 bp, synthetic construct
  source 1..13: mol_type = other DNA
taattgtgtcgga

SEQ ID NO: 32 - DNA, 10 bp, synthetic construct
  source 1..10: mol_type = other DNA
agtcagtcag

SEQ ID NO: 33 - DNA, 20 bp, synthetic construct
  source 1..20: mol_type = other DNA
ggactachvgggtwtctaat

SEQ ID NO: 34 - DNA, 29 bp, synthetic construct
  source 1..29: mol_type = other DNA
aatgatacggcgaccaccgagatctacac

SEQ ID NO: 35 - DNA, 10 bp, synthetic construct
  source 1..10: mol_type = other DNA
tatggtaatt

SEQ ID NO: 36 - DNA, 19 bp, synthetic construct
  source 1..19: mol_type = other DNA
gtgccagcmgccgcggtaa

SEQ ID NO: 37 - DNA, 31 bp, synthetic construct
  source 1..31: mol_type = other DNA
tatggtaattgtgtgccagcmgccgcggtaa

SEQ ID NO: 38 - DNA, 32 bp, synthetic construct
  source 1..32: mol_type = other DNA
agtcagtcagccggactachvgggtwtctaat

SEQ ID NO: 39 - DNA, 32 bp, synthetic construct
  source 1..32: mol_type = other DNA
attagawacccbdgtagtccggctgactgact

SEQ ID NO: 40 - DNA, 1471 bp, Mucispirillum schaedleri
  source 1..1471: mol_type = unassigned DNA
aacgaacgctggcggcgtgcttaacacatgcaagtcagggagaaagtctcttcggggatgattaaaccggcgcacgggtgagtaacacgtgagtgacctgccttttagactggaacaacttaccgaaaggtgagctaatgccggatgagttatataagtgcatgtttatataggaaaagttggggagacctgacgctgaaagatggactcgcgtcccattagctagttggtagggtaatggcctaccaaggcgacgatgggtagccggcctgagagggtggccggccacactgggactgagacacggcccagactcctacgggaggcagcagtggggaattttgcgcaatgctcgtaagagtgacgcagcgacgccgcgtgaatgacgaaggccttcgggtcgtaaagttctttcgacagggaagaaaatgcctataagtaactgtgtatgtattgacggtacctgtataagcagccccggctaactccgtgccagcagccgcggtaatacggagggggcgagcgttgttcggagtgactgggcgtaaagagcacgtaggcggtgttgtaagtcattagtcaaagactagagctcaactttagtaaggctagtgatactataatactagagtatcagagaggattgcagaattcctggtgtagcggtgaaatgcgtagatatcaggaggaataccgttagcgaaggcggcaatctggctggaaactgacgctgaggtgcgaaagcgtgggtagcaaacaggattagataccctggtagtccacgctgtaaacgatggatgctaggtgttgggcttttaagttcagtgccgcagcaaacgcgataagcatcccgcctggggagtacgtttgcaagaatgaaactcaaaggaattgacgggggcccgcacaagcggtggagcacgtggtttaattcgatgctaaccgaagaaccttacctgggtttgacatccacagaaggcgttagagataatgctgtgcctgatttatcaggagctgtgagacaggtgctgcatggctgtcgtcagctcgtgccgtgaggtgttgggttaagtcccgcaacgagcgcaacccctatttccagttgctaacggttgaagctgagcactctggagagactgccagcgataagctggaggaaggtggggacgacgtcaagtcatcatggcccttatgtccagggctacacacgtgctacaatggcataatcagagggaagcatctccgcaaggataagcgaatctcataaattatgtctcagttcagattgcagtctgcaactcgactgcatgaagtcggaatcgctagtaatcgcagatcagcaaagctgcggtgaatacgttcccgggccttgtacacaccgcccgtcacaccacgggagtcggtcgcgcctgaagccggtggcctatcagtaatgggggagccgtctatggcgagattggtaactggggtg

SEQ ID NO: 41 - DNA, 1468 bp, Mucispirillum schaedleri
  source 1..1468: mol_type = unassigned DNA
aacgaacgctggcggcgtgcttaacacatgcaagtcagggagaaagtctntttggggatgattaaaccggcgcacgggtgagtaacacgtgagtgacctgccttttagactggaacaacttaccgaaaggtgagctaatgccggataagttatataagtgcatgtttatataggaaaagttggggagacctgacgctgaaagatggactcgcgtcccattagctagttgggagggtaatggcctaccaaggcgacgatgggtagccggcctgagagggtggccggccacactgggactgagacacggcccagactcctacgggaggcagcagtggggaattttgcgcaatgctcgtaagagtgacgcagcgacgccgcgtgaatgacgaaggccttcgggtcgtaaagttctttcgacagggaagaaaatgcctataagtaactgtgtatgtattgacggtacctgtataagcagccccggctaactccgtgccagcagccgcggtaatacggagggggcgagcgttgttcggagtgactgggcgtaaagagcacgtaggcggtgttgtaagtcattagtcaaagactagagctcaactttagtaaggctagtgatactataatactagagtatcagagaggattgcagaattcctggtgtagcggtgaaatgcgtagatatcaggaggaataccgttagcgaaggcggcaatctggctggaaactgacgctgagtgcgaaagcgtggtagcaacaggattagataccctggtagtccacgctgtaaacgatggatgctaggtgttgggcttttaagttcagtgccgcagcaaacgcgataagcatcccgcctggggagtacgtttgcaagaatgaaactcaaaggaattgacgggggcccgcacaagcggtggagcacgtggtttaattcgatgctaaccgaagaaccttacctgggtttgacatccacagaaggcgttagagataatgctgtgcctgatttatcaggagctgtgagacaggtgctgcatggctgtcgtcagctcgtgccgtgaggtgttgggttaagtcccgcaacgagcgcaacccctatttccagttgctaacggttgaagctgagcactctggagagactgccagcgataagctggaggaaggtggggacgatgtcaagtcatcatggcccttatgtccagggctacacacgtgctacaatggcataatcagagggaagcatctccgcaaggataagcgaatctcataaattatgtctcagttcagattgcagtctgcaactcgactgcatgaagtcggaatcgctagtaatcgcagatcagcaaagctgcggtgaatacgttcccgggccttgtacacaccgcccgtcacaccacgggagtcggtcgcgcctgaagccggtggcctatcagtaatgggggagccgtctatggcgagattggtaactggggtg

SEQ ID NO: 42 - DNA, 1471 bp, Mucispirillum schaedleri; identical to SEQ ID NO: 40
  source 1..1471: mol_type = unassigned DNA

SEQ ID NO: 43 - DNA, 1471 bp, Mucispirillum schaedleri
  source 1..1471: mol_type = unassigned DNA
aacgaacgctggcggcgtgcttaacacatgcaagtcagggagcaaagtctcttcggggatgattaaaccggcgcacgggtgagtaacacgtgagtgacctgccttttagactggaacaacttaccgaaaggtgagctaatgccggatgagttatataagtgcatgtttatataggaaaagttggggagacctgacgctgaaagatggactcgcgtcccattagctagttggtagggtaatggcctaccaaggcgacgatgggtagccggcctgagagggtggccggccacactgggactgagacacgcccagactctacgggaggcagcagtggggaattttgcgcaatgctcgtaagagtgacgccagcgacgccgcgtgaatgacgaaggccttcgggtcgtaaagttctttcgacagggaagaaaatgcctataagtaactgtgtatgtattgacggtacctgtataagcagccccggctaactccgtgccagcagccgcggtaatacggagggggcgagcgttgttcggagtgactgggcgtaaagagcacgtaggcggtgttgtaagtcattagtcaaagactagagctcaactttagtaaggctagtgatactataatactagagtatcagagaggattgcagaattcctggtgtagcggtgaaatgcgtagatatcaggaggaataccgttagcgaaggcggcaatctggctggaaactgacgctgaggtgcgaaagcgtgggtagcaaacaggattagataccctggtagtccacgctgtaaacgatggatgctaggtgttgggcttttaagttcagtgccgcagcaaacgcgataagcatcccgcctggggagtacgtttgcaagaatgaaactcaaaggaattgacgggggcccgcacaagcggtggagcacgtggtttaattcgatgctaaccgaagaaccttacctgggtttgacatccacagaaggcgttagagataatgctgtgcctgatttatcaggagctgtgagacaggtgctgcatggctgtcgtcagctcgtgccgtgaggtgttgggttaagtcccgcaacgagcgcaacccctatttccagttgctaacggttgaagctgagcactctggagagactgccagcgataagctggaggaaggtggggacgacgtcaagtcatcatggcccttatgtccagggctacacacgtgctacaatggcataatcagagggaagcatctccgcaaggataagcgaatctcataaattatgtctcagttcagattgcagtctgcaactcgactgcatgaagtcggaatcgctagtaatcgcagatcagcaaagctgcggtgaatacgttcccgggccttgtacacaccgcccgtcacaccacgggagtcggtcgcgcctgaagccggtggcctatcagtaatgggggagccgtctatggcgagattggtaactggggtg

SEQ ID NO: 44 - DNA, 1477 bp, Mucispirillum schaedleri
  source 1..1477: mol_type = unassigned DNA
agacgnyganctgcgsgtgcttaacacatgcaagtcaggnagnaaagntccatttggagcgagtaaacncghgcacgggtgagtaacatgtgagtaacctnccttttagactggaayaacttaccgaaaggtgagctaatgccggatgmattatrtatctgcatgggtatatagaaaaagctggggnracctgacgctgagagatggactcgcgtcccattagctagttggtggggtagtaggcctaccaaggcgacgatgggtagccggcctgagagggtggccggccacactgggactgagacacggcccagactcctacgggaggcagcagtggggaattttgcgcaatgctcgaaagagtgacgcagcgacgccgcgtgaatgangaaggccthcgggtcgtaaagttctttcgacagggaagaattgtgtatannagtaactggctatatattgacggtacctgtatangcagccccggctaactccgtgccagcagccgcggtaatacggagggggcgagcgttgbtcggagtgactgggcgtaaagagcacgtaggcggtgttgtaagtcattagtcaaagactagagctcaactttagtaaggctagtgatactatagtactagagtatcagagaggattgcagaattcctggtgtagcggtgaaatgcgtagatatcaggaggaataccattagcgaaggcggcgatctggctggaaactgacgctgaggtgcgaaagcgtgggtagcaaacaggattagataccctggtagtccacgctgtaaacgatggatgctaggtgttgggctattaagttcagtgccgcagcaaacgcgataagcatcccncctggggagntacgtttgcaagaatknanactcaaaggaattgacggnggnccscacaagcggtggagcacgtggtttaattcgatgctaaccgaagaaccttacctgggtttgacatccacagaatactatagagatatggtagtgcctggtttaccaggaactgtgagacaggtgctgcatggctgtcgtcagctcgtgccgtgaggtgttgggntaagtcccgcaacgagcgcaacccctatttccagttgctaacgggttaagctgagcactctggagggactgccagcgataagctggaggaaggtggggacgacgtcaagtcatcatggcccttatgtccagggctacacacgtgctacaatggcataatcagagggaagcagctccgagaggataagcgaatctcagaaagtatgtctcagttcagattgcagtctgcaactcgactgcatgaagtcggaatcgctagtaatcgcagatcagcaaagctgcggtgaatacgttccccgggccttgtacacaccgcccgtcacaccacgggagtcggtncgcgcctgangccggtggnctatcagtmatgggnracctcntatggcgagattggtaactgggntg

SEQ ID NO: 45 - DNA, 1451 bp, Mucispirillum schaedleri
  source 1..1451: mol_type = unassigned DNA
aacacatgcaagtcagggarwaaagtttcttcgggacgagntaaaccggcgcacgggtgagtaacacgtgagtaacctgccttttagactggaacaacttaccgaaaggtgagctaatgccggatgaattatgtaactgcatggttatatagaaaaagctggggcgacctggtgctaaaagatgactcgcgtcccattagctagttggtggggtagaagcctaccaaggcgacgatgggtagccggcctgagagggtggccggccacactgggactgagacacggcccagactcctacgggaggcagcagtggggaattttgcgcaatgctcgagagagtgacgcagcgacgccgcgtgaatgacgaaggccttcgggtcgtaaagttctttcgacagggaagaatgtgtatggtagtaactgactatacagtgacggtacctgtataagcagccccggctaactccgtgccagcagccgcggtaatacggagggggcgagcgttgttcggagtgactgggcgtaaagagcacgtaggcggtgttgtaagtcattagtcaaaggctagagctcaactttagtaaggctagtgatactataatactagagtatcagagaggattgcagaattcctggtgtagcggtgaaatgcgtagatatcaggaggaataccggtagcgaaggcggcaatctggctggaaactgacgctgaggtgcgaaagcgtgggtagcaaacaggattagataccctggtagtccacgctgtaaacgatggatgctaggtgttgggctaataagttcagtgccgcagcaaacgcgataagcatcccgcctggggagtacgtttgcaagaatgaaactcaaaggaattgacgggggccgcacaagcggtggagcacgtggtttaattcgatgctaaccgaagaaccttacctgggtttgacatccacagnaatactatagagatatggtagagcctgatttatcaggaactgtgagacaggtgctgcatggctgtcgtcagctcgtgccgtgaggtgttgggttaagtcccgcaacgagcgcaacccctatttccagttgctaacgggtagagctgagcactctggagagactgccagcgataagctggaggaaggtggggacgacgtcaagtcatcatggcccttatgtccagggctacacacgtgctacaatggcataatcagagggaagcaactccgagaggataagcgaatctcataaagtatgtctcagttcagattgcagtctgcaactcgactgcatgaagtcggaatcgctagtaatcgcagatcagcaaagctgcggtgaatacgttcccgggccttgtacacaccgcccgtcacaccacgggagtcggtcgcgcctgaagccggtngscctacccnttasnrgaggganmcgtctatggcnngayykgtaactggggt

SEQ ID NO: 46 - DNA, 1471 bp, Mucispirillum schaedleri
  source 1..1471: mol_type = unassigned DNA
aacgaacgctggcggcgtgcttaacacatgcaagtcagggagaaagtctctttggggatgattaaaccggcgcacgggtgagtaacacgtgagtgacctgccttttagactggaacaacttaccgaaaggtgagctaatgccggataagttatataagtgcatgtttatataggaaaagttggggagacctgacgctgaaagatggactcgcgtcccattagctagttgggagggtaatggcctaccaaggcgacgatgggtagccggcctgagagggtggccggccacactgggactgagacacggcccagactcctacgggaggcagcagtggggaattttgcgcaatgctcgtaagagtgacgcagcgacgccgcgtgaatgacgaaggccttcgggtcgtaaagttctttcgacagggaagaaaatgcctataagtaactgtgtatgtattgacggtacctgtataagcagccccggctaactccgtgccagcagccgcggtaatacggagggggcgagcgttgttcggagtgactgggcgtaaagagcacgtaggcggtgttgtaagtcattagtcaaagactagagctcaactttagtaaggctagtgatactataatactagagtatcagagaggattgcagaattcctggtgtagcggtgaaatgcgtagatatcaggaggaataccgttagcgaaggcggcaatctggctggaaactgacgctgaggtgcgaaagcgtgggtagcaaacaggattagataccctggtagtccacgctgtaaacgatggatgctaggtgttgggcttttaagttcagtgccgcagcaaacgcgataagcatcccgcctggggagtacgtttgcaagaatgaaactcaaaggaattgacgggggcccgcacaagcggtggagcacgtggtttaattcgatgctaaccgaagaaccttacctgggtttgacatccacagaaggcgttagagataatgctgtgcctgatttatcaggagctgtgagacaggtgctgcatggctgtcgtcagctcgtgccgtgaggtgttgggttaagtcccgcaacgagcgcaacccctatttccagttgctaacggttgaagctgagcactctggagagactgccagcgataagctggaggaaggtggggacgatgtcaagtcatcatggcccttatgtccagggctacacacgtgctacaatggcataatcagagggaagcatctccgcaaggataagcgaatctcataaattatgtctcagttcagattgcagtctgcaactcgactgcatgaagtcggaatcgctagtaatcgcagatcagcaaagctgcggtgaatacgttcccgggccttgtacacaccgcccgtcacaccacgggagtcggtcgcgcctgaagccggtggcctatcagtaatgggggagccgtctatggcgagattggtaactggggtg

SEQ ID NO: 47 - DNA, 1471 bp, Mucispirillum schaedleri; identical to SEQ ID NO: 40
  source 1..1471: mol_type = unassigned DNA

SEQ ID NO: 48 - DNA, 1471 bp, Mucispirillum schaedleri; identical to SEQ ID NO: 40
  source 1..1471: mol_type = unassigned DNA

SEQ ID NO: 49 - DNA, 1471 bp, Mucispirillum schaedleri; identical to SEQ ID NO: 46
  source 1..1471: mol_type = unassigned DNA